(19) United States
(12) Patent Application Publication
(10) Pub. No.: US 2025/0051751 A1
(43) Pub. Date: Feb. 13, 2025

(54) GLUCOSE ISOMERASES

(71) Applicant: C-LEcta GmbH, Leipzig (DE)

(72) Inventors: Sebastian BARTSCH, Leipzig (DE); Andreas VOGEL, Leipzig (DE)

(73) Assignee: C-LEcta GmbH, Leipzig (DE)

(21) Appl. No.: 18/718,823

(22) PCT Filed: Dec. 13, 2022
(86) PCT No.: PCT/EP2022/085534

§ 371 Date: Jun. 12, 2024

(30) Foreign Application Priority Data
Dec. 14, 2021 (EP) 21214446.3

(51) Int. Cl. C12N 9/92; C12P 19/24

(52) U.S. Cl. CPC C12N 9/92; C12P 19/24; C12Y 503/01005

(57) ABSTRACT

The invention relates to glucose isomerase enzymes (glucose isomerases) with improved enzymatic activity at low pH values and tolerance of calcium ions in the enzymatic reactions.

Priority is claimed of European patent application no. 21 214 446.3 which was filed on Dec. 14, 2021.

The invention relates to glucose isomerase enzymes (glucose isomerases) with improved enzymatic activity at low pH values and tolerance of calcium ions in the enzymatic reactions.

Glucose isomerases, also called xylose isomerases, classify as EC 5.3.1.5 according to the Enzyme Class number of the International Union of Biochemistry and Molecular Biology. They catalyze the formation of an aldose molecule from a ketose molecule, and the formation of a ketose molecule from an aldose molecule within a reaction equilibrium between both directions.

Glucose isomerases currently available on the market have been developed in the first place to be used for the preparation of high fructose corn syrup and are usually improved by methods of enzyme engineering and other methods aiming at efficiently converting glucose to fructose at high glucose concentration, elevated temperatures, high pH values, and the like.

The usability of such enzymes in industrial processes involving conversion of glucose into fructose is there-fore limited to the specific reaction conditions they were developed for, i.e. available glucose isomerases available in the state of the art usually cannot be efficiently used for enzymatic formation of fructose in liquid matrices presenting low glucose concentration and/or at lower temperature and/or at lower pH values.

The structure of a functional glucose isomerase comprises four identical copies of one polypeptide sequence, forming a homo-tetrameric enzyme. The four monomers of the glucose isomerase are distinguished from each other by specifying terminology, for example by the terms “chain A”, “chain B”, “chain C”, or “chain D”). Each monomer of a glucose isomerase is characterized by binding sites for two identical divalent catalytic cations. Kinetic studies in the prior art show that magnesium cations (Mg²⁺), cobalt cations (Co²⁺), manganese cations (Mn²⁺), and iron (II) cations (Fe²⁺) activate enzymatic catalysis and are suitable as catalytic cations of glucose isomerases. Other divalent cations, like nickel ions (Ni²⁺), calcium cations (Ca²⁺), zinc ions (Zn²⁺), copper ions (Cu²⁺), and mercury ions (Hg²⁺) can also coordinate with the monomers of the glucose isomerases, however, they are inhibiting catalytic activity of the glucose isomerases. Accordingly, glucose isomerases are sensitive to the presence of calcium ions (Ca²⁺ ions) in liquid matrices that would compete with catalytic cations, thereby limiting the industrial applicability the glucose isomerases in such matrices, for example for the treatment of milk dairy products and other products having a high natural abundance of calcium ions.

Likewise, glucose isomerases of the prior art exhibit only low or no enzymatic activities at acidic pH values. In particular, liquid matrices obtained from raw materials with slightly acidic values between pH 5 and pH 7, often cannot be processed efficiently by known glucose isomerases. Accordingly, glucose isomerases are sensitive to the pH values in liquid matrices, thereby limiting the industrial applicability the glucose isomerases, for example for the treatment of milk dairy products and other products having a natural pH value below 7.0.

S. H. Bhosale et al., Microbiological Reviews, 60(2), 1996, 280-300 is a review about glucose isomerases. According to the document, these enzymes have an optimal activity at temperatures ranging between 60° C. and 80° C. and at pH between 7.0 and 9.0. However, the stability of the enzymes at high temperatures declines as a result of inactivation, and lowering pH results in a rapid decrease of activity in respect to the isomerization reactions. The review further explains that calcium ions are inhibitory to glucose isomerases. Although the review describes a substitution in the glucose isomerase of A. missouriensis that shifts the pH optimum to 6.25, the review is silent about the actual enzymatic activity of the variant at that pH value. The reference furthermore provides hints for a lower magnesium ion binding affinity of the variant which may have implications on enzyme activity. The review is also silent on variants showing improved enzymatic activity in a medium containing high calcium content and/or at low temperatures.

Patent publication US 2011/318801 A1 discloses Zymomonas cells recombinant for xylose isomerase, represented by SEQ ID NO 205. The Zymomonas cells are used for converting xylose into ethanol in a fermentation process, in which the xylose isomerase is intracellularly expressed by the Zymomonas cells and helps metabolizing xylose by alcoholic fermentation. Although the Zymomonas cells are grown at 30-37° C. and in a medium with pH 4.5 to 7.5 during fermentation, the publication gives no hint neither about the actual intracellular pH values during fermentation at which the xylose isomerase is active, nor about an isolated glucose isomerase enzyme that would provide a catalytic activity at the low pH values described for the fermentation medium. Furthermore, the document does not demonstrate that the isolated enzymes obtained therein show activity when converting glucose into fructose and vice-versa in media containing high calcium concentrations or at low temperatures.

Database Uniprot accession nos. A0A415C1R8, A0A1X3ABY7, A0A4P6YUS9, and A0A366K6E9 relate to full-xylose isomerases comprising, respectively, I163V, P18L, L198I, and N197S substitutions in respect to SEQ ID NO:1. No functional data for the enzymes are given, let alone an improvement in enzymatic activity when converting glucose into fructose or vice versa have been demonstrated, nor in media containing high concentrations of calcium, low temperatures and/or acidic pH.

As the available glucose isomerases from the prior art are not satisfactory to meet the reaction requirements of industrial applications of relevance, there is a need for glucose isomerases which are advantageous over the known glucose isomerases. In particular, there is a demand for glucose isomerases with an increased tolerance of calcium cations during enzymatic activity, and a high enzymatic activity in the presence of calcium cations and/or at acidic pH values.

Accordingly, a problem underlying the invention is the provision of a glucose isomerase which is suitable for use in industrial conversion of an aldose molecule to a ketose molecule, or of a ketose molecule to an aldose molecule, which has advantages compared to the prior art. The glucose isomerase should preferably exhibit an enzymatic activity at neutral and at acidic pH values, preferably within the range of pH 5.0 to pH 7.0, and/or provide an improved calcium tolerance, resulting in an improved activity in the absence and/or presence of calcium cations, preferably within the range from 0 mM Ca²⁺ to 60 mM Ca²⁺.

This problem has been solved by the subject-matter of the patent claims.

In summary, the invention is directed to the provision of improved engineered glucose isomerase variants characterized by a broader industrial applicability spectrum. In particular, the invention relates to variants of the glucose isomerase of the organism Bifidobacterium adolescentis of SEQ ID NO:1 with substitutions of amino acids at sequence positions within defined subsequences of the amino acid sequence of the wild-type enzyme.

It has been surprisingly found that improved engineered glucose isomerase variants can be provided that are characterized by a high activity at low pH values within the range of at least pH 5.0 to pH 7.0. Further, it has been surprisingly found that improved engineered glucose isomerase variants can be provided that are characterized by a high activity and at calcium concentrations within the range of 0 mM up to 60 mM.

A first aspect of the invention relates to a glucose isomerase comprising an amino acid sequence which has at least 65% identity to SEQ ID NO: 1 and which compared to SEQ ID NO: 1 comprises at least one, two, three, four, five, six, seven, eight, nine, ten, eleven, twelve, thirteen, fourteen, fifteen, sixteen or seventeen substitutions at substitution positions, which are independently of one another located in
subsequence R1 ranging from position L266 to R346 of SEQ ID NO:1; and/or
subsequence R2 ranging from position L231 to Q243 of SEQ ID NO:1; and/or
subsequence R3 ranging from position N185 to W199 of SEQ ID NO: 1; and/or
subsequence R4 ranging from position L139 to Y164 of SEQ ID NO: 1; and/or
subsequence R5 ranging from position G17 to R67 of SEQ ID NO:1.

Preferably, the glucose isomerase according to the invention comprises an amino acid sequence which has at least 65% identity to SEQ ID NO: 1 and which compared to SEQ ID NO: 1 comprises at least one, two, three, four, five, six, seven, eight, nine, ten, eleven, twelve, thirteen, fourteen, fifteen, sixteen or seventeen substitutions at substitution positions independently of one another selected from the groups consisting of:
A273, Q300, M309, P313, Q331, A342, and K343 in subsequence R1; and/or
M241 and F242 in subsequence R2; and/or
V187, G190, and L198 in subsequence R3; and/or
W140, T147, and I163 in subsequence R4; and/or
P18 and A47 in subsequence R5.

Preferably, the glucose isomerase according to the invention comprises an amino acid sequence which has at least 65% identity to SEQ ID NO: 1 and which compared to SEQ ID NO: 1 comprises at least one, two, three, four, five, six, seven, eight, nine, ten or eleven substitutions at substitution positions independently of one another selected from the groups consisting of:
A273, Q300, P313, Q331, and K343 in subsequence R1; and/or
M241 and F242 in subsequence R2; and/or
V187, and G190 in subsequence R3; and/or
W140 in subsequence R4; and/or
A47 in subsequence R5.

Preferably, the glucose isomerase according to the invention comprises an amino acid sequence which has at least 65% identity to SEQ ID NO: 1 and which compared to SEQ ID NO: 1 comprises at least one substitution at substitution positions independently of one another selected from the groups consisting of:
P313 in subsequence R1; and/or
F242 in subsequence R2; and/or
V187 in subsequence R3; and/or
A47 in subsequence R5.

Preferably, the glucose isomerase according to the invention comprises an amino acid sequence which has at least 80% identity to SEQ ID NO: 1 and which compared to SEQ ID NO: 1 comprises at least one, two, three, four, five, six, seven, eight, nine, ten, eleven, twelve or thirteen substitutions at substitution positions independently of one another selected from the groups consisting of:
A273, Q300, M309, P313, Q331, A342, and K343 in subsequence R1; and/or
M241 and F242 in subsequence R2; and/or
V187, and G190 in subsequence R3; and/or
W140 in subsequence R4; and/or
A47 in subsequence R5.

The glucose isomerase of the organism Bifidobacterium adolescentis comprises four monomers of the amino acid sequence with SEQ ID NO: 1 that form the active enzyme. For the purpose of the specification, all improved glucose isomerase variants likewise comprise four monomers of the same variant amino acid sequences of such SEQ ID NO:X corresponding to the variant enzyme. It is understood in the meaning of the invention that a glucose isomerase “comprising an amino acid sequence according to a SEQ ID NO:X” describes a glucose isomerase containing four copies of the amino acid sequence with the corresponding SEQ ID NO:X.

The structure of the glucose isomerase of SEQ ID NO: 1 has not been described in the prior art. A homology model for the glucose isomerase with monomers of the amino acid sequence SEQ ID NO:1 was thus generated using the software YASARA structure Version 19.4.2.L.64, using the included macro hm_build.mcr, and using the (default) standard settings, but with a maximum number of templates to be used of 20. The crystal structures with the pdb-codes (https://www.rcsb.org/) of 1A0E, 6INT, 4XKM and 1A0C were used by the software to build the final model, mainly based on the tetrameric structures 1A0E (xylose isomerase from Thermotoga neapolitana https://www.rcsb.org/structure/1A0E). Without wishing to be bound by any scientific theory, it is assumed that the amino acid sequence according to SEQ ID NO. 1, i.e. the wild-type enzyme from Bifidobacterium adolescentis, exhibits a fold-structure of the type PDB 1A0E.

Furthermore, the glucose isomerase of the amino acid sequence with SEQ ID NO: 1, or glucose isomerase variants with engineered amino acid sequences in each of the monomers of the active enzyme carry two “catalytic cations”, being either cobalt cations (Co²⁺), manganese cations (Mn²⁺), iron (II) cations (Fe²⁺), or magnesium cations (Mg²⁺), preferably magnesium cations (Mg²⁺).

The catalytic cations of the glucose isomerase of SEQ ID NO:1 and the improved variants according to the invention are coordinated; the catalytic cation (I) is coordinated by the residues D308 and D310 (among other residues) of SEQ ID NO: 1 or corresponding positions of variant amino acid sequences, and the catalytic cation (II) is coordinated by the residues E233, D297, D341 (amongst other residues) of SEQ ID NO: 1 or corresponding positions of variant amino acid sequences. The “other residues” enclose the positions E269 and D341 of SEQ ID NO: 1, or corresponding amino acid sequences from variants. All these positions are located closely to and are coordinating the metal ion and are preferably not positions of the amino acid sequence of SEQ ID NO:1 that are to be substituted according to the invention. Thus, positions E233, E269, D297, D308, D310, D341 of SEQ ID NO: 1 are preferably not modified according to the invention.

In the structural model of SEQ ID NO:1 four different “Spherical Spaces” can be defined, Spherical Space 1, Spherical Space 2, Spherical Space 3, and Spherical Space 4.

Spherical Space 1 (radius of 14.9 Å around catalytic cation (I)): Based on the structure model of SEQ ID NO: 1, in the homo-tetrameric structure of the glucose isomerase,
the amino acids positions 44, 45, 46, 47, 48, 49, 50, 51, 52, 53, 54, 55, 56, 57, 58, 59, 67, 100, 101, 102, 105, 139, 140, 142, 143, 144, 145, 146, 147, 187, 188, 189, 190, 191, 192, 193, 231, 232, 233, 234, 235, 236, 237, 238, 239, 240, 242, 243, 245, 246, 247, 248, 266, 267, 268, 269, 270, 271, 272, 273, 274, 275, 276, 277, 278, 279, 280, 283, 284, 295, 296, 297, 298, 299, 300, 301, 302, 303, 304, 305, 306, 307, 308, 309, 310, 311, 312, 313, 314, 315, 318, 319, 322, 338, 339, 340, 341, 342, 343, 344, 345, 346, 348, 354, 357, 358, 361, and 365 of the amino acid sequence SEQ ID NO: 1, which are located within monomer Chain A;
the amino acid positions 60, 61, and 241 of the amino acid sequence SEQ ID NO:1, which are located within monomer Chain C; and
the amino acid positions 431, 432, and 435 of the amino acid sequence SEQ ID NO:1, which are located within monomer Chain B;
   are all located within a sphere having a radius of 14.9 Å around the catalytic cation (I) that is coordinated by residues D308 and D310, hereinafter also referred to as Spherical Space 1. Preferably the amino acid positions A47, W140, V187, G190, M241, F242, A273, Q300, M309, P313, A342, and K343 are all located within the Spherical Space 1.

Spherical Space 2 (radius of 11 Å around catalytic cation (II)): Based on the structure model of SEQ ID NO: 1, in the homo-tetrameric structure of the glucose isomerase,
the amino acids positions 45, 46, 47, 48, 49, 50, 54, 100, 101, 102, 139, 140, 142, 146, 185, 186, 187, 188, 189, 190, 191, 231, 232, 233, 234, 235, 236, 243, 266, 267, 268, 269, 270, 271, 272, 273, 275, 280, 294, 295, 296, 297, 298, 299, 300, 308, 309, 310, 311, 313, 337, 338, 339, 340, 341, 342, 343 of the amino acid sequence SEQ ID NO: 1, which are located within monomer Chain A; and
the amino acid position 60 of the amino acid sequence SEQ ID NO:1, which is located within monomer Chain C;
   are all located within a sphere having a radius of 11 Å around catalytic cation (II)) that is coordinated by residues E233 and D297, hereinafter also referred to as Spherical Space 2. Preferably, the amino acid positions A47, W140, V187, G190, A273, Q300, M309, P313, A342, and K343 are all located within the Spherical Space 2.

Spherical Space 3 (radius of 13 Å around C-alpha of A342): Based on the structure model of SEQ ID NO: 1, in the homo-tetrameric structure of the glucose isomerase,
the amino acids positions 44, 45, 46, 47, 48, 49, 50, 51, 52, 53, 54, 56, 67, 90, 91, 94, 96, 98, 99, 100, 101, 102, 105, 139, 140, 146, 187, 189, 233, 235, 238, 267, 268, 269, 270, 271, 272, 273, 275, 280, 295, 296, 297, 298, 299, 300, 301, 302, 307, 308, 309, 310, 311, 312, 313, 314, 315, 318, 319, 322, 338, 339, 340, 341, 342, 343, 344, 345, 346, 348, 349, 353, 354, 355, 356, 357, 358, 359, 360, 361, 362, 363, 364, 365, 431, 432, 433, 435 of the amino acid sequence SEQ ID NO:1, which are located within monomer Chain A, and
the amino acid position 60 of the amino acid sequence SEQ ID NO: 1, which is located within monomer Chain C;
   are all located within a sphere having a radius of 13 Å with center in C-alpha of residue A342 in monomer Chain A, hereinafter also referred to as Spherical Space 3. Preferably, the amino acid positions A47, W140, V187, A273, Q300, M309, P313, A342, and K343 are all located within the Spherical Space 3.

Spherical Space 4 (radius of 7 Å around C-alpha of A342): Based on the structure model of SEQ ID NO:1, in the homo-tetrameric structure of the glucose isomerase,
the amino acids positions 47, 50, 269, 271, 297, 298, 299, 300, 309, 310, 311, 312, 313, 339, 340, 341, 342, 343, 344, 345, 358 of the amino acid sequence SEQ ID NO:1, which are located within monomer Chain A, and
the amino acid position 60 of the amino acid sequence SEQ ID NO: 1, which is located within monomer Chain C;
   are all located within a sphere having a radius of 7 Å with center in C-alpha of residue A342 in monomer Chain A, hereinafter also referred to as Spherical Space 4. Preferably, the amino acid positions A47, Q300, M309, P313, A342, and K343 are all located within the Spherical Space 4.

As outlined above, the variants according to the invention have substitutions at substitution positions; based upon the native three-dimensional structure of the glucose isomerase according to SEQ ID NO:1 these substitution positions are located within a certain sphere with a certain center, and are not described as substitution positions from the prior art. These substitution positions are linked to one another as a common structural feature. There is a technical relationship among those substitution positions provided by the functional improvements obtained by specific substitutions in the respective substitution positions resulting in an inventive contribution over the prior art.

Preferably, the variants according to the invention have substitutions at substitution positions, which are located within Spherical Space 1, Spherical Space 2, Spherical Space 3, and/or Spherical Space 4 based upon the native three-dimensional structure of the glucose isomerase according to SEQ ID NO:1.

In a preferred embodiment, the invention relates to a glucose isomerase comprising an amino acid sequence which has at least 65% identity to SEQ ID NO: 1 and which compared to SEQ ID NO: 1 comprises at least one, two, three, four, five, six, seven, eight, nine, ten, eleven, twelve, thirteen, fourteen, fifteen, sixteen or seventeen substitutions at substitution positions, which are independently of one another located in
subsequence R1 ranging from position L266 to R346 of SEQ ID NO:1; and/or
subsequence R2 ranging from position L231 to Q243 of SEQ ID NO:1; and/or
subsequence R3 ranging from position N185 to W199 of SEQ ID NO:1; and/or
subsequence R4 ranging from position L139 to Y164 of SEQ ID NO:1; and/or
subsequence R5 ranging from position G17 to R67 of SEQ ID NO:1,
   wherein the substitution positions of the invention are located in Spherical Space 1, Spherical Space 2, Spherical Space 3, and/or Spherical Space 4.

Preferably, the substitution positions of the invention are located in substitution positions
A273, Q300, M309, P313, Q331, A342, and K343 in subsequence R1; and/or
M241 and F242 in subsequence R2; and/or
V187, and G190 in subsequence R3; and/or
W140 in subsequence R4; and/or
A47 in subsequence R5.

Preferably, the glucose isomerase according to the invention comprises an amino acid sequence which has at least 65% identity to SEQ ID NO: 1 and which compared to SEQ ID NO: 1 comprises at least one substitution at substitution positions independently of one another selected from the groups consisting of:
P313 in subsequence R1; and/or
F242 in subsequence R2; and/or
V187 in subsequence R3; and/or
A47 in subsequence R5.

In a preferred embodiment, the invention relates to a glucose isomerase comprising an amino acid sequence which has at least 65% identity to SEQ ID NO: 1 and which compared to SEQ ID NO: 1 comprises at least one, two, three, four, five, six, seven, eight, nine, ten, eleven, twelve, thirteen, fourteen, fifteen, sixteen or seventeen substitutions at substitution positions, which are independently of one another selected from 44, 45, 46, 47, 48, 49, 50, 51, 52, 53, 54, 55, 56, 57, 58, 59, 60, 61, 67, 90, 91, 94, 96, 98, 99, 100, 101, 102, 105, 139, 140, 142, 143, 144, 145, 146, 147, 185, 186, 187, 188, 189, 190, 191, 192, 193, 231, 232, 233, 234, 235, 236, 237, 238, 239, 240, 241, 242, 243, 245, 246, 247, 248, 266, 267, 268, 269, 270, 271, 272, 273, 274, 275, 276, 277, 278, 279, 280, 283, 284, 294, 295, 296, 297, 298, 299, 300, 301, 302, 303, 304, 305, 306, 307, 308, 309, 310, 311, 312, 313, 314, 315, 318, 319, 322, 337, 338, 339, 340, 341, 342, 343, 344, 345, 346, 348, 349, 353, 354, 355, 356, 357, 358, 359, 360, 361, 362, 363, 364, 365 431, 432, 433, and 435 of the amino acid sequence SEQ ID NO: 1 in either of the monomeric chains, and preferably selected from substitution positions A47, W140, V187, G190, M241, F242, A273, Q300, M309, P313, A342, and K343.

The inventors of the present invention have identified that certain subregions of the subsequences R1 to R5 that are overlapping with the spherical spaces of the structural models of SEQ ID NO:1 described above are highly conserved or have a high degree of similarity compared to state-of-the-art homologous proteins. Specifically, the sequence positions 44, 45, 46, 47, 48, 49, 50, 51, 52, 53, 54, 55, 56, 57, 58, 59, 60, 61, 67, 90, 91, 94, 96, 98, 99, 100, 101, 102, 105, 139, 140, 142, 143, 144, 145, 146, 147, 185, 186, 187, 188, 189, 190, 191, 192, 193, 231, 232, 233, 234, 235, 236, 237, 238, 239, 240, 241, 242, 243, 245, 246, 247, 248, 266, 267, 268, 269, 270, 271, 272, 273, 274, 275, 276, 277, 278, 279, 280, 283, 284, 294, 295, 296, 297, 298, 299, 300, 301, 302, 303, 304, 305, 306, 307, 308, 309, 310, 311, 312, 313, 314, 315, 318, 319, 322, 337, 338, 339, 340, 341, 342, 343, 344, 345, 346, 348, 349, 353, 354, 355, 356, 357, 358, 359, 360, 361, 362, 363, 364, 365 431, 432, 433, and 435 of the amino acid sequence SEQ ID NO: 1 locate in either of the subsequences and in either of the Spherical Spaces according to the invention and are highly conserved or have a high degree of similarity compared to state-of-the-art homologous proteins. The analysis of an alignment of glucose isomerase variants homologous to SEQ ID NO: 1 reveals that all variants currently known in the state of the art are highly conserved in the abovementioned positions within a range of 65% identity, with a small number of very specific amino acids occurring throughout the variants.

In another preferred embodiment, the invention relates to a glucose isomerase comprising an amino acid sequence which has at least 65% identity to SEQ ID NO: 1 and which compared to SEQ ID NO: 1 comprises at least one, two, three, four, five, six, seven, eight, nine, ten, eleven, or twelve substitutions at substitution positions, wherein the substitution positions are independently of one another selected from the substitution positions A47, W140, V187, G190, M241, F242, A273, Q300, M309, P313, A342, and K343, and wherein the glucose isomerase furthermore comprises the following amino acids F44 or Y44; A45, G45, or S45; V46 or 146; W48 or Y48; W49; H50; T51 or S51; F52 or M52; A53, D53, E53, N53, or Q53; Q54 or A54; E55, G55, P55, Q55, R55, or T55; L56; T57, V57, I57 or N57; D58; P59 or N59; F60; G61; R67; F90 or L90; F91, L91, M91, or Y91; I94 or L94; A96, N96 or V96; F98, or Y98; F99, I99, L99 or Y99; C100, A100, or S100; F101; H102; D105 or N105; L139; T142; S143, Q143, A143 or C143; N144, S144, or D144; L145, M145 or T145; F146; T147, G147, N147, or S147; N185; Y186; F188; W189; G191; R192; E193; L231, or M231; I232, or L232; E233; P234; K235; A236, or P236; K237, or Q237; E238; P239; C240, M240, T240, A240, or S240; Q243; D245, or E245; F246, Y246, T246 or Y246; D247; A248, or V248; L266, M266, or Q266; N267; L268, or I268; E269; G270, or E270; N271; H272; N274; L275; A276; G277, M277, S277, T277, R277, or D277; H278; S279, or T279; Y280, or F280; E283; I284, or V284; G294; S295; L296, or I296; D297; A298; N299; G301; D302, or N302; K303, or M303; L304, M304 or F304; I305, or V305, or L305; G306; W307; D308; D310; E311, or Q311; 3F12, or Y312; S314, T314, or V314; A315, D315, N315, or T315; D318, E318, or A318; A319, C319, T319, or S319; V322, A322, or I322; G337; L338; N339; F340; D341; P344; R345; R346; S348, or A348; F349, W349 or Y349; D353; L354, or V354; F355, L355, or Y355; R356, A356, G356, K356, L356, Q356, T356 or Y356; A357, S357 T357, V357, or G357; H358, or Y358; I359, V359 or M359; A360, V360 or I360; G361; M362, or I362; D363; A364, S364, or T364; F365, or Y365; L431, S341, M341, I341 or A341; E432; C433, D433, Q433, S433, A433, E433, L433, R433, T433 or V433; and K435, R435 or M435 with the numbering referring to an aligning position of SEQ ID NO: 1.

Preferably, the invention relates to a glucose isomerase comprising an amino acid sequence which has at least 80% identity to SEQ ID NO: 1 and which compared to SEQ ID NO: 1 comprises at least one, two, three, four, five, six, seven, eight, nine, ten, eleven, or twelve substitutions at substitution positions, wherein the substitution positions are independently of one another selected from the substitution positions A47, W140, V187, G190, M241, F242, A273, Q300, M309, P313, A342, and K343, and wherein the glucose isomerase furthermore comprises the following amino acids F44; A45, or G45; V46 or 146; W48 or Y48; W49; H50; T51 or S51; F52 or M52; A53, D53, or N53; Q54 or A54; E55, G55, P55, Q55, R55, or T55; L56; T57, or V57; D58; P59; F60; G61; R67; F90 or L90; F91, L91, or Y91; L94; A96 or V96; F98, or Y98; F99; C100, A100, or S100; F101; H102; D105; L139; T142; S143, or Q143; N144, or S144; L145; F146; T147; N185; Y186; F188; W189; G191; R192; E193; L231; I232; E233; P234; K235; A236, or P236; K237; E238; P239; C240, M240, or T240; Q243; D245; F246, or Y246; D247; A248; L266, or Q266; N267; L268; E269; G270; N271; H272; N274; L275; A276; G277, M277, S277, or T277; H278; S279, or T279; Y280; E283; I284, or V284; G294; S295; L296; D297; A298; N299; G301; D302; K303, or M303; L304; I305, or V305, or L305; G306; W307; D308; D310; E311; 3F12; S314, or T314; A315, D315, or N315; D318, or E318; A319, C319, or T319; V322; G337; L338; N339; F340; D341; P344; R345; R346; S348; F349; D353; L354; F355; R356; A357, S357, or T357; H358; I359, or V359; A360, or V360; G361; M362; D363; A364, S364, or T364; F365, or Y365; L431, or S341; E432; C433, D433, Q433, or S433; and K435, or R435, with the numbering referring to an aligning position of SEQ ID NO:1.

Preferably, the invention relates to a glucose isomerase comprising an amino acid sequence which has at least 80% identity to SEQ ID NO: 1 and which compared to SEQ ID NO: 1 comprises at least one, two, three, four, five, six, seven, eight, nine, ten, eleven, or twelve substitutions at substitution positions, wherein the substitution positions are independently of one another selected from the substitution positions A47, W140, V187, G190, M241, F242, A273, Q300, M309, P313, A342, and K343, and wherein the glucose isomerase furthermore consists of comprises the following amino acids F44; G45; V46; W48; W49; H50; T51; F52; D53; Q54; E55; L56; V57; D58; P59; F60; G61; R67; F90; F91; L94; V96; Y98; F99; C100; F101; H102; D105; L139; T142; S143; S144; L145; F146; T147; N185; Y186; F188; W189; G191; R192; E193; L231; I232; E233; P234; K235; A236; K237; E238; P239; T240; Q243; D245; F246; D247; A248; L266; N267; L268; E269; G270; N271; H272; N274; L275; A276; G277; H278; T279; Y280; E283; I284; G294; S295; L296; D297; A298; N299; G301; D302; K303; L304; I305; G306; W307; D308; D310; E311; 3F12; T314; D315; E318; T319; V322; G337; L338; N339; F340; D341; P344; R345; R346; S348; F349; D353; L354; F355; R356; S357; H358; I359; A360; G361; M362; D363; S364; F365; L431; E432; S433; and K435, with the numbering referring to an aligning position of SEQ ID NO: 1.

It will be understood by a person skilled in the art how to identify amino acids positions of any wild type or other sequence of a glucose isomerase enzyme that is corresponding to the abovementioned positions in SEQ ID NO: 1. It is within the meaning of the invention that the aligning position of an amino acid of a glucose isomerase with a certain identity to SEQ ID NO: 1 shall be considered as corresponding to or referring to the sequence numbering of SEQ ID NO: 1, even when the specific number of such amino acid may be different in consideration of the overall length of the respective wild type or other glucose isomerase sequence.

Surprisingly, the inventors of the present invention have also identified that substituting certain amino acids at the substitution positions according to the invention within these highly conserved regions, which are not known or expected from the prior art, resulted in variant enzymes showing significant improvement in enzymatic activity. Preferably, the substitution positions for those variants are one or more substitutions in substitution positions selected from the group consisting of A273, Q300, M309, P313, Q331, A342, and K343 in subsequence R1; and/or M241 and F242 in subsequence R2; and/or V187, and G190 in subsequence R3; and/or W140 in subsequence R4; and/or A47 in subsequence R5. Specifically, the variant enzymes of the present invention showed high activity at low pH values ranging from at least pH 5.0 to pH 7.0, at low temperatures, and at high-calcium concentrations ranging from 0 mM up to 60 mM.

For example, the analysis of an alignment of glucose isomerase variants homologous to SEQ ID NO:1 reveals that all variants currently known in the state of the art are highly conserved in the claimed positions. For example, glucose isomerases within a range of 65% identity to SEQ ID NO: 1 described in the state of the art have identical conserved residues in 100% of residues at positions 187 (V, valine), 313 (P, proline), and 47 (A, alanine).

The substitutions according to the invention compared to SEQ ID NO: 1 are located at substitution positions in either of the subsequences R1 to R5 of the primary sequence of SEQ ID NO:1. Preferably, the substitution positions are located in the subsequences R1 to R3 and R5, more preferably in the subsequences R1 to R3.

Most preferably, the amino acid positions of the substitutions are located in subregions of the subsequences R1-R5:
for R1: subregions R1-A, R1-B (with subregions R1-B1 and R1-B2), R1-C, and R1-D, preferably R1-B and R-1D;
for R2: no subregions;
for R3: subregions R3-A, and R3-B, preferably R3-A;
for R4: subregions R4-A (with subregions R4-A1 and R4-A2), and R4-B, preferably R4-A, more preferably subregions R4-A1;
for R5: subregions R5-A, and R5-B, preferably R5-B.

It is within the meaning of the invention that one subsequence may have one or more subregions, wherein different subregions of a subsequence may overlap each other, or wherein one subregion may be a sub-stretch of another subregion. Each subregion according to the invention carries a specific name.

The subsequences R1-R5 partially locate in one or more of the Spherical Spaces 1 to 4 of the enzyme structure of SEQ ID NO: 1. Specifically, the subregions as outlined above locate in one or more of the Spherical Spaces 1 to 4 of the enzyme structure of SEQ ID NO:1.

Preferably, the amino acid positions of the substitutions according to the invention are located in the spherical space of a sphere having a radius of 14.9 Å around the catalytic cation (I) coordinated by residues D308, D310 that comprises a part of the subregions R1-A, R1-B (R1-B1 and R1-B2), R1-D, R2, R3-A, R4-A, and R5-B of SEQ ID NO:1, and that in particular comprises:
amino acid positions 44, 45, 46, 47, 48, 49, 50, 51, 52, 53, 54, 55, 56, 57, 58, 59, 60, 61, and 67 of SEQ ID NO: 1, which are located within the subregions R5-B;
amino acid positions 139, 140, 142, 143, 144, 145, 146, and 147 of SEQ ID NO:1, which are located within the subregions R4-A;
amino acid positions 187, 188, 189, 190, 191, and 192 of SEQ ID NO: 1, which are located within the subregions R3-A;
amino acid positions 231, 232, 233, 234, 235, 236, 237, 238, 239, 240, 241, 242, and 243 of SEQ ID NO:1, which are located within the subregions R2;
amino acid positions 266, 267, 268, 269, 270, 271, 272, 273, 274, 275, 276, 277, 278, 279, and 280 of SEQ ID NO: 1, which are located within the subregions R1-A;
amino acid positions 295, 296, 297, 298, 299, 300, 301, 302, 303, 304, 305, 306, 307, 308, 309, 310, 311, 312, 313, 314, and 315 of SEQ ID NO: 1, which are located within the subregions R1-B (R1-B1 and R1-B2); and
amino acid positions 338, 339, 340, 341, 342, 343, 344, 345, and 346 of SEQ ID NO:1, which are located within the subregions R1-D.

Preferably, the amino acid positions of the substitutions according to the invention are located in the spherical space of a sphere having a radius of 11 Å around catalytic cation (II)) coordinated by residues E233, D297 that comprises a part of the subregions R1-A, R1-B1, R1-B2, R1-D, R3-A, R4-A1, and R5-B of SEQ ID NO: 1, and that in particular comprises:
amino acid positions 45, 46, 47, 48, 49, 50, 54, and 60 of SEQ ID NO:1, which are located within the subregion R5-B;
amino acid positions 139 and 140 of SEQ ID NO:1, which are located within the subregion R4-A1;
amino acid positions 185, 186, 187, 188, 189, 190, and 191 of SEQ ID NO:1, which are located within the subregion R3-A;
amino acid positions 266, 267, 268, 269, 270, 271, 272, 273, 275, and 280 of SEQ ID NO:1, which are located within the subregion R1-A;
amino acid positions 295, 296, 297, 298, 299, 300, 308, 309, 310, 311, and 313 of SEQ ID NO:1, which are located within the subregion R1-B (R1-B1 and R1-B2); and
amino acid positions 337, 338, 339, 340, 341, 342, and 343 of SEQ ID NO:1, which are located within the subregion R1-D.

Preferably, the amino acid positions of the substitutions according to the invention are located in the spherical space of a sphere having a radius of 13 Å with center in C-alpha of residue A342 that comprises a part of the subregions R1-A, R1-B1, R1-B2, R1-D, R3-A, R4-A1, and R5-B of SEQ ID NO:1, and that in particular comprises:
amino acid positions 44, 45, 46, 47, 48, 49, 50, 51, 52, 53, 54, 56, 60, and 67 of SEQ ID NO:1, which are located within the subregion R5-B;
amino acid positions 139 and 140 of SEQ ID NO:1, which are located within the subregion R4-A1;
amino acid positions 187 and 189 of SEQ ID NO:1, which are located within the subregion R3-A;
amino acid positions 267, 268, 269, 270, 271, 272, 273, 275, and 280 of SEQ ID NO:1, which are located within the subregion R1-A;
amino acid positions 295, 296, 297, 298, 299, 300, 301, 302, 307, 308, 309, 310, 311, 312, 313, 314, and 315 of SEQ ID NO:1, which are located within the subregion R1-B (R1-B1 and R1-B2); and
amino acid positions 338, 339, 340, 341, 342, 343, 344, 345, and 346 of SEQ ID NO:1, which are located within the subregion R1-D.

Preferably, the amino acid positions of the substitutions according to the invention are located in the spherical space of a sphere having a radius of 7 Å with center in C-alpha of residue A342 that comprises a part of the subregions R1-B1, R1-B2, R1-D, and R5-B of SEQ ID NO: 1, and that in particular comprises:
amino acid positions 47 and 50 of SEQ ID NO:1, which are located within the subregion R5-B;
amino acid positions 297, 298, 299, 300, 309, 310, 311, 312, and 313 of SEQ ID NO:1, which are located within the subregion R1-B (R1-B1 and R1-B2); and
amino acid positions 339, 340, 341, 342, 343, 344, and 345 of SEQ ID NO:1, which are located within the subregion R1-D.

Preferably, the amino acid positions of the substitutions are located in subregions of subsequences R1-R5, that are entirely located within one or more of Spherical Spaces 1 to 4 of the enzyme structure of SEQ ID NO:1, as outlined above;
for R1:
   subregions R1-A, R1-S1-B; and R1-S-D; or
   subregions R1-S2-A, R1-S2-B1, R1-S2-B2, R1-S2-B3, and R1-S-D; or
   subregions R1-S3-A, R1-B1, R1-B2, and R1-S3-D; or
   subregions R1-S4-B1, R1-S4-B2, and R1-S4-D;
for R2: no subregion;
for R3: subregions R3-S1; R3-S2; and R3-S3;
for R4: subregion R4-A1;
for R5: subregions R5-S1; R5-S2; R5-S3; and R5-S4.

The subregions of subsequences R1-R5, as outlined above, are entirely located in certain spherical spaces of the enzyme structure of SEQ ID NO:1, whereby in particular
the spherical space of a sphere having a radius of 14.9 Å around the catalytic cation (I) coordinated by residues D308, D310, also referred to as Spherical Space 1 in the context of the present invention, entirely comprises the subregions R1-A, R1-S1-B, R1-S-D, R2, R3-S1, R4-A1, and R5-S1 of SEQ ID NO:1;
the spherical space of a sphere having a radius of 11 Å around catalytic cation (II)) coordinated by residues E233, D297, also referred to as Spherical Space 2 in the context of the present application, entirely comprises the subregions R1-S2-A, R1-S2-B1, R1-S2-B2, R1-S2-B3, R1-S-D, R3-S2, R4-A1, and R5-S2 of SEQ ID NO: 1;
the spherical space of a sphere having a radius of 13 Å with center in C-alpha of residue A342, also referred as to Spherical Space 3 in the context of the present application, entirely comprises the subregions R1-S3-A, R1-B1, R1-B2, R1-S3-D, R3-S3, R4-A1, and R5-S3 of SEQ ID NO: 1; and
the spherical space of a sphere having a radius of 7 Å with center in C-alpha of residue A342, also referred as to Spherical Space 4 in the context of the present invention, entirely comprises the subregions R1-S4-B1, R1-S4-B2, R1-S4-D, and R5-S4 of SEQ ID NO: 1.

The glucose isomerase according to the invention comprises an amino acid sequence with a defined identity to the amino acid sequence of SEQ ID NO: 1.

This means that any glucose isomerase according to the invention may comprise said amino acid sequence as a subsequence of its overall amino acid sequence, or may essentially consist of said amino acid sequence. When the glucose isomerase according to the invention comprises said amino acid sequence as a subsequence of its overall amino acid sequence, said overall amino acid sequence may be extended, i.e. may comprise additional amino acid residues, at the N-terminus and/or at the C-terminus of said subsequence. Such extension may be advantageous, for example, when the glucose isomerase is to be immobilized on a solid support, e.g. for purification purposes.

In the meaning of this invention, the percent identity is calculated as: Sequence Identity [%]=number of Matches/L×100, wherein L is the number of aligned positions, i.e. identities and non-identities (including gaps, if any). Identity is preferably calculated using BLASTP (see, for example, Altschul S F et al. (1997) “Gapped BLAST and PSI-BLAST: a new generation of protein database search programs”, Nucleic Acids Res. 25:3389-3402; or Altschul S F (2005) “Protein database searches using compositionally adjusted substitution matrices.” FEBS J. 272:5101-5109); preferably with the following algorithm parameters: Matrix: BLOSUM62; Gap Costs: Existence: 11 Extension: 1, Expect threshold: 10 and Word size: 6. Results are filtered for sequences with more than 35% query coverage. BlastP can be accessed online at the NCBI Homepage (https://blast.ncbi.nlm.nih.gov/Blast.cgi?PROGRAM=blastp&PAGE_TYPE=BlastSearch&LINK_LOC=blasthome). Other program settings can be adjusted as desired, for example using the following settings;
Field “Enter Query Sequence”: Query subrange: none;
Field “Choose Search Set”: Database: non-redundant protein sequences (nr); optional parameters: none
Field “Program Selection”: Algorithm: blastp (protein-protein BLAST);
Algorithm parameters: Field “General parameters”: Max target sequences: 20000; Short queries: Automatically adjust parameters for short input sequences; Expect threshold: 10; Word size: 6; Max matches in a query range: 0;
Algorithm parameters: Field “Scoring parameters”: Matrix: BLOSUM62; Gap Costs: Existence: 11 Extension: 1; Compositional adjustments: Conditional compositional score matrix adjustment;
Algorithm parameters: Field “Filters and Masking”: Filter: none; Mask: none.

In addition to the default parameters for calculation of Percent identity in the meaning of this invention, when aligning a sequence to be examined (“query sequence”) with a reference sequence (“subject sequence”), the subject sequence must be represented with at least 50% over the length of the individual alignment (“sequence coverage” at least 50%); alignments with a lower sequence coverage of the subject sequence are excluded for the determination of sequence identity for the purposes of this application. However, the query sequence may be longer than the length of the alignment.

Further, the glucose isomerase according to the invention may carry at least one substitution at any of amino acid positions 1 to 449 of SEQ ID NO:1. For the purpose of the specification, “substitution” is synonymous to “mutation”. Likewise, the terms “substitution position” and “mutation position” synonymously refer to the amino acid position of the substitution or mutation in comparison to SEQ ID NO:1.

Furthermore, any substitution in any position of the glucose isomerase according to the invention may be a substitution of any amino acid of SEQ ID NO:1 by any differing amino acid selected from the group of natural proteinogenic amino acids, i.e. A, R, N, D, C, Q, E, G, H, I, L, K, M, F, T, W, Y, V, P or S.

Preferably, at least one amino acid of the glucose isomerase of SEQ ID NO: 1 is substituted by (i) a positively charged amino acid selected from R, K, and H; or (ii) a negatively charged amino acid selected from E and D; or (iii) an aliphatic nonpolar amino acid selected from G, A, V, L, I, and P; or (iv) an aromatic amino acid selected from W, F, and Y; or (v) an aliphatic polar non-charged amino acid selected from N, Q, S, T, C, and M. For the purpose of the specification, the status of charge is determined at a pH value of 7.0. Thus, irrespective of the actual pH value of the environment of the glucose isomerase, R, K, and H are to be considered positively charged amino acids, whereas E and D are to be considered negatively charged amino acids.

Further, the glucose isomerase according to the invention may carry at least one substitution, wherein (i) a positively charged amino acid is substituted by another positively charged amino acid; or (ii) a negatively charged amino acid is substituted by another negatively charged amino acid; or (iii) an aliphatic nonpolar amino acid is substituted by another aliphatic nonpolar amino acid; or (iv) an aromatic amino acid is substituted by another aromatic amino acid; or (v) an aliphatic polar non-charged amino acid is substituted by another aliphatic polar non-charged amino acid.

Further, the glucose isomerase according to the invention may carry at least one substitution, wherein (i) a positively charged amino acid is substituted by an aliphatic nonpolar amino acid, or by a negatively charged amino acid, or by an aromatic amino acid, or by an aliphatic polar non-charged amino acid; or (ii) a negatively charged amino acid is substituted by an aliphatic nonpolar amino acid, or by an aromatic amino acid, or by an aliphatic polar non-charged amino acid, or a positively charged amino acid; or (iii) an aliphatic nonpolar amino acid is substituted by an aromatic amino acid, or by a negatively charged amino acid, or by an aliphatic polar non-charged amino acid, or by a positively charged amino acid; or (iv) an aromatic amino acid is substituted by an aliphatic nonpolar amino acid, or by a negatively charged amino acid, or by an aliphatic polar non-charged amino acid, or by a positively charged amino acid; or (v) an aliphatic polar non-charged amino acid is substituted by an aliphatic nonpolar amino acid, or by a negatively charged amino acid, or by an aromatic amino acid, or by a positively charged amino acid.

It is in the meaning of the invention that the glucose isomerase according to the invention comprising at least one, at least two, at least three, at least four, at least five, at least six, at least seven, at least eight, at least nine, at least ten, at least eleven, at least twelve, at least thirteen, at least fourteen, at least fifteen, at least sixteen, or at least seventeen substitutions, comprises one or more, two or more, three or more, four or more, five or more, six or more, seven or more, eight or more, nine or more, ten or more, eleven or more, twelve or more, thirteen or more, fourteen or more, fifteen or more, sixteen or more, or seventeen or more substitutions. Furthermore, it is in the scope of the invention that the glucose isomerase according to the invention carries, one, two, three, four, five, six, seven, eight, nine, ten, eleven, twelve, thirteen, fourteen, fifteen, sixteen or seventeen substitutions, or more.

Preferably, the glucose isomerase according to the inventions is substituted compared to SEQ ID NO:1 in 1 to 164 amino acid positions, preferably in 1 to 100 amino acid positions, more preferably in 1 to 50 amino acid positions, even more preferably in 1 to 25 amino acid positions, most preferably in 1 to 17 amino acid positions, and utmost preferably 1 to 9 amino acid positions.

Preferably, the glucose isomerase according to the invention does not carry any substitution in substitution positions, which are integral part of the catalytic cascade and which substitution would prevent the enzymatic activity of the glucose isomerase. In particular, the glucose isomerase of SEQ ID NO: 1 does not carry substitutions in positions E233, E269, D297, D308, D310, and D341.

For the purpose of the description, specific substitution positions disclosed herein are described in accordance with common practice in the field of amino acid substitutions, wherein the format [non-substituted amino acid-sequence position] is chosen and the sequence position is directly flanked with the amino acids letter (one letter code) of the not yet mutated amino acid on the left side, wherein no specific amino acid is selected as a substituent for such amino acid position. For clarification only, substitution “V187” describes the substitution of the amino acid valine (V) in sequence position 187 by any substituent.

For the purpose of the description, specific substitutions disclosed herein are described in accordance with common practice in the field of amino acid substitutions, wherein the format [non-substituted amino acid-sequence position-substituted amino acid] is chosen and the sequence position is directly flanked with the amino acids letter (one letter code) on the left or right side. For clarification only, substitution “V187C” describes the substitution of the amino acid valine (V) in sequence position 187 by the amino acid cysteine (C), i.e. V is replaced by C.

A first aspect of the invention relates to a glucose isomerase comprising an amino acid sequence which has at least 65% identity, or at least 80% identity, to SEQ ID NO: 1 and which compared to SEQ ID NO: 1 comprises at least one, two, three, four, five, six, seven, eight, nine, ten, eleven, twelve, thirteen, fourteen, fifteen, sixteen or seventeen substitutions at substitution positions, which are located in
subsequence R1 ranging from position L266 to R346 of SEQ ID NO:1; and/or
subsequence R2 ranging from position L231 to Q243 of SEQ ID NO:1; and/or
subsequence R3 ranging from position N185 to W199 of SEQ ID NO:1; and/or
subsequence R4 ranging from position L139 to Y164 of SEQ ID NO:1; and/or
subsequence R5 ranging from position G17 to R67 of SEQ ID NO:1.

Preferably, the amino acid sequence of the glucose isomerase comprises at least one, two, three, four, five, or six substitutions at substitution positions in subsequence R1; preferably in
subsequence R1-A ranging from position L266 to Y280;
   preferably ranging from position L266 to A273, more preferably ranging from position N267 to A273, and most preferably in position A273; and/or
subsequence R1-B ranging from position S295 to D315;
   preferably in subsequence R1-B1 ranging from position S295 to D302, more preferably ranging from position A298 to Q300, and most preferably in position Q300; and/or
   subsequence R1-B2 ranging from position W307 to D315, more preferably ranging from position D308 to P313, and most preferably in positions M309 and/or P313; and/or
subsequence R1-C ranging from position G330 to P334; preferably in position Q331; and/or
subsequence R1-D ranging from position G337 to R346; preferably ranging from position L338 to K343, more preferably ranging from position N339 to K343, and most preferably in positions A342 and/or K343.

Preferably, the amino acid sequence of the glucose isomerase comprises at least one, two, three, four, five or six substitutions at substitution positions in subregions of subsequence R1 that are entirely located within the Spherical Space 1; preferably in
subregion R1-A ranging from position L266 to Y280 as outlined above, most preferably in position A273; and/or
subregion R1-S1-B ranging from position S295 to T319, most preferably in positions Q300, M309, and/or P313; and/or
subregion R1-S-D ranging from position L338 to K343, most preferably in positions A342 and/or K343.

Preferably, the amino acid sequence of the glucose isomerase comprises at least one, two, three, four, five or six substitutions at substitution positions in subregions of subsequence R1 that are entirely located within the Spherical Space 2; preferably in
subregion R1-S2-A ranging from position L266 to A273, most preferably in position A273; and/or
subregion R1-S2-B1 ranging from position S295 to Q300, most preferably in position Q300; and/or
subregion R1-S2-B2 ranging from position D308 to D311, most preferably in position M309; and/or
subregion R1-S2-B3 consisting of position P313; and/or
subregion R1-S-D ranging from position L338 to K343 as outlined above, most preferably in positions A342 and/or K343.

Preferably, the amino acid sequence of the glucose isomerase comprises at least one, two, three, four or five substitutions at substitution positions in subregions of subsequence R1 that are entirely located within the Spherical Space 3; preferably in
subregion R1-S3-A ranging from position N267 to A273, most preferably in position A273; and/or
subregion R1-B1 ranging from position S295 to D302 as outlined above, most preferably in position Q300; and/or
subregion R1-B2 ranging from position W307 to D315 as outlined above, most preferably in positions M309 and/or P313; and/or
subregion R1-S3-D ranging from position L338 to R346, most preferably in positions A342 and/or K343.

Preferably, the amino acid sequence of the glucose isomerase comprises at least one, two, three, four or five substitutions at substitution positions in subregions of subsequence of R1 that are entirely located within the Spherical Space 4; preferably in
subregion R1-S4-B1 ranging from position D297 to Q300, most preferably in position Q300; and/or
subregion R1-S4-B2 ranging from position M309 to P313, most preferably in positions M309 and/or P313; and/or
subregion R1-S4-D ranging from position N339 to R345, most preferably in positions A342 and/or K343.

Preferably, the amino acid sequence of the glucose isomerase comprises at least one or two substitutions, wherein the substitution positions are in subsequence R2, preferably in positions M241 and/or F242.

Preferably, the amino acid sequence of the glucose isomerase comprises at least one or two substitutions at substitution positions in subregions of subsequence R2 which are entirely located within the Spherical Space 1, preferably in positions M241 and/or F242.

Preferably, the amino acid sequence of the glucose isomerase comprises at least one, two or three substitutions, wherein the substitution positions are in subsequence R3; preferably in
subsequence R3-A ranging from position N185 to R192, more preferably ranging from position V187 to G191, still more preferably in positions V187 and/or G190; and most preferably in position V187; and/or
subsequence R3-B consisting of position L198.

Preferably, the amino acid sequence of the glucose isomerase comprises at least one or two substitutions at substitution positions in subregions of subsequence R3 that are entirely located within the Spherical Space 1, preferably in subregion R3-S1 ranging from position V187 to E193, more preferably in positions V187 and/or G190, most preferably in position V187.

Preferably, the amino acid sequence of the glucose isomerase comprises at least one or two substitutions at substitution positions in subregions of subsequence R3 that are entirely located within the Spherical Space 2, preferably in subregion R3-S2 ranging from position N185 to G191, more preferably in positions V187 and/or G190, most preferably in position V187.

Preferably, the amino acid sequence of the glucose isomerase comprises at least one substitution at substitution positions in subregions of subsequence R3 that are entirely located within the Spherical Space 3; preferably in subregion R3-S3, consisting of position V187.

Preferably, the amino acid sequence of the glucose isomerase comprises sequence comprises at least one, two or three substitutions, wherein the substitution positions are in subsequence R4; preferably in
subsequence R4-A ranging from position L139 to T147,
   preferably in subsequence R4-A1 ranging from position L139 to W140, and most preferably in position W140; and/or
   subsequence R4-A2 ranging from position T142 to T147, and most preferably in position T147; and/or
subsequence R4-B ranging from position A161 to Y164, and more preferably in position I163.

Preferably, the amino acid sequence of the glucose isomerase comprises at least one substitution at substitution positions in subregions of subsequence R4 that are entirely located within the Spherical Space 1, preferably in subregion R4-A1 ranging from position L139 to W140, more preferably in position W140.

Preferably, the amino acid sequence of the glucose isomerase comprises at least one substitution at substitution positions in subregions of subsequence R4, preferably in subregions of subsequence R4-alpha, that are entirely located within the Spherical Space 2, preferably in subregion R4-A1 ranging from position L139 to W140, more preferably in position W140.

Preferably, the amino acid sequence of the glucose isomerase comprises at least one substitution at substitution positions in subregions of subsequence R4 that are entirely located within the Spherical Space 3, preferably in subsequence R4-A1 ranging from position L139 to W140, more preferably in position W140.

Preferably, the amino acid sequence of the glucose isomerase comprises sequence comprises at least one or two substitutions, wherein the substitution positions are in subsequence R5; preferably in
subregion R5-A ranging from position G17 to Q19, more preferably in position P18; and/or
subregion R5-B ranging from position F44 to R67, more preferably ranging from position F44 to Q54, even more preferably ranging from position G45 to H50, and most preferably in position A47.

Preferably, the amino acid sequence of the glucose isomerase comprises at least one substitution at substitution positions in subregions of subsequence R5 that are entirely located within the Spherical Space 1; preferably in subregion R5-S1 ranging from position F44 to G61, most preferably in position A47.

Preferably, the amino acid sequence of the glucose isomerase comprises at least one substitution at substitution positions in subregions of subsequence R5 that are entirely located within the Spherical Space 2; preferably in subregion R5-S2 ranging from position G45 to H50, most preferably in position A47.

Preferably, the amino acid sequence of the glucose isomerase comprises at least one substitution at substitution positions in subregions of subsequence R5 that are entirely located within the Spherical Space 3; preferably in subregion R5-S3 ranging from position F44 to G54, most preferably in position A47.

Preferably, the amino acid sequence of the glucose isomerase comprises at least one substitution at substitution positions in subregions of subsequence R5 that are entirely located within the Spherical Space 4; preferably in subregion R5-S4 consisting of position A47.

Preferably, the amino acid sequence of the glucose isomerase comprises at least one, two, three, four, five, six, seven, eight, nine, ten, eleven, twelve, thirteen, fourteen, fifteen, sixteen or seventeen substitutions at substitution positions in any subsequence independently of one another selected from the groups consisting of R1, R2, R3, R4, and/or R5; or R1 and R2 and R3 and R4 and R5; or R1 and R2 and R3 and R5; or R1 and R3 and R5; or R1 and R5; or R1; or R3; or R5; or R4.

Preferably, the amino acid sequence of the glucose isomerase comprises at least one, two, three, four, five, six, seven, eight, nine, ten, eleven, twelve, thirteen, fourteen, fifteen, sixteen or seventeen substitutions, wherein the substitution positions are in any subsequence or subregion, and
wherein the substitutions in subsequence R1 are independently of one another selected from the groups consisting of:
   R1-A, R1-B, R1-C and/or R1-D; or
   R1-A, and R1-B, and R1-C and R1-D; or
   R1-A, R1-B, and R1-D; or
   R1-B and R1-D; or
   R1-A, R1-B1, R1-B2, R1-C, and R1-D; or
   R1-A, R1-B1, R1-B2, and R1-D; or
   R1-B1, R1-B2, R1-C, and R1-D; or
   R1-B1, R1-B2, and R1-D; and/or
wherein the substitutions in subsequence R3 are independently of one another selected from the group consisting of:
   R3-A and R3-B; or
   R3-A; or
   R3-B; and/or
wherein the substitutions in subsequence R4 are independently of one another selected from the groups consisting of:
   R4-A and R4-B; or
   R4-A1, R4-A2, and R4-B; or
   R4-A1, and R4-A2; or
   R4-A1, and R4-B; or
   R4-A2, and R4-B; or
   R4-A1, R4-A2, or R4-B; and/or
wherein the substitutions in subsequence R5 are independently of one another selected from the groups consisting of
   R5-A and R5-B; or
   R5-A; or
   R5-B.

Preferably, the amino acid sequence of the glucose isomerase comprises at least one, two, three, four, five, six, seven, eight, nine, ten, eleven, twelve, thirteen, fourteen, fifteen, sixteen or seventeen substitutions, wherein the substitution positions are in any subsequence or subregion, and wherein the substitutions are independently of one another selected from the groups consisting of R1-B, R1-D and R5-B; or R1-B, R1-D and R3-A; or R1-B, R1-D, R3-A and R5-B; or R1-B, R1-D, R3-A, R2 and R5-B.

Preferably, the amino acid sequence of the glucose isomerase comprises at least one, two, three, four, five, six, seven, eight, nine, ten, eleven, twelve or thirteen substitutions, wherein the substitution positions are in any subregion, and wherein the substitutions are independently of one another selected from the group of subregions consisting of R1-A, R1-S1-B, R1-S-D, R2, R3-S1, R4-A1, and R5-S1; or R1-S2-A, R1-S2-B1, R1-S2-B2, R1-S2-B3, R1-S-D, R3-S2, R4-A1, and R5-S2; or R1-S3-A, R1-B1, R1-B2, R1-S3-D, R3-S3, R4-A1, and R5-S3; or R1-S4-B1, R1-S4-B2, R1-S4-D, and R5-S4.

Preferably, the amino acid sequence of the glucose isomerase comprises at least one, two, three, four, five, six, seven, eight, nine, ten, eleven, twelve, thirteen, fourteen, fifteen, sixteen or seventeen substitutions, wherein the substitution positions are in any subregion, and the substitutions are independently of one another selected from the groups consisting of
S295 to D315, G337 to R346, and F44 to R67; preferably S295 to D302, W307 to D315, L338 to K343, and F44 to Q54; more preferably A298 to Q300, D308 to P313, N339 to K343, G45 to H5, and most preferably Q300, M309, P313, A342, K343 and A47; or
S295 to D315, G337 to R346, and N185 to R192; preferably S295 to D302, W307 to D315, L338 to K343, and V187 to G191; more preferably A298 to Q300, D308 to P313, N339 to K343, V187, and G190, and most preferably Q300, M309, P313, A342, K343, and V187; or
S295 to D315, G337 to R346, N185 to R192, and F44 to R67; preferably S295 to D302, W307 to D315, L338 to K343, V187 to G191, and F44 to Q54; more preferably A298 to Q300, D308 to P313, N339 to K343, V187, G190, and G45 to H50, and most preferably Q300, M309, P313, A342, K343, V187 and A47; or
S295 to D315, G337 to R346, N185 to R192, M241, F242, and F44 to R67; preferably S295 to D302, W307 to D315, L338 to K343, V187 to G191, M241, F242, and F44 to Q54; more preferably A298 to Q300, D308 to D315, N339 to K343, V187, G190, G45 to H5, M241, F242, and most preferably Q300, M309, P313, A342, K343, V187, M241, F242 and A47.

Preferably, the amino acid sequence of the glucose isomerase comprises at least one substitution in subregion R3-A.

Preferably, the amino acid sequence of the glucose isomerase comprises at least one substitution in substitution positions N185 to R192, preferably in substitution position V187.

Preferably, the amino acid sequence of the glucose isomerase comprises at least one, two, three, four, or five substitutions, wherein the substitution positions are in any subregions of R1-B and R1-D.

Preferably, the amino acid sequence of the glucose isomerase comprises at least one, two, three, four, or five substitutions, wherein the substitution positions are in substitution positions S295 to D315, and G337 to R346; preferably S295 to D302, W307 to D315, and L338 to K343; more preferably A298 to Q300, D308 to P313, and N339 to K343, and most preferably Q300, M309, P313, A342, and K343.

Preferably, the amino acid sequence of the glucose isomerase comprises at least one, two, or three substitutions, wherein the substitution positions are in subregion R1-B.

Preferably, the amino acid sequence of the glucose isomerase comprises at least one, two, or three substitutions, wherein the substitution positions are in substitution positions S295 to D315; preferably S295 to D302, and W307 to D315; more preferably A298 to Q300, and D308 to P313, and most preferably in Q300, M309, and P313.

Preferably, the amino acid sequence of the glucose isomerase comprises at least one, or two substitutions, wherein the substitution positions are in subregion R1-D.

Preferably, the amino acid sequence of the glucose isomerase comprises at least one, or two substitutions, wherein the substitution positions are in substitution positions G337 to R346; preferably L338 to K343; more preferably and N339 to K343, and most preferably in A342, and K343.

Preferably, the amino acid sequence of the glucose isomerase comprises at least one, two, three, four, five, or six substitutions, wherein the substitution positions are independently of one another selected from the groups consisting of Q300, M309, P313, A342, K343 and A47.

Preferably, the amino acid sequence of the glucose isomerase comprises at least one, two, three, four, five, or six substitutions, wherein the substitution positions are independently of one another selected from the groups consisting of Q300, M309, P313, A342, K343, and V187.

Preferably, the amino acid sequence of the glucose isomerase comprises at least one, two, three, four, five, six or seven substitutions, wherein the substitution positions are independently of one another selected from the groups consisting of Q300, M309, P313, A342, K343, V187 and A47.

Preferably, the amino acid sequence of the glucose isomerase comprises at least one, two, three, four, five, six, seven, eight or nine substitutions, wherein the substitution positions are independently of one another selected from the groups consisting of Q300, M309, P313, A342, K343, V187, M241, F242 and A47.

Preferably, the amino acid sequence of the glucose isomerase comprises at least one, two, three, four, five, six, seven, eight, nine, ten, eleven, twelve, thirteen, fourteen, fifteen, sixteen or seventeen substitutions, wherein the substitution positions are in any subsequence, and substitutions are independently of one another selected from the group consisting of subsequences R1, R2, R3, R4, and/or R5, and wherein the amino acid sequence in addition comprises at least one further substitution in any subsequence independently of one another selected from the group consisting of subsequences R1, R2, R3, R4, and/or R5, preferably in any subregion independently of one another selected from the group consisting of R1-A, R1-B, R1-C, R1-D, R2, R3-A, R3-B, R4-A, R4-B, R5-A and R5-B, and more preferably in any subsequence independently of one another selected from the group consisting of R1-A, R1-B1, R1-B2, R1-C, R1-D, R2, R3-A, R3-B, R4-A1, R4-A2, R4-B, R5-A and R5-B.

Preferably, the amino acid sequence of the glucose isomerase comprises at least one, two, three, four, five, six, seven, eight, nine, ten, eleven, twelve, thirteen, fourteen, fifteen, sixteen or seventeen substitutions, wherein the substitution positions are in any subsequence independently of one another selected from the group consisting of R1, R2, R3, R4, and/or R5, and wherein the amino acid sequence in addition comprises at least one further substitution in any substitution position of a subsequence, wherein the at least one substitution position is independently of one another selected from the group consisting of P18, A47, W140, T147, I163, V187, G190, L198, M241, F242, A273, Q300, M309, P313, Q331, A342, and K343.

Preferably, the amino acid sequence of the glucose isomerase comprises at least one, two, three, four, five, six, seven, eight, nine, ten, eleven, twelve, thirteen, fourteen, fifteen, sixteen or seventeen substitutions, wherein the substitution positions are independently of one another selected from the groups consisting of: A273, Q300, M309, P313, Q331, A342, and K343 in subsequence R1, and/or M241, and F242 in the subsequence R2, and/or V187, G190, L198, in subsequence R3, and/or W140, T147, and I163, in subsequence R4, and/or P18, and A47 in subsequence R5.

Preferably, the amino acid sequence of the glucose isomerase comprises at least one, two, three, four, five, six, seven, eight, nine, ten, eleven, twelve, thirteen, fourteen, fifteen, sixteen or seventeen substitutions,
wherein the substitution positions in subsequences R1 are independently of one another selected from the groups consisting of A273, Q300, M309, P313, Q331, A342, and K343; or A273, Q300, M309, P313, Q331, and A342; or A273, Q300, M309, P313, A342, and K343; or Q300, M309, P313, A342, and K343; or Q300, M309, P313, or A342, and K343; or A273, Q300, M309, P313, Q331, A342, or K343; and/or
wherein the substitution positions in subsequences R2 are independently of one another selected from the groups consisting of M241 and F242; or M241, or F242; and/or
wherein the substitution positions in subsequence R3 are independently of one another selected from the groups consisting of: V187, G190, L198; or V187, G190; or L198, or V187, or G190; and/or
wherein the substitution positions in subsequence R4 are independently of one another selected from the groups consisting of: W140, T147, and I163; or W140 and T147; or W140 and I163; or T147 and I163; or W140, T147, or I163; and/or
wherein the substitution positions in subsequence R5 are independently of one another selected from the groups consisting of: P18 and A47; or P18 or A47.

Preferably, the amino acid sequence of the glucose isomerase comprises at least one, two, three, four, five, six, seven, eight, nine, ten, eleven, twelve, thirteen, fourteen, fifteen, sixteen or seventeen substitutions, wherein the substitution positions are in any subsequence, and wherein the substitutions are independently of one another selected from the group consisting of subregions R1-A, R1-B, R1-D, R2, R3-A, R4-A1, and R5-B.

Preferably, the amino acid sequence of the glucose isomerase comprises at least one, two, three, four, five, six, seven, eight, nine, ten, eleven, or twelve substitutions, wherein
from subregion R1-A, the substitution position is A273; and/or
from subregion R1-B, the substitution positions are independently of one another selected from the group consisting of P313, Q300, and M309; and/or
from subregion R1-D, the substitution positions are independently of one another selected from the group consisting of A342, and K343; and/or
from subsequence R2, the substitution positions are independently of one another selected from the group consisting of M241 and F242; and/or
from subregion R3-A, the substitution positions are independently of one another selected from the group consisting of V187, and G190; and/or
from subregion R4-A1, the substitution position is W140; and/or
from subregion R5-B, the substitution position is A47.

Preferably, the amino acid sequence of the glucose isomerase comprises at least one, two, three, four, five, six, seven, eight, nine, ten, eleven, twelve, thirteen, fourteen, fifteen, sixteen or seventeen substitutions, wherein the substitution positions are in any subsequence, and wherein the substitutions are independently of one another selected from the group consisting of subregions R1-A, R1-B, R1-D, R3-A, R4-A1, and R5-B.

Preferably, the amino acid sequence of the glucose isomerase comprises at least one, two, three, four, five, six, seven, eight, nine or ten substitutions, wherein
from subregion R1-A, the substitution position is A273; and/or
from subregion R1-B, the substitution positions are independently of one another selected from the group consisting of P313, Q300, and M309; and/or
from subregion R1-D, the substitution positions are independently of one another selected from the group consisting of A342, and K343; and/or
from subregion R3-A, the substitution positions are independently of one another selected from the group consisting of V187, and G190; and/or
from subregion R4-A1, the substitution position is W140; and/or
from subregion R5-B, the substitution position is A47.

Preferably, the amino acid sequence of the glucose isomerase comprises at least one, two, three, four, five, six, seven, eight or nine substitutions, wherein
from subregion R1-A, the substitution position is A273; and/or
from subregion R1-B, the substitution positions are independently of one another selected from the group consisting of P313, Q300, and M309; and/or
from subregion R1-D, the substitution positions are independently of one another selected from the group consisting of A342, and K343; and/or
from subregion R3-A, the substitution positions are independently of one another selected from the group consisting of V187; and/or
from subregion R4-A1, the substitution position is W140; and/or
from subregion R5-B, the substitution position is A47.

Preferably, the amino acid sequence of the glucose isomerase comprises at least one, two, three, four, five, six, seven, eight, nine, ten, eleven, twelve, thirteen, fourteen, fifteen, sixteen or seventeen substitutions, wherein the substitution positions are in any subsequence, and wherein the substitutions are independently of one another selected from the group consisting of subregions R1-B, R1-D, and R5-B.

Preferably, the amino acid sequence of the glucose isomerase comprises at least one, two, three, four, five or six substitutions, wherein
from subregion R1-B, the substitution positions are independently of one another selected from the group consisting of P313, Q300, and M309; and/or
from subregion R1-D, the substitution positions are independently of one another selected from the group consisting of A342, and K343; and/or
from subregion R5-B, the substitution position is A47.

Preferably, the amino acid sequence of the glucose isomerase comprises at least one, two, three, four, five, six, seven, eight, nine, ten, eleven, twelve, thirteen, fourteen, fifteen, sixteen or seventeen substitutions, wherein the substitution positions are in any subsequence, and wherein the substitutions are independently of one another selected from the group consisting of subregions R1-B, R2, R3-A, and R5-B.

Preferably, the amino acid sequence of the glucose isomerase comprises at least one substitution, wherein
from subregion R1-B, the substitution position is P313; and/or
from subsequence R2, the substitution position is F242; and/or
from subregion R3-A, the substitution position is V187; and/or
from subregion R5-B, the substitution position is A47.

Preferably, the amino acid sequence of the glucose isomerase comprises at least one, two, three, four, five, six, seven, eight, nine, ten, eleven, twelve or thirteen substitutions, wherein the substitution positions are in any subsequence, and wherein the substitutions are independently of one another selected from the group consisting of subregions R1-A, R1-S1-B, R1-S-D, R2, R3-S1, R4-A1, and R5-S1, preferably selected from the group consisting of subregions R1-S1-B1, R2, R3-S1, and R5-S1.

Preferably, the amino acid sequence of the glucose isomerase comprises at least one, two, three, four, five, six, seven, eight, nine, ten, eleven or twelve substitutions, wherein
from subregion R1-A, the substitution position is A273; and/or
from subregion R1-S1-B, the substitution positions are independently of one another selected from the group consisting of P313, Q300, and M309; and/or
from subregion R1-S1-D, the substitution positions are independently of one another selected from the group consisting of A342, and K343; and/or
from subregion R2, the substitution positions are independently of one another selected from the group consisting of M241, and F242; and/or
from subregion R3-S1, the substitution positions are independently of one another selected from the group consisting of V187, and G190, preferably the substitution position is V187; and/or
from subregion R4-A1, the substitution position is W140; and/or
from subregion R5-S1, the substitution position is A47.

Preferably, the amino acid sequence of the glucose isomerase comprises at least one substitution, wherein
from subregion R1-S1-B, the substitution position is P313; and/or
from subregion R2, the substitution position is F242; and/or
from subregion R3-S1, the substitution position is V187; and/or
from subregion R5-S1, the substitution position is A47.

Preferably, the amino acid sequence of the glucose isomerase comprises at least one, two, three, four, five, six, seven, eight, nine or ten substitutions, wherein the substitution positions are in any subsequence, and wherein the substitutions are independently of one another selected from the group consisting of subregions R1-S2-A, R1-S2-B1, R1-S2-B2, R1-S2-B3, R1-S-D, R3-S2, R4-A1, and R5-S2, preferably selected from the group consisting of subregions R1-S2-B3, R3-S2, and R5-S2.

Preferably, the amino acid sequence of the glucose isomerase comprises at least one, two, three, four, five, six, seven, eight or nine substitutions, wherein
from subregion R1-S2-A, the substitution position is A273; and/or
from subregion R1-S2-B1, the substitution position is Q300; and/or
from subregion R1-S2-B2, the substitution positions is M309; and/or
from subregion R1-S2-B3, the substitution positions is P313; and/or
from subregion R1-S-D, the substitution positions are independently of one another selected from the group consisting of A342, and K343; and/or
from subregion R3-S2, the substitution positions are independently of one another selected from the group consisting of V187, and G190, preferably the substitution position is V187; and/or
from subregion R4-A1, the substitution position is W140; and/or
from subregion R5-S2, the substitution position is A47.

Preferably, the amino acid sequence of the glucose isomerase comprises at least one substitution, wherein
from subregion R1-S2-B3, the substitution position is P313; and/or
from subregion R3-S2, the substitution position is V187; and/or
from subregion R5-S2, the substitution position is A47.

Preferably, the amino acid sequence of the glucose isomerase comprises at least one, two, three, four, five or six, seven, eight or nine substitutions, wherein the substitution positions are in any subsequence, and wherein the substitutions are independently of one another selected from the group consisting of subregions R1-S3-A, R1-B1, R1-B2, R1-S3-D, R3-S3, R4-A1, and R5-S3, preferably selected from the group consisting of subregions R1-B2, R3-S3, and R5-S3.

Preferably, the amino acid sequence of the glucose isomerase comprises at least one, two, three, four, five, six, seven, eight or nine substitutions, wherein
from subregion R1-S3-A, the substitution position is A273; and/or
from subregion R1-B1, the substitution position is Q300; and/or
from subregion R1-B2, the substitution positions are independently of one another selected from the group consisting of M309, and P313; and/or
from subregion R1-S3-D, the substitution positions are independently of one another selected from the group consisting of A342, and K343; and/or
from subregion R3-S3, the substitution position is V187; and/or
from subregion R4-A1, the substitution position is W140; and/or
from subregion R5-S3, the substitution position is A47.

Preferably, the amino acid sequence of the glucose isomerase comprises at least one substitution, wherein
from subregion R1-B2, the substitution position is P313; and/or
from subregion R3-S3, the substitution position is V187; and/or
from subregion R5-S3, the substitution position is A47.

Preferably, the amino acid sequence of the glucose isomerase comprises at least one, two, three, four, five or six substitutions, wherein the substitution positions are in any subsequence, and wherein the substitutions are independently of one another selected from the group consisting of subregions R1-S4-B1; R1-S4-B2; R1-S4-D; and R5-S4, preferably selected from the group consisting of subregions R1-S4-B2 and R5-S4.

Preferably, the amino acid sequence of the glucose isomerase comprises at least one, two, three, four, five or six substitutions, wherein
from subregion R1-S4-B1, the substitution position is Q300; and/or
from subregion R1-S4-B2, the substitution positions are independently of one another selected from the group consisting of M309, and P313; and/or
from subregion R1-S4-D, the substitution positions are independently of one another selected from the group consisting of A342, and K343; and/or
from subregion R5-S4, the substitution position is A47.

Preferably, the amino acid sequence of the glucose isomerase comprises at least one substitution, wherein
from subregion R1-S4-B2, the substitution position is P313; and/or
from subregion R5-S4, the substitution position is A47.

Preferably, the amino acid sequence of the glucose isomerase comprises at least one, two, three, four, five, six, seven, eight, nine, ten, eleven, twelve, thirteen, fourteen, fifteen, sixteen or seventeen substitutions at substitution positions independently of one another selected from the group consisting of P18, A47, W140, T147, I163, V187, G190, L198, M241, F242, A273, Q300, M309, P313, Q331, A342, and K343; preferably selected from the group consisting of P18, A47, W140, T147, I163, V187, G190, M241, F242, Q300, M309, P313, Q331, A342, and K343; more preferably selected from the group consisting of A47, W140, V187, G190, M241, F242, Q300, M309, P313, Q331, A342, and K343; and most preferably selected from the group consisting of A47, V187, M241, F242, Q300, M309, P313, A342, and K343, and preferably selected from the group consisting of A47, V187, F242, and P313.

Preferably, the amino acid sequence of the glucose isomerase comprises at least one, two, three, four, five, six, seven, eight, nine, ten, eleven, twelve, thirteen, fourteen, fifteen, sixteen or seventeen substitutions at substitution positions independently of one another selected from the groups consisting of P18, A47, W140, T147, I163, V187, G190, M241, F242, Q300, M309, P313, Q331, A342, and K343; or P18, A47, W140, I163, V187, L198, M241, F242, Q300, P313, Q331, A342, and K343; or P18, A47, W140, T147, I163, V187, G190, L198, M241, F242, A273, Q300, M309, P313, Q331, A342, and K343; or P18, A47, W140, T147, I163, V187, G190, L198, M241, F242, A273, Q300, M309, K343, P313, Q331, and A342.

Preferably, the amino acid sequence of the glucose isomerase comprises at least one, two, three, four, five, six, seven, eight, nine, ten, eleven, twelve, thirteen, fourteen, fifteen, sixteen or seventeen substitutions at substitution positions independently of one another selected from the groups consisting of P18, A47, W140, I163, V187, M241, F242, Q300, P313, Q331, A342, and K343; or A47, V187, M241, F242, Q300, M309, P313, A342, and K343.

Preferably, the amino acid sequence of the glucose isomerase comprises at least one, two, three, four, five, six, seven, eight, nine, ten, eleven, or twelve substitutions at substitution positions independently of one another selected from the group consisting of A47, W140, V187, G190, M241, F242, A273, P313, Q300, M309, A342, and K343.

Preferably, the amino acid sequence of the glucose isomerase comprises at least one substitution at substitution positions independently of one another selected from the group consisting of A47, V187, F242, and P313.

Preferably, the amino acid sequence of the glucose isomerase comprises at least one, two, three, four, five, six, seven, eight, nine, ten, eleven or twelve substitutions, wherein
from the amino acid positions 44, 45, 46, 47, 48, 49, 50, 51, 52, 53, 54, 55, 56, 57, 58, 59, 60, 61, 67, 100, 101, 102, 105, 139, 140, 142, 143, 144, 145, 146, 147, 187, 188, 189, 190, 191, 192, 193, 231, 232, 233, 234, 235, 236, 237, 238, 239, 240, 241, 242, 243, 245, 246, 247, 248, 266, 267, 268, 269, 270, 271, 272, 273, 274, 275, 276, 277, 278, 279, 280, 283, 284, 295, 296, 297, 298, 299, 300, 301, 302, 303, 304, 305, 306, 307, 308, 309, 310, 311, 312, 313, 314, 315, 318, 319, 322, 338, 339, 340, 341, 342, 343, 344, 345, 346, 348, 354, 357, 358, 361, 365, 431, 432, and 435 of SEQ ID NO: 1 the substitution positions are independently of one another selected from the group consisting of A47, W140, V187, G190, M241, F242, A273, Q300, M309, P313, A342, and K343, preferably A47, V187, F242, and P313; and/or
from the amino acid positions 45, 46, 47, 48, 49, 50, 54, 60, 100, 101, 102, 139, 140, 142, 146, 185, 186, 187, 188, 189, 190, 191, 231, 232, 233, 234, 235, 236, 243, 266, 267, 268, 269, 270, 271, 272, 273, 275, 280, 294, 295, 296, 297, 298, 299, 300, 308, 309, 310, 311, 313, 337, 338, 339, 340, 341, 342, and 343 of SEQ ID NO:1 the substitution positions are independently of one another selected from the group consisting of A47, W140, V187, G190, A273, Q300, M309, P313, A342, and K343, preferably A47, V187, and P313; and/or
from the amino acid positions 44, 45, 46, 47, 48, 49, 50, 51, 52, 53, 54, 56, 60, 67, 90, 91, 94, 96, 98, 99, 100, 101, 102, 105, 139, 140, 146, 187, 189, 233, 235, 238, 267, 268, 269, 270, 271, 272, 273, 275, 280, 295, 296, 297, 298, 299, 300, 301, 302, 307, 308, 309, 310, 311, 312, 313, 314, 315, 318, 319, 322, 338, 339, 340, 341, 342, 343, 344, 345, 346, 348, 349, 353, 354, 355, 356, 357, 358, 359, 360, 361, 362, 363, 364, 365, 431, 432, 433, and 435 of SEQ ID NO:1 the substitution positions are independently of one another selected from the group consisting of A47, W140, V187, A273, Q300, M309, P313, A342, and K343, preferably A47, V187, and P313; and/or
from the amino acid positions 47, 50, 269, 271, 297, 298, 299, 300, 309, 310, 311, 312, 313, 339, 340, 341, 342, 343, 344, 345, and 358 of SEQ ID NO: 1 the substitution positions are independently of one another selected from the group consisting of A47, Q300, M309, P313, A342 and K343, preferably A47 and P313.

Preferably, the amino acid sequence of the glucose isomerase comprises at least one, two, three, four, five, six, seven, eight, nine, ten, eleven or twelve substitutions, wherein
from the amino acid positions 44, 45, 46, 47, 48, 49, 50, 51, 52, 53, 54, 55, 56, 57, 58, 59, 60, 61, 67, 139, 140, 187, 188, 189, 190, 191, 192, 231, 232, 233, 234, 235, 236, 237, 238, 239, 240, 241, 242, 243, 266, 267, 268, 269, 270, 271, 272, 273, 274, 275, 276, 277, 278, 279, 280, 295, 296, 297, 298, 299, 300, 301, 302, 303, 304, 305, 306, 307, 308, 309, 310, 311, 312, 313, 314, 315, 338, 339, 340, 341, 342, 343, 344, 345, and 346, of SEQ ID NO: 1 the substitution positions are independently of one another selected from the group consisting of V187, A47, W140, G190, M241, F242, A273, Q300, M309, P313, A342, and K343, preferably V187, A47, F242, and P313; and/or
from the amino acid positions 45, 46, 47, 48, 49, 50, 54, 60, 139, 140, 187, 188, 189, 190, 191, 231, 232, 233, 234, 235, 236, 243, 266, 267, 268, 269, 270, 271, 272, 273, 275, 280, 295, 296, 297, 298, 299, 300, 308, 309, 310, 311, 313, 338, 339, 340, 341, 342, and 343 of SEQ ID NO: 1 the substitution positions are independently of one another selected from the group consisting of V187, A47, W140, G190, A273, Q300, M309, P313, A342, and K343, preferably V187, A47, and P313; and/or
from the amino acid positions 44, 45, 46, 47, 48, 49, 50, 51, 52, 53, 54, 56, 60, 67, 139, 140, 187, 189, 233, 235, 238, 267, 268, 269, 270, 271, 272, 273, 275, 280, 295, 296, 297, 298, 299, 300, 301, 302, 307, 308, 309, 310, 311, 312, 313, 314, 315, 338, 339, 340, 341, 342, 343, 344, 345, and 346 of SEQ ID NO:1 the substitution positions are independently of one another selected from the group consisting of V187, A47, W140, A273, Q300, M309, P313, A342, and K343, preferably V187, A47, and P313;
from the amino acid positions 47, 50, 269, 271, 297, 298, 299, 300, 309, 310, 311, 312, 313, 339, 340, 341, 342, 343, 344, and 345 of SEQ ID NO: 1 the substitution positions are independently of one another selected from the group consisting of A47, Q300, M309, P313, A342 and K343, preferably A47 and P313.

Preferably, the amino acid sequence of the glucose isomerase comprises at least one, two, three, four, five, six, seven, eight, nine, ten, eleven, twelve, thirteen, fourteen, fifteen, sixteen or seventeen substitutions, wherein the at least one further substitution is independently of one another selected from the group consisting of substitution positions P18, A47, W140, T147, I163, V187, G190, L198, M241, F242, A273, Q300, M309, P313, Q331, A342, and K343.

Preferably, the amino acid sequence of the glucose isomerase comprises at least one, two, three, four, five, six, seven, eight, nine, ten, eleven, twelve, thirteen, fourteen, fifteen, sixteen or seventeen substitutions at substitution positions independently of one another selected from the group consisting of A47, W140, V187, G190, M241, F242, A273, Q300, M309, P313, A342, K343; preferably selected from the group consisting of A47, W140, V187, G190, A273, Q300, M309, P313, A342, K343; more preferably selected from the group consisting of A47, W140, V187, A273, Q300, M309, P313, A342, K343, and most preferably selected from the group consisting of A47, Q300, M309, P313, A342, K343.

Preferably, the amino acid sequence of the glucose isomerase comprises at least one substitution at substitution positions independently of one another selected from the group consisting of A47, V187, F242, and P313; preferably selected from the group consisting of A47, V187, and P313.

Preferably, the amino acid sequence of the glucose isomerase comprises at least two, three, four, five, six, seven, eight, nine, ten, eleven, twelve, thirteen, fourteen, fifteen, sixteen or seventeen substitutions, wherein the substitution positions are in any subsequence and independently of one another selected from the groups consisting of subregions R1 and R2, R1 and R3, R1 and R4, R1 and R5, R2 and R3, R2 and R4, R2 and R5, R3 and R4, R3 and R5, and R4 and R5.

Preferably, the amino acid sequence of the glucose isomerase comprises at least two, three, four, five, six, seven, eight, nine, ten, eleven, twelve, thirteen, fourteen, fifteen, sixteen or seventeen substitutions, wherein the substitution positions are in any subsequence and independently of one another selected from the groups consisting of subregions,
wherein the substitutions in subsequence R1 are independently of one another selected from the groups consisting of subregions R1-A and R1-B, R1-A and R1-C, R1-A and R1-D, R1-B and R1-C, R1-B and R1-D, and R1-C and R1-D; or R1-A and R1-B, R1-A and R1-D, R1-B and R1-D; or R1-A and R1-B1, R1-A and R1-B2, R1-A and R1-C, R1-A and R1-D, R1-B1 and R1-B2, R1-B1 and R1-C, R1-B1 and R1-D, R1-B2 and R1-C, R1-B2 and R1-D; or R1-A and R1-B1, R1-A and R1-B2, R1-A and R1-D, R1-B1 and R1-B2, R1-B1 and R1-D, R1-B2 and R1-D; or R1-C and R1-B1, R1-C and R1-B2, R1-C and R1-D, R1-B1 and R1-B2, R1-B1 and R1-D, R1-B2 and R1-D; or R1-B1 and R1-B2, R1-B1 and R1-D, R1-B2 and R1-D; and/or
wherein the substitutions in subsequence R3 are independently of one another selected from the groups consisting of subregions R3-A and R3-B; and/or
wherein the substitutions in subsequence R4 are independently of one another selected from the groups consisting of subregions R4-A and R4-B; or R4-A1, and R4-A2, R4-A1 and R4-B, R4-A2 and R4-B; or R4-A1 and R4-A2; or R4-A1 and R4-B; or R4-A2 and R4-B; or R4-A1 and R4-A2, R4-A1 and R4-B, R4-A2 and R4-B; and/or
wherein the substitutions in subsequence R4 are independently of one another selected from the groups consisting of subregions R5-A and R5-B.

Preferably, the amino acid sequence of the glucose isomerase comprises at least two, three, four, five, six, seven, eight, nine, ten, eleven, twelve, thirteen, fourteen, fifteen, sixteen or seventeen substitutions, wherein the substitution positions are in any subsequence and independently of one another selected from the groups consisting of subregions, wherein the substitutions are individually and in-dependently selected from the groups consisting of subregions R1-B and R1-D, R1-B and R5-B, R1-D and R5-B; or R1-B and R1-D, R1-B and R3-A, R1-D and R3-A; or R1-B and R1-D, R1-B and R3-A, R1-B and R5-B, R1-D and R3-A, R1-D and R5-B, R3-A and R5-B; or R1-B and R1-D, R1-B and R3-A, R1-B and R2, R1-B and R5-B, R1-D and R3-A, R1-D and R2, R1-D and R5-B, R3-A and R2, R3-A and R5-B, R2 and R5-B.

Preferably, the amino acid sequence of the glucose isomerase comprises at least two, three, four, five, six, seven, eight, nine, ten, eleven, twelve, thirteen, fourteen, fifteen, sixteen or seventeen substitutions at substitution positions independently of one another selected from the group consisting of P18, A47, W140, T147, I163, V187, G190, L198, M241, F242, A273, Q300, M309, P313, Q331, A342, and K343; preferably consisting of V187, A47, W140, G190, M241, F242, A273, Q300, M309, P313, A342; most preferably consisting of A47, V187, M241, F242, Q300, M309, P313, A342 and K343, even most preferably consisting of A47, V187, F242, and P313.

Preferably, the amino acid sequence of the glucose isomerase comprises at least two, three, four, five, six, seven, eight, nine, ten, eleven, twelve, thirteen, fourteen, fifteen, sixteen or seventeen substitutions wherein the amino acid sequence comprises at least two substitution positions selected from the group consisting of
P18+A47, P18+W140, P18+T147, P18+I163, P18+V187, P18+G190, P18+L198, P18+F242, P18+M241, P18+A273, P18+Q300, P18+M309, P18+P313, P18+Q331, P18+A342, P18+K343, A47+W140, A47+T147, A47+I163, A47+V187, A47+G190, A47+L198, A47+F242, A47+M241, A47+A273, A47+Q300, A47+M309, A47+P313, A47+Q331, A47+A342, A47+K343, W140+T147, W140+I163, W140+V187, W140+G190, W140+L198, W140+F242, W140+M241, W140+A273, W140+Q300, W140+M309, W140+P313, W140+Q331, W140+A342, W140+K343, T147+I163, T147+V187, T147+G190, T147+L198, T147+F242, T147+M241, T147+A273, T147+Q300, T147+M309, T147+P313, T147+Q331, T147+A342, T147+K343, I163+V187, I163+G190, I163+L198, I163+F242, I163+M241, I163+A273, I163+Q300, I163+M309, I163+P313, I163+Q331, I163+A342, I163+K343, V187+G190, V187+L198, V187+F242, V187+M241, V187+A273, V187+Q300, V187+M309, V187+P313, V187+Q331, V187+A342, V187+K343, G190+L198, G190+F242, G190+M241, G190+A273, G190+Q300, G190+M309, G190+P313, G190+Q331, G190+A342, G190+K343, L198+F242, L198+M241, L198+A273, L198+Q300, L198+M309, L198+P313, L198+Q331, L198+A342, L198+K343, F242+M241, F242+A273, F242+Q300, F242+M309, F242+P313, F242+Q331, F242+A342, F242+K343, M241+A273, M241+Q300, M241+M309, M241+P313, M241+Q331, M241+A342, M241+K343, A273+Q300, A273+M309, A273+P313, A273+Q331, A273+A342, A273+K343, Q300+M309, Q300+P313, Q300+Q331, Q300+A342, Q300+K343, M309+P313, M309+Q331, M309+A342, M309+K343, P313+Q331, P313+A342, P313+K343, Q331+A342, Q331+K343, and A342+K343; and/or
V187+Q300, V187+P18, V187+A47, V187+W140, V187+T147, V187+I163, V187+G190, V187+M241, V187+F242, V187+M309, V187+P313, V187+Q331, V187+A342, V187+K343, P18+A47, P18+W140, P18+T147, P18+I163, P18+G190, P18+M241, P18+F242, P18+Q300, P18+M309, P18+P313, P18+Q331, P18+A342, P18+K343, A47+W140, A47+T147, A47+I163, A47+G190, A47+M241, A47+F242, A47+Q300, A47+M309, A47+P313, A47+Q331, A47+A342, A47+K343, W140+T147, W140+I163, W140+G190, W140+M241, W140+F242, W140+Q300, W140+M309, W140+P313, W140+Q331, W140+A342, W140+K343, T147+I163, T147+G190, T147+M241, T147+F242, T147+Q300, T147+M309, T147+P313, T147+Q331, T147+A342, T147+K343, I163+G190, I163+M241, I163+F242, I163+Q300, I163+M309, I163+P313, I163+Q331, I163+A342, I163+K343, G190+M241, G190+F242, G190+Q300, G190+M309, G190+P313, G190+Q331, G190+A342, G190+K343, M241+F242, M241+Q300, M241+M309, M241+P313, M241+Q331, M241+A342, M241+K343, F242+Q300, F242+M309, F242+P313, F242+Q331, F242+A342, F242+K343, Q300+M309, Q300+P313, Q300+Q331, Q300+A342, Q300+K343, M309+P313, M309+Q331, M309+A342, M309+K343, P313+Q331, P313+A342, P313+K343, Q331+A342, Q331+K343, and A342+K343; and/or
V187+Q300, V187+P18, V187+A47, V187+W140, V187+I163, V187+L198, V187+M241, V187+F242, V187+M309, V187+P313, V187+Q331, V187+A342, V187+K343, P18+A47, P18+W140, P18+I163, P18+L198, P18+M241, P18+F242, P18+Q300, P18+M309, P18+P313, P18+Q331, P18+A342, P18+K343, A47+W140, A47+I163, A47+L198, A47+M241, A47+F242, A47+Q300, A47+M309, A47+P313, A47+Q331, A47+A342, A47+K343, W140+I163, W140+L198, W140+M241, W140+F242, W140+Q300, W140+M309, W140+P313, W140+Q331, W140+A342, W140+K343, I163+L198, I163+M241, I163+F242, I163+Q300, I163+M309, I163+P313, I163+Q331, I163+A342, I163+K343, L198+M241, L198+F242, L198+Q300, L198+M309, L198+P313, L198+Q331, L198+A342, L198+K343, M241+F242, M241+Q300, M241+M309, M241+P313, M241+Q331, M241+A342, M241+K343, F242+Q300, F242+M309, F242+P313, F242+Q331, F242+A342, F242+K343, Q300+M309, Q300+P313, Q300+Q331, Q300+A342, Q300+K343, M309+P313, M309+Q331, M309+A342, M309+K343, P313+Q331, P313+A342, P313+K343, Q331+A342, Q331+K343, and A342+K343; and/or
Q300+P18, Q300+A47, Q300+W140, Q300+T147, Q300+I163, Q300+V187, Q300+G190, Q300+L198, Q300+M241, Q300+F242, Q300+A273, Q300+M309, Q300+K343, Q300+P313, Q300+Q331, Q300+A342, P18+A47, P18+W140, P18+T147, P18+I163, P18+V187, P18+G190, P18+L198, P18+M241, P18+F242, P18+A273, P18+M309, P18+K343, P18+P313, P18+Q331, P18+A342, A47+W140, A47+T147, A47+I163, A47+V187, A47+G190, A47+L198, A47+M241, A47+F242, A47+A273, A47+M309, A47+K343, A47+P313, A47+Q331, A47+A342, W140+T147, W140+I163, W140+V187, W140+G190, W140+L198, W140+M241, W140+F242, W140+A273, W140+M309, W140+K343, W140+P313, W140+Q331, W140+A342, T147+I163, T147+V187, T147+G190, T147+L198, T147+M241, T147+F242, T147+A273, T147+M309, T147+K343, T147+P313, T147+Q331, T147+A342, I163+V187, I163+G190, I163+L198, I163+M241, I163+F242, I163+A273, I163+M309, I163+K343, I163+P313, I163+Q331, I163+A342, V187+G190, V187+L198, V187+M241, V187+F242, V187+A273, V187+M309, V187+K343, V187+P313, V187+Q331, V187+A342, G190+L198, G190+M241, G190+F242, G190+A273, G190+M309, G190+K343, G190+P313, G190+Q331, G190+A342, L198+M241, L198+F242, L198+A273, L198+M309, L198+K343, L198+P313, L198+Q331, L198+A342, M241+F242, M241+A273, M241+M309, M241+K343, M241+P313, M241+Q331, M241+A342, F242+A273, F242+M309, F242+K343, F242+P313, F242+Q331, F242+A342, A273+M309, A273+K343, A273+P313, A273+Q331, A273+A342, M309+K343, M309+P313, M309+Q331, M309+A342, K343+P313, K343+Q331, K343+A342, P313+Q331, P313+A342, Q331+A342.

Preferably, the amino acid sequence of the glucose isomerase comprises at least two, three, four, five, six, seven, eight, nine, ten, eleven, twelve, thirteen, fourteen, fifteen, sixteen or seventeen substitutions wherein the amino acid sequence comprises at least two substitution positions selected from the group consisting of A47+V187, A47+M241, A47+F242, A47+Q300, A47+M309, A47+P313, A47+A342, A47+K343, V187+M241, V187+F242, V187+Q300, V187+M309, V187+P313, V187+A342, V187+K343, M241+F242, M241+Q300, M241+M309, M241+P313, M241+A342, M241+K343, F242+Q300, F242+M309, F242+P313, F242+A342, F242+K343, Q300+M309, Q300+P313, Q300+A342, Q300+K343, M309+P313, M309+A342, M309+K343, P313+A342, P313+K343, and A342+K343.

Preferably, the amino acid sequence of the glucose isomerase comprises at least two, three, four, five, six, seven, eight, nine, ten, eleven, twelve, thirteen, fourteen, fifteen, sixteen or seventeen substitutions, wherein the substitution positions are in any subsequence and independently of one another selected from the groups consisting of subsequences R1 and R2 and R3, R1 and R2 and R4, R1 and R2 and R5, R1 and R3 and R4, R1 and R3 and R5, R1 and R4 and R5, R2 and R3 and R4, R2 and R3 and R5, R2 and R4 and R5, and R3 and R4 and R5.

Preferably, the amino acid sequence of the glucose isomerase comprises at least two, three, four, five, six, seven, eight, nine, ten, eleven, twelve, thirteen, fourteen, fifteen, sixteen or seventeen substitutions, wherein the substitution positions are in any subsequence and independently of one another selected from the groups consisting of subregions,
wherein the substitutions in subsequence R1 are independently of one another selected from the groups consisting of
   R1-A and R1-B and R1-C, R1-A and R1-B and R1-D, R1-A and R1-C and R1-D, R1-B and R1-C and R1-D; or
   R1-A and R1-B and R1-D; or
   R1-A and R1-B1 and R1-B2, R1-A and R1-B1 and R1-D, R1-A and R1-B2 and R1-D, R1-B1 and R1-B2 and R1-D, R1-A and R1-B1 and R1-C, R1-A and R1-B2 and R1-C, R1-B1 and R1-B2 and R1-C, R1-B1 and R1-C and R1-D, R1-B2 and R1-C and R1-D; or
   R1-A and R1-B1 and R1-B2, R1-A and R1-B1 and R1-D, R1-A and R1-B2 and R1-D, R1-B1 and R1-B2 and R1-D; or
   R1-C and R1-B1 and R1-B2, R1-C and R1-B1 and R1-D, R1-C and R1-B2 and R1-D, R1-B1 and R1-B2 and R1-D; or
   R1-B1 and R1-B2 and R1-D1; and/or
wherein the substitutions in subsequence R4 are independently of one another selected from the groups consisting of R4-A1 and R4-A2 and R4-B.

Preferably, the amino acid sequence of the glucose isomerase comprises at least two, three, four, five, six, seven, eight, nine, ten, eleven, twelve, thirteen, fourteen, fifteen, sixteen or seventeen substitutions, wherein the substitution positions are in any subsequence and independently of one another selected from the groups consisting of subregions,
R1-B and R1-D and R5-B; or
R1-B and R1-D and R3-A, R1-B and R1-D and R5-B, R1-B and R3-A and R5-B, R1-D and R3-A and R5-B; or
R1-B and R1-D and R3-A, R1-B and R1-D and R2, R1-B and R1-D and R5-B, R1-B and R3-A and R2, R1-B and R3-A and R5-B, R1-B and R2 and R5-B, R1-D and R3-A and R2, R1-D and R3-A and R5-B, R1-D and R2 and R5-B, R3-A and R2 and R5-B.

Preferably, the amino acid sequence of the glucose isomerase comprises at least three, four, five, six, seven, eight, nine, ten, eleven, twelve, thirteen, fourteen, fifteen, sixteen or seventeen substitutions at substitution positions independently of one another selected from the group consisting of P18, A47, W140, T147, I163, V187, G190, L198, M241, F242, A273, Q300, M309, P313, Q331, A342, and K343; preferably consisting of A47, V187, M241, F242, Q300, M309, P313, A342 and K343.

Preferably, the amino acid sequence of the glucose isomerase comprises at least three, four, five, six, seven, eight, nine, ten, eleven, twelve, thirteen, fourteen, fifteen, sixteen or seventeen substitutions wherein the amino acid sequence comprises at least three substitution positions selected from the group consisting of V187+A47+M241, V187+A47+F242, V187+A47+Q300, V187+A47+M309, V187+A47+P313, V187+A47+A342, V187+A47+K343, V187+M241+F242, V187+M241+Q300, V187+M241+M309, V187+M241+P313, V187+M241+A342, V187+M241+K343, V187+F242+Q300, V187+F242+M309, V187+F242+P313, V187+F242+A342, V187+F242+K343, V187+Q300+M309, V187+Q300+P313, V187+Q300+A342, V187+Q300+K343, V187+M309+P313, V187+M309+A342, V187+M309+K343, V187+P313+A342, V187+P313+K343, V187+A342+K343, A47+M241+F242, A47+M241+Q300, A47+M241+M309, A47+M241+P313, A47+M241+A342, A47+M241+K343, A47+F242+Q300, A47+F242+M309, A47+F242+P313, A47+F242+A342, A47+F242+K343, A47+Q300+M309, A47+Q300+P313, A47+Q300+A342, A47+Q300+K343, A47+M309+P313, A47+M309+A342, A47+M309+K343, A47+P313+A342, A47+P313+K343, A47+A342+K343, M241+F242+Q300, M241+F242+M309, M241+F242+P313, M241+F242+A342, M241+F242+K343, M241+Q300+M309, M241+Q300+P313, M241+Q300+A342, M241+Q300+K343, M241+M309+P313, M241+M309+A342, M241+M309+K343, M241+P313+A342, M241+P313+K343, M241+A342+K343, F242+Q300+M309, F242+Q300+P313, F242+Q300+A342, F242+Q300+K343, F242+M309+P313, F242+M309+A342, F242+M309+K343, F242+P313+A342, F242+P313+K343, F242+A342+K343, Q300+M309+P313, Q300+M309+A342, Q300+M309+K343, Q300+P313+A342, Q300+P313+K343, Q300+A342+K343, M309+P313+A342, M309+P313+K343, M309+A342+K343, and P313+A342+K343.

Preferably, the amino acid sequence of the glucose isomerase comprises at least four, five, six, seven, eight, nine, ten, eleven, twelve, thirteen, fourteen, fifteen, sixteen or seventeen substitutions at substitution positions independently of one another selected from the group consisting of P18, A47, W140, T147, I163, V187, G190, L198, M241, F242, A273, Q300, M309, P313, Q331, A342, and K343; preferably consisting of A47, V187, M241, F242, Q300, M309, P313, A342 and K343.

Preferably, the amino acid sequence of the glucose isomerase comprises at least one substitution at substitution position V187, and further comprises at least one, two, three, four, five or six substitutions at substitution positions independently of one another selected from the group consisting of A47, M241, F242, Q300, M309, P313, A342 and K343.

Preferably, the amino acid sequence of the glucose isomerase comprises at least one substitution at substitution position A47, and further comprises at least one, two, three, four, five or six substitutions at substitution positions independently of one another selected from the group consisting of V187, M241, F242, Q300, M309, P313, A342 and K343.

Preferably, the amino acid sequence of the glucose isomerase comprises at least one substitution at substitution position M241, and further comprises at least one, two, three, four, five or six substitutions at substitution positions independently of one another selected from the group consisting of V187, A47, F242, Q300, M309, P313, A342 and K343.

Preferably, the amino acid sequence of the glucose isomerase comprises at least one substitution at substitution position F242, and further comprises at least one, two, three, four, five or six substitutions at substitution positions independently of one another selected from the group consisting of V187, A47, M241, Q300, M309, P313, A342 and K343.

Preferably, the amino acid sequence of the glucose isomerase comprises at least one substitution at substitution position Q300, and further comprises at least one, two, three, four, five or six substitutions at substitution positions independently of one another selected from the group consisting of V187, A47, M241, F242, M309, P313, A342 and K343.

Preferably, the amino acid sequence of the glucose isomerase comprises at least one substitution at substitution position M309, and further comprises at least one, two, three, four, five or six substitutions at substitution positions independently of one another selected from the group consisting of V187, A47, M241, F242, Q300, P313, A342 and K343.

Preferably, the amino acid sequence of the glucose isomerase comprises at least one substitution at substitution position P313, and further comprises at least one, two, three, four, five or six substitutions at substitution positions independently of one another selected from the group consisting of V187, A47, M241, F242, Q300, M309, A342 and K343.

Preferably, the amino acid sequence of the glucose isomerase comprises at least one substitution at substitution position A342, and further comprises at least one, two, three, four, five or six substitutions at substitution positions independently of one another selected from the group consisting of V187, A47, M241, F242, Q300, M309, P313 and K343.

Preferably, the amino acid sequence of the glucose isomerase comprises at least one substitution at substitution position K343, and further comprises at least one, two, three, four, five or six substitutions at substitution positions independently of one another selected from the group consisting of V187, A47, M241, F242, Q300, M309, P313, and A342.

Preferably, the amino acid sequence of the glucose isomerase comprises a substitution in position 18, the substitution being selected from the group consisting of P18S, P18A, P18R, P18N, P18D, P18C, P18Q, P18E, P18G, P18H, P18I, P18L, P18K, P18M, P18F, P18T, P18W, P18V or P18Y; more preferably P18S, P18T, P18Y; and most preferably P18S.

Preferably, the amino acid sequence of the glucose isomerase comprises a substitution in position 47, the substitution being selected from the group consisting of A47S, A47C, A47R, A47N, A47D, A47Q, A47E, A47G, A47H, A47I, A47L, A47K, A47M, A47F, A47P, A47T, A47W, A47V, or A47Y; preferably A47S, A47Q or A47R; and more preferably A47S.

Preferably, the amino acid sequence of the glucose isomerase comprises a substitution in position 140, the substitution being selected from the group consisting of W140H, W140C, W140A, W140R, W140N, W140D, W140Q, W140E, W140G, W140I, W140L, W140K, W140M, W140F, W140P, W140S, W140T, W140V or W140Y; preferably W140H, W140R, W140K; more preferably W140H.

Preferably, the amino acid sequence of the glucose isomerase comprises a substitution in position 147, the substitution being selected from the group consisting of T147R, T147C, T147A, T147N, T147D, T147E, T147G, T147H, T147I, T147L, T147K, T147M, T147F, T147P, T147S, T147W, T147V or T147Y; preferably T147R.

Preferably, the amino acid sequence of the glucose isomerase comprises a substitution in position 163, the substitution being selected from the group consisting of I163V, I163C, I163A, I163R, I163N, I163D, I163Q, I163E, I163G, I163H, I163L, I163K, I163M, I163F, I163P, I163S, I163T, I163W, or I163Y; preferably I163V.

Preferably, the amino acid sequence of the glucose isomerase comprises a substitution in position 187, the substitution being selected from the group consisting of V187C, V187A, V187R, V187N, V187D, V187Q, V187E, V187G, V187H, V187I, V187L, V187K, V187M, V187F, V187P, V187S, V187T, V187W, or V187Y; V187C, V187T, V187W, V187S, V187Y; preferably V187C, V187T, or V187W; and more preferably V187C.

Preferably, the amino acid sequence of the glucose isomerase comprises a substitution in position 190, the substitution being selected from the group consisting of G190N, G190C, G190A, G190R, G190D, G190Q, G190E, G190H, G190I, G190L, G190K, G190M, G190F, G190P, G190S, G190T, G190W or G190Y, preferably G190Q or G109N and more preferably G190N.

Preferably, the amino acid sequence of the glucose isomerase comprises a substitution in position 198, the substitution being selected from the group consisting of L198I, L198C, L198A, L198R, L198N, L198D, L198Q, L198E, L198G, L198H, L198K, L198M, L198F, L198P, L198S, L198T, L198W, L198V or L198Y; preferably L198I, L198V; more preferably L198I.

Preferably, the amino acid sequence of the glucose isomerase comprises a substitution in position 241, the substitution being selected from the group consisting of M241V, M241C, M241A, M241R, M241N, M241D, M241Q, M241E, M241G, M241H, M241I, M241L, M241K, M241F, M241P, M241S, M241T, M241W, or M241Y; preferably M241V, M241C, M241R, M241D, M241Q, M241E, M241G, M241H, M241I, M241K, M241F, M241P, M241W, or M241 Y; more preferably M241P, M241V, M241C, M241I, M241S, M241T, M241L; preferably M241V or M241C; most preferably M241V.

Preferably, the amino acid sequence of the glucose isomerase comprises a substitution in position 242, the substitution being selected from the group consisting of F242C, F242A, F242R, F242N, F242D, F242Q, F242E, F242G, F242H, F242I, F242L, F242K, F242M, F242P, F242S, F242T, F242W, F242V or F242Y; preferably F242C, F242A, F242R, F242N, F242D, F242Q, F242E, F242G, F242I, F242K, F242M, F242P, F242S, F242T, F242W, F242V, or F242Y; more preferably F242C, F242V, F242I, F242L, F242P; most preferably F242C or F242V; most preferably F242C.

Preferably, the amino acid sequence of the glucose isomerase comprises a substitution in position 273, the substitution being selected from the group consisting of A273S, A273C, A273R, A273N, A273D, A273Q, A273E, A273G, A273H, A273I, A273L, A273K, A273M, A273F, A273P, A273T, A273W, A273V or A273Y; preferably A273S, A273T or A273Y; more preferably A273S.

Preferably, the amino acid sequence of the glucose isomerase comprises a substitution in position 300, the substitution being selected from the group consisting of Q300Y, Q300C, Q300A, Q300R, Q300N, Q300D, Q300E, Q300G, Q300H, Q300I, Q300L, Q300K, Q300M, Q300F, Q300P, Q300S, Q300T, Q300W or Q300V; preferably Q300Y, Q300C, Q300A, Q300R, Q300N, Q300D, Q300G, Q300H, Q300I, Q300L, Q300K, Q300M, Q300F, Q300P, Q300S, Q300T, Q300W, or Q300V; more preferably Q300Y, Q300S, Q300C or Q300F; most preferably Q300Y.

Preferably, the amino acid sequence of the glucose isomerase comprises a substitution in position 309, the substitution being selected from the group consisting of M309I, M309C, M309A, M309R, M309N, M309D, M309Q, M309E, M309G, M309H, M309L, M309K, M309F, M309P, M309S, M309T, M309W, M309V or M309Y; preferably M309I, M309V, M309L, M309P; more preferably M309I or M309P; most preferably M309I.

Preferably, the amino acid sequence of the glucose isomerase comprises a substitution in position 313, the substitution being selected from the group consisting of P313S, P313C, P313A, P313R, P313N, P313D, P313Q, P313E, P313G, P313H, P313I, P313L, P313K, P313M, P313F, P313T, P313W, P313V or P313Y; preferably P313S, P313T, P313Y, P313V, P313L, P313I and P313G; more preferably P313S, P313V and P313G; most preferably P313S.

Preferably, the amino acid sequence of the glucose isomerase comprises a substitution in position 331, the substitution being selected from the group consisting of Q331G, Q331C, Q331A, Q331R, Q331N, Q331D, Q331E, Q331H, Q331I, Q331L, Q331K, Q331M, Q331F, Q331P, Q331S, Q331T, Q331W, Q331V or Q331Y; preferably Q331G.

Preferably, the amino acid sequence of the glucose isomerase comprises a substitution in position 342, the substitution being selected from the group consisting of A342S, A342C, A342R, A342N, A342D, A342Q, A342E, A342G, A342H, A342I, A342L, A342K, A342M, A342F, A342P, A342T, A342W, A342V or A342Y; preferably A342S, A342T, A342Y; more preferably A342S.

Preferably, the amino acid sequence of the glucose isomerase comprises a substitution in position 343, the substitution being selected from the group consisting of K343R, K343C, K343A, K343N, K343D, K343Q, K343E, K343G, K343H, K343I, K343L, K343M, K343F, K343P, K343S, K343T, K343W, K343V or K343Y; preferably K343R, K343N, K343Q; more preferably K343R or K343N; most preferably K343R.

Preferably, the amino acid sequence of the glucose isomerase comprises at least one, two, three, four, five, six, seven, eight, nine, ten, eleven, twelve, thirteen, fourteen, fifteen, sixteen or seventeen substitutions at substitution positions independently of one another selected from the group consisting of
position 18, the substitution being selected from the group consisting of P18S, P18A, P18R, P18N, P18D, P18C, P18Q, P18E, P18G, P18H, P18I, P18L, P18K, P18M, P18F, P18T, P18W, P18V or P18Y; more preferably P18S, P18T, P18Y; and most preferably P18S; and/or
position 47, the substitution being selected from the group consisting of A47S, A47C, A47R, A47N, A47D, A47Q, A47E, A47G, A47H, A47I, A47L, A47K, A47M, A47F, A47P, A47T, A47W, A47V, or A47Y; preferably A47S, A47Q or A47R; and more preferably A47S; and/or
position 140, the substitution being selected from the group consisting of W140H, W140C, W140A, W140R, W140N, W140D, W140Q, W140E, W140G, W140I, W140L, W140K, W140M, W140F, W140P, W140S, W140T, W140V or W140Y; preferably W140H, W140R, W140K; more preferably W140H; and/or
position 147, the substitution being selected from the group consisting of T147R, T147C, T147A, T147N, T147D, T147E, T147G, T147H, T147I, T147L, T147K, T147M, T147F, T147P, T147S, T147W, T147V or T147Y; preferably T147R; and/or
position 163, the substitution being selected from the group consisting of I163V, I163C, I163A, I163R, I163N, I163D, I163Q, I163E, I163G, I163H, I163L, I163K, I163M, I163F, I163P, I163S, I163T, I163W, or I163Y; preferably I163V; and/or
position 187, the substitution being selected from the group consisting of V187C, V187A, V187R, V187N, V187D, V187Q, V187E, V187G, V187H, V187I, V187L, V187K, V187M, V187F, V187P, V187S, V187T, V187W, or V187Y; preferably V187C or V187T, or V187W; and more preferably V187C; and/or
position 190, the substitution being selected from the group consisting of G190N, G190C, G190A, G190R, G190D, G190Q, G190E, G190H, G190I, G190L, G190K, G190M, G190F, G190P, G190S, G190T, G190W, or G190Y; preferably G190Q or G109N; and/or
position 198, the substitution being selected from the group consisting of L198I, L198C, L198A, L198R, L198N, L198D, L198Q, L198E, L198G, L198H, L198K, L198M, L198F, L198P, L198S, L198T, L198W, L198V or L198Y; preferably L198I; and/or
position 241, the substitution being selected from the group consisting of M241V, M241C, M241A, M241R, M241N, M241D, M241Q, M241E, M241G, M241H, M241I, M241L, M241K, M241F, M241P, M241S, M241T, M241W, or M241Y; preferably M241V, M241C, M241R, M241D, M241Q, M241E, M241G, M241H, M241I, M241K, M241F, M241P, M241W, or M241Y; more preferably M241V or M241C; most preferably M241V; and/or
position 242, the substitution being selected from the group consisting of F242C, F242A, F242R, F242N, F242D, F242Q, F242E, F242G, F242H, F242I, F242L, F242K, F242M, F242P, F242S, F242T, F242W, F242V or F242Y preferably F242C, F242A, F242R, F242N, F242D, F242Q, F242E, F242G, F242I, F242K, F242M, F242P, F242S, F242T, F242W, F242V, or F242Y; more preferably F242C or F242V; most preferably F242C; and/or
position 273, the substitution being selected from the group consisting of A273S, A273C, A273R, A273N, A273D, A273Q, A273E, A273G, A273H, A273I, A273L, A273K, A273M, A273F, A273P, A273T, A273W, A273V or A273Y; preferably A273S, A273T or A273Y; and/or
position 300, the substitution being selected from the group consisting of Q300Y, Q300C, Q300A, Q300R, Q300N, Q300D, Q300E, Q300G, Q300H, Q300I, Q300L, Q300K, Q300M, Q300F, Q300P, Q300S, Q300T, Q300W or Q300V; preferably Q300Y, Q300C, Q300A, Q300R, Q300N, Q300D, Q300G, Q300H, Q300I, Q300L, Q300K, Q300M, Q300F, Q300P, Q300S, Q300T, Q300W, or Q300V; more preferably Q300Y, Q300S, Q300C or Q300F; most preferably Q300Y; and/or
position 309, the substitution being selected from the group consisting of M309I, M309C, M309A, M309R, M309N, M309D, M309Q, M309E, M309G, M309H, M309L, M309K, M309F, M309P, M309S, M309T, M309W, M309V or M309Y; preferably M309I, M309V, M309L, M309P; more preferably M309I or M309P; most preferably M309I; and/or
position 313, the substitution being selected from the group consisting of P313S, P313C, P313A, P313R, P313N, P313D, P313Q, P313E, P313G, P313H, P313I, P313L, P313K, P313M, P313F, P313T, P313W, P313V or P313Y; preferably P313S, P313V and P313G; more preferably P313S; and/or
position 331, the substitution being selected from the group consisting of Q331G, Q331C, Q331A, Q331R, Q331N, Q331D, Q331E, Q331H, Q331I, Q331L, Q331K, Q331M, Q331F, Q331P, Q331S, Q331T, Q331W, Q331V or Q331Y; preferably Q331G; and/or
position 342, the substitution being selected from the group consisting of A342S, A342C, A342R, A342N, A342D, A342Q, A342E, A342G, A342H, A342I, A342L, A342K, A342M, A342F, A342P, A342T, A342W, A342V or A342Y; preferably A342S, A342T, A342Y; more preferably A342S; and/or
position 343, the substitution being selected from the group consisting of K343R, K343C, K343A, K343N, K343D, K343Q, K343E, K343G, K343H, K343I, K343L, K343M, K343F, K343P, K343S, K343T, K343W, K343V or K343Y; preferably K343R, K343N, K343Q; more preferably K343R or K343N; most preferably K343R.

Preferably, the amino acid sequence of the glucose isomerase comprises at least one, two, three, four, five, six, seven, eight, nine, ten, eleven, twelve substitutions at substitution positions independently of one another selected from the group consisting of
position 47, the substitution being selected from the group consisting of A47S, A47C, A47R, A47N, A47D, A47Q, A47E, A47G, A47H, A47I, A47L, A47K, A47M, A47F, A47P, A47T, A47W, A47V, or A47Y; preferably A47S, A47Q or A47R; and more preferably A47S; and/or
position 140, the substitution being selected from the group consisting of W140H, W140C, W140A, W140R, W140N, W140D, W140Q, W140E, W140G, W140I, W140L, W140K, W140M, W140F, W140P, W140S, W140T, W140V or W140Y; preferably W140H, W140R, W140K; more preferably W140H; and/or
position 187, the substitution being selected from the group consisting of V187C, V187A, V187R, V187N, V187D, V187Q, V187E, V187G, V187H, V187I, V187L, V187K, V187M, V187F, V187P, V187S, V187T, V187W, or V187Y; preferably V187C or V187T, or V187W; and more preferably V187C; and/or
position 190, the substitution being selected from the group consisting of G190N, G190C, G190A, G190R, G190D, G190Q, G190E, G190H, G190I, G190L, G190K, G190M, G190F, G190P, G190S, G190T, G190W, or G190Y; preferably G190Q or G109N; and/or
position 241, the substitution being selected from the group consisting of M241V, M241C, M241A, M241R, M241N, M241D, M241Q, M241E, M241G, M241H, M241I, M241L, M241K, M241F, M241P, M241S, M241T, M241W, or M241Y; preferably M241V, M241C, M241R, M241D, M241Q, M241E, M241G, M241H, M241I, M241K, M241F, M241P, M241W, or M241Y; more preferably M241V or M241C; most preferably M241V; and/or
position 242, the substitution being selected from the group consisting of F242C, F242A, F242R, F242N, F242D, F242Q, F242E, F242G, F242H, F242I, F242L, F242K, F242M, F242P, F242S, F242T, F242W, F242V or F242Y preferably F242C, F242A, F242R, F242N, F242D, F242Q, F242E, F242G, F242I, F242K, F242M, F242P, F242S, F242T, F242W, F242V, or F242Y; more preferably F242C or F242V; most preferably F242C; and/or
position 273, the substitution being selected from the group consisting of A273S, A273C, A273R, A273N, A273D, A273Q, A273E, A273G, A273H, A273I, A273L, A273K, A273M, A273F, A273P, A273T, A273W, A273V or A273Y; preferably A273S, A273T or A273Y; and/or
position 300, the substitution being selected from the group consisting of Q300Y, Q300C, Q300A, Q300R, Q300N, Q300D, Q300E, Q300G, Q300H, Q300I, Q300L, Q300K, Q300M, Q300F, Q300P, Q300S, Q300T, Q300W or Q300V; preferably Q300Y, Q300C, Q300A, Q300R, Q300N, Q300D, Q300G, Q300H, Q300I, Q300L, Q300K, Q300M, Q300F, Q300P, Q300S, Q300T, Q300W, or Q300V; more preferably Q300Y, Q300S, Q300C or Q300F; most preferably Q300Y; and/or
position 309, the substitution being selected from the group consisting of M309I, M309C, M309A, M309R, M309N, M309D, M309Q, M309E, M309G, M309H, M309L, M309K, M309F, M309P, M309S, M309T, M309W, M309V or M309Y; preferably M309I, M309V, M309L, M309P; more preferably M309I or M309P; most preferably M309I; and/or
position 313, the substitution being selected from the group consisting of P313S, P313C, P313A, P313R, P313N, P313D, P313Q, P313E, P313G, P313H, P313I, P313L, P313K, P313M, P313F, P313T, P313W, P313V or P313Y; preferably P313S, P313V and P313G; more preferably P313S; and/or
position 342, the substitution being selected from the group consisting of A342S, A342C, A342R, A342N, A342D, A342Q, A342E, A342G, A342H, A342I, A342L, A342K, A342M, A342F, A342P, A342T, A342W, A342V or A342Y; preferably A342S, A342T, A342Y; more preferably A342S; and/or
position 343, the substitution being selected from the group consisting of K343R, K343C, K343A, K343N, K343D, K343Q, K343E, K343G, K343H, K343I, K343L, K343M, K343F, K343P, K343S, K343T, K343W, K343V or K343Y; preferably K343R, K343N, K343Q; more preferably K343R or K343N; most preferably K343R.

Preferably, the amino acid sequence of the glucose isomerase comprises at least one, two, three, four, five, six, seven, eight, nine, ten, eleven, twelve substitutions at substitution positions independently of one another selected from the group consisting of
position 47, the substitution being selected from the group consisting of A47S, A47N, A47Q, A47T, A47C, or A47M; preferably A47S; and/or
position 187, the substitution being selected from the group consisting of V187C, V187T, V187N, V187Q, V187S or V187M; preferably V187C or V187T, and more preferably V187C; and/or
position 242, the substitution being selected from the group consisting of F242C, F242T, F242N, F242Q, F242S or F242M; preferably F242C; and/or
position 313, the substitution being selected from the group consisting of P313S, P313N, P313Q, P313T, P313C or P313M; preferably P313S.

Preferably, the amino acid sequence of the glucose isomerase comprises at least one, two, three, four, five, six, seven, eight, nine, ten, eleven, twelve, thirteen, fourteen, fifteen, sixteen or seventeen substitutions at substitution positions independently of one another selected from the group consisting of
position 18, the substitution being selected from of P18S, P18T or P18Y; more preferably the substitution being P18S; and/or
position 47, the substitution being selected from the group consisting of A47S, A47Q or A47R; preferably A47S; and/or
position 140, the substitution being W140H; and/or
position 147, the substitution being T147R; and/or
position 163, the substitution being I163V; and/or
position 187, the substitution being selected from the group consisting of V187C, or V187T, or V187W; preferably V187C; and/or
position 190, the substitution being G190N; and/or
position 198, the substitution being L198I; and/or
position 241, the substitution being M241V or M241C; preferably M241V; and/or
position 242, the substitution being F242C or F242V; preferably F242C; and/or
position 273, the substitution being A273S; and/or
position 300, the substitution being selected from the group consisting of Q300Y, Q300S, Q300C or Q300F; preferably Q300Y or Q300S, most preferably Q300Y; and/or
position 309, the substitution being M309I; and/or
position 313, the substitution being selected from the group consisting of P313S, P313V and P313G; preferably P313S; and/or
position 331, the substitution being Q331G; and/or
position 342, the substitution being A342S; and/or
position 343, the substitution being K343R or K343N; preferably K343R.

Preferably, the amino acid sequence of the glucose isomerase comprises at least one, two, three, four, five, six, seven, eight, nine, ten, eleven, twelve, thirteen, fourteen, fifteen, sixteen or seventeen substitutions independently of one another selected from P18S, A47S, W140H, T147R, I163V, V187C, V187T, V187W, G190N, L198I, M241V, F242C, F242V, A273S, Q300Y, Q300S, Q300C, Q300F, M309I, P313S, P313V, P313G, Q331G, A342S, K343N and K343R; preferably selected from the group consisting of P18S, A47S, W140H, T147R, I163V, V187C, V187T, V187W, G190N, M241V, F242C, F242V, Q300Y, Q300S, Q300C, Q300F, M309I, P313S, P313V, P313G, Q331G, A342S, K343N and K343R; more preferably selected from the group consisting of A47S, W140H, V187C, V187T, V187W, G190N, M241V, F242C, F242V, Q300Y, Q300S, Q300C, Q300F, M309I, P313S, P313V, P313G, Q331G, A342S, K343N and K343R; and most preferably selected from the group consisting of A47S, V187C, V187T, V187W, M241V, F242C, F242V, Q300Y, Q300S, Q300C, Q300F, M309I, P313S, P313V, P313G, A342S, K343N and K343R.

Preferably, the amino acid sequence of the glucose isomerase comprises at least one, two, three, four, five, six, seven, eight, nine, ten, eleven or twelve substitutions independently of one another selected from V187C, V187T, V187W, P313S, P313V, P313G, Q300Y, Q300S, Q300C, Q300F, A47S, W140H, G190N, M241V, F242C, F242V, A273S, M309I, A342S, K343N and K343R; preferably selected from the group consisting of V187C, V187T, V187W, P313S, P313V, P313G, Q300Y, Q300S, Q300C, Q300F, A47S, M241V, F242C, F242V, M309I, A342S, K343N and K343R; and more preferably selected from the group consisting of V187C, V187T, V187W, P313S, P313V, P313G, Q300Y, Q300S, Q300C, and Q300F.

Preferably, the amino acid sequence of the glucose isomerase comprises at least one, two, three, four, five, six, seven, eight, nine, ten, eleven, twelve, thirteen, fourteen, fifteen, sixteen or seventeen substitutions independently of one another selected from
V187C, P18S, A47S, W140H, T147R, I163V, G190N, M241V, F242C, Q300Y, M309I, P313S, Q331G, A342S, and K343R; or
V187C, P18S, A47S, W140H, I163V, L198I, M241V, F242C, Q300Y, M309I, P313S, Q331G, K343R, and A342S; or
V187C, P18S, A47S, W140H, I163V, G190N, L198I, M241V, F242C, A273S, Q300Y, M309I, P313V, Q331G, A342S, and K343R; or
Q300Y, P18S, A47S, W140H, T147R, I163V, V187C, G190N, L198I, M241V, F242C, A273S, M309I, K343R, P313V, Q331G, and A342S.

Preferably, the amino acid sequence of the glucose isomerase comprises at least one, two, three, four, five, six, seven, eight, nine, ten, eleven, twelve, thirteen, fourteen, fifteen, sixteen or seventeen substitutions independently of one another selected from
P18S, A47S, W140H, I163V, V187C, M241V, F242C, Q300Y, P313V, Q331G, A342S, and K343R; or
A47S, V187C, V187T, G V187W, M241V, F242C, F242V, Q300Y, Q300S, Q300C, Q300F, M309I, P313S, P313V, P313G, A342S, K343N and K343R; preferably A47S, V187C, M241V, F242C, Q300Y, M309I, P313S, A342S, and K343R

Preferably, the amino acid sequence of the glucose isomerase comprises at least one, two, three, four, five, six, seven, eight, nine, ten, eleven, twelve, thirteen, fourteen, fifteen, sixteen or seventeen substitutions independently of one another selected from P18S, P18T, P18Y, A47S, A47Q, A47R, W140H, W140R, W140K, T147R, I163V, V187C, V187T, V187W, G190Q, G109N, L198I, L198V, M241P, M241V, M241C, M241I, M241S, M241T, M241L, F242C, F242V, F242I, F242L, F242P, A273S, A273T, A273Y, Q300Y, Q300S, Q300C, Q300F, M309I, M309V, M309L, M309P, P313S, P313T, P313Y, P313V, P313L, P313I, P313G, Q331G, A342S, A342T, A342Y, K343R, K343N, K343Q; preferably of P18S, A47S, W140H, T147R, I163V, V187C, G190N, L198I, M241V, M241C, F242C, F242V, A273S, Q300Y, M309I, P313S, P313V, P313G, Q331G, A342S, K343R, K343N.

Preferably, the amino acid sequence of the glucose isomerase comprises at least one substitution selected from A47S, V187C, V187T, F242C, and P313S; preferably selected from the group consisting of A47S, V187C, F242C, and P313S; more preferably selected from the group consisting of A47S, V187C, and P313S.

Preferably, the amino acid sequence of the glucose isomerase comprises at least one substitution wherein the at least one substitution is V187C.

Preferably, the amino acid sequence of the glucose isomerase comprises at least two, three, four, five, six, seven, eight, nine, ten, eleven, twelve, thirteen, fourteen, fifteen, sixteen or seventeen substitutions at substitution positions, wherein the amino acid sequence comprises at least two substitutions selected from
V187C+Q300Y, V187C+P18S, V187C+A47S, V187C+W140H, V187C+T147R, V187C+I163V, V187C+G190N, V187C+M241V, V187C+F242C, V187C+M309I, V187C+P313S, V187C+Q331G, V187C+A342S, V187C+K343R, P18S+A47S, P18S+W140H, P18S+T147R, P18S+I163V, P18S+G190N, P18S+M241V, P18S+F242C, P18S+Q300Y, P18S+M309I, P18S+P313S, P18S+Q331G, P18S+A342S, P18S+K343R, A47S+W140H, A47S+T147R, A47S+I163V, A47S+G190N, A47S+M241V, A47S+F242C, A47S+Q300Y, A47S+M309I, A47S+P313S, A47S+Q331G, A47S+A342S, A47S+K343R, W140H+T147R, W140H+I163V, W140H+G190N, W140H+M241V, W140H+F242C, W140H+Q300Y, W140H+M309I, W140H+P313S, W140H+Q331G, W140H+A342S, W140H+K343R, T147R+I163V, T147R+G190N, T147R+M241V, T147R+F242C, T147R+Q300Y, T147R+M309I, T147R+P313S, T147R+Q331G, T147R+A342S, T147R+K343R, I163V+G190N, I163V+M241V, I163V+F242C, I163V+Q300Y, I163V+M309I, I163V+P313S, I163V+Q331G, I163V+A342S, I163V+K343R, G190N+M241V, G190N+F242C, G190N+Q300Y, G190N+M309I, G190N+P313S, G190N+Q331G, G190N+A342S, G190N+K343R, M241V+F242C, M241V+Q300Y, M241V+M309I, M241V+P313S, M241V+Q331G, M241V+A342S, M241V+K343R, F242C+Q300Y, F242C+M309I, F242C+P313S, F242C+Q331G, F242C+A342S, F242C+K343R, Q300Y+M309I, Q300Y+P313S, Q300Y+Q331G, Q300Y+A342S, Q300Y+K343R, M309I+P313S, M309I+Q331G, M309I+A342S, M309I+K343R, P313S+Q331G, P313S+A342S, P313S+K343R, Q331G+A342S, Q331G+K343R, and A342S+K343R; and/or
V187C+Q300Y, V187C+P18S, V187C+A47S, V187C+W140H, V187C+I163V, V187C+L198I, V187C+M241V, V187C+F242C, V187C+M309I, V187C+P313S, V187C+Q331G, V187C+A342S, V187C+K343R, P18S+A47S, P18S+W140H, P18S+I163V, P18S+L198I, P18S+M241V, P18S+F242C, P18S+Q300Y, P18S+M309I, P18S+P313S, P18S+Q331G, P18S+A342S, P18S+K343R, A47S+W140H, A47S+I163V, A47S+L198I, A47S+M241V, A47S+F242C, A47S+Q300Y, A47S+M309I, A47S+P313S, A47S+Q331G, A47S+A342S, A47S+K343R, W140H+I163V, W140H+L198I, W140H+M241V, W140H+F242C, W140H+Q300Y, W140H+M309I, W140H+P313S, W140H+Q331G, W140H+A342S, W140H+K343R, I163V+L198I, I163V+M241V, I163V+F242C, I163V+Q300Y, I163V+M309I, I163V+P313S, I163V+Q331G, I163V+A342S, I163V+K343R, L198I+M241V, L198I+F242C, L198I+Q300Y, L198I+M309I, L198I+P313S, L198I+Q331G, L198I+A342S, L198I+K343R, M241V+F242C, M241V+Q300Y, M241V+M309I, M241V+P313S, M241V+Q331G, M241V+A342S, M241V+K343R, F242C+Q300Y, F242C+M309I, F242C+P313S, F242C+Q331G, F242C+A342S, F242C+K343R, Q300Y+M309I, Q300Y+P313S, Q300Y+Q331G, Q300Y+A342S, Q300Y+K343R, M309I+P313S, M309I+Q331G, M309I+A342S, M309I+K343R, P313S+Q331G, P313S+A342S, P313S+K343R, Q331G+A342S, Q331G+K343R, and A342S+K343R.
V187C+P18S, V187C+A47S, V187C+N, V187C+W140H, V187C+I163V, V187C+G190N, V187C+L198I, V187C+M241V, V187C+F242C, V187C+A273S, V187C+Q300Y, V187C+M309I, V187C+P313V, V187C+Q331G, V187C+A342S, V187C+K343R, P18S+A47S, P18S+W140H, P18S+I163V, P18S+G190N, P18S+L198I, P18S+M241V, P18S+F242C, P18S+A273S, P18S+Q300Y, P18S+M309I, P18S+P313V, P18S+Q331G, P18S+A342S, P18S+K343R, A47S+W140H, A47S+I163V, A47S+G190N, A47S+L198I, A47S+M241V, A47S+F242C, A47S+A273S, A47S+Q300Y, A47S+M309I, A47S+P313V, A47S+Q331G, A47S+A342S, A47S+K343R, W140H+I163V, W140H+G190N, W140H+L198I, W140H+M241V, W140H+F242C, W140H+A273S, W140H+Q300Y, W140H+M309I, W140H+P313V, W140H+Q331G, W140H+A342S, W140H+K343R, I163V+G190N, I163V+L198I, I163V+M241V, I163V+F242C, I163V+A273S, I163V+Q300Y, I163V+M309I, I163V+P313V, I163V+Q331G, I163V+A342S, I163V+K343R, G190N+L198I, G190N+M241V, G190N+F242C, G190N+A273S, G190N+Q300Y, G190N+M309I, G190N+P313V, G190N+Q331G, G190N+A342S, G190N+K343R, L198I+M241V, L198I+F242C, L198I+A273S, L198I+Q300Y, L198I+M309I, L198I+P313V, L198I+Q331G, L198I+A342S, L198I+K343R, M241V+F242C, M241V+A273S, M241V+Q300Y, M241V+M309I, M241V+P313V, M241V+Q331G, M241V+A342S, M241V+K343R, F242C+A273S, F242C+Q300Y, F242C+M309I, F242C+P313V, F242C+Q331G, F242C+A342S, F242C+K343R, A273S+Q300Y, A273S+M309I, A273S+P313V, A273S+Q331G, A273S+A342S, A273S+K343R, Q300Y+M309I, Q300Y+P313V, Q300Y+Q331G, Q300Y+A342S, Q300Y+K343R, M309I+P313V, M309I+Q331G, M309I+A342S, M309I+K343R, P313V+Q331G, P313V+A342S, P313V+K343R, Q331G+A342S, Q331G+K343R, and A342S+K343R.
Q300Y+P18S, Q300Y+A47S, Q300Y+W140H, Q300Y+T147R, Q300Y+I163V, Q300Y+V187C, Q300Y+G190N, Q300Y+L198I, Q300Y+M241V, Q300Y+F242C, Q300Y+A273S, Q300Y+M309I, Q300Y+K343R, Q300Y+P313V, Q300Y+Q331G, Q300Y+A342S, P18S+A47S, P18S+W140H, P18S+T147R, P18S+I163V, P18S+V187C, P18S+G190N, P18S+L198I, P18S+M241V, P18S+F242C, P18S+A273S, P18S+M309I, P18S+K343R, P18S+P313V, P18S+Q331G, P18S+A342S, A47S+W140H, A47S+T147R, A47S+I163V, A47S+V187C, A47S+G190N, A47S+L198I, A47S+M241V, A47S+F242C, A47S+A273S, A47S+M309I, A47S+K343R, A47S+P313V, A47S+Q331G, A47S+A342S, W140H+T147R, W140H+I163V, W140H+V187C, W140H+G190N, W140H+L198I, W140H+M241V, W140H+F242C, W140H+A273S, W140H+M309I, W140H+K343R, W140H+P313V, W140H+Q331G, W140H+A342S, T147R+I163V, T147R+V187C, T147R+G190N, T147R+L198I, T147R+M241V, T147R+F242C, T147R+A273S, T147R+M309I, T147R+K343R, T147R+P313V, T147R+Q331G, T147R+A342S, I163V+V187C, I163V+G190N, I163V+L198I, I163V+M241V, I163V+F242C, I163V+A273S, I163V+M309I, I163V+K343R, I163V+P313V, I163V+Q331G, I163V+A342S, V187C+G190N, V187C+L198I, V187C+M241V, V187C+F242C, V187C+A273S, V187C+M309I, V187C+K343R, V187C+P313V, V187C+Q331G, V187C+A342S, G190N+L198I, G190N+M241V, G190N+F242C, G190N+A273S, G190N+M309I, G190N+K343R, G190N+P313V, G190N+Q331G, G190N+A342S, L198I+M241V, L198I+F242C, L198I+A273S, L198I+M309I, L198I+K343R, L198I+P313V, L198I+Q331G, L198I+A342S, M241V+F242C, M241V+A273S, M241V+M309I, M241V+K343R, M241V+P313V, M241V+Q331G, M241V+A342S, F242C+A273S, F242C+M309I, F242C+K343R, F242C+P313V, F242C+Q331G, F242C+A342S, A273S+M309I, A273S+K343R, A273S+P313V, A273S+Q331G, A273S+A342S, M309I+K343R, M309I+P313V, M309I+Q331G, M309I+A342S, K343R+P313V, K343R+Q331G, K343R+A342S, P313V+Q331G, P313V+A342S, P313V+, Q331G+A342S.

Preferably, the amino acid sequence of the glucose isomerase comprises at least two, three, four, five, six, seven, eight, nine, ten, eleven, twelve, thirteen, fourteen, fifteen, sixteen or seventeen substitutions at substitution positions wherein the amino acid sequence comprises at least two substitution select-ed from A47S+V187C, A47S+V187W, A47S+V187T, A47S+M241V, A47S+F242C, A47S+F242V, A47S+Q300Y, A47S+Q300S, A47S+Q300C, A47S+Q300F, A47S+M309I, A47S+P313S, A47S+P313V, A47S+A342S, A47S+K343R, A47S+K343N, V187W+M241V, V187W+F242C, V187W+F242V, V187W+Q300Y, V187W+Q300S, V187W+Q300C, V187W+Q300F, V187W+M309I, V187W+P313S, V187W+P313V, V187W+P313G, V187W+A342S, V187W+K343R, V187W+K343N, V187T+M241V, V187T+F242C, V187T+F242V, V187T+Q300Y, V187T+Q300S, V187T+Q300C, V187T+Q300F, V187T+M309I, V187T+P313S, V187T+P313V, V187T+P313G, V187T+A342S, V187T+K343R, V187T+K343N, V187C+M241V, V187C+F242C, V187C+F242V, V187C+Q300Y, V187C+Q300S, V187C+Q300C, V187C+Q300F, V187C+M309I, V187C+P313S, V187C+P313V, V187C+P313G, V187C+A342S, V187C+K343R, V187C+K343N, M241V+F242C, M241V+Q300Y, M241V+Q300F, M241V+Q300C, M241V+Q300S, M241V+M309I, M241V+P313S, M241V+P313V, M241V+P313G, M241V+A342S, M241V+K343R, M241V+K343N, F242C+Q300Y, F242C+Q300S, F242C+Q300C, F242C+Q300F, F242C+M309I, F242C+P313S, F242C+P313V, F242C+P313G, F242C+A342S, F242C+K343R, F242C+K343N, F242V+Q300Y, F242V+Q300S, F242V+Q300C, F242V+Q300F, F242V+M309I, F242V+P313S, F242V+P313V, F242V+P313G, F242V+A342S, F242V+K343R, F242V+K343N, Q300Y+M309I, Q300Y+P313S, Q300Y+P313V, Q300Y+P313G, Q300Y+A342S, Q300Y+K343R, Q300Y+K343N, Q300S+M309I, Q300S+P313S, Q300S+P313V, Q300S+P313G, Q300S+A342S, Q300S+K343R, Q300S+K343N, Q300C+M309I, Q300C+P313S, Q300C+P313V, Q300C+P313G, Q300C+A342S, Q300C+K343R, Q300C+K343N, Q300F+M309I, Q300F+P313S, Q300F+P313V, Q300F+P313G, Q300F+A342S, Q300F+K343R, Q300F+K343N, M309I+P313S, M309I+A342S, M309I+K343R, M309I+K343K, P313S+A342S, P313S+K343R, P313S+K343N, P313V+A342S, P313V+K343R, P313V+K343N, P313G+A342S, P313G+K343R, P313G+K343N, A342S+K343N and A342S+K343R.

Preferably, the amino acid sequence of the glucose isomerase comprises at least two, three, four, five, six, seven, eight, nine, ten, eleven, twelve, thirteen, fourteen, fifteen, sixteen or seventeen substitutions at substitution positions, wherein the substitution positions are in any subsequence or subregion and independently of one another are selected from V187 and Q300, wherein preferably the substitution are independently of one another selected from V187Q and Q300Y.

Preferably, the amino acid sequence of the glucose isomerase comprises at least three, four, five, six, seven, eight, nine, ten, eleven, twelve, thirteen, fourteen, fifteen, sixteen or seventeen substitutions at substitution positions wherein the amino acid sequence comprises at least three substitutions selected from
V187C+P313S+K343R, V187C+M241V+F242C, V187C+Q300S+M309I, V187C+A342S+A47S, V187C+P313V+A47S, V187C+F242C+K343R, V187C+P313S+A47S, V187T+F242C+P313S, and V187C+K343R+A47S; preferably selected from the group consisting of V187C+P313S+K343R, V187C+M241V+F242C, V187C+Q300S+M309I, V187C+A342S+A47S, V187C+P313V+A47S, V187C+F242C+K343R, and V187C+P313S+A47S; more preferably selected from the group consisting of V187C+P313S+K343R, V187C+M241V+F242C, V187C+Q300S+M309I, and V187C+A342S+A47S; even more preferably selected from the group consisting of V187C+P313S+K343R, and V187C+M241V+F242C; and/or
V187C+Q300S+M309I, V187C+M241V+F242C, V187T+F242C+P313S, V187C+P313S+K343R, V187C+P313V+A47S, V187C+A342S+A47S, V187C+F242C+K343R, and V187C+P313S+A47S; preferably selected from the group consisting of V187C+Q300S+M309I, V187C+M241V+F242C, V187T+F242C+P313S, V187C+P313S+K343R, V187C+P313V+A47S, and V187C+A342S+A47S; more preferably selected from the group consisting of V187C+Q300S+M309I, V187C+M241V+F242C, V187T+F242C+P313S, and V187C+P313S+K343R; even more preferably selected from the group consisting of V187C+Q300S+M309I, and V187C+M241V+F242C; and/or
V187C+P313S+K343R, V187C+Q300S+M309I, V187C+M241V+F242C, V187C+P313V+A47S, V187C+A342S+A47S, V187C+F242C+K343R, V187T+F242C+P313S, and V187C+P313S+A47S; preferably selected from the group consisting of V187C+P313S+K343R, V187C+Q300S+M309I, V187C+M241V+F242C, V187C+P313V+A47S, V187C+A342S+A47S, and V187C+F242C+K343R; more preferably selected from the group consisting of V187C+P313S+K343R, V187C+Q300S+M309I, V187C+M241V+F242C, and V187C+P313V+A47S; even more preferably selected from the group consisting of V187C+P313S+K343R, and V187C+Q300S+M309I; and/or
V187C+P313S+K343R, V187C+M241V+F242C, V187C+Q300S+M309I, V187C+A342S+A47S, V187C+P313V+A47S, V187C+F242C+K343R, V187T+F242C+P313S, and V187C+P313S+A47S; preferably selected from the group consisting of V187C+P313S+K343R, V187C+M241V+F242C, V187C+Q300S+M309I, V187C+A342S+A47S, V187C+P313V+A47S, and V187C+F242C+K343R; more preferably selected from the group consisting of V187C+P313S+K343R, V187C+M241V+F242C, V187C+Q300S+M309I, and V187C+A342S+A47S; even more preferably selected from the group consisting of V187C+P313S+K343R, and V187C+M241V+F242C; and/or
V187C+P313S+K343R, V187C+A342S+A47S, V187C+M241V+F242C, V187C+F242C+K343R, V187C+P313V+A47S, V187C+Q300S+M309I, V187T+F242C+P313S, V187C+K343R+A47S, and V187C+P313S+A47S; preferably selected from the group consisting of V187C+P313S+K343R, V187C+A342S+A47S, V187C+M241V+F242C, V187C+F242C+K343R, V187C+P313V+A47S, V187C+Q300S+M309I, V187T+F242C+P313S, V187C+K343R+A47S, and V187C+P313S+A47S; more preferably selected from the group consisting of V187C+P313S+K343R, V187C+A342S+A47S, V187C+M241V+F242C, and V187C+F242C+K343R; even more preferably select-ed from the group consisting of V187C+P313S+K343R, and V187C+A342S+A47S; most preferably at the three amino acid positions V187C+P313S+K343R.

Preferably, the amino acid sequence of the glucose isomerase comprises at least four, five, six, seven, eight, nine, ten, eleven, twelve, thirteen, fourteen, fifteen, sixteen or seventeen substitutions at substitution positions wherein the amino acid sequence comprises at least four substitution selected from V187C+Q300S+M309I+P313S, V187W+F242C+Q300S+A47S, V187C+F242C+Q300S+M309I, V187C+M241V+F242C+Q300Y, V187C+F242C+Q300Y+M309I, V187W+M241V+F242C+P313V, V187C+F242C+A342S+A47S, V187C+M241V+F242C+A47S, V187C+M309I+P313S+A47S, V187C+F242C+P313S+A342S, V187W+F242C+P313S+A47S, V187C+F242C+M309I+A47S, V187C+M241V+F242C+P313G, V187C+F242C+Q300Y+A47S, V187C+A342S+K343R+A47S, V187C+M309I+P313V+A47S, V187C+P313S+A342S+A47S, V187C+P313V+K343R+A47S, and V187C+M241V+F242C+A342S, preferably selected from the group consisting of V187C+F242C+Q300S+M309I, V187W+M241V+F242C+P313V and V187C+Q300S+M309I+P313S.

Preferably, the amino acid sequence of the glucose isomerase comprises at least five, six, seven, eight, nine, ten, eleven, twelve, thirteen, fourteen, fifteen, sixteen or seventeen substitutions at substitution positions wherein the amino acid sequence comprises at least five substitution selected from V187C+F242C+M309I+K343R+A47S, V187C+F242C+M309I+A342S+A47S, V187C+Q300S+M309I+K343R+A47S, V187C+M241V+F242C+M309I+A47S, V187C+M241V+F242C+Q300S+M309I, V187C+M241V+F242C+A342A+A47S, V187C+M241V+F242C+K343R+A47S, V187C+M241V+F242C+Q300Y+A47S, V187C+F242C+Q300Y+K343N+A47S, V187C+F242C+A342S+K343N+A47S, V187C+M241V+F242C+A342S+A47S, V187C+M241V+F242C+P313S+A47S, V187C+M241V+F242C+Q300S+A47S, V187C+M241V+F242C+M309I+A342S and V187C+F242C+M309I+P313S+A47S preferably selected from the group consisting of V187C+F242C+M309I+K343R+A47S, and V187C+M241V+F242C+M309I+A47S.

Preferably, the amino acid sequence of the glucose isomerase comprises at least six, seven, eight, nine, ten, eleven, twelve, thirteen, fourteen, fifteen, sixteen or seventeen substitutions at substitution positions wherein the amino acid sequence comprises at least Six substitution selected from V187C+M241V+F242C+Q300S+M309I+A47S, V187C+F242C+M309I+P313S+K343R+A47S and V187C+F242C+M309I+A342S+K343R+A47S.

Preferably, the amino acid sequence of the glucose isomerase comprises at least seven, eight, nine, ten, eleven, twelve, thirteen, fourteen, fifteen, sixteen or seventeen substitutions at substitution positions wherein the amino acid sequence comprises at least seven substitutions selected from V187C+M241V+F242C+M309I+P313S+K343R+A47S and V187C+M241V+F242C+M309I+A342S+K343R+A47S.

Preferably, the amino acid sequence of the glucose isomerase comprises at least one, two, three, four, five, six, seven, eight, nine, ten, eleven, twelve, thirteen, fourteen, fifteen, sixteen or seventeen substitutions, wherein the amino acid sequence comprises a substitution in at least one further, at least two further, at least three further, at least four further, at least five further, at least six further, at least seven further, at least eight further, at least nine further, at least ten further, at least eleven further, at least twelve further, at least thirteen further, at least fourteen further, at least fifteen further, or at least sixteen further substitution positions selected from the group consisting of substitution positions P18, A47, W140, T147, I163, V187, G190, L198, M241, F242, A273, Q300, M309, P313, Q331, A342, and K343; preferably consisting of A47, V187, M241, F242, Q300, M309, P313, A342 and K343.

Preferably, the amino acid sequence of the glucose isomerase comprises at least one, two, three, four, five, six, seven, eight, nine, ten, eleven, twelve, thirteen, fourteen, fifteen, sixteen or seventeen substitutions at substitution positions, wherein the amino acid sequence comprises a substitution in at least one further, at least two further, at least three further, at least four further, at least five further, at least six further, at least seven further, at least eight further, at least nine further, at least ten further, at least eleven further, at least twelve further, at least thirteen further, at least fourteen further, at least fifteen further, or at least sixteen further substitution positions selected from the group consisting of
position 18, the substitution being selected from the group consisting of P18S, P18A, P18R, P18N, P18D, P18C, P18Q, P18E, P18G, P18H, P18I, P18L, P18K, P18M, P18F, P18T, P18W, P18V or P18Y; more preferably P18S, P18T, P18Y; and most preferably P18S; and/or
position 47, the substitution being selected from the group consisting of A47S, A47C, A47R, A47N, A47D, A47Q, A47E, A47G, A47H, A47I, A47L, A47K, A47M, A47F, A47P, A47T, A47W, A47V, or A47Y; preferably A47S, A47Q or A47R; and more preferably A273S, A273T or A273Y; and/or
position 140, the substitution being selected from the group consisting of W140H, W140C, W140A, W140R, W140N, W140D, W140Q, W140E, W140G, W140I, W140L, W140K, W140M, W140F, W140P, W140S, W140T, W140V or W140Y; preferably W140H, W140R, or W140K; and/or
position 147, the substitution being selected from the group consisting of T147R, T147C, T147A, T147N, T147D, T147E, T147G, T147H, T147I, T147L, T147K, T147M, T147F, T147P, T147S, T147W, T147V or T147Y; preferably T147R; and/or
position 163, the substitution being selected from the group consisting of I163V, I163C, I163A, I163R, I163N, I163D, I163Q, I163E, I163G, I163H, I163L, I163K, I163M, I163F, I163P, I163S, I163T, I163W, or I163Y; preferably I163V; and/or
position 187, the substitution being selected from the group consisting of V187C, V187A, V187R, V187N, V187D, V187Q, V187E, V187G, V187H, V187I, V187L, V187K, V187M, V187F, V187P, V187S, V187T, V187W, or V187Y; preferably V187C or V187T, or V187W; and more preferably V187C; and/or
position 190, the substitution being selected from the group consisting of G190N, G190C, G190A, G190R, G190D, G190Q, G190E, G190H, G190I, G190L, G190K, G190M, G190F, G190P, G190S, G190T, G190W, or G190Y; preferably G190Q or G109N; and/or
position 198, the substitution being selected from the group consisting of L198I, L198C, L198A, L198R, L198N, L198D, L198Q, L198E, L198G, L198H, L198K, L198M, L198F, L198P, L198S, L198T, L198W, L198V or L198Y; preferably L198I; and/or
position 241, the substitution being selected from the group consisting of M241V, M241C, M241A, M241R, M241N, M241D, M241Q, M241E, M241G, M241H, M241I, M241L, M241K, M241F, M241P, M241S, M241T, M241W, or M241Y; preferably M241V, M241C, M241R, M241D, M241Q, M241E, M241G, M241H, M241I, M241K, M241F, M241P, M241W, or M241Y; more preferably M241V or M241C; most preferably M241V or M241C; and/or
position 242, the substitution being selected from the group consisting of F242C, F242A, F242R, F242N, F242D, F242Q, F242E, F242G, F242H, F242I, F242L, F242K, F242M, F242P, F242S, F242T, F242W, F242V or F242Y; preferably F242C, F242A, F242R, F242N, F242D, F242Q, F242E, F242G, F242I, F242K, F242M, F242P, F242S, F242T, F242W, F242V, or F242Y; more preferably F242C or F242V; more preferably F242C; and/or
position 273, the substitution being selected from the group consisting of A273S, A273C, A273R, A273N, A273D, A273Q, A273E, A273G, A273H, A273I, A273L, A273K, A273M, A273F, A273P, A273T, A273W, A273V or A273Y; preferably A273S; and/or
position 300, the substitution being selected from the group consisting of Q300Y, Q300C, Q300A, Q300R, Q300N, Q300D, Q300E, Q300G, Q300H, Q300I, Q300L, Q300K, Q300M, Q300F, Q300P, Q300S, Q300T, Q300W or Q300V; preferably Q300Y, Q300C, Q300A, Q300R, Q300N, Q300D, Q300G, Q300H, Q300I, Q300L, Q300K, Q300M, Q300F, Q300P, Q300S, Q300T, Q300W, or Q300V; more preferably Q300Y, Q300S, Q300C or Q300F; most preferably Q300Y, Q300S, Q300C or Q300F; more preferably Q300Y; and/or
position 309, the substitution being selected from the group consisting of M309I, M309C, M309A, M309R, M309N, M309D, M309Q, M309E, M309G, M309H, M309L, M309K, M309F, M309P, M309S, M309T, M309W, M309V or M309Y; preferably M309I, M309V, M309L, M309P; more preferably M309I or M309P; most preferably M309I; and/or
position 313, the substitution being selected from the group consisting of P313S, P313C, P313A, P313R, P313N, P313D, P313Q, P313E, P313G, P313H, P313I, P313L, P313K, P313M, P313F, P313T, P313W, P313V or P313Y; preferably P313S, P313V and P313G; more preferably P313S; and/or
position 331, the substitution being selected from the group consisting of Q331G, Q331C, Q331A, Q331R, Q331N, Q331D, Q331E, Q331H, Q331I, Q331L, Q331K, Q331M, Q331F, Q331P, Q331S, Q331T, Q331W, Q331V or Q331Y; preferably Q331G; and/or
position 342, the substitution being selected from the group consisting of A342S, A342C, A342R, A342N, A342D, A342Q, A342E, A342G, A342H, A342I, A342L, A342K, A342M, A342F, A342P, A342T, A342W, A342V or A342Y; preferably A342S, A342T, A342Y; more preferably A342S; and/or
position 343, the substitution being selected from the group consisting of K343R, K343C, K343A, K343N, K343D, K343Q, K343E, K343G, K343H, K343I, K343L, K343M, K343F, K343P, K343S, K343T, K343W, K343V or K343Y; preferably K343R, K343N, K343Q; more preferably K343R or K343N; most preferably K343R.

Preferably, the amino acid sequence of the glucose isomerase comprises at least one, two, three, four, five, six, seven, eight, nine, ten, eleven, twelve, thirteen, fourteen, fifteen, sixteen or seventeen substitutions, wherein the amino acid sequence comprises a substitution in at least one further, at least two further, at least three further, at least four further, at least five further, at least six further, at least seven further, at least eight further, at least nine further, at least ten further, at least eleven further, at least twelve further, at least thirteen further, at least fourteen further, at least fifteen further, or at least sixteen further substitution positions selected from the group consisting of
position 18, the substitution being P18S, P18T or P18Y; more preferably P18S; and/or
position 47, the substitution being selected from the group consisting of A47S, A47Q or A47R; preferably A47S; and/or
position 140, the substitution being W140H; and/or
position 147, the substitution being T147R; and/or
position 163, the substitution being I163V; and/or
position 187, the substitution being selected from the group consisting of V187C, V187T, or V187W; preferably V187C; and/or
position 190, the substitution being G190N; and/or
position 198, the substitution being L198I; and/or
position 241, the substitution being M241V or M241C; preferably M241V; and/or
position 242, the substitution being F242C or F242V; preferably F242C; and/or
position 273, the substitution being A273S; and/or
position 300, the substitution being selected from the group consisting of Q300Y, Q300S, Q300C or Q300F; preferably Q300Y; and/or
position 309, the substitution being M309I; and/or
position 313, the substitution being selected from the group consisting of P313S, P313V and P313G; preferably P313S; and/or
position 331, the substitution being Q331G; and/or
position 342, the substitution being A342S; and/or
position 343, the substitution being K343R or K343N; preferably K343R.

Preferably, the amino acid sequence of the glucose isomerase comprises a substitution in at least one, at least two, at least three, at least four, at least five, at least six, at least seven, at least eight, at least nine, at least ten, at least eleven, at least twelve, at least thirteen, at least fourteen, at least fifteen, at least or sixteen, at least seventeen amino acid positions.

Preferably, the amino acid sequence of the glucose isomerase comprises a substitution in one to seventeen, two to seventeen, three to seventeen, four to seventeen, five to seventeen, six to seventeen, seven to seventeen, eight to seventeen, nine to seventeen, ten to seventeen, eleven to seventeen, twelve to seventeen, thirteen to seven-teen, fourteen to seventeen, fifteen to seventeen, sixteen to seventeen, or seventeen amino acid positions.

Preferably, the amino acid sequence of the glucose isomerase comprises a substitution in one amino acid position, in two amino acid positions, in three amino acid positions, in four amino acid positions, in five amino acid positions, in six amino acid positions, in seven amino acid positions, in eight amino acid positions, in nine amino acid positions, in ten amino acid positions, in eleven amino acid positions, in twelve amino acid positions, in thirteen amino acid positions, in fourteen amino acid positions, in fifteen amino acid positions, or in sixteen, or in seventeen amino acid positions.

Preferably, the amino acid sequence of the glucose isomerase comprises or consists of any one of amino acid sequences with at least 67%, or at least 68%, or at least 70%, or at least 72%, or at least 74%, or at least 76%, or at least 78%, or at least 80%, or at least 82%, or at least 84%, or at least 86%, or at least 88%, or at least 90%, or at least 92%, or at least 94%, or at least 96%, or at least 97%, or at least 98%, or at least 99%, or at least 99.9%, or at least 100% identity to SEQ ID NO: 1.

Preferably, the glucose isomerase according to the invention comprises an amino acid sequence which has at least 65% identity or at least 80% identity to SEQ ID NO: 1 and which compared to SEQ ID NO: 1 comprises at least one, two, three, four, five, six, seven, eight, nine, ten, eleven or twelve substitutions at substitution positions, which are independently of one another located in
subregion R1-A ranging from position L266 to Y280; and/or
subregion R1-S1-B ranging from position S295 to T319; and/or;
subregion R1-S-D ranging from position L338 to K343; and/or
subregion R2 ranging from position L231 to Q243; and/or
subregion R3-S1 ranging from position V187 to E193; and/or
subregion R4-A1, ranging from position L139 to W140; and/or
subregion R5-S1, ranging from position F44 to G61.

Preferably, the glucose isomerase according to the invention comprises an amino acid sequence which has at least 65% identity to SEQ ID NO: 1 and which compared to SEQ ID NO: 1 comprises at least one, two, three, four, five, six, seven, eight, nine, or ten substitutions at substitution positions independently of one another selected from the groups consisting of:
A273 in subregion R1-A; and/or
Q300 and P313 in subregion R1-S1-B; and/or
K343 in subregion R1-S-D; and/or
M241 and F242 in subregion R2; and/or
V187 and G190, preferably V187, in subregion R3-S1; and/or
W140 in subregion R4-A1; and/or
A47 in subregion R5-S1;
   and, optionally, further comprises at least one or two substitutions at substitutions positions independently of one another selected from the groups consisting of;
M309 in subregion R1-S1-B; and/or
A342 in subregion R1-S-D.

Preferably, the glucose isomerase according to the invention comprises an amino acid sequence which has at least 80% identity to SEQ ID NO: 1 and which compared to SEQ ID NO: 1 comprises at least one, two, three, four, five, six, seven, eight, nine, ten, eleven, or twelve substitutions at substitution positions independently of one another selected from the groups consisting of:
A273 in subregion R1-A;
Q300, M309, and P313 in subregion R1-S1-B;
A342 and K343 in subregion R1-S-D; and/or
M241 and F242 in subregion R2; and/or
V187 and G190, preferably V187, in subregion R3-S1; and/or
W140 in subregion R4-A1; and/or
A47 in subregion R5-S1.

Preferably, the glucose isomerase according to the invention comprises an amino acid sequence which has at least 65% identity or at least 80% identity to SEQ ID NO: 1 and which compared to SEQ ID NO: 1 comprises at least one, two, three, four, five, six, seven, eight, nine or ten substitutions at substitution positions, which are independently of one another located in
subregion R1-S2-A, ranging from position L266 to A273; and/or
subregion R1-S2-B1, ranging from position S295 to Q300; and/or
subregion R1-S2-B2, ranging from position D308 to D311; and/or
subregion R1-S2-B3, consisting of position P313; and/or
subregion R1-S-D ranging from position L338 to K343; and/or
subregion R3-S2 ranging from position N185 to G191; and/or
subregion R4-A1, ranging from position L139 to W140; and/or
subregion R5-S2, ranging from position G45 to H50.

Preferably, the glucose isomerase according to the invention comprises an amino acid sequence which has at least 65% identity to SEQ ID NO: 1 and which compared to SEQ ID NO: 1 comprises at least one, two, three, four, five, six, seven or eight substitutions at substitution positions independently of one another selected from the groups consisting of:
A273 in subregion R1-S2-A; and/or
Q300 in subregion R1-S2-B1; and/or
P313 in subregion R1-S1-B3; and/or
K343 in subregion R1-S-D; and/or
V187 and G190, preferably V187, in subregion R3-S2; and/or
W140 in subregion R4-A1; and/or
A47 in subregion R5-S2.
   and, optionally, further comprises at least one or two substitutions at substitutions positions independently of one another selected from the groups consisting of:
M309 in subregion R1-S1-B; and/or
A342 in subregion R1-S-D.

Preferably, the glucose isomerase according to the invention comprises an amino acid sequence which has at least 80% identity to SEQ ID NO: 1 and which compared to SEQ ID NO: 1 comprises at least one, two, three, four, five, six, seven, eight, nine or ten substitutions at substitution positions independently of one another selected from the groups consisting of:
A273 in subregion R1-S2-A; and/or
Q300 in subregion R1-S2-B1; and/or
M309 in subregion R1-S2-B2; and/or
P313 in subregion R1-S1-B3; and/or
A342 and K343 in subregion R1-S-D; and/or
V187 and G190, preferably V187, in subregion R3-S2; and/or
W140 in subregion R4-A1; and/or
A47 in subregion R5-S2.

Preferably, the glucose isomerase according to the invention comprises an amino acid sequence which has at least 65% identity or at least 80% identity to SEQ ID NO: 1 and which compared to SEQ ID NO: 1 comprises at least one, two, three, four, five, six, seven, eight or nine substitutions at substitution positions, which are independently of one another located in
subregion R1-S3-A ranging from position N267 to A273; and/or
subregion R1-B1, ranging from position S295 to D302; and/or
subregion R1-B2, ranging from position W307 to D315; and/or
subregion R1-S3-D ranging from position L338 to K346; and/or
subregion R3-S3 consisting of position V187; and/or
subregion R4-A1, ranging from position L139 to W140; and/or
subregion R5-S3, ranging from position F44 to G54.

Preferably, the glucose isomerase according to the invention comprises an amino acid sequence which has at least 65% identity to SEQ ID NO: 1 and which compared to SEQ ID NO: 1 comprises at least one, two, three, four, five, six or seven substitutions at substitution positions independently of one another selected from the groups consisting of:
A273 in subregion R1-S3-A; and/or
Q300 in subregion R1-B1; and/or
P313 in subregion R1-B2; and/or
K343 in subregion R1-S3-D; and/or
V187 in subregion R3-S3; and/or
W140 in subregion R4-A1; and/or
A47 in subregion R5-S3.
   and, optionally, further comprises at least one or two substitutions at substitutions positions independently of one another selected from the groups consisting of:
M309 in subregion R1-S1-B; and/or
A342 in subregion R1-S-D.

Preferably, the glucose isomerase according to the invention comprises an amino acid sequence which has at least 80% identity to SEQ ID NO: 1 and which compared to SEQ ID NO: 1 comprises at least one, two, three, four, five, six, seven, eight or nine substitutions at substitution positions independently of one another selected from the groups consisting of:
A273 in subregion R1-S3-A; and/or
Q300 in subregion R1-B1; and/or
M309 and P313 in subregion R1-B2; and/or
A342 and K343 in subregion R1-S3-D; and/or
V187 in subregion R3-S3; and/or
W140 in subregion R4-A1; and/or
A47 in subregion R5-S3.

Preferably, the glucose isomerase according to the invention comprises an amino acid sequence which has at least 65% identity or at least 80% identity to SEQ ID NO: 1 and which compared to SEQ ID NO: 1 comprises at least one, two, three, four, five or six substitutions at substitution positions, which are independently of one another located in
subregion R1-S4-B1, ranging from position D297 to Q300; and/or
subregion R1-S4-B2, ranging from position M309 to P313; and/or
subregion R1-S4-D ranging from position N339 to R345; and/or
subregion R5-S4, consisting of position A47.

Preferably, the glucose isomerase according to the invention comprises an amino acid sequence which has at least 65% identity to SEQ ID NO: 1 and which compared to SEQ ID NO: 1 comprises at least one, two, three or four substitutions at substitution positions independently of one another selected from the groups consisting of:
Q300 in subregion R1-S4-B1; and/or
P313 in subregion R1-S4-B2; and/or
K343 in subregion R1-S4-D; and/or
A47 in subregion R5-S4.
   and, optionally, further comprises at least one or two substitutions at substitutions positions independently of one another selected from the groups consisting of:
M309 in subregion R1-S1-B; and/or
A342 in subregion R1-S-D.

Preferably, the glucose isomerase according to the invention comprises an amino acid sequence which has at least 80% identity to SEQ ID NO: 1 and which compared to SEQ ID NO: 1 comprises at least one, two, three, four, five, six, seven, eight or nine substitutions at substitution positions independently of one another selected from the groups consisting of:
Q300 in subregion R1-S4-B1; and/or
M309 and P313 in subregion R1-S4-B2; and/or
A342 and K343 in subregion R1-S4-D; and/or
A47 in subregion R5-S4.

It is within the meaning of the invention that any identity of at least a certain percentage shall be interpreted as an identity of at least the certain percentage to 100%. Example: An identity of “at least 80%” means an identity “from 80% to 100%”.

Preferably, the amino acid sequence of the glucose isomerase comprises or consists of any one of amino acid sequences with at least 67%, or at least 68%, or at least 70%, or at least 72%, or at least 74%, or at least 76%, or at least 78%, or at least 80%, or at least 82%, or at least 84%, or at least 86%, or at least 88%, or at least 90%, or at least 92%, or at least 94%, or at least 96%, or at least 98%, or at least 100% identity to SEQ ID NO: 2.

Preferably, the amino acid sequence of the glucose isomerase comprises or consists of any one of amino acid sequences with at least 67%, or at least 68%, or at least 70%, or at least 72%, or at least 74%, or at least 76%, or at least 78%, or at least 80%, or at least 82%, or at least 84%, or at least 86%, or at least 88%, or at least 90%, or at least 92%, or at least 94%, or at least 96%, or at least 98%, or at least 100% identity to SEQ ID NO: 7.

Preferably, the amino acid sequence of the glucose isomerase comprises or consists of any one of amino acid sequences with at least 80%, preferably at least 85%, more preferably at least 90%, yet more preferably at least 95%, most preferably at least 99.9%, or utmost preferably 100%, to each one of SEQ ID NO: 2, 3, 4, 5, 6, 7, 8, 9, 10, 11 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, or 73.

Preferably, the amino acid sequence of the glucose isomerase comprises or consists of any one of amino acid sequences with at least 80%, preferably at least 85%, more preferably at least 90%, yet more preferably at least 95%, most preferably at least 99.9%, or utmost preferably 100%, to each one of SEQ ID NO: 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, or 73.

Preferably, the glucose isomerase according to the invention is capable of catalyzing conversion of an aldose molecule into a ketose molecule; preferably of glucose into fructose, and/or a ketose molecule into an aldose molecule; preferably of fructose into glucose.

In a preferred embodiment of the invention, the glucose isomerase according to the invention exhibits an in-creased enzymatic activity in the absence of Ca²⁺ (A₀) in comparison to SEQ ID NO:1 with respect to the conversion of fructose into glucose or vice versa, preferably with respect to the conversion of fructose into glucose.

Preferably, the glucose isomerase according to the invention exhibits an enzymatic activity A₀ of at least 0.1%, preferably of at least 0.2%, more preferably at least 10%, still more preferably at least 30%, yet more preferably at least 50%, even more preferably at least 100%, and most preferably at least 150% higher than the enzymatic activity of SEQ ID NO: 1.

Preferably, the glucose isomerase according to the invention exhibits an enzymatic activity A₀ of at least 1.001-fold, preferably of at least 1.002-fold, more preferably at least 1.1-fold, still more preferably at least 1.3-fold, yet more preferably at least 1.5-fold, even more preferably at least 2.0-fold, and most preferably at least 2.5-fold higher than the enzymatic activity of SEQ ID NO: 1 with respect to the conversion of glucose into fructose.

Preferably, the glucose isomerase according to the invention exhibits an enzymatic activity A₀ of at least 1.001-fold, preferably of at least 1.002-fold, more preferably at least 1.1-fold, still more preferably at least 1.3-fold, yet more preferably at least 1.5-fold, even more preferably at least 2.0-fold, and most preferably at least 2.5-fold higher than the enzymatic activity of SEQ ID NO: 2 with respect to the conversion of glucose into fructose.

Preferably, the glucose isomerase according to the invention exhibits an enzymatic activity A₀ of at least 1.001-fold, preferably of at least 1.002-fold, more preferably at least 1.1-fold, still more preferably at least 1.3-fold, yet more preferably at least 1.5-fold, even more preferably at least 2.0-fold, and most preferably at least 2.5-fold higher than the enzymatic activity of SEQ ID NO: 7 with respect to the conversion of glucose into fructose.

In another preferred embodiment of the invention, the glucose isomerase according to the invention exhibits
an increased enzymatic activity in the presence of Ca²⁺ (A_{Ca2+}) in comparison to SEQ ID NO: 1 with respect to the conversion of fructose into glucose or vice versa, preferably with respect to the conversion of fructose into glucose; and/or
an increased residual enzymatic activity in the presence of Ca²⁺ (RA A_{Ca2+}) in comparison to SEQ ID NO:1 with respect to the conversion of fructose into glucose or vice versa, preferably with respect to the conversion of fructose into glucose.

Preferably, the enzymatic activity A_{Ca2+} of the glucose isomerase according to the invention is determined at a Ca²⁺ concentration of at least 0.5 mM, preferably at least 60 mM, more preferably at least 30 mM, still more preferably at least 20 mM, most preferably at least 10 mM, and utmost preferably at least 5 mM.

Preferably, the glucose isomerase according to the invention exhibits an increased enzymatic activity A_{Ca2+} in comparison to SEQ ID NO: 1 within the range of at 5 mM±0.5 mM, or within the range of 4.0 mM to 5 mM, 3 mM to 5 mM, 2 mM to 5 mM, 1 mM to 5 mM, or 0.5 mM to 5 mM; preferably within the range of 5 mM to 10 mM, 4.0 mM to 10 mM, 3 mM to 10 mM, 2 mM to 10 mM, 1 mM to 10 mM, or 0.5 mM to 10 mM; more preferably within the range of 5 mM to 20 mM, 4.0 mM to 20 mM, 3 mM to 20 mM, 2 mM to 20 mM, 1 mM to 20 mM, or 0.5 mM to 20 mM; even more preferably within the range of 5 mM to 30 mM, 4.0 mM to 30 mM, 3 mM to 30 mM, 2 mM to 30 mM, 1 mM to 30 mM, or 0.5 mM to 30 mM; and most preferably within the range of 5 mM to 60 mM, 4.0 mM to 60 mM, 3 mM to 60 mM, 2 mM to 60 mM, 1 mM to 60 mM, or 0.5 mM to 60 mM.

Preferably, the glucose isomerase according to the invention exhibits an enzymatic activity A_{Ca2+} of at least 0.1%; preferably of at least 0.2%, more preferably at least 10%, still more preferably at least 30%, yet more preferably at least 50%, even more preferably at least 100%, most preferably at least 150%, and in particular at least 200% higher than the enzymatic activity of SEQ ID NO: 1.

Preferably, the glucose isomerase according to the invention exhibits an enzymatic activity A_{Ca2+} of at least 1.001-fold, preferably of at least 1.002-fold, more preferably at least 1.1-fold, still more preferably at least 1.3-fold, yet more preferably at least 1.5-fold, even more preferably at least 2-fold, most preferably at least 2.5-fold, and in particular at least 3-fold higher than the enzymatic activity of SEQ ID NO: 1.

Preferably, the glucose isomerase according to the invention exhibits an enzymatic activity A_{Ca2+} of at least 1.001-fold, preferably of at least 1.002-fold, more preferably at least 1.1-fold, still more preferably at least 1.3-fold, yet more preferably at least 1.5-fold, even more preferably at least 2-fold, most preferably at least 2.5-fold, and in particular at least 3-fold higher than the enzymatic activity of SEQ ID NO: 2.

Preferably, the glucose isomerase according to the invention exhibits an enzymatic activity A_{Ca2+} of at least 1.001-fold, preferably of at least 1.002-fold, more preferably at least 1.1-fold, still more preferably at least 1.3-fold, yet more preferably at least 1.5-fold, even more preferably at least 2-fold, most preferably at least 2.5-fold, and in particular at least 3-fold higher than the enzymatic activity of SEQ ID NO: 7.

Preferably, the glucose isomerase according to the invention exhibits a residual enzymatic activity R A_{Ca2+} in the presence of Ca²⁺ of at least 4.0%, preferably of at least 10%, more preferably of at least 15%, even more preferably of at least 20%, yet more preferably of at least 25% most preferably of at least 30%, and utmost preferably at least 35% in comparison to the enzymatic activity in the absence of Ca²⁺.

In another preferred embodiment of the invention, the glucose isomerase according to the invention exhibits an in-creased enzymatic activity at neutral and acidic pH (A_{pH}) in comparison to SEQ ID NO: 1 with respect to the conversion of fructose into glucose or vice versa, preferably with respect to the conversion of fructose into glucose.

Preferably, the enzymatic activity A_{pH} of the glucose isomerase is deter-mined at a pH value of 7.0±0.5, or 6.5±0.5, or 5.8±0.5, or within the range from 5.0 to 7.0, 5.3 to 7.0, 5.5 to 7.0, 5.8 to 7.0, 6.0 to 7.0, 6.5 to 7.0; preferably within the range from 5.0 to 7.5, 5.3 to 7.5, 5.5 to 7.5, 5.8 to 7.5, 6.0 to 7.5, 6.5 to 7.5; more preferably within the range from 5.0 to 8.0, 5.3 to 8.0, 5.5 to 8.0, 5.8 to 8.0, 6.0 to 8.0, 6.5 to 8.0.

Preferably, the glucose isomerase according to the invention exhibits an increased enzymatic activity A_{pH} at a pH value of 7.0±0.5, or 6.5±0.5, or 5.8±0.5, or at any pH value within the range from 5.0 to 7.0, 5.3 to 7.0, 5.5 to 7.0, 5.8 to 7.0, 6.0 to 7.0, 6.5 to 7.0; preferably within the range from 5.0 to 7.5, 5.3 to 7.5, 5.5 to 7.5, 5.8 to 7.5, 6.0 to 7.5, 6.5 to 7.5; more preferably within the range from 5.0 to 8.0, 5.3 to 8.0, 5.5 to 8.0, 5.8 to 8.0, 6.0 to 8.0, 6.5 to 8.0.

Preferably, the glucose isomerase according to the invention exhibits an enzymatic activity A_{pH} of at least 0.1%; preferably of at least 0.2%, more preferably at least 10%, still more preferably at least 30%, yet more preferably at least 50%, even more preferably at least 100%, most preferably at least 150%, and in particular at least 200% higher than the enzymatic activity of SEQ ID NO: 1.

Preferably, the glucose isomerase according to the invention exhibits an enzymatic activity A_{pH} of at least 1.001-fold, preferably of at least 1.002-fold, more preferably at least 1.1-fold, still more preferably at least 1.3-fold, yet more preferably at least 1.5-fold, even more preferably at least 2-fold, most preferably at least 2.5-fold, and in particular at least 3-fold higher than the enzymatic activity of SEQ ID NO: 1.

The glucose isomerase according to any of claims 97 to 99, which exhibits an enzymatic activity A_{pH} of at least 1.001-fold, preferably of at least 1.002-fold, more preferably at least 1.1-fold, still more preferably at least 1.3-fold, yet more preferably at least 1.5-fold, even more preferably at least 2-fold, most preferably at least 2.5-fold, and in particular at least 3-fold higher than the enzymatic activity of SEQ ID NO: 2.

The glucose isomerase according to any of claims 97 to 99, which exhibits an enzymatic activity A_{pH} of at least 1.001-fold, preferably of at least 1.002-fold, more preferably at least 1.1-fold, still more preferably at least 1.3-fold, yet more preferably at least 1.5-fold, even more preferably at least 2-fold, most preferably at least 2.5-fold, and in particular at least 3-fold higher than the enzymatic activity of SEQ ID NO: 7.

In another preferred embodiment of the invention, the glucose isomerase according to the invention exhibits an in-creased enzymatic activity at low temperatures (A_{Temp}) in comparison to SEQ ID NO: 1 with respect to the conversion of fructose into glucose or vice versa, preferably with respect to the conversion of fructose into glucose.

Preferably, the enzymatic activity A_{Temp} of the glucose isomerase according to the invention is determined at a temperature of 35° C.±1.0° C., or 25° C.±1.0° C., or 20° C.±1.0° C., or 15° C.±1.0° C., or within the range from 15° C. to 25° C., preferably in the range from 15° C. to 35° C., more preferably in the range from 10° C. to 35° C., and most preferably in the range from 5° C. to 35° C.

Preferably, the glucose isomerase according to the invention exhibits an increased enzymatic activity A_{Temp} at a temperature of 35° C.±1.0° C., or 25° C.±1.0° C., or 20° C.±1.0° C., or 15° C.±1.0° C., or within the range from 15° C. to 25° C., preferably in the range from 15° C. to 35° C., more preferably in the range from 10° C. to 35° C., and most preferably in the range from 5° C. to 35° C.

Preferably, the glucose isomerase according to the invention exhibits an enzymatic activity A_{Temp} of at least 0.1%; preferably of at least 0.2%, more preferably at least 10%, still more preferably at least 300%, yet more preferably at least 50%, even more preferably at least 100%, most preferably at least 150%, and utmost preferably at least 200% higher than the enzymatic activity A_{Temp} of SEQ ID NO: 1.

Preferably, the glucose isomerase according to the invention exhibits an enzymatic activity A_{Temp} of at least 1.001-fold, preferably of at least 1.002-fold, more preferably at least 1.1-fold, still more preferably at least 1.3-fold, yet more preferably at least 1.5-fold, even more preferably at least 2.0-fold, most preferably at least 2.5-fold, and utmost preferably at least 3.0-fold higher than the enzymatic activity A_{pH} of SEQ ID NO: 1.

Preferably, the glucose isomerase according to the invention exhibits an enzymatic activity A_{Temp} of at least 1.001-fold, preferably of at least 1.002-fold, more preferably at least 1.1-fold, still more preferably at least 1.3-fold, yet more preferably at least 1.5-fold, even more preferably at least 2.0-fold, most preferably at least 2.5-fold, and utmost preferably at least 3.0-fold higher than the enzymatic activity A_{pH} of SEQ ID NO: 2.

Preferably, the glucose isomerase according to the invention exhibits an enzymatic activity A_{Temp} of at least 1.001-fold, preferably of at least 1.002-fold, more preferably at least 1.1-fold, still more preferably at least 1.3-fold, yet more preferably at least 1.5-fold, even more preferably at least 2.0-fold, most preferably at least 2.5-fold, and utmost preferably at least 3.0-fold higher than the enzymatic activity A_{pH} of SEQ ID NO: 7.

In a preferred embodiment, the enzymatic activity of the glucose isomerase according to the invention is measured by
(i) incubating an aliquot of the glucose isomerase in 50 mM MES buffer ((2-N-morpholino) ethane sulfonic acid) containing 50 mM fructose and allowing the glucose isomerase to enzymatically convert fructose into glucose;
(ii) collecting 20 μl samples at different points in time during conversion and stopping the reaction within each sample by adding 20 μL HCl (0.25 M) followed by 60 μL potassium phosphate buffer (50 mM, pH 7.0);
(iii) quantifying the glucose in each sample; preferably by using a photometric assay; and
(iv) determining enzymatic activity on the basis of the quantified glucose in the samples as collected at different points in time during conversion; preferably in accordance with the Glucose Isomerase Activity Assay as defined herein and the Glucose Detection Assay as defined herein.

The glucose isomerase of SEQ ID NO:1 and all variants thereof are analyzed in a Glucose Isomerase Activity Assay according to the description disclosed herein. The glucose isomerases according to the invention are manufactured in the presence of magnesium cations, accordingly the homo-tetrameric variants carry magnesium cations as catalytic cations.

### Glucose Isomerase Activity Assay:

The enzymatic activity of a respective glucose isomerase was measured from crude extracts from lysed expression cells containing the recombinant specific glucose isomerase by using fructose as a substrate and measuring the amount of glucose produced in a biotransformation reaction over a defined time period under any specific assay condition (Specific Assay Conditions outlined below). Specifically, the biotransformation reaction of a respective glucose isomerase was performed by incubating 20 μL of the respective crude extract with 80 μL of fructose containing substrate buffer under the Specific Assay Conditions (corresponding to final reaction concentrations upon start of the enzymatic reaction). From the biotransformation reaction 20 μL samples were taken at different points in time (usually 2, 4, 20 h after reaction start). The reaction was stopped by addition of 20 μL HCl (0.25 M) followed by addition of 60 μL potassium phosphate buffer (50 mM, pH 7). The amount of glucose formed from fructose in the stopped biotransformation sample was then quantified with the Glucose Detection Assay in an end point detection (detection of all glucose formed).

The “enzymatic activity” is expressed as the ratio of glucose formed per reaction time, whereby for calculation of conversion only such samples may be used for analysis where the conversion of fructose to glucose in that specific samples is within a range of 5-25% conversion, to ensure reliable detection of the turnover.

The term “relative enzymatic activity” describes the ratio of the enzymatic activity of a first glucose isomerase in comparison to a second glucose isomerase; the relative enzymatic activity can be expressed as “percentage” (%) or as an “x-fold increase” or “x-fold decrease” of the first enzymatic activity over the second enzymatic activity.

The term “residual enzymatic activity” describes the ratio of the enzymatic activity of one glucose isomerase at a first reaction condition in comparison to the enzymatic activity of such one glucose isomerase at a second reaction condition; the residual enzymatic activity can be expressed as “percentage” (%) of the enzymatic activity at the second reaction condition in comparison to the second reaction condition.

For the purpose of the specification, when comparing enzymatic activities, relative enzymatic activities or residual enzymatic activities are compared with one another on the basis of same stoichiometric quantities of glucose isomerases, i.e. molar amounts.

### Glucose Detection Assay:

For quantification of the glucose contained in a test sample, for example for glucose formed during a Glucose Isomerase Activity Assay, the commercial assay kit (K-GLUHK-220A) from Megazymes has been used, which is a photometric assay detecting the formation of NADPH formed upon enzymatic oxidative conversion of glucose to glucose-6-phosphate in the sample, whereby the NADPH absorption signal correlates with the glucose concentration of the sample. The assay reagent is prepared according to the assay kit manual. In a transparent flat bottom microtiter plate, 100 μL of the assay reagent is mixed with 15 μL of an appropriately diluted sample from a stopped biotransformation reaction, whereby the dilution factor is selected such that during the Glucose Detection Assay the glucose concentration is less than 5.0 mM. The mixture is incubated at 30° C. for 10 min and the absorption is measured at 340 nm using a microtiter plate photometer. The glucose concentration in [mM] detected in the Glucose Detection Assay is calculated according to the assay kit manual by comparison to a calibration standard, the glucose concentration in [mM] in the test sample is calculated from the concentration in the Glucose Detection Assay in consideration of the preparation and dilution of such test sample.

### Specific Assay Conditions:

The following specific reaction conditions for determining enzymatic activity in the Glucose Isomerase Activity Assay have been used in various biotransformation reactions and are in accordance with the invention (MES=(2-N-morpholino) ethane sulfonic acid):
Condition 1: 50 mM fructose, 50 mM MES buffer, pH 7) at 35° C.;
Condition 2: 50 mM fructose, 50 mM MES buffer, pH 7), 5.0 mM CaCl₂) at 35° C.;
Condition 3: 50 mM fructose, 50 mM MES buffer, pH 5.8) at 35° C.;
Condition 4: 50 mM fructose, 50 mM MES buffer, pH 5.8), 5.0 mM CaCl₂) at 35° C.;
Condition 5: 50 mM fructose, 50 mM MES buffer, pH 5.8) at 25° C.;
Condition 6: 50 mM fructose, 50 mM MES buffer, pH 5.8), 5.0 mM CaCl₂) at 25° C.;
Condition 7: 50 mM fructose, 50 mM MES buffer, pH 5.8), 10 mM CaCl₂ at 25° C.;
Condition 8: 200 mM fructose, 50 mM MES buffer, pH 6.5), 0 mM CaCl₂ at 40° C.;
Condition 9: 200 mM fructose, 50 mM MES buffer, pH 6.5), 5 mM CaCl₂ at 40° C.;
Condition 10: 200 mM fructose, 50 mM MES buffer, pH 6.5), 10 mM CaCl₂) at 40° C.;
Condition 11: 200 mM fructose, 50 mM MES buffer, pH 6.5), 20 mM CaCl₂) at 40° C.;
Condition 12: 200 mM fructose, 50 mM MES buffer, pH 6.5), 30 mM CaCl₂ at 40° C.;
Condition 13: 200 mM fructose, 50 mM MES buffer, pH 6.5), 60 mM CaCl₂) at 40° C.;
Condition 14: 50 mM fructose, 50 mM MES buffer, pH 5.8), 5.0 mM CaCl₂) at 15° C.;
Condition 15: 50 mM fructose, 50 mM MES buffer, pH 5.8), 5.0 mM CaCl₂) at 20° C.

In a preferred embodiment of the invention, the enzymatic activity of the glucose isomerase according to the invention is measured in accordance with the Glucose Isomerase Activity Assay as defined herein and the Glucose Detection Assay as defined herein.

Preferably, for measuring the enzymatic activity in the absence of Ca²⁺ (A₀) of the glucose isomerase according to the invention, in step (i) the pH value is pH 5.8 or pH 7.0, preferably pH 5.8; the Ca²⁺ concentration is 0 mM; and the temperature is 25° C. or 35° C., preferably 35° C.

Preferably, for measuring enzymatic activity in the presence Ca²⁺ (A_{Ca2+}) of the glucose isomerase according to the invention, in step (i) the pH value is pH 5.8 or pH 7, preferably pH 5.8; the Ca²⁺ concentration is 5 mM or 10 mM, preferably 10 mM; and the temperature is 25° C. or 35° C., preferably 35° C.

Preferably, for measuring enzymatic activity at acidic pH (A_{pH}) of the glucose isomerase according to the invention, in step (i) the pH value is pH 5.8 or pH 7, preferably pH 5.8; the Ca²⁺ concentration is 0 mM, 5 mM, 10 mM, 20 mM, 30 mM, or 60 mM, preferably 0 mM, 5 mM, or 10 mM, more preferably 0 mM, or 5 mM; and the temperature is 15° C., 20° C., 25° C., or 35° C., preferably 25° C., or 35° C., and most preferably 35° C.

In a preferred embodiment, the enzymatic activity of the glucose isomerase according to the invention is measured according to the Specific Assay Conditions (i.e. any one of Conditions 1 to 15 as defined herein).

In a preferred embodiment, the glucose isomerase according to the invention exhibits in comparison to SEQ ID NO: 1 an
(i) increased enzymatic activity in the absence of Ca²⁺ (A₀) as well as increased enzymatic activity in the presence Ca²⁺ (A_{Ca2+}); and/or
(ii) increased enzymatic activity in the absence of Ca²⁺ (A₀) as well as increased enzymatic activity at acidic pH (A_{pH}); and/or
(iii) increased enzymatic activity in the presence Ca²⁺ (A_{Ca2+}) as well as increased enzymatic activity at acidic pH (A_{pH}); and/or
(iv) increased enzymatic activity in the absence of Ca²⁺ (A₀) as well as increased enzymatic activity in the presence Ca²⁺ (A_{Ca2+}) as well as increased enzymatic activity at acidic pH (A_{pH}); and/or
(v) increased enzymatic activity in the presence Ca²⁺ (A_{Ca2+}) as well as increased enzymatic activity at acidic pH (A_{pH}) as well as an increased enzymatic activity at low temperature (A_{Temp}).

In a preferred embodiment, the glucose isomerase according to the invention exhibits in comparison to SEQ ID NO: 7 an
(i) increased enzymatic activity in the absence of Ca²⁺ (A₀) as well as increased enzymatic activity in the presence Ca²⁺ (A_{Ca2+}); and/or
(ii) increased enzymatic activity in the absence of Ca²⁺ (A₀) as well as increased enzymatic activity at acidic pH (A_{pH}); and/or
(iii) increased enzymatic activity in the presence Ca²⁺ (A_{Ca2+}) as well as increased enzymatic activity at acidic pH (A_{pH}); and/or
(iv) increased enzymatic activity in the absence of Ca²⁺ (A₀) as well as increased enzymatic activity in the presence Ca²⁺ (A_{Ca2+}) as well as increased enzymatic activity at acidic pH (A_{pH}); and/or
(v) increased enzymatic activity in the presence Ca²⁺ (A_{Ca2+}) as well as increased enzymatic activity at acidic pH (A_{pH}) as well as an increased enzymatic activity at low temperature (A_{Temp}).

Preferably, the glucose isomerase according to the invention exhibits an im-proved enzymatic activity in comparison to SEQ ID NO: 1 under one or more Specific Assay Conditions (i.e. any one of Conditions 1 to 15 as defined herein).

Preferably, the glucose isomerase according to the invention exhibits an improved enzymatic activity in comparison to SEQ ID NO:7 under one or more Specific Assay Conditions (i.e. any one of Conditions 1 to 15 as defined herein).

Preferably, the glucose isomerase according to the invention comprises at least one, at least two, at least three, at least four, at least five, at least six, at least seven, at least eight, at least nine, at least ten, or at least eleven substitutions at substitution positions independently of one another selected from V187, Q300, P313, F242, W140, G190, A342, A47, A273, K343, and M309, preferably V187, P313, F242, and A47, and exhibits an increased enzymatic activity in the absence of Ca²⁺ (A₀) in comparison to SEQ ID NO: 1 with respect to the conversion of fructose into glucose or vice versa, preferably with respect to the conversion of fructose into glucose.

Preferably, the glucose isomerase according to the invention comprises at least one substitution selected from
V187C, V187T, V187N, V187Q, V187S, V187M,
F242C, F242T, F242N, F242Q, F242S, F242M,
P313S, P313T, P313N, P313Q, P313M, P313C,
A47S, A47T, Q47N, A47Q, A47M, A47C,
   and exhibits an increased enzymatic activity in the absence of Ca²⁺ (A₀) in comparison to SEQ ID NO:1 with respect to the conversion of fructose into glucose or vice versa, preferably with respect to the conversion of fructose into glucose.

Preferably, the glucose isomerase according to the invention comprises at least one, at least two, at least three, at least four, at least five, at least six, at least seven, at least eight, at least nine, at least ten, or at least eleven substitutions independently of one another selected from V187C, Q300Y, P313V, Q300S, F242C, P313S, W140H, Q300F, P313L, Q300C, G190N, F242V, A342S, A47S, A273S, K343R, and M309I, preferably V187C, F242C, P313S, and A47S, and exhibits an increased enzymatic activity in the absence of Ca²⁺ (A₀) in comparison to SEQ ID NO: 1 with respect to the conversion of fructose into glucose or vice versa, preferably with respect to the conversion of fructose into glucose.

Preferably, the glucose isomerase according to the invention comprises at least one, at least two, at least three, at least four, at least five, at least six, at least seven, at least eight, at least nine, at least ten, or at least eleven substitutions at substitution positions independently of one another selected from V187, Q300, P313, G190, F242, K343, M309, A342, A47, W140, A273, preferably V187, F242, P313, and A47; and exhibits (i) an increased enzymatic activity in the presence of Ca²⁺ (A_{Ca2+}) in comparison to SEQ ID NO: 1 with respect to the conversion of fructose into glucose or vice versa, preferably with respect to the conversion of fructose into glucose; and/or (ii) an increased residual enzymatic activity in the presence of Ca²⁺ (R A_{Ca2+}) in comparison to SEQ ID NO:1 with respect to the conversion of fructose into glucose or vice versa, preferably with respect to the conversion of fructose into glucose.

Preferably, the glucose isomerase according to the invention comprises at least one substitution selected from
V187C, V187T, V187N, V187Q, V187S, V187M,
F242C, F242T, F242N, F242Q, F242S, F242M,
P313S, P313T, P313N, P313Q, P313M, P313C,
A47S, A47T, Q47N, A47Q, A47M, A47C,
   and exhibits (i) an increased enzymatic activity in the presence of Ca²⁺ (A_{Ca2+}) in comparison to SEQ ID NO:1 with respect to the conversion of fructose into glucose or vice versa, preferably with respect to the conversion of fructose into glucose; and/or (ii) an increased residual enzymatic activity in the presence of Ca²⁺ (R A_{Ca2+}) in comparison to SEQ ID NO: 1 with respect to the conversion of fructose into glucose or vice versa, preferably with respect to the conversion of fructose into glucose.

Preferably, the glucose isomerase according to the invention comprises at least one, at least two, at least three, at least four, at least five, at least six, at least seven, at least eight, at least nine, at least ten, or at least eleven substitutions independently of one another selected from the group consisting of the substitutions V187C, Q300Y, P313V, Q300S, G190N, F242C, P313L, Q300F, K343R, M309I, Q300C, A342S, P313S, F242V, A47S, W140H, and A273S, preferably V187C, F242C, P313S, and A47S, and exhibits an (i) an increased enzymatic activity in the presence of Ca²⁺ (A_{Ca2+}) in comparison to SEQ ID NO: 1 with respect to the conversion of fructose into glucose or vice versa, preferably with respect to the conversion of fructose into glucose; and/or (ii) an increased residual enzymatic activity in the presence of Ca²⁺ (R A_{Ca2+}) in comparison to SEQ ID NO: 1 with respect to the conversion of fructose into glucose or vice versa, preferably with respect to the conversion of fructose into glucose.

Preferably, the glucose isomerase according to the invention comprises at least one, at least two, at least three, at least four, at least five, at least six, at least seven, at least eight substitutions at substitution positions independently of one another selected from V187, F242, M309, W140, P313, Q300, K343, A47 and M241, preferably V187, F242, P313, and A47; and exhibits an increased enzymatic activity at neutral and acidic pH (A_{pH}) in comparison to SEQ ID NO: 1 with respect to the conversion of fructose into glucose or vice versa, preferably with respect to the conversion of fructose into glucose.

Preferably, the glucose isomerase according to the invention comprises at least one substitution selected from
V187C, V187T, V187N, V187Q, V187S, V187M,
F242C, F242T, F242N, F242Q, F242S, F242M,
P313S, P313T, P313N, P313Q, P313M, P313C,
A47S, A47T, Q47N, A47Q, A47M, A47C,
   and exhibits an increased enzymatic activity at neutral and acidic pH (A_{pH}) in comparison to SEQ ID NO: 1 with respect to the conversion of fructose into glucose or vice versa, preferably with respect to the conversion of fructose into glucose.

Preferably, the glucose isomerase according to the invention comprises at least one, at least two, at least three, at least four, at least five, at least six, at least seven, at least eight or at least nine substitutions independently of one another selected from V187C, F242C, M309I, F242V, W140H, P313S, P313V, P313L, Q300Y, Q331G, K343R, and M241V, preferably V187C, F242C, P313S, and A47S, and exhibits an increased enzymatic activity at neutral and acidic pH (A_{pH}) in comparison to SEQ ID NO:1 with respect to the conversion of fructose into glucose or vice versa, preferably with respect to the conversion of fructose into glucose.

Preferably, the amino acid sequence of the glucose isomerase comprises or consists of any one of amino acid sequences with at least 67%, or at least 68%, or at least 70%, or at least 72%, or at least 74%, or at least 76%, or at least 78%, or at least 80%, or at least 82%, or at least 84%, or at least 86%, or at least 88%, or at least 90%, or at least 92%, or at least 94%, or at least 96%, or at least 98%, or at least 100% identity to SEQ ID NO: 7, and exhibits in comparison to SEQ ID NO: 1 an
(i) increased enzymatic activity in the absence of Ca²⁺ (A₀) as well as increased enzymatic activity in the presence Ca²⁺ (A_{Ca2+}); and/or
(ii) increased enzymatic activity in the absence of Ca²⁺ (A₀) as well as increased enzymatic activity at acidic pH (A_{pH}); and/or
(iii) increased enzymatic activity in the presence Ca²⁺ (A_{Ca2+}) as well as increased enzymatic activity at acidic pH (A_{pH}); and/or
(iv) increased enzymatic activity in the absence of Ca²⁺ (A₀) as well as increased enzymatic activity in the presence Ca²⁺ (A_{Ca2+}) as well as increased enzymatic activity at acidic pH (A_{pH}); and/or
(v) increased enzymatic activity in the presence Ca²⁺ (A_{Ca2+}) as well as increased enzymatic activity at acidic pH (A_{pH}) as well as an increased enzymatic activity at low temperature (A_{Temp}).

Preferably, the amino acid sequence of the glucose isomerase comprises an amino acid sequence of SEQ ID NO: 7, and exhibits in comparison to SEQ ID NO:1 an
(i) increased enzymatic activity in the absence of Ca²⁺ (A₀) as well as increased enzymatic activity in the presence Ca²⁺ (A_{Ca2+}); and/or
(ii) increased enzymatic activity in the absence of Ca²⁺ (A₀) as well as increased enzymatic activity at acidic pH (A_{pH}); and/or
(iii) increased enzymatic activity in the presence Ca²⁺ (A_{Ca2+}) as well as increased enzymatic activity at acidic pH (A_{pH}); and/or
(iv) increased enzymatic activity in the absence of Ca²⁺ (A₀) as well as increased enzymatic activity in the presence Ca²⁺ (A_{Ca2+}) as well as increased enzymatic activity at acidic pH (A_{pH}); and/or
(v) increased enzymatic activity in the presence Ca²⁺ (A_{Ca2+}) as well as increased enzymatic activity at acidic pH (A_{pH}) as well as an increased enzymatic activity at low temperature (A_{Temp}).

Preferably, the amino acid sequence of the glucose isomerase comprises or consists of any one of amino acid sequences with at least 67%, or at least 68%, or at least 70%, or at least 72%, or at least 74%, or at least 76%, or at least 78%, or at least 80%, or at least 82%, or at least 84%, or at least 86%, or at least 88%, or at least 90%, or at least 92%, or at least 94%, or at least 96%, or at least 98%, or at least 100% identity to SEQ ID NO: 11, and exhibits in comparison to SEQ ID NO: 1 an
(i) increased enzymatic activity in the absence of Ca²⁺ (A₀) as well as increased enzymatic activity in the presence Ca²⁺ (A_{Ca2+}); and/or
(ii) increased enzymatic activity in the absence of Ca²⁺ (A₀) as well as increased enzymatic activity at acidic pH (A_{pH}); and/or
(iii) increased enzymatic activity in the presence Ca²⁺ (A_{Ca2+}) as well as increased enzymatic activity at acidic pH (A_{pH}); and/or
(iv) increased enzymatic activity in the absence of Ca²⁺ (A₀) as well as increased enzymatic activity in the presence Ca²⁺ (A_{Ca2+}) as well as increased enzymatic activity at acidic pH (A_{pH}); and/or
(v) increased enzymatic activity in the presence Ca²⁺ (A_{Ca2+}) as well as increased enzymatic activity at acidic pH (A_{pH}) as well as an increased enzymatic activity at low temperature (A_{Temp}).

Preferably, the amino acid sequence of the glucose isomerase comprises an amino acid sequence of SEQ ID NO: 11, and exhibits in comparison to SEQ ID NO:1 an
(i) increased enzymatic activity in the absence of Ca²⁺ (A₀) as well as increased enzymatic activity in the presence Ca²⁺ (A_{Ca2+}); and/or
(ii) increased enzymatic activity in the absence of Ca²⁺ (A₀) as well as increased enzymatic activity at acidic pH (A_{pH}); and/or
(iii) increased enzymatic activity in the presence Ca²⁺ (A_{Ca2+}) as well as increased enzymatic activity at acidic pH (A_{pH}); and/or
(iv) increased enzymatic activity in the absence of Ca²⁺ (A₀) as well as increased enzymatic activity in the presence Ca²⁺ (A_{Ca2+}) as well as increased enzymatic activity at acidic pH (A_{pH}); and/or
(v) increased enzymatic activity in the presence Ca²⁺ (A_{Ca2+}) as well as increased enzymatic activity at acidic pH (A_{pH}) as well as an increased enzymatic activity at low temperature (A_{Temp}).

Preferably, the amino acid sequence of the glucose isomerase comprises or consists of any one of amino acid sequences with at least 67%, or at least 68%, or at least 70%, or at least 72%, or at least 74%, or at least 76%, or at least 78%, or at least 80%, or at least 82%, or at least 84%, or at least 86%, or at least 88%, or at least 90%, or at least 92%, or at least 94%, or at least 96%, or at least 98%, or at least 100% identity to SEQ ID NO: 19, and exhibits in comparison to SEQ ID NO: 1 an
(i) increased enzymatic activity in the absence of Ca²⁺ (A₀) as well as increased enzymatic activity in the presence Ca²⁺ (A_{Ca2+}); and/or
(ii) increased enzymatic activity in the absence of Ca²⁺ (A₀) as well as increased enzymatic activity at acidic pH (A_{pH}); and/or
(iii) increased enzymatic activity in the presence Ca²⁺ (A_{Ca2+}) as well as increased enzymatic activity at acidic pH (A_{pH}); and/or
(iv) increased enzymatic activity in the absence of Ca²⁺ (A₀) as well as increased enzymatic activity in the presence Ca²⁺ (A_{Ca2+}) as well as increased enzymatic activity at acidic pH (A_{pH}); and/or
(v) increased enzymatic activity in the presence Ca²⁺ (A_{Ca2+}) as well as increased enzymatic activity at acidic pH (A_{pH}) as well as an increased enzymatic activity at low temperature (A_{Temp}).

Preferably, the amino acid sequence of the glucose isomerase comprises an amino acid sequence of SEQ ID NO: 19, and exhibits in comparison to SEQ ID NO:1 an
(i) increased enzymatic activity in the absence of Ca²⁺ (A₀) as well as increased enzymatic activity in the presence Ca²⁺ (A_{Ca2+}); and/or
(ii) increased enzymatic activity in the absence of Ca²⁺ (A₀) as well as increased enzymatic activity at acidic pH (A_{pH}); and/or
(iii) increased enzymatic activity in the presence Ca²⁺ (A_{Ca2+}) as well as increased enzymatic activity at acidic pH (A_{pH}); and/or
(iv) increased enzymatic activity in the absence of Ca²⁺ (A₀) as well as increased enzymatic activity in the presence Ca²⁺ (A_{Ca2+}) as well as increased enzymatic activity at acidic pH (A_{pH}); and/or
(v) increased enzymatic activity in the presence Ca²⁺ (A_{Ca2+}) as well as increased enzymatic activity at acidic pH (A_{pH}) as well as an increased enzymatic activity at low temperature (A_{Temp}).

Preferably, the amino acid sequence of the glucose isomerase comprises or consists of any one of amino acid sequences with at least 67%, or at least 68%, or at least 70%, or at least 72%, or at least 74%, or at least 76%, or at least 78%, or at least 80%, or at least 82%, or at least 84%, or at least 86%, or at least 88%, or at least 90%, or at least 92%, or at least 94%, or at least 96%, or at least 98%, or at least 100% identity to SEQ ID NO: 3, and exhibits in comparison to SEQ ID NO: 1 an
(i) increased enzymatic activity in the absence of Ca²⁺ (A₀) as well as increased enzymatic activity in the presence Ca²⁺ (A_{Ca2+}); and/or
(ii) increased enzymatic activity in the absence of Ca²⁺ (A₀) as well as increased enzymatic activity at acidic pH (A_{pH}); and/or
(iii) increased enzymatic activity in the presence Ca²⁺ (A_{Ca2+}) as well as increased enzymatic activity at acidic pH (A_{pH}); and/or
(iv) increased enzymatic activity in the absence of Ca²⁺ (A₀) as well as increased enzymatic activity in the presence Ca²⁺ (A_{Ca2+}) as well as increased enzymatic activity at acidic pH (A_{pH}); and/or
(v) increased enzymatic activity in the presence Ca²⁺ (A_{Ca2+}) as well as increased enzymatic activity at acidic pH (A_{pH}) as well as an increased enzymatic activity at low temperature (A_{Temp}).

Preferably, the amino acid sequence of the glucose isomerase comprises an amino acid sequence of SEQ ID NO: 3, and exhibits in comparison to SEQ ID NO:1 an
(i) increased enzymatic activity in the absence of Ca²⁺ (A₀) as well as increased enzymatic activity in the presence Ca²⁺ (A_{Ca2+}); and/or
(ii) increased enzymatic activity in the absence of Ca²⁺ (A₀) as well as increased enzymatic activity at acidic pH (A_{pH}); and/or
(iii) increased enzymatic activity in the presence Ca²⁺ (A_{Ca2+}) as well as increased enzymatic activity at acidic pH (A_{pH}); and/or
(iv) increased enzymatic activity in the absence of Ca²⁺ (A₀) as well as increased enzymatic activity in the presence Ca²⁺ (A_{Ca2+}) as well as increased enzymatic activity at acidic pH (A_{pH}); and/or
(v) increased enzymatic activity in the presence Ca²⁺ (A_{Ca2+}) as well as increased enzymatic activity at acidic pH (A_{pH}) as well as an increased enzymatic activity at low temperature (A_{Temp}).

Preferably, the glucose isomerase according to the invention comprises at least two, at least three, at least four, at least five, at least six, at least seven, at least eight, or at least nine substitutions at substitution positions independently of one another selected from V187, A47, M241, F242, Q300, M309, P313, A342 and K343, and exhibits:
(i) an increased enzymatic activity in the absence of Ca²⁺ (A₀) in comparison to SEQ ID NO:7 with respect to the conversion of fructose into glucose or vice versa, preferably with respect to the conversion of fructose into glucose; and/or
(ii) an increased enzymatic activity in the presence of Ca²⁺ (A_{Ca2+}) in comparison to SEQ ID NO:7 with respect to the conversion of fructose into glucose or vice versa, preferably with respect to the conversion of fructose into glucose; and/or
(iii) an increased residual enzymatic activity in the presence of Ca²⁺ (RA_{Ca2+}) in comparison to SEQ ID NO:7 with respect to the conversion of fructose into glucose or vice versa, preferably with respect to the conversion of fructose into glucose; and/or
(iv) an increased enzymatic activity at neutral and acidic pH (A_{pH}) in comparison to SEQ ID NO:7 with respect to the conversion of fructose into glucose or vice versa, preferably with respect to the conversion of fructose into glucose; and/or
(v) an increased enzymatic activity at low temperatures (A_{Temp}) in comparison to SEQ ID NO:7 with respect to the conversion of fructose into glucose or vice versa, preferably with respect to the conversion of fructose into glucose.

Preferably, the glucose isomerase according to the invention comprises a substitution in at least two, at least three, at least four, at least five, at least six, at least seven, at least eight or at least nine substitutions independently of one another selected from V187C, V187W, V187T, A47S, M241V, F242C, Q300Y, Q300S, M309I, P313V, P313S, A342S, K343R, and K343N, and exhibits:
(i) an increased enzymatic activity in the absence of Ca²⁺ (A₀) in comparison to SEQ ID NO:7 with respect to the conversion of fructose into glucose or vice versa, preferably with respect to the conversion of fructose into glucose; and/or
(ii) an increased enzymatic activity in the presence of Ca²⁺ (A_{Ca2+}) in comparison to SEQ ID NO:7 with respect to the conversion of fructose into glucose or vice versa, preferably with respect to the conversion of fructose into glucose; and/or
(iii) an increased residual enzymatic activity in the presence of Ca²⁺ (RA_{Ca2+}) in comparison to SEQ ID NO:7 with respect to the conversion of fructose into glucose or vice versa, preferably with respect to the conversion of fructose into glucose; and/or
(iv) an increased enzymatic activity at neutral and acidic pH (A_{pH}) in comparison to SEQ ID NO:7 with respect to the conversion of fructose into glucose or vice versa, preferably with respect to the conversion of fructose into glucose; and/or
(v) an increased enzymatic activity at low temperatures (A_{Temp}) in comparison to SEQ ID NO:7 with respect to the conversion of fructose into glucose or vice versa, preferably with respect to the conversion of fructose into glucose.

Preferably, the amino acid sequence of the glucose isomerase comprises or consists of any one of amino acid sequences with at least 80%, preferably at least 85%, more preferably at least 90%, yet more preferably at least 95%, most preferably at least 99.9%, or utmost preferably 100%, to each one of SEQ ID NO: 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, or 73 and exhibits:
(i) an increased enzymatic activity in the absence of Ca²⁺ (A₀) in comparison to SEQ ID NO:7 with respect to the conversion of fructose into glucose or vice versa, preferably with respect to the conversion of fructose into glucose; and/or
(ii) an increased enzymatic activity in the presence of Ca²⁺ (A_{Ca2+}) in comparison to SEQ ID NO:7 with respect to the conversion of fructose into glucose or vice versa, preferably with respect to the conversion of fructose into glucose; and/or
(iii) an increased residual enzymatic activity in the presence of Ca²⁺ (RA_{Ca2+}) in comparison to SEQ ID NO:7 with respect to the conversion of fructose into glucose or vice versa, preferably with respect to the conversion of fructose into glucose; and/or
(iv) an increased enzymatic activity at neutral and acidic pH (A_{pH}) in comparison to SEQ ID NO:7 with respect to the conversion of fructose into glucose or vice versa, preferably with respect to the conversion of fructose into glucose; and/or
(v) an increased enzymatic activity at low temperatures (A_{Temp}) in comparison to SEQ ID NO:7 with respect to the conversion of fructose into glucose or vice versa, preferably with respect to the conversion of fructose into glucose.

Preferably, the amino acid sequence of the glucose isomerase comprises or consists of any one of amino acid sequences selected from the group consisting of SEQ ID NO: 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, or 73, and exhibits:
(i) an increased enzymatic activity in the absence of Ca²⁺ (A₀) in comparison to SEQ ID NO:7 with respect to the conversion of fructose into glucose or vice versa, preferably with respect to the conversion of fructose into glucose; and/or
(ii) an increased enzymatic activity in the presence of Ca²⁺ (A_{Ca2+}) in comparison to SEQ ID NO:7 with respect to the conversion of fructose into glucose or vice versa, preferably with respect to the conversion of fructose into glucose; and/or
(iii) an increased residual enzymatic activity in the presence of Ca²⁺ (RA_{Ca2+}) in comparison to SEQ ID NO:7 with respect to the conversion of fructose into glucose or vice versa, preferably with respect to the conversion of fructose into glucose; and/or
(iv) an increased enzymatic activity at neutral and acidic pH (A_{pH}) in comparison to SEQ ID NO:7 with respect to the conversion of fructose into glucose or vice versa, preferably with respect to the conversion of fructose into glucose; and/or
(v) an increased enzymatic activity at low temperatures (A_{Temp}) in comparison to SEQ ID NO:7 with respect to the conversion of fructose into glucose or vice versa, preferably with respect to the conversion of fructose into glucose.

Preferably, the amino acid sequence of the glucose isomerase comprises or consists of any one of amino acid sequences with at least 80%, preferably at least 85%, more preferably at least 90%, yet more preferably at least 95%, most preferably at least 99.9%, or utmost preferably 100%, to each one of the sequences selected from the groups consisting of:
SEQ ID NO: 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, and 73; or
SEQ ID NO: 28, 29, 32, 36, 37, 38, 39, 40, 41, 42, 44, 45, 46, 47, 53, 54, 56, 57, 58, 59, 60, 61, 62, 63, 64, 65, 66, 67, 68, 69, 70, 71, 72, and 73; or
SEQ ID NO: 26, 32, 34, 35, 43, 44, 51, 62, 64, 66, 70, and 72; or
SEQ ID NO: 27, 30, 33, 34, 37, 41, 42, 43, 44, 45, 47, 48, 49, 50, 51, 53, 54, 55, 56, 58, 59, 60, 61, 63, 64, 65, 66, 68, 69, 70, 71, 72, and 73;
   and exhibits:
(i) an increased enzymatic activity in the absence of Ca²⁺ (A₀) in comparison to SEQ ID NO:7 with respect to the conversion of fructose into glucose or vice versa, preferably with respect to the conversion of fructose into glucose; and/or
(ii) an increased enzymatic activity in the presence of Ca²⁺ (A_{Ca2+}) in comparison to SEQ ID NO:7 with respect to the conversion of fructose into glucose or vice versa, preferably with respect to the conversion of fructose into glucose; and/or
(iii) an increased residual enzymatic activity in the presence of Ca²⁺ (RA_{Ca2+}) in comparison to SEQ ID NO:7 with respect to the conversion of fructose into glucose or vice versa, preferably with respect to the conversion of fructose into glucose; and/or
(iv) an increased enzymatic activity at neutral and acidic pH (A_{pH}) in comparison to SEQ ID NO:7 with respect to the conversion of fructose into glucose or vice versa, preferably with respect to the conversion of fructose into glucose; and/or
(v) an increased enzymatic activity at low temperatures (A_{Temp}) in comparison to SEQ ID NO:7 with respect to the conversion of fructose into glucose or vice versa, preferably with respect to the conversion of fructose into glucose.

Preferably, the amino acid sequence of the glucose isomerase comprises or consists of any one of amino acid sequences selected from the groups consisting of
SEQ ID NO: 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, and 73; or
SEQ ID NO: 28, 29, 32, 36, 37, 38, 39, 40, 41, 42, 44, 45, 46, 47, 53, 54, 56, 57, 58, 59, 60, 61, 62, 63, 64, 65, 66, 67, 68, 69, 70, 71, 72, and 73; or
SEQ ID NO: 26, 32, 34, 35, 43, 44, 51, 62, 64, 66, 70, and 72; or
SEQ ID NO: 27, 30, 33, 34, 37, 41, 42, 43, 44, 45, 47, 48, 49, 50, 51, 53, 54, 55, 56, 58, 59, 60, 61, 63, 64, 65, 66, 68, 69, 70, 71, 72, and 73;
   and exhibits:
(i) an increased enzymatic activity in the absence of Ca²⁺ (A₀) in comparison to SEQ ID NO:7 with respect to the conversion of fructose into glucose or vice versa, preferably with respect to the conversion of fructose into glucose; and/or
(ii) an increased enzymatic activity in the presence of Ca²⁺ (A_{Ca2+}) in comparison to SEQ ID NO:7 with respect to the conversion of fructose into glucose or vice versa, preferably with respect to the conversion of fructose into glucose; and/or
(iii) an increased residual enzymatic activity in the presence of Ca²⁺ (RA_{Ca2+}) in comparison to SEQ ID NO:7 with respect to the conversion of fructose into glucose or vice versa, preferably with respect to the conversion of fructose into glucose; and/or
(iv) an increased enzymatic activity at neutral and acidic pH (A_{pH}) in comparison to SEQ ID NO:7 with respect to the conversion of fructose into glucose or vice versa, preferably with respect to the conversion of fructose into glucose; and/or
(v) an increased enzymatic activity at low temperatures (A_{Temp}) in comparison to SEQ ID NO:7 with respect to the conversion of fructose into glucose or vice versa, preferably with respect to the conversion of fructose into glucose.

It will be appreciated that the disclosures of any of the embodiments or preferred embodiments of the glucose isomerase under this first aspect of the invention refer to each other and are also deemed embodiments or preferred embodiments of each other.

A second aspect of the invention relates to a process for the conversion of an aldose molecule into a ketose molecule said method comprising the step of contacting said aldose molecule with a glucose isomerase according to the first aspect of the invention.

In a preferred embodiment, the invention relates to a process for the conversion of an aldose molecule, wherein the aldose molecule is glucose.

In a preferred embodiment, the process according to the invention takes place in a liquid reaction medium, said liquid reaction medium being preferably characterized by:
a pH of at least 4.5; more preferably of at least 5.8; and/or
a pH of at most 8.0; more preferably of at most 7.0; and/or
a pH of in the range of 5.0 to 8.0, or 5.3 to 8.0, or 5.5 to 8.0, or 5.8 to 8.0, or 6.0 to 8.0, or 6.4 to 8.0, or 6.6 to 8.0, or 6.9 to 8.0, preferably within the range of 5.0 to 7.5, or 5.3 to 7.5, or 5.5 to 7.5, or 5.8 to 7.5, or 6.0 to 7.5, or 6.4 to 7.5, or 6.6 to 7.5, or 6.9 to 7.5, more preferably in the range of 5.0 to 7.0, or 5.3 to 7.0, or 5.5 to 7.0, or 5.8 to 7.0, or 6.0 to 7.0, or 6.4 to 7.0, or 6.6 to 7.0, or 6.9 to 7.0; and/or
a calcium ion concentration of at most 60.0 mM; preferably at most 30.0 mM; preferably at most 20.0 mM; most preferably of at most 10.0 mM; and/or
a calcium ion concentration of 0 mM or in the range of 0.5 mM to 60 mM, 1 mM to 60 mM, 2 mM to 60 mM, 3 mM to 60 mM, 4.0 mM to 60 mM, 5 mM to 60 mM, 10 mM to 60 mM, 20 mM to 60 mM, or 30 mM to 60 mM, or 0.5 mM to 30 mM, 1 mM to 30 mM, 2 mM to 30 mM, 3 mM to 30 mM, 4.0 mM to 30 mM, 5 mM to 30 mM, 10 mM to 30 mM, 20 mM to 30 mM, 0.5 mM to 20 mM, 1 mM to 20 mM, 2 mM to 20 mM, 3 mM to 20 mM, 4.0 mM to 20 mM, 5 mM to 20 mM, 10 mM to 20 mM, or 0.5 mM to 10 mM, 1 mM to 10 mM, 2 mM to 10 mM, 3 mM to 10 mM, 4.0 mM to 10 mM, 5 mM to 10 mM;
a temperature of at least at least 2.0° C.; preferably at least 4.0° C.; more preferably of at least 10° C.; and/or
a temperature of at most at most 100.0° C., or at most 50.0° C.; preferably at most 45.0° C.; and/or
a temperature within the range of 2.0° C. to 99.5° C., or 3.0° C. to 95.0° C., or 4.0° C. to 90.0° C., or 5.0° C. to 85.0° C., or 6.0° C. to 80.0° C., or 6.0° C. to 75.0° C., or 8.0° C. to 70.0° C., or 9.0° C. to 65.0° C., or 10.0° C. to 60.0° C., or 10.0° C. to 55.0° C., or 10.0° C. to 50.0° C., or 10.0° C. to 45.0° C., preferably within the range of 2.0° C. to 99.5° C., or 3.0° C. to 95.0° C., or 4.0° C. to 90.0° C., or 5.0° C. to 85.0° C., or 6.0° C. to 80.0° C., or 6.0° C. to 75.0° C., or 8.0° C. to 70.0° C., or 9.0° C. to 65.0° C., or 10.0° C. to 60.0° C., or 10.0° C. to 55.0° C., or 10.0° C. to 50.0° C., or 10.0° C. to 45.0° C.; more preferably within the range of 2.0° C. to 99.5° C., or 3.0° C. to 95.0° C., or 4.0° C. to 90.0° C., or 5.0° C. to 85.0° C., or 6.0° C. to 80.0° C., or 6.0° C. to 75.0° C., or 8.0° C. to 70.0° C., or 9.0° C. to 65.0° C., or 10.0° C. to 60.0° C., or 10.0° C. to 55.0° C., or 10.0° C. to 50.0° C., or 10.0° C. to 45.0° C.; and/or
an aldose concentration at least 0.01% (w/w), preferably at least 0.1% (w/w) more preferably of at least 0.2% (w/w); and/or
an aldose concentration at most 50% (w/w), preferably at most 10% (w/w) more preferably of at most 5% (w/w); and/or
an aldose concentration in the range of 0.6±0.5% (w/w), or 0.8±0.5% (w/w), or 1.0±0.5% (w/w), or 2.0±0.5% (w/w), or 2.5±0.5% (w/w), 3.0±0.5% (w/w), or 3.5±0.5% (w/w), 4.0±0.5% (w/w), or 4.5±0.5% (w/w), or 5.0±0.5% (w/w), or 5.5±0.5% (w/w), or 6.0±0.5% (w/w), or 6.5±0.5% (w/w), 7.0±0.5% (w/w), or 7.5±0.5% (w/w), 8.0±0.5% (w/w), or 8.5±0.5% (w/w), 9.0±0.5% (w/w), or 9.5±0.5% (w/w), or 10.0±0.5% (w/w), 12.0±0.5% (w/w), or 14.0±0.5% (w/w), 16.0±0.5% (w/w), 18.0±0.5% (w/w), or 20.0±0.5% (w/w), or 25.0±0.5% (w/w), or 30.0±0.5% (w/w), or 35.0±0.5% (w/w), or 40.0±0.5% (w/w), or 45.0±0.5% (w/w), or 49.5±0.5 mM; preferably within the range of 0.6±0.2% (w/w), or 0.8±0.2% (w/w), or 1.0±0.2% (w/w), or 2.0±0.2% (w/w), or 2.5±0.2% (w/w), 3.0±0.2% (w/w), or 3.5±0.2% (w/w), 4.0±0.2% (w/w), or 4.5±0.2% (w/w), or 5.0±0.2% (w/w), or 5.5±0.2% (w/w), or 6.0±0.2% (w/w), or 6.5±0.2% (w/w), 7.0±0.2% (w/w), or 7.5±0.2% (w/w), 8.0±0.2% (w/w), or 8.5±0.2% (w/w), 9.0±0.2% (w/w), or 9.5±0.2% (w/w), or 10.0±0.2% (w/w), 12.0±0.2% (w/w), or 14.0±0.2% (w/w), 16.0±0.2% (w/w), 18.0±0.2% (w/w), or 20.0±0.2% (w/w), or 25.0±0.2% (w/w), or 30.0±0.2% (w/w), or 35.0±0.2% (w/w), or 40.0±0.2% (w/w), or 45.0±0.2% (w/w), or 49.5±0.2 mM; more preferably within the range of 0.6±0.1% (w/w), or 0.8±0.1% (w/w), or 1.0±0.1% (w/w), or 2.0±0.1% (w/w), or 2.5±0.1% (w/w), 3.0±0.1% (w/w), or 3.5±0.1% (w/w), 4.0±0.1% (w/w), or 4.5±0.1% (w/w), or 5.0±0.1% (w/w), or 5.5±0.1% (w/w), or 6.0±0.1% (w/w), or 6.5±0.1% (w/w), 7.0±0.1% (w/w), or 7.5±0.1% (w/w), 8.0±0.1% (w/w), or 8.5±0.1% (w/w), 9.0±0.1% (w/w), or 9.5±0.1% (w/w), or 10.0±0.1% (w/w), 12.0±0.1% (w/w), or 14.0±0.1% (w/w), 16.0±0.1% (w/w), 18.0±0.1% (w/w), or 20.0±0.1% (w/w), or 25.0±0.1% (w/w), or 30.0±0.1% (w/w), or 35.0±0.1% (w/w), or 40.0±0.1% (w/w), or 45.0±0.1% (w/w), or 49.5±0.1 mM.

A third aspect of the invention relates to the use of a glucose isomerase to produce in a liquid reaction medium
a ketose molecule by conversion of an aldose molecule; and/or
an aldose molecule by conversion of a ketose molecule;
   wherein the glucose isomerase is a glucose isomerase according to the first aspect of the invention; and wherein the liquid reaction medium is defined according to the second aspect of the invention.

In a preferred embodiment of the third aspect of the invention,
the ketose molecule fructose is converted to the aldose molecule glucose; and/or
the aldose molecule glucose is converted to the ketose molecule fructose;

In a preferred embodiment of the third aspect of the invention, the aldose molecule glucose is converted to the ketose molecule fructose.

In a preferred embodiment of the third aspect of the invention, the liquid reaction medium is characterized by:
a pH of in the range of 5.0 to 8.0, or 5.3 to 8.0, or 5.5 to 8.0, or 5.8 to 8.0, or 6.0 to 8.0, or 6.4 to 8.0, or 6.6 to 8.0, or 6.9 to 8.0, preferably within the range of 5.0 to 7.5, or 5.3 to 7.5, or 5.5 to 7.5, or 5.8 to 7.5, or 6.0 to 7.5, or 6.4 to 7.5, or 6.6 to 7.5, or 6.9 to 7.5, more preferably in the range of 5.0 to 7.0, or 5.3 to 7.0, or 5.5 to 7.0, or 5.8 to 7.0, or 6.0 to 7.0, or 6.4 to 7.0, or 6.6 to 7.0, or 6.9 to 7.0; and/or
a calcium ion concentration of 0 mM or in the range between 0.5 mM to 60 mM, 1 mM to 60 mM, 2 mM to 60 mM, 3 mM to 60 mM, 4.0 mM to 60 mM, 5 mM to 60 mM, 10 mM to 60 mM, 20 mM to 60 mM, or 30 mM to 60 mM, or 0.5 mM to 30 mM, 1 mM to 30 mM, 2 mM to 30 mM, 3 mM to 30 mM, 4.0 mM to 30 mM, 5 mM to 30 mM, 10 mM to 30 mM, 20 mM to 30 mM, 0.5 mM to 20 mM, 1 mM to 20 mM, 2 mM to 20 mM, 3 mM to 20 mM, 4.0 mM to 20 mM, 5 mM to 20 mM, 10 mM to 20 mM, or 0.5 mM to 10 mM, 1 mM to 10 mM, 2 mM to 10 mM, 3 mM to 10 mM, 4.0 mM to 10 mM, 5 mM to 10 mM.

The Table 1 below shows a summary of exemplary enzyme mutants according to the present invention, whereby the amino acid substitutions in which each individual variant differs from the amino acids of SEQ ID NO: 1 are indicated.

SEQ ID NO: 1 is the reference wild-type enzyme from Bifidobacterium adolescents and SEQ ID NO: 2 to 74 are engineered variants carrying from 1 to 7 substitutions in one of the 17 substitution positions in comparison to SEQ ID NO:1, with the substitution positions being compiled in Table 1:

TABLE 1
single mutants - substitution(s) in comparison to SEQ ID NO: 1

| SEQ ID NO: | A47 | V187 | M241 | F242 | Q300 | M309 | P313 | A342 | K343 | further |
|---|---|---|---|---|---|---|---|---|---|---|
| 1 | — | — | — | — | — | — | — | — | — | — |
| 2 | | | | | | | | | | P18S |
| 3 | S | | | | | | | | | |
| 4 | | | | | | | | | | W140H |
| 5 | | | | | | | | | | T147R |
| 6 | | | | | | | | | | I163V |
| 7 | | C | | | | | | | | |
| 8 | | | | | | | | | | G190N |
| 9 | | | | | | | | | | L198I |
| 10 | | | V | | | | | | | |
| 11 | | | | C | | | | | | |
| 12 | | | | V | | | | | | |
| 13 | | | | | | | | | | A273S |
| 14 | | | | | Y | | | | | |
| 15 | | | | | S | | | | | |
| 16 | | | | | C | | | | | |
| 17 | | | | | F | | | | | |
| 18 | | | | | | I | | | | |
| 19 | | | | | | | S | | | |
| 20 | | | | | | | V | | | |
| 21 | | | | | | | L | | | |
| 22 | | | | | | | | | | Q331G |
| 23 | | | | | | | | S | | |
| 24 | | | | | | | | | R | |

TABLE 2
multiple mutants - substitution(s) in comparison to SEQ ID NO: 1

| SEQ | | | | | | | | | |
|---|---|---|---|---|---|---|---|---|---|
| ID NO: | A47 | V187 | M241 | F242 | Q300 | M309 | P313 | A342 | K343 |
| 25 | | C | | | Y | | | | |
| 26 | | C | | | | | S | | R |
| 27 | S | C | | | | | | S | |
| 28 | | C | V | C | | | | | |
| 29 | | C | | C | | | | | R |
| 30 | S | C | | | | | V | | |
| 31 | | C | | | S | I | | | |
| 32 | | T | | C | | | S | | |
| 33 | S | C | | | | | | | R |
| 34 | S | C | | | | | S | | |
| 35 | | C | | | S | I | S | | |
| 36 | | C | | C | S | I | | | |
| 37 | S | W | | C | S | | | | |
| 38 | | C | V | C | Y | | | | |
| 39 | | W | V | C | | | V | | |
| 40 | | C | | C | Y | I | | | |
| 41 | S | C | | C | | | | S | |
| 42 | S | C | V | C | | | | | |
| 43 | S | C | | | | I | S | | |
| 44 | S | W | | C | | | S | | |
| 45 | S | C | | C | | I | | | |
| 46 | | C | V | C | | | G | | |
| 47 | S | C | | C | Y | | | | |
| 48 | S | C | | | | | V | | R |
| 49 | S | C | | | | | | S | R |
| 50 | S | C | | | | I | V | | |
| 51 | S | C | | | | | S | S | |
| 52 | | C | | | S | | | S | |
| 53 | S | C | | C | | I | | | R |
| 54 | S | C | | C | | I | | S | |
| 55 | S | C | | | S | I | | | R |
| 56 | S | C | V | C | | I | | | |
| 57 | | C | V | C | S | I | | | |
| 58 | S | C | V | C | | | | | R |
| 59 | S | C | V | C | Y | | | | |
| 60 | S | C | | C | Y | | | | N |
| 61 | S | C | | C | | | | S | N |
| 62 | | C | | C | | | S | S | |
| 63 | S | C | V | C | | | | S | |
| 64 | S | C | V | C | | | S | | |
| 65 | S | C | V | C | S | | | | |
| 66 | S | C | | C | | I | S | | |
| 67 | | C | V | C | | | | S | |
| 68 | S | C | V | C | S | I | | | |
| 69 | S | C | V | C | | | | S | |
| 70 | S | C | | C | | I | S | | R |
| 71 | S | C | | C | | I | | S | R |
| 72 | S | C | V | C | | I | S | | R |
| 73 | S | C | V | C | | I | | S | R |

Preferred embodiments of the invention are summarized as clauses 1 to 162 hereinafter:

Clause 1: A glucose isomerase comprising an amino acid sequence which has at least 65% identity to SEQ ID NO: 1 and which compared to SEQ ID NO: 1 comprises at least one, two, three, four, five, six, seven, eight, nine, ten, eleven, twelve, thirteen, fourteen, fifteen, sixteen or seventeen substitutions at substitution positions, which are located in subsequence R1 ranging from position L266 to R346 of SEQ ID NO:1; and/or subsequence R2 ranging from position L231 to Q243 of SEQ ID NO:1; and/or subsequence R3 ranging from position N185 to W199 of SEQ ID NO:1; and/or subsequence R4 ranging from position L139 to Y164 of SEQ ID NO:1; and/or subsequence R5 ranging from position G17 to R67 of SEQ ID NO:1.

Clause 2: The glucose isomerase of clause 1, wherein the amino acid sequence comprises at least one, two, three, four, five, or six substitutions at substitution positions in subsequence R1; preferably in subsequence R1-A ranging from position L266 to Y280; preferably ranging from position L266 to A273, more preferably ranging from position N267 to A273, and most preferably in position A273; and/or subsequence R1-B ranging from position S295 to D315, preferably in subsequence R1-B1 ranging from position S295 to D302, more preferably ranging from position A298 to Q300, and most preferably in position Q300; and/or subsequence R1-B2 ranging from position W307 to D315, more preferably ranging from position D308 to P313, and most preferably in positions M309 and/or P313; and/or subsequence R1-C ranging from position G330 to P334; preferably in position Q331; and/or subsequence R1-D ranging from position G337 to R346; preferably ranging from position L338 to K343, more preferably ranging from position N339 to K343, and most preferably in positions A342 and/or K343.

Clause 3: The glucose isomerase of any of the preceding clauses, wherein the amino acid sequence comprises at least one or two substitutions, wherein the substitution positions are in subsequence R2, preferably in positions M241 and/or F242.

Clause 4: The glucose isomerase according to any to any of the preceding clauses, wherein the amino acid sequence comprises at least one, two or three substitutions, wherein the substitution positions are in subsequence R3; preferably in subsequence R3-A ranging from position N185 to R192, more preferably ranging from position V187 to G191, still more preferably in positions V187 and/or G190; and most preferably in position V187; and/or subsequence R3-B consisting of position L198.

Clause 5: The glucose isomerase of any of the preceding clauses, wherein the amino acid sequence comprises at least one, two or three substitutions, wherein the substitution positions are in subsequence R4; preferably in subsequence R4-A ranging from position L139 to T147, preferably in subsequence R4-A1 ranging from position L139 to W140, and most preferably in position W140, and/or subsequence R4-A2 ranging from position T142 to T147, and most preferably in position T147; and/or subsequence R4-B ranging from position A161 to Y164, and more preferably in position I163.

Clause 6: The glucose isomerase of any of the preceding clauses, wherein the amino acid sequence comprises at least one or two substitutions, wherein the substitution positions are in subsequence R5; preferably in subsequence R5-A ranging from position G17 to Q19, more preferably in position P18; and/or subsequence R5-B ranging from position F44 to R67, more preferably ranging from position F44 to Q54, even more preferably ranging from position G45 to H50, and most preferably in position A47.

Clause 7: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions at substitution positions which are located in subsequence R3-A ranging from position N185 to R192 of SEQ ID NO: 1, and preferably independently of one another selected from the group consisting of substitution positions V187, and G190; and/or subsequence R1-A ranging from position L266 to Y280 of SEQ ID NO: 1, preferably substitution position A273; and/or subsequence R1-B ranging from position S295 to D315 of SEQ ID NO:1, and preferably independently of one another selected from the groups consisting of substitution positions P313, Q300, and M309; and/or subsequence R1-D ranging from position G337 to R346 of SEQ ID NO: 1, and preferably independently of one another selected from the group consisting of substitution positions A342, and K343; and/or subsequence R2 ranging from position L231 to Q243 of SEQ ID NO:1, and preferably independently of one another selected from the groups consisting of substitution positions M241 and F242; and/or subsequence R4-A1 ranging from position L139 to W140 of SEQ ID NO:1, preferably substitution position W140; and/or subsequence R5-B ranging from position F44 to R67 of SEQ ID NO: 1, preferably substitution position A47.

Clause 8: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions at substitution positions wherein from subsequence R3-A ranging from position N185 to R192 of SEQ ID NO: 1, the substitution positions are independently of one another selected from the group consisting of V187, and G190; and/or from subsequence R1-A ranging from position L266 to Y280 of SEQ ID NO: 1, the substitution position is A273; and/or from subsequence R1-B ranging from position S295 to D315 of SEQ ID NO: 1, the substitution positions are independently of one another selected from the group consisting of P313, Q300, and M309; and/or from subsequence R1-D ranging from position G337 to R346 of SEQ ID NO:1, the substitution positions are independently of one another selected from the group consisting of A342, and K343; and/or from subsequence R2 ranging from position L231 to Q243 of SEQ ID NO:1, the substitution positions are independently of one another selected from the group consisting of M241 and F242; and/or from subsequence R4-A1 ranging from position L139 to W140 of SEQ ID NO:1, the substitution position is W140; and/or from subsequence R5-B ranging from position F44 to R67 of SEQ ID NO:1, the substitution position is A47.

Clause 9: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions at substitution positions wherein from subsequence R3-A ranging from position N185 to R192 of SEQ ID NO: 1, the substitution positions are independently of one another selected from the group consisting of V187, and G190; and/or from subsequence R1-A ranging from position L266 to Y280 of SEQ ID NO:1, the substitution position is A273; and/or from subsequence R1-B ranging from position S295 to D315 of SEQ ID NO: 1, the substitution positions are independently of one another selected from the group consisting of P313, Q300, and M309; and/or from subsequence R1-D ranging from position G337 to R346 of SEQ ID NO:1, the substitution positions are independently of one another selected from the group consisting of A342, and K343; and/or from subsequence R4-A1 ranging from position L139 to W140 of SEQ ID NO: 1, the substitution position is W140; and/or from subsequence R5-B ranging from position F44 to R67 of SEQ ID NO: 1, the substitution position is A47.

Clause 10: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions at substitution positions wherein from subsequence R3-A ranging from position N185 to R192 of SEQ ID NO: 1, the substitution position is V187; and/or from subsequence R1-A ranging from position L266 to Y280 of SEQ ID NO:1, the substitution position is A273; and/or from subsequence R1-B ranging from position S295 to D315 of SEQ ID NO:1, the substitution positions are independently of one another selected from the group consisting of P313, Q300, and M309; and/or from subsequence R1-D ranging from position G337 to R346 of SEQ ID NO:1, the substitution positions are independently of one another selected from the group consisting of A342, and K343; and/or from subsequence R4-A1 ranging from position L139 to W140 of SEQ ID NO: 1, the substitution position is W140; and/or from subsequence R5-B ranging from position F44 to R67 of SEQ ID NO: 1, the substitution position is A47.

Clause 11: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions at substitution positions wherein from subsequence R1-B ranging from position S295 to D315 of SEQ ID NO:1, the substitution positions are independently of one another selected from the group consisting of P313, Q300, and M309; and/or from subsequence R1-D ranging from position G337 to R346 of SEQ ID NO: 1, the substitution positions are independently of one another selected from the group consisting of A342, and K343; and/or from subsequence R5-B ranging from position F44 to R67 of SEQ ID NO: 1, the substitution position is A47.

Clause 12: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions at substitution positions in any subsequence independently of one another selected from the groups consisting of: R1, R2, R3, R4, and/or R5; or R1 and R2 and R3 and R4 and R5; or R1 and R2 and R3 and R5; or R1 and R3 and R5; or R1 and R5; or R1; or—R3; or R5; or R4.

Clause 13: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions, wherein the substitution positions are in any subsequence, and (i) wherein the substitutions in subsequence R1 are independently of one another selected from the groups consisting of R1-A, R1-B, R1-C and/or R1-D; or R1-A, and R1-B, and R1-C and R1-D; or R1-A, R1-B, and R1-D; or R1-B and R1-D; or R1-A, R1-B1, R1-B2, R1-C, and R1-D; or R1-A, R1-B1, R1-B2, and R1-D; or R1-B1, R1-B2, R1-C, and R1-D; or R1-B1, R1-B2, and R1-D; and/or (ii) wherein the substitutions in subsequence R3 are independently of one another selected from the group consisting of R3-A and R3-B; or R3-A; or R3-B; and/or (iii) wherein the substitutions in subsequence R4 are independently of one another selected from the groups consisting of: R4-A and R4-B; or R4-A1, R4-A2, and R4-B; or R4-A1, and R4-A2; or R4-A1, and R4-B; or R4-A2, and R4-B; or R4-A1, R4-A2, or R4-B; and/or (iv) wherein the substitutions in subsequence R5 are independently of one another selected from the groups consisting of R5-A and R5-B; or R5-A; or R5-B.

Clause 14: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions, wherein the substitution positions are in any subsequence, and wherein the substitutions are independently of one another selected from the groups consisting of R1-B, R1-D and R5-B; or R1-B, R1-D and R3-A; or R1-B, R1-D, R3-A and R5-B; or R1-B, R1-D, R3-A, R2 and R5-B.

Clause 15: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions, wherein the substitution positions are in any subsequence, and the substitutions are independently of one another selected from the groups consisting of (i) S295 to D315, G337 to R346, and F44 to R67; preferably S295 to D302, W307 to D315, L338 to K343, and F44 to Q54; more preferably A298 to Q300, D308 to P313, N339 to K343, G45 to H5, and most preferably Q300, M309, P313, A342, K343 and A47; or (ii) S295 to D315, G337 to R346, and N185 to R192; preferably S295 to D302, W307 to D315, L338 to K343, and V187 to G191; more preferably A298 to Q300, D308 to P313, N339 to K343, V187, and G190, and most preferably Q300, M309, P313, A342, K343, and V187; or (iii) S295 to D315, G337 to R346, N185 to R192, and F44 to R67; preferably S295 to D302, W307 to D315, L338 to K343, V187 to G191, and F44 to Q54; more preferably A298 to Q300, D308 to P313, N339 to K343, V187, G190, and G45 to H5, and most preferably Q300, M309, P313, A342, K343, V187 and A47; or (iv) S295 to D315, G337 to R346, N185 to R192, M241, F242, and F44 to R67; preferably S295 to D302, W307 to D315, L338 to K343, V187 to G191, M241, F242, and F44 to Q54; more preferably A298 to Q300, D308 to D315, N339 to K343, V187, G190, G45 to H5, M241, F242, and most preferably Q300, M309, P313, A342, K343, V187, M241, F242 and A47.

Clause 16: The glucose isomerase of any of the preceding clauses, wherein the amino acid sequence comprises at least one substitution in R3-A.

Clause 17: The glucose isomerase of any of the preceding clauses, wherein the amino acid sequence comprises at least one substitution in substitution positions N185 to R192, preferably in substitution position V187.

Clause 18: The glucose isomerase of any of the preceding clauses, wherein the amino acid sequence comprises at least one, two, three, four, or five substitutions, wherein the substitution positions are in any subsequence of R1-B and R1-D.

Clause 19: The glucose isomerase of any of the preceding clauses, wherein the amino acid sequence comprises at least one, two, three, four, or five substitutions, wherein the substitution positions are in substitution positions S295 to D315, and G337 to R346; preferably S295 to D302, W307 to D315, and L338 to K343; more preferably A298 to Q300, D308 to P313, and N339 to K343, and most preferably Q300, M309, P313, A342, and K343.

Clause 20: The glucose isomerase of any of the preceding clauses, wherein the amino acid sequence comprises at least one, two, or three substitutions, wherein the substitution positions are in subsequence R1-B.

Clause 21: The glucose isomerase of any of the preceding clauses, wherein the amino acid sequence comprises at least one, two, or three substitutions, wherein the substitution positions are in substitution positions S295 to D315; preferably S295 to D302, and W307 to D315; more preferably A298 to Q300, and D308 to P313, and most preferably in Q300, M309, and P313.

Clause 22: The glucose isomerase of any of the preceding clauses, wherein the amino acid sequence comprises at least one, or two substitutions, wherein the substitution positions are in subsequence R1-D.

Clause 23: The glucose isomerase of any of the preceding clauses, wherein the amino acid sequence comprises at least one, or two substitutions, wherein the substitution positions are in substitution positions G337 to R346; preferably L338 to K343; more preferably and N339 to K343, and most preferably in A342, and K343.

Clause 24: The glucose isomerase of any of the preceding clauses, wherein the amino acid sequence comprises at least one, two, three, four, five, or six substitutions, wherein the substitution positions are independently of one another selected from the groups consisting of Q300, M309, P313, A342, K343 and A47.

Clause 25: The glucose isomerase of any of the preceding clauses, wherein the amino acid sequence comprises at least one, two, three, four, five, or six substitutions, wherein the substitution positions are independently of one another selected from the groups consisting of Q300, M309, P313, A342, K343, and V187.

Clause 26: The glucose isomerase of any of the preceding clauses, wherein the amino acid sequence comprises at least one, two, three, four, five, six or seven substitutions, wherein the substitution positions are independently of one another selected from the groups consisting of Q300, M309, P313, A342, K343, V187 and A47.

Clause 27: The glucose isomerase of any of the preceding clauses, wherein the amino acid sequence comprises at least one, two, three, four, five, six, seven, eight or nine substitutions, wherein the substitution positions are independently of one another selected from the groups consisting of Q300, M309, P313, A342, K343, V187, M241, F242 and A47.

Clause 28: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions, wherein the substitution positions are in any subsequence, and substitutions are independently of one another selected from the group consisting of R1, R2, R3, R4, and/or R5, and wherein the amino acid sequence in addition comprises at least one further substitution in any subsequence independently of one another selected from the group consisting of R1, R2, R3, R4, and/or R5, preferably in any subsequence independently of one another selected from the group consisting of R1-A, R1-B, R1-C, R1-D, R2, R3-A, R3-B, R4-A, R4-B, R5-A and R5-B, and more preferably in any subsequence independently of one another selected from the group consisting of R1-A, R1-B1, R1-B2, R1-C, R1-D, R2, R3-A, R3-B, R4-A1, R4-A2, R4-B, R5-A and R5-B.

Clause 29: The glucose isomerase of any of the preceding clauses wherein the amino acid sequence comprises at least one, two, three, four, five, six, seven, eight, nine, ten, eleven, twelve, thirteen, fourteen, fifteen, sixteen or seventeen substitutions, wherein the substitution positions are in any subsequence independently of one another selected from the group consisting of R1, R2, R3, R4, and/or R5, and wherein the amino acid sequence in addition comprises at least one further substitution in any substitution position of a subsequence, wherein the at least one substitution position is independently of one another selected from the group consisting of P18, A47, W140, T147, I163, V187, G190, L198, M241, F242, A273, Q300, M309, P313, Q331, A342, and K343.

Clause 30: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions, wherein the substitution positions are independently of one another selected from the groups consisting of: A273, Q300, M309, P313, Q331, A342, and K343 in subsequence R1, and/or M241, and F242 in the subsequence R2, and/or V187, G190, L198, in subsequence R3, and/or W140, T147, and I163, in subsequence R4, and/or P18, and A47 in subsequence R5.

Clause 31: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions, (i) wherein the substitution positions in subsequences R1 are independently of one another selected from the groups consisting of: A273, Q300, M309, P313, Q331, A342, and K343; or A273, Q300, M309, P313, Q331, and A342; or A273, Q300, M309, P313, A342, and K343; or Q300, M309, P313, A342, and K343; or Q300, M309, P313, or A342, and K343; or A273, Q300, M309, P313, Q331, A342, or K343; and/or (ii) wherein the substitution positions in subsequences R2 are independently of one another selected from the groups consisting of: M241 and F242; or M241, or F242; and/or (iii) wherein the substitution positions in subsequence R3 are independently of one another selected from the groups consisting of: V187, G190, L198; or V187, G190; or L198, or V187, or G190; and/or (iv) wherein the substitution positions in subsequence R4 are independently of one another selected from the groups consisting of: W140, T147, and I163; or W140 and T147; or W140 and I163; or T147 and I163; or W140, T147, or I163; and/or (v) wherein the substitution positions in subsequence R5 are independently of one another selected from the groups consisting of: P18 and A47; or P18 or A47.

Clause 32: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions at substitution positions independently of one another selected from the group consisting of P18, A47, W140, T147, I163, V187, G190, L198, M241, F242, A273, Q300, M309, P313, Q331, A342, and K343; preferably selected from the group consisting of P18, A47, W140, T147, I163, V187, G190, M241, F242, Q300, M309, P313, Q331, A342, and K343; more preferably selected from the group consisting of A47, W140, V187, G190, M241, F242, Q300, M309, P313, Q331, A342, and K343; and most preferably selected from the group consisting of A47, V187, M241, F242, Q300, M309, P313, A342, and K343.

Clause 33: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions at substitution positions independently of one another selected from the groups consisting of P18, A47, W140, T147, I163, V187, G190, M241, F242, Q300, M309, P313, Q331, A342, and K343; or P18, A47, W140, I163, V187, L198, M241, F242, Q300, P313, Q331, A342, and K343; or P18, A47, W140, T147, I163, V187, G190, L198, M241, F242, A273, Q300, M309, P313, Q331, A342, and K343; or P18, A47, W140, T147, I163, V187, G190, L198, M241, F242, A273, Q300, M309, 29K343, P313, Q331, and A342.

Clause 34: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions at substitution positions independently of one another selected from the groups consisting of P18, A47, W140, I163, V187, M241, F242, Q300, P313, Q331, A342, and K343; or A47, V187, M241, F242, Q300, M309, P313, A342, and K343.

Clause 35: The glucose isomerase of any of preceding clauses, wherein the substitution positions are independently of one another selected from the group consisting of V187, P313, Q300, A47, W140, G190, M241, F242, A273, M309, A342, and K343; and preferably selected from the group consisting of V187, P313, Q300, A47, M241, F242, M309, A342, and K343, and more preferably selected from the group consisting of V187, P313, and Q300.

Clause 36: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions, wherein (i) from the amino acid positions 44, 45, 46, 47, 48, 49, 50, 51, 52, 53, 54, 55, 56, 57, 58, 59, 60, 61, 67, 100, 101, 102, 105, 139, 140, 142, 143, 144, 145, 146, 147, 187, 188, 189, 190, 191, 192, 193, 231, 232, 233, 234, 235, 236, 237, 238, 239, 240, 241, 242, 243, 245, 246, 247, 248, 266, 267, 268, 269, 270, 271, 272, 273, 274, 275, 276, 277, 278, 279, 280, 283, 284, 295, 296, 297, 298, 299, 300, 301, 302, 303, 304, 305, 306, 307, 308, 309, 310, 311, 312, 313, 314, 315, 318, 319, 322, 338, 339, 340, 341, 342, 343, 344, 345, 346, 348, 354, 357, 358, 361, 365, 431, 432, and 435 of SEQ ID NO: 1 the substitution positions are independently of one another selected from the group consisting of A47, W140, V187, G190, M241, F242, A273, Q300, M309, P313, A342, and K343; and/or (ii) from the amino acid positions 45, 46, 47, 48, 49, 50, 54, 60, 100, 101, 102, 139, 140, 142, 146, 185, 186, 187, 188, 189, 190, 191, 231, 232, 233, 234, 235, 236, 243, 266, 267, 268, 269, 270, 271, 272, 273, 275, 280, 294, 295, 296, 297, 298, 299, 300, 308, 309, 310, 311, 313, 337, 338, 339, 340, 341, 342, and 343 of SEQ ID NO: 1 the substitution positions are independently of one another selected from the group consisting of A47, W140, V187, G190, A273, Q300, M309, P313, A342, and K343; and/or (iii) from the amino acid positions 44, 45, 46, 47, 48, 49, 50, 51, 52, 53, 54, 56, 60, 67, 90, 91, 94, 96, 98, 99, 100, 101, 102, 105, 139, 140, 146, 187, 189, 233, 235, 238, 267, 268, 269, 270, 271, 272, 273, 275, 280, 295, 296, 297, 298, 299, 300, 301, 302, 307, 308, 309, 310, 311, 312, 313, 314, 315, 318, 319, 322, 338, 339, 340, 341, 342, 343, 344, 345, 346, 348, 349, 353, 354, 355, 356, 357, 358, 359, 360, 361, 362, 363, 364, 365, 431, 432, 433, and 435 of SEQ ID NO: 1 the substitution positions are independently of one another selected from the group consisting of A47, W140, V187, A273, Q300, M309, P313, A342, and K343; and/or (iv) from the amino acid positions 47, 50, 269, 271, 297, 298, 299, 300, 309, 310, 311, 312, 313, 339, 340, 341, 342, 343, 344, 345, and 358 of SEQ ID NO:1 the substitution positions are independently of one another selected from the group consisting of A47, Q300, M309, P313, A342 and K343.

Clause 37: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions, wherein (i) from the amino acid positions 44, 45, 46, 47, 48, 49, 50, 51, 52, 53, 54, 55, 56, 57, 58, 59, 60, 61, 67, 139, 140, 187, 188, 189, 190, 191, 192, 231, 232, 233, 234, 235, 236, 237, 238, 239, 240, 241, 242, 243, 266, 267, 268, 269, 270, 271, 272, 273, 274, 275, 276, 277, 278, 279, 280, 295, 296, 297, 298, 299, 300, 301, 302, 303, 304, 305, 306, 307, 308, 309, 310, 311, 312, 313, 314, 315, 338, 339, 340, 341, 342, 343, 344, 345, and 346, of SEQ ID NO: 1 the substitution positions are independently of one another selected from the group consisting of V187, A47, W140, G190, M241, F242, A273, Q300, M309, P313, A342, and K343; and/or (ii) from the amino acid positions 45, 46, 47, 48, 49, 50, 54, 60, 139, 140, 187, 188, 189, 190, 191, 231, 232, 233, 234, 235, 236, 243, 266, 267, 268, 269, 270, 271, 272, 273, 275, 280, 295, 296, 297, 298, 299, 300, 308, 309, 310, 311, 313, 338, 339, 340, 341, 342, and 343 of SEQ ID NO:1 the substitution positions are independently of one another selected from the group consisting of V187, A47, W140, G190, A273, Q300, M309, P313, A342, and K343; and/or (iii) from the amino acid positions 44, 45, 46, 47, 48, 49, 50, 51, 52, 53, 54, 56, 60, 67, 139, 140, 187, 189, 233, 235, 238, 267, 268, 269, 270, 271, 272, 273, 275, 280, 295, 296, 297, 298, 299, 300, 301, 302, 307, 308, 309, 310, 311, 312, 313, 314, 315, 338, 339, 340, 341, 342, 343, 344, 345, and 346 of SEQ ID NO: 1 the substitution positions are independently of one another selected from the group consisting of V187, A47, W140, A273, Q300, M309, P313, A342, and K343; and/or (iv) from the amino acid positions 47, 50, 269, 271, 297, 298, 299, 300, 309, 310, 311, 312, 313, 339, 340, 341, 342, 343, 344, and 345 of SEQ ID NO: 1 the substitution positions are independently of one another selected from the group consisting of A47, Q300, M309, P313, A342 and K343.

Clause 38: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions at substitution positions independently of one another selected from the group consisting of A47, W140, V187, G190, M241, F242, A273, Q300, M309, P313, A342, K343; preferably selected from the group consisting of A47, W140, V187, G190, A273, Q300, M309, P313, A342, K343; more preferably selected from the group consisting of A47, W140, V187, A273, Q300, M309, P313, A342, K343, and most preferably selected from the group consisting of A47, Q300, M309, P313, A342, K343.

Clause 39: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions, wherein the at least one further substitution is independently of one another selected from the group consisting of substitution positions P18, A47, W140, T147, I163, V187, G190, L198, M241, F242, A273, Q300, M309, P313, Q331, A342, and K343.

Clause 40: The glucose isomerase of any of the preceding clauses, wherein the amino acid sequence comprises at least two, three, four, five, six, seven, eight, nine, ten, eleven, twelve, thirteen, fourteen, fifteen, sixteen or seventeen substitutions, wherein the substitution positions are in any subsequence and independently of one another selected from the groups consisting of subsequences R1 and R2, R1 and R3, R1 and R4, R1 and R5, R2 and R3, R2 and R4, R2 and R5, R3 and R4, R3 and R5, and R4 and R5.

Clause 41: The glucose isomerase of any of the preceding clauses, wherein the amino acid sequence comprises at least two, three, four, five, six, seven, eight, nine, ten, eleven, twelve, thirteen, fourteen, fifteen, sixteen or seventeen substitutions, wherein the substitution positions are in any subsequence and independently of one another selected from the groups consisting of subsequences, (i) wherein the substitutions in subsequence R1 are independently of one another selected from the groups consisting of R1-A and R1-B, R1-A and R1-C, R1-A and R1-D, R1-B and R1-C, R1-B and R1-D, and R1-C and R1-D; or R1-A and R1-B, R1-A and R1-D, R1-B and R1-D; or R1-A and R1-B1, R1-A and R1-B2, R1-A and R1-C, R1-A and R1-D, R1-B1 and R1-B2, R1-B1 and R1-C, R1-B1 and R1-D, R1-B2 and R1-C, R1-B2 and R1-D; or R1-A and R1-B1, R1-A and R1-B2, R1-A and R1-D, R1-B1 and R1-B2, R1-B1 and R1-D, R1-B2 and R1-D; or R1-C and R1-B1, R1-C and R1-B2, R1-C and R1-D, R1-B1 and R1-B2, R1-B1 and R1-D, R1-B2 and R1-D; or R1-B1 and R1-B2, R1-B1 and R1-D, R1-B2 and R1-D; and/or (ii) wherein the substitutions in subsequence R3 are independently of one another selected from the groups consisting of R3-A and R3-B; and/or (iii) wherein the substitutions in subsequence R4 are independently of one another selected from the groups consisting of R4-A and R4-B; or R4-A1, and R4-A2, R4-A1 and R4-B, R4-A2 and R4-B; or R4-A1 and R4-A2; or R4-A1 and R4-B; or R4-A2 and R4-B; or R4-A1 and R4-A2, R4-A1 and R4-B, R4-A2 and R4-B; and/or (iv) wherein the substitutions in subsequence R4 are independently of one another selected from the groups consisting of R5-A and R5-B.

Clause 42: The glucose isomerase of any of the preceding clauses, wherein the amino acid sequence comprises at least two, three, four, five, six, seven, eight, nine, ten, eleven, twelve, thirteen, fourteen, fifteen, sixteen or seventeen substitutions, wherein the substitution positions are in any subsequence and independently of one another selected from the groups consisting of subsequences, wherein the substitutions are individually and in-dependently selected from the groups consisting of R1-B and R1-D, R1-B and R5-B, R1-D and R5-B; or R1-B and R1-D, R1-B and R3-A, R1-D and R3-A; or R1-B and R1-D, R1-B and R3-A, R1-B and R5-B, R1-D and R3-A, R1-D and R5-B, R3-A and R5-B; or R1-B and R1-D, R1-B and R3-A, R1-B and R2, R1-B and R5-B, R1-D and R3-A, R1-D and R2, R1-D and R5-B, R3-A and R2, R3-A and R5-B, R2 and R5-B.

Clause 43: The glucose isomerase of any of the preceding clauses, wherein the amino acid sequence comprises at least two, three, four, five, six, seven, eight, nine, ten, eleven, twelve, thirteen, fourteen, fifteen, sixteen or seventeen substitutions at substitution positions independently of one another selected from the group consisting of P18, A47, W140, T147, I163, V187, G190, L198, M241, F242, A273, Q300, M309, P313, Q331, A342, and K343; preferably consisting of V187, A47, W140, G190, M241, F242, A273, Q300, M309, P313, A342; and most preferably consisting of A47, V187, M241, F242, Q300, M309, P313, A342 and K343.

Clause 44: The glucose isomerase of clause 43, wherein the amino acid sequence comprises one substitution at substitution position V187, and wherein the amino acid sequence further comprises at least one, two, three, four, five or six substitutions at substitution positions independently of one another selected from the group consisting of A47, M241, F242, Q300, M309, P313, A342 and K343.

Clause 45: The glucose isomerase of any of the preceding clauses, wherein the amino acid sequence comprises at least two, three, four, five, six, seven, eight, nine, ten, eleven, twelve, thirteen, fourteen, fifteen, sixteen or seventeen substitutions wherein the amino acid sequence comprises at least two substitution positions selected from the group consisting of (i) P18+A47, P18+W140, P18+T147, P18+I163, P18+V187, P18+G190, P18+L198, P18+F242, P18+M241, P18+A273, P18+Q300, P18+M309, P18+P313, P18+Q331, P18+A342, P18+K343, A47+W140, A47+T147, A47+I163, A47+V187, A47+G190, A47+L198, A47+F242, A47+M241, A47+A273, A47+Q300, A47+M309, A47+P313, A47+Q331, A47+A342, A47+K343, W140+T147, W140+I163, W140+V187, W140+G190, W140+L198, W140+F242, W140+M241, W140+A273, W140+Q300, W140+M309, W140+P313, W140+Q331, W140+A342, W140+K343, T147+I163, T147+V187, T147+G190, T147+L198, T147+F242, T147+M241, T147+A273, T147+Q300, T147+M309, T147+P313, T147+Q331, T147+A342, T147+K343, I163+V187, I163+G190, I163+L198, I163+F242, I163+M241, I163+A273, I163+Q300, I163+M309, I163+P313, I163+Q331, I163+A342, I163+K343, V187+G190, V187+L198, V187+F242, V187+M241, V187+A273, V187+Q300, V187+M309, V187+P313, V187+Q331, V187+A342, V187+K343, G190+L198, G190+F242, G190+M241, G190+A273, G190+Q300, G190+M309, G190+P313, G190+Q331, G190+A342, G190+K343, L198+F242, L198+M241, L198+A273, L198+Q300, L198+M309, L198+P313, L198+Q331, L198+A342, L198+K343, F242+M241, F242+A273, F242+Q300, F242+M309, F242+P313, F242+Q331, F242+A342, F242+K343, M241+A273, M241+Q300, M241+M309, M241+P313, M241+Q331, M241+A342, M241+K343, A273+Q300, A273+M309, A273+P313, A273+Q331, A273+A342, A273+K343, Q300+M309, Q300+P313, Q300+Q331, Q300+A342, Q300+K343, M309+P313, M309+Q331, M309+A342, M309+K343, P313+Q331, P313+A342, P313+K343, Q331+A342, Q331+K343, and A342+K343; and/or (ii) V187+Q300, V187+P18, V187+A47, V187+W140, V187+T147, V187+I163, V187+G190, V187+M241, V187+F242, V187+M309, V187+P313, V187+Q331, V187+A342, V187+K343, P18+A47, P18+W140, P18+T147, P18+I163, P18+G190, P18+M241, P18+F242, P18+Q300, P18+M309, P18+P313, P18+Q331, P18+A342, P18+K343, A47+W140, A47+T147, A47+I163, A47+G190, A47+M241, A47+F242, A47+Q300, A47+M309, A47+P313, A47+Q331, A47+A342, A47+K343, W140+T147, W140+I163, W140+G190, W140+M241, W140+F242, W140+Q300, W140+M309, W140+P313, W140+Q331, W140+A342, W140+K343, T147+I163, T147+G190, T147+M241, T147+F242, T147+Q300, T147+M309, T147+P313, T147+Q331, T147+A342, T147+K343, I163+G190, I163+M241, I163+F242, I163+Q300, I163+M309, I163+P313, I163+Q331, I163+A342, I163+K343, G190+M241, G190+F242, G190+Q300, G190+M309, G190+P313, G190+Q331, G190+A342, G190+K343, M241+F242, M241+Q300, M241+M309, M241+P313, M241+Q331, M241+A342, M241+K343, F242+Q300, F242+M309, F242+P313, F242+Q331, F242+A342, F242+K343, Q300+M309, Q300+P313, Q300+Q331, Q300+A342, Q300+K343, M309+P313, M309+Q331, M309+A342, M309+K343, P313+Q331, P313+A342, P313+K343, Q331+A342, Q331+K343, and A342+K343; and/or (iii) V187+Q300, V187+P18, V187+A47, V187+W140, V187+I163, V187+L198, V187+M241, V187+F242, V187+M309, V187+P313, V187+Q331, V187+A342, V187+K343, P18+A47, P18+W140, P18+I163, P18+L198, P18+M241, P18+F242, P18+Q300, P18+M309, P18+P313, P18+Q331, P18+A342, P18+K343, A47+W140, A47+I163, A47+L198, A47+M241, A47+F242, A47+Q300, A47+M309, A47+P313, A47+Q331, A47+A342, A47+K343, W140+I163, W140+L198, W140+M241, W140+F242, W140+Q300, W140+M309, W140+P313, W140+Q331, W140+A342, W140+K343, I163+L198, I163+M241, I163+F242, I163+Q300, I163+M309, I163+P313, I163+Q331, I163+A342, I163+K343, L198+M241, L198+F242, L198+Q300, L198+M309, L198+P313, L198+Q331, L198+A342, L198+K343, M241+F242, M241+Q300, M241+M309, M241+P313, M241+Q331, M241+A342, M241+K343, F242+Q300, F242+M309, F242+P313, F242+Q331, F242+A342, F242+K343, Q300+M309, Q300+P313, Q300+Q331, Q300+A342, Q300+K343, M309+P313, M309+Q331, M309+A342, M309+K343, P313+Q331, P313+A342, P313+K343, Q331+A342, Q331+K343, and A342+K343; and/or (iv) Q300+P18, Q300+A47, Q300+W140, Q300+T147, Q300+I163, Q300+V187, Q300+G190, Q300+L198, Q300+M241, Q300+F242, Q300+A273, Q300+M309, Q300+K343, Q300+P313, Q300+Q331, Q300+A342, P18+A47, P18+W140, P18+T147, P18+I163, P18+V187, P18+G190, P18+L198, P18+M241, P18+F242, P18+A273, P18+M309, P18+K343, P18+P313, P18+Q331, P18+A342, A47+W140, A47+T147, A47+I163, A47+V187, A47+G190, A47+L198, A47+M241, A47+F242, A47+A273, A47+M309, A47+K343, A47+P313, A47+Q331, A47+A342, W140+T147, W140+I163, W140+V187, W140+G190, W140+L198, W140+M241, W140+F242, W140+A273, W140+M309, W140+K343, W140+P313, W140+Q331, W140+A342, T147+I163, T147+V187, T147+G190, T147+L198, T147+M241, T147+F242, T147+A273, T147+M309, T147+K343, T147+P313, T147+Q331, T147+A342, I163+V187, I163+G190, I163+L198, I163+M241, I163+F242, I163+A273, I163+M309, I163+K343, I163+P313, I163+Q331, I163+A342, V187+G190, V187+L198, V187+M241, V187+F242, V187+A273, V187+M309, V187+K343, V187+P313, V187+Q331, V187+A342, G190+L198, G190+M241, G190+F242, G190+A273, G190+M309, G190+K343, G190+P313, G190+Q331, G190+A342, L198+M241, L198+F242, L198+A273, L198+M309, L198+K343, L198+P313, L198+Q331, L198+A342, M241+F242, M241+A273, M241+M309, M241+K343, M241+P313, M241+Q331, M241+A342, F242+A273, F242+M309, F242+K343, F242+P313, F242+Q331, F242+A342, A273+M309, A273+K343, A273+P313, A273+Q331, A273+A342, M309+K343, M309+P313, M309+Q331, M309+A342, K343+P313, K343+Q331, K343+A342, P313+Q331, P313+A342, Q331+A342.

Clause 46: The glucose isomerase of any of the preceding clauses, wherein the amino acid sequence comprises at least two, three, four, five, six, seven, eight, nine, ten, eleven, twelve, thirteen, fourteen, fifteen, sixteen or seventeen substitutions wherein the amino acid sequence comprises at least two substitution positions selected from the group consisting of A47+V187, A47+M241, A47+F242, A47+Q300, A47+M309, A47+P313, A47+A342, A47+K343, V187+M241, V187+F242, V187+Q300, V187+M309, V187+P313, V187+A342, V187+K343, M241+F242, M241+Q300, M241+M309, M241+P313, M241+A342, M241+K343, F242+Q300, F242+M309, F242+P313, F242+A342, F242+K343, Q300+M309, Q300+P313, Q300+A342, Q300+K343, M309+P313, M309+A342, M309+K343, P313+A342, P313+K343, and A342+K343.

Clause 47: The glucose isomerase of any of the preceding clauses, wherein the amino acid sequence comprises at least two, three, four, five, six, seven, eight, nine, ten, eleven, twelve, thirteen, fourteen, fifteen, sixteen or seventeen substitutions, wherein the substitution positions are in any subsequence and independently of one another selected from the groups consisting of subsequences R1 and R2 and R3, R1 and R2 and R4, R1 and R2 and R5, R1 and R3 and R4, R1 and R3 and R5, R1 and R4 and R5, R2 and R3 and R4, R2 and R3 and R5, R2 and R4 and R5, and R3 and R4 and R5.

Clause 48: The glucose isomerase of any of the preceding clauses, wherein the amino acid sequence comprises at least two, three, four, five, six, seven, eight, nine, ten, eleven, twelve, thirteen, fourteen, fifteen, sixteen or seventeen substitutions, wherein the substitution positions are in any subsequence and independently of one another selected from the groups consisting of subsequences, (i) wherein the substitutions in subsequence R1 are independently of one another selected from the groups consisting of R1-A and R1-B and R1-C, R1-A and R1-B and R1-D, R1-A and R1-C and R1-D, R1-B and R1-C and R1-D; or R1-A and R1-B and R1-D; or R1-A and R1-B1 and R1-B2, R1-A and R1-B1 and R1-D, R1-A and R1-B2 and R1-D, R1-B1 and R1-B2 and R1-D, R1-A and R1-B1 and R1-C, R1-A and R1-B2 and R1-C, R1-B1 and R1-B2 and R1-C, R1-B1 and R1-C and R1-D, R1-B2 and R1-C and R1-D; or R1-A and R1-B1 and R1-B2, R1-A and R1-B1 and R1-D, R1-A and R1-B2 and R1-D, R1-B1 and R1-B2 and R1-D; or R1-C and R1-B1 and R1-B2, R1-C and R1-B1 and R1-D, R1-C and R1-B2 and R1-D, R1-B1 and R1-B2 and R1-D; or R1-B1 and R1-B2 and R1-D1; and/or (ii) wherein the substitutions in subsequence R4 are independently of one another selected from the groups consisting of R4-A1 and R4-A2 and R4-B.

Clause 49: The glucose isomerase of any of the preceding clauses, wherein the amino acid sequence comprises at least two, three, four, five, six, seven, eight, nine, ten, eleven, twelve, thirteen, fourteen, fifteen, sixteen or seventeen substitutions, wherein the substitution positions are in any subsequence and independently of one another selected from the groups consisting of subsequences, R1-B and R1-D and R5-B; or R1-B and R1-D and R3-A, R1-B and R1-D and R5-B, R1-B and R3-A and R5-B, R1-D and R3-A and R5-B; or R1-B and R1-D and R3-A, R1-B and R1-D and R2, R1-B and R1-D and R5-B, R1-B and R3-A and R2, R1-B and R3-A and R5-B, R1-B and R2 and R5-B, R1-D and R3-A and R2, R1-D and R3-A and R5-B, R1-D and R2 and R5-B, R3-A and R2 and R5-B.

Clause 50: The glucose isomerase of any of the preceding clauses, wherein the amino acid sequence comprises at least three, four, five, six, seven, eight, nine, ten, eleven, twelve, thirteen, fourteen, fifteen, sixteen or seventeen substitutions at substitution positions independently of one another selected from the group consisting of P18, A47, W140, T147, I163, V187, G190, L198, M241, F242, A273, Q300, M309, P313, Q331, A342, and K343; preferably consisting of A47, V187, M241, F242, Q300, M309, P313, A342 and K343.

Clause 51: The glucose isomerase of any of the preceding clauses, wherein the amino acid sequence comprises at least three, four, five, six, seven, eight, nine, ten, eleven, twelve, thirteen, fourteen, fifteen, sixteen or seventeen substitutions wherein the amino acid sequence comprises at least three substitution positions selected from the group consisting of V187+A47+M241, V187+A47+F242, V187+A47+Q300, V187+A47+M309, V187+A47+P313, V187+A47+A342, V187+A47+K343, V187+M241+F242, V187+M241+Q300, V187+M241+M309, V187+M241+P313, V187+M241+A342, V187+M241+K343, V187+F242+Q300, V187+F242+M309, V187+F242+P313, V187+F242+A342, V187+F242+K343, V187+Q300+M309, V187+Q300+P313, V187+Q300+A342, V187+Q300+K343, V187+M309+P313, V187+M309+A342, V187+M309+K343, V187+P313+A342, V187+P313+K343, V187+A342+K343, A47+M241+F242, A47+M241+Q300, A47+M241+M309, A47+M241+P313, A47+M241+A342, A47+M241+K343, A47+F242+Q300, A47+F242+M309, A47+F242+P313, A47+F242+A342, A47+F242+K343, A47+Q300+M309, A47+Q300+P313, A47+Q300+A342, A47+Q300+K343, A47+M309+P313, A47+M309+A342, A47+M309+K343, A47+P313+A342, A47+P313+K343, A47+A342+K343, M241+F242+Q300, M241+F242+M309, M241+F242+P313, M241+F242+A342, M241+F242+K343, M241+Q300+M309, M241+Q300+P313, M241+Q300+A342, M241+Q300+K343, M241+M309+P313, M241+M309+A342, M241+M309+K343, M241+P313+A342, M241+P313+K343, M241+A342+K343, F242+Q300+M309, F242+Q300+P313, F242+Q300+A342, F242+Q300+K343, F242+M309+P313, F242+M309+A342, F242+M309+K343, F242+P313+A342, F242+P313+K343, F242+A342+K343, Q300+M309+P313, Q300+M309+A342, Q300+M309+K343, Q300+P313+A342, Q300+P313+K343, Q300+A342+K343, M309+P313+A342, M309+P313+K343, M309+A342+K343, and P313+A342+K343.

Clause 52: The glucose isomerase of any of the preceding clauses, wherein the amino acid sequence comprises at least four, five, six, seven, eight, nine, ten, eleven, twelve, thirteen, fourteen, fifteen, sixteen or seventeen substitutions at substitution positions independently of one another selected from the group consisting of P18, A47, W140, T147, I163, V187, G190, L198, M241, F242, A273, Q300, M309, P313, Q331, A342, and K343; preferably consisting of A47, V187, M241, F242, Q300, M309, P313, A342 and K343.

Clause 53: The glucose isomerase of any of the preceding clauses, wherein the amino acid sequence comprises a substitution in position 18, the substitution being selected from the group consisting of P18S, P18A, P18R, P18N, P18D, P18C, P18Q, P18E, P18G, P18H, P18I, P18L, P18K, P18M, P18F, P18T, P18W, P18V or P18Y; more preferably P18S, P18T, P18Y; and most preferably P18S.

Clause 54: The glucose isomerase of any of the preceding clauses, wherein the amino acid sequence comprises a substitution in position 47, the substitution being selected from the group consisting of A47S, A47C, A47R, A47N, A47D, A47Q, A47E, A47G, A47H, A47I, A47L, A47K, A47M, A47F, A47P, A47T, A47W, A47V, or A47Y; preferably A47S, A47Q or A47R; and more preferably A47S.

Clause 55: The glucose isomerase of any of the preceding clauses, wherein the amino acid sequence comprises a substitution in position 140, the substitution being selected from the group consisting of W140H, W140C, W140A, W140R, W140N, W140D, W140Q, W140E, W140G, W140I, W140L, W140K, W140M, W140F, W140P, W140S, W140T, W140V or W140Y; preferably W140H, W140R, W140K; more preferably W140H.

Clause 56: The glucose isomerase of any of the preceding clauses, wherein the amino acid sequence comprises a substitution in position 147, the substitution being selected from the group consisting of T147R, T147C, T147A, T147N, T147D, T147E, T147G, T147H, T147I, T147L, T147K, T147M, T147F, T147P, T147S, T147W, T147V or T147Y; preferably T147R.

Clause 57: The glucose isomerase of any of the preceding clauses, wherein the amino acid sequence comprises a substitution in position 163, the substitution being selected from the group consisting of I163V, I163C, I163A, I163R, I163N, I163D, I163Q, I163E, I163G, I163H, I163L, I163K, I163M, I163F, I163P, I163S, I163T, I163W, or I163Y; preferably I163V.

Clause 58: The glucose isomerase of any of the preceding clauses, wherein the amino acid sequence comprises a substitution in position 187, the substitution being selected from the group consisting of V187C, V187A, V187R, V187N, V187D, V187Q, V187E, V187G, V187H, V187I, V187L, V187K, V187M, V187F, V187P, V187S, V187T, V187W, or V187Y; V187C, V187T, V187W, V187S, V187Y; preferably V187C, V187T, or V187W; and more preferably V187C.

Clause 59: The glucose isomerase of any of the preceding clauses, wherein the amino acid sequence comprises a substitution in position 190, the substitution being selected from the group consisting of G190N, G190C, G190A, G190R, G190D, G190Q, G190E, G190H, G190I, G190L, G190K, G190M, G190F, G190P, G190S, G190T, G190W or G190Y, preferably G190Q or G109N and more preferably G190N.

Clause 60: The glucose isomerase of any of the preceding clauses, wherein the amino acid sequence comprises a substitution in position 198, the substitution being selected from the group consisting of L198I, L198C, L198A, L198R, L198N, L198D, L198Q, L198E, L198G, L198H, L198K, L198M, L198F, L198P, L198S, L198T, L198W, L198V or L198Y; preferably L198I, L198V; more preferably L198I.

Clause 61: The glucose isomerase of any of the preceding clauses, wherein the amino acid sequence comprises a substitution in position 241, the substitution being selected from the group consisting of M241V, M241C, M241A, M241R, M241N, M241D, M241Q, M241E, M241G, M241H, M241I, M241L, M241K, M241F, M241P, M241S, M241T, M241W, or M241Y; preferably M241V, M241C, M241R, M241D, M241Q, M241E, M241G, M241H, M241I, M241K, M241F, M241P, M241W, or M241Y; preferably M241P, M241V, M241C, M241I, M241T; preferably M241V or M241C; more preferably M241V.

Clause 62: The glucose isomerase of any of the preceding clauses, wherein the amino acid sequence comprises a substitution in position 242, the substitution being selected from the group consisting of F242C, F242A, F242R, F242N, F242D, F242Q, F242E, F242G, F242H, F242I, F242L, F242K, F242M, F242P, F242S, F242T, F242W, F242V or F242Y; preferably F242C, F242A, F242R, F242N, F242D, F242Q, F242E, F242G, F242I, F242K, F242M, F242P, F242S, F242T, F242W, F242V, or F242Y; more preferably F242C, F242V, F242I, F242P; more preferably F242C or F242V; most preferably F242C.

Clause 63: The glucose isomerase of any of the preceding clauses, wherein the amino acid sequence comprises a substitution in position 273, the substitution being selected from the group consisting of A273S, A273C, A273R, A273N, A273D, A273Q, A273E, A273G, A273H, A273I, A273L, A273K, A273M, A273F, A273P, A273T, A273W, A273V or A273Y; preferably A273S, A273T or A273Y; more preferably A273S.

Clause 64: The glucose isomerase of any of the preceding clauses, wherein the amino acid sequence comprises a substitution in position 300, the substitution being selected from the group consisting of Q300Y, Q300C, Q300A, Q300R, Q300N, Q300D, Q300E, Q300G, Q300H, Q300I, Q300L, Q300K, Q300M, Q300F, Q300P, Q300S, Q300T, Q300W or Q300V; preferably Q300Y, Q300C, Q300A, Q300R, Q300N, Q300D, Q300G, Q300H, Q300I, Q300L, Q300K, Q300M, Q300F, Q300P, Q300S, Q300T, Q300W, or Q300V; more preferably Q300Y, Q300S, Q300C or Q300F; most preferably Q300Y.

Clause 65: The glucose isomerase of any of the preceding clauses, wherein the amino acid sequence comprises a substitution in position 309, the substitution being selected from the group consisting of M309I, M309C, M309A, M309R, M309N, M309D, M309Q, M309E, M309G, M309H, M309L, M309K, M309F, M309P, M309S, M309T, M309W, M309V or M309Y; preferably M309I, M309V, M309L, M309P; more preferably M309I or M309P; most preferably M309I.

Clause 66: The glucose isomerase of any of the preceding clauses, wherein the amino acid sequence comprises a substitution in position 313, the substitution being selected from the group consisting of P313S, P313C, P313A, P313R, P313N, P313D, P313Q, P313E, P313G, P313H, P313I, P313L, P313K, P313M, P313F, P313T, P313W, P313V or P313Y; preferably P313S, P313T, P313Y, P313V, P313L, P313I and P313G; more preferably P313S, P313V and P313G; most preferably P313S.

Clause 67: The glucose isomerase of any of the preceding clauses, wherein the amino acid sequence comprises a substitution in position 331, the substitution being selected from the group consisting of Q331G, Q331C, Q331A, Q331R, Q331N, Q331D, Q331E, Q331H, Q331I, Q331L, Q331K, Q331M, Q331F, Q331P, Q331S, Q331T, Q331W, Q331V or Q331Y; preferably Q331G.

Clause 68: The glucose isomerase of any of the preceding clauses, wherein the amino acid sequence comprises a substitution in position 342, the substitution being selected from the group consisting of A342S, A342C, A342R, A342N, A342D, A342Q, A342E, A342G, A342H, A342I, A342L, A342K, A342M, A342F, A342P, A342T, A342W, A342V or A342Y; preferably A342S, A342T, A342Y; more preferably A342S.

Clause 69: The glucose isomerase of any of the preceding clauses, wherein the amino acid sequence comprises a substitution in position 343, the substitution being selected from the group consisting of K343R, K343C, K343A, K343N, K343D, K343Q, K343E, K343G, K343H, K343I, K343L, K343M, K343F, K343P, K343S, K343T, K343W, K343V or K343Y; preferably K343R, K343N, K343Q; more preferably K343R or K343N; most preferably K343R.

Clause 70: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions at substitution positions independently of one another selected from the group consisting of: position 18, the substitution being selected from the group consisting of P18S, P18A, P18R, P18N, P18D, P18C, P18Q, P18E, P18G, P18H, P18I, P18L, P18K, P18M, P18F, P18T, P18W, P18V or P18Y; more preferably P18S, P18T, P18Y; and most preferably P18S; and/or position 47, the substitution being selected from the group consisting of A47S, A47C, A47R, A47N, A47D, A47Q, A47E, A47G, A47H, A47I, A47L, A47K, A47M, A47F, A47P, A47T, A47W, A47V, or A47Y; preferably A47S, A47Q or A47R; and more preferably A47S; and/or position 140, the substitution being selected from the group consisting of W140H, W140C, W140A, W140R, W140N, W140D, W140Q, W140E, W140G, W140I, W140L, W140K, W140M, W140F, W140P, W140S, W140T, W140V or W140Y; preferably W140H, W140R, W140K; more preferably W140H; and/or position 147, the substitution being selected from the group consisting of T147R, T147C, T147A, T147N, T147D, T147E, T147G, T147H, T147I, T147L, T147K, T147M, T147F, T147P, T147S, T147W, T147V or T147Y; preferably T147R; and/or position 163, the substitution being selected from the group consisting of I163V, I163C, I163A, I163R, I163N, I163D, I163Q, I163E, I163G, I163H, I163L, I163K, I163M, I163F, I163P, I163S, I163T, I163W, or I163Y; preferably I163V; and/or position 187, the substitution being selected from the group consisting of V187C, V187A, V187R, V187N, V187D, V187Q, V187E, V187G, V187H, V187I, V187L, V187K, V187M, V187F, V187P, V187S, V187T, V187W, or V187Y; preferably V187C or V187T, or V187W; and more preferably V187C; and/or position 190, the substitution being selected from the group consisting of G190N, G190C, G190A, G190R, G190D, G190Q, G190E, G190H, G190I, G190L, G190K, G190M, G190F, G190P, G190S, G190T, G190W, or G190Y; preferably G190Q or G109N; and/or position 198, the substitution being selected from the group consisting of L198I, L198C, L198A, L198R, L198N, L198D, L198Q, L198E, L198G, L198H, L198K, L198M, L198F, L198P, L198S, L198T, L198W, L198V or L198Y; preferably L198I; and/or position 241, the substitution being selected from the group consisting of M241V, M241C, M241A, M241R, M24 IN, M241D, M241Q, M241E, M241G, M241H, M241I, M241L, M241K, M241F, M241P, M241S, M241T, M241W, or M241Y; preferably M241V, M241C, M241R, M241D, M241Q, M241E, M241G, M241H, M241I, M241K, M241F, M241P, M241W, or M241Y; more preferably M241V or M241C; most preferably M241V; and/or position 242, the substitution being selected from the group consisting of F242C, F242A, F242R, F242N, F242D, F242Q, F242E, F242G, F242H, F242I, F242L, F242K, F242M, F242P, F242S, F242T, F242W, F242V or F242Y; preferably F242C, F242A, F242R, F242N, F242D, F242Q, F242E, F242G, F242I, F242K, F242M, F242P, F242S, F242T, F242W, F242V, or F242Y; more preferably F242C or F242V; most preferably F242C; and/or position 273, the substitution being selected from the group consisting of A273S, A273C, A273R, A273N, A273D, A273Q, A273E, A273G, A273H, A273I, A273L, A273K, A273M, A273F, A273P, A273T, A273W, A273V or A273Y; preferably A273S, A273T or A273Y; and/or position 300, the substitution being selected from the group consisting of Q300Y, Q300C, Q300A, Q300R, Q300N, Q300D, Q300E, Q300G, Q300H, Q300I, Q300L, Q300K, Q300M, Q300F, Q300P, Q300S, Q300T, Q300W or Q300V; preferably Q300Y, Q300C, Q300A, Q300R, Q300N, Q300D, Q300G, Q300H, Q300I, Q300L, Q300K, Q300M, Q300F, Q300P, Q300S, Q300T, Q300W, or Q300V; more preferably Q300Y, Q300S, Q300C or Q300F; most preferably Q300Y; and/or position 309, the substitution being selected from the group consisting of M309I, M309C, M309A, M309R, M309N, M309D, M309Q, M309E, M309G, M309H, M309L, M309K, M309F, M309P, M309S, M309T, M309W, M309V or M309Y; preferably M309I, M309V, M309L, M309P; more preferably M309I or M309P; most preferably M309I; and/or position 313, the substitution being selected from the group consisting of P313S, P313C, P313A, P313R, P313N, P313D, P313Q, P313E, P313G, P313H, P313I, P313L, P313K, P313M, P313F, P313T, P313W, P313V or P313Y; preferably P313S, P313V and P313G; more preferably P313S; and/or position 331, the substitution being selected from the group consisting of Q331G, Q331C, Q331A, Q331R, Q33 IN, Q331D, Q331E, Q331H, Q331I, Q331L, Q331K, Q331M, Q331F, Q331P, Q331S, Q331T, Q331W, Q331V or Q331Y; preferably Q331G; and/or position 342, the substitution being selected from the group consisting of A342S, A342C, A342R, A342N, A342D, A342Q, A342E, A342G, A342H, A342I, A342L, A342K, A342M, A342F, A342P, A342T, A342W, A342V or A342Y; preferably A342S, A342T, A342Y; more preferably A342S; and/or position 343, the substitution being selected from the group consisting of K343R, K343C, K343A, K343N, K343D, K343Q, K343E, K343G, K343H, K343I, K343L, K343M, K343F, K343P, K343S, K343T, K343W, K343V or K343Y; preferably K343R, K343N, K343Q; more preferably K343R or K343N; most preferably K343R.

Clause 71: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions at substitution positions independently of one another selected from the group consisting of: position 47, the substitution being selected from the group consisting of A47S, A47C, A47R, A47N, A47D, A47Q, A47E, A47G, A47H, A47I, A47L, A47K, A47M, A47F, A47P, A47T, A47W, A47V, or A47Y; preferably A47S, A47Q or A47R; and more preferably A47S; and/or position 140, the substitution being selected from the group consisting of W140H, W140C, W140A, W140R, W140N, W140D, W140Q, W140E, W140G, W140I, W140L, W140K, W140M, W140F, W140P, W140S, W140T, W140V or W140Y; preferably W140H, W140R, W140K; more preferably W140H; and/or position 187, the substitution being selected from the group consisting of V187C, V187A, V187R, V187N, V187D, V187Q, V187E, V187G, V187H, V187I, V187L, V187K, V187M, V187F, V187P, V187S, V187T, V187W, or V187Y; preferably V187C or V187T, or V187W; and more preferably V187C; and/or position 190, the substitution being selected from the group consisting of G190N, G190C, G190A, G190R, G190D, G190Q, G190E, G190H, G190I, G190L, G190K, G190M, G190F, G190P, G190S, G190T, G190W, or G190Y; preferably G190Q or G109N; and/or position 241, the substitution being selected from the group consisting of M241V, M241C, M241A, M241R, M241N, M241D, M241Q, M241E, M241G, M241H, M241I, M241L, M241K, M241F, M241P, M241S, M241T, M241W, or M241Y; preferably M241V, M241C, M241R, M241D, M241Q, M241E, M241G, M241H, M241I, M241K, M241F, M241P, M241W, or M241Y; more preferably M241V or M241C; most preferably M241V; and/or position 242, the substitution being selected from the group consisting of F242C, F242A, F242R, F242N, F242D, F242Q, F242E, F242G, F242H, F242I, F242L, F242K, F242M, F242P, F242S, F242T, F242W, F242V or F242Y; preferably F242C, F242A, F242R, F242N, F242D, F242Q, F242E, F242G, F242I, F242K, F242M, F242P, F242S, F242T, F242W, F242V, or F242 Y; more preferably F242C or F242V; most preferably F242C; and/or position 273, the substitution being selected from the group consisting of A273S, A273C, A273R, A273N, A273D, A273Q, A273E, A273G, A273H, A273I, A273L, A273K, A273M, A273F, A273P, A273T, A273W, A273V or A273Y; preferably A273S, A273T or A273Y; and/or position 300, the substitution being selected from the group consisting of Q300Y, Q300C, Q300A, Q300R, Q300N, Q300D, Q300E, Q300G, Q300H, Q300I, Q300L, Q300K, Q300M, Q300F, Q300P, Q300S, Q300T, Q300W or Q300V; preferably Q300Y, Q300C, Q300A, Q300R, Q300N, Q300D, Q300G, Q300H, Q300I, Q300L, Q300K, Q300M, Q300F, Q300P, Q300S, Q300T, Q300W, or Q300V; more preferably Q300Y, Q300S, Q300C or Q300F; most preferably Q300Y; and/or position 309, the substitution being selected from the group consisting of M309I, M309C, M309A, M309R, M309N, M309D, M309Q, M309E, M309G, M309H, M309L, M309K, M309F, M309P, M309S, M309T, M309W, M309V or M309Y; preferably M309I, M309V, M309L, M309P; more preferably M309I or M309P; most preferably M309I; and/or position 313, the substitution being selected from the group consisting of P313S, P313C, P313A, P313R, P313N, P313D, P313Q, P313E, P313G, P313H, P313I, P313L, P313K, P313M, P313F, P313T, P313W, P313V or P313Y; preferably P313S, P313V and P313G; more preferably P313S; and/or position 342, the substitution being selected from the group consisting of A342S, A342C, A342R, A342N, A342D, A342Q, A342E, A342G, A342H, A342I, A342L, A342K, A342M, A342F, A342P, A342T, A342W, A342V or A342Y; preferably A342S, A342T, A342Y; more preferably A342S; and/or position 343, the substitution being selected from the group consisting of K343R, K343C, K343A, K343N, K343D, K343Q, K343E, K343G, K343H, K343I, K343L, K343M, K343F, K343P, K343S, K343T, K343W, K343V or K343Y; preferably K343R, K343N, K343Q; more preferably K343R or K343N; most preferably K343R.

Clause 72: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions at substitution positions independently of one another selected from the group consisting of: position 18, the substitution being selected from of P18S, P18T or P18Y; more preferably the substitution being P18S; and/or position 47, the substitution being selected from the group consisting of A47S, A47Q or A47R; preferably A47S; and/or position 140, the substitution being W140H; and/or position 147, the substitution being T147R; and/or position 163, the substitution being I163V; and/or position 187, the substitution being selected from the group consisting of V187C, or V187T, or V187W; preferably V187C; and/or position 190, the substitution being G190N; and/or position 198, the substitution being L198I; and/or position 241, the substitution being M241V or M241C; preferably M241V; and/or position 242, the substitution being F242C or F242V; preferably F242C; and/or position 273, the substitution being A273S; and/or position 300, the substitution being selected from the group consisting of Q300Y, Q300S, Q300C or Q300F; preferably Q300Y or Q300S, most preferably Q300Y; and/or position 309, the substitution being M309I; and/or position 313, the substitution being selected from the group consisting of P313S, P313V and P313G; preferably P313S; and/or position 331, the substitution being Q331G; and/or position 342, the substitution being A342S; and/or position 343, the substitution being K343R or K343N; preferably K343R.

Clause 73: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions independently of one another selected from the groups consisting of P18S, A47S, W140H, T147R, I163V, V187C, V187T, V187W, G190N, L198I, M241V, F242C, F242V, A273S, Q300Y, Q300S, Q300C, Q300F, M309I, P313S, P313V, P313G, Q331G, A342S, K343N and K343R; preferably selected from the group consisting of P18S, A47S, W140H, T147R, I163V, V187C, V187T, V187W, G190N, M241V, F242C, F242V, Q300Y, Q300S, Q300C, Q300F, M309I, P313S, P313V, P313G, Q331G, A342S, K343N and K343R; more preferably selected from the group consisting of A47S, W140H, V187C, V187T, V187W, G190N, M241V, F242C, F242V, Q300Y, Q300S, Q300C, Q300F, M309I, P313S, P313V, P313G, Q331G, A342S, K343N and K343R; and most preferably selected from the group consisting of A47S, V187C, V187T, V187W, M241V, F242C, F242V, Q300Y, Q300S, Q300C, Q300F, M309I, P313S, P313V, P313G, A342S, K343N and K343R.

Clause 74: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions independently of one another selected from the groups consisting of V187C, V187T, V187W, P313S, P313V, P313G, Q300Y, Q300S, Q300C, Q300F, A47S, W140H, G190N, M241V, F242C, F242V, A273S, M309I, A342S, K343N and K343R; and preferably selected from the group consisting of V187C, V187T, V187W, P313S, P313V, P313G, Q300Y, Q300S, Q300C, Q300F, A47S, M241V, F242C, F242V, M309I, A342S, K343N and K343R; and preferably selected from the group consisting of V187C, V187T, V187W, P313S, P313V, P313G, Q300Y, Q300S, Q300C, and Q300F.

Clause 75: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions independently of one another selected from the groups consisting of V187C, P18S, A47S, W140H, T147R, I163V, G190N, M241V, F242C, Q300Y, M309I, P313S, Q331G, A342S, and K343R; or V187C, P18S, A47S, W140H, I163V, L198I, M241V, F242C, Q300Y, M309I, P313S, Q331G, K343R, and A342S; or V187C, P18S, A47S, W140H, I163V, G190N, L198I, M241V, F242C, A273S, Q300Y, M309I, P313V, Q331G, A342S, and K343R; or Q300Y, P18S, A47S, W140H, T147R, I163V, V187C, G190N, L198I, M241V, F242C, A273S, M309I, K343R, P313V, Q331G, and A342S.

Clause 76: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions independently of one another selected from the groups consisting of P18S, A47S, W140H, I163V, V187C, M241V, F242C, Q300Y, P313V, Q331G, A342S, and K343R; or A47S, V187C, V187T, G V187W, M241V, F242C, F242V, Q300Y, Q300S, Q300C, Q300F, M309I, P313S, P313V, P313G, A342S, K343N and K343R; preferably A47S, V187C, M241V, F242C, Q300Y, M309I, P313S, A342S, and K343R.

Clause 77: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions independently of one another selected from the groups consisting of P18S, P18T, P18Y, A47S, A47Q, A47R, W140H, W140R, W140K, T147R, I163V, V187C, V187T, V187W, G190Q, G109N, L198I, L198V, M241P, M241V, M241C, M241I, M241S, M241T, M241L, F242C, F242V, F242I, F242L, F242P, A273S, A273T, A273Y, Q300Y, Q300S, Q300C, Q300F, M309I, M309V, M309L, M309P, P313S, P313T, P313Y, P313V, P313L, P313I, P313G, Q331G, A342S, A342T, A342Y, K343R, K343N, K343Q; preferably of P18S, A47S, W140H, T147R, I163V, V187C, G190N, L198I, M241V, M241C, F242C, F242V, A273S, Q300Y, M309I, P313S, P313V, P313G, Q331G, A342S, K343R, K343N.

Clause 78: The glucose isomerase of any of the preceding clauses, wherein the amino acid sequence comprises at least two, three, four, five, six, seven, eight, nine, ten, eleven, twelve, thirteen, fourteen, fifteen, sixteen or seventeen substitutions at substitution positions, wherein the amino acid sequence comprises at least two substitution selected from the group consisting of (i) V187C+Q300Y, V187C+P18S, V187C+A47S, V187C+W140H, V187C+T147R, V187C+I163V, V187C+G190N, V187C+M241V, V187C+F242C, V187C+M309I, V187C+P313S, V187C+Q331G, V187C+A342S, V187C+K343R, P18S+A47S, P18S+W140H, P18S+T147R, P18S+I163V, P18S+G190N, P18S+M241V, P18S+F242C, P18S+Q300Y, P18S+M309I, P18S+P313S, P18S+Q331G, P18S+A342S, P18S+K343R, A47S+W140H, A47S+T147R, A47S+I163V, A47S+G190N, A47S+M241V, A47S+F242C, A47S+Q300Y, A47S+M309I, A47S+P313S, A47S+Q331G, A47S+A342S, A47S+K343R, W140H+T147R, W140H+I163V, W140H+G190N, W140H+M241V, W140H+F242C, W140H+Q300Y, W140H+M309I, W140H+P313S, W140H+Q331G, W140H+A342S, W140H+K343R, T147R+I163V, T147R+G190N, T147R+M241V, T147R+F242C, T147R+Q300Y, T147R+M309I, T147R+P313S, T147R+Q331G, T147R+K343R, T147R+A342S, I163V+G190N, I163V+M241V, I163V+F242C, I163V+Q300Y, I163V+M309I, I163V+P313S, I163V+Q331G, I163V+A342S, I163V+K343R, G190N+M241V, G190N+F242C, G190N+Q300Y, G190N+M309I, G190N+P313S, G190N+Q331G, G190N+A342S, G190N+K343R, M241V+F242C, M241V+Q300Y, M241V+M309I, M241V+P313S, M241V+Q331G, M241V+A342S, M241V+K343R, F242C+Q300Y, F242C+M309I, F242C+P313S, F242C+Q331G, F242C+A342S, F242C+K343R, Q300Y+M309I, Q300Y+P313S, Q300Y+Q331G, Q300Y+A342S, Q300Y+K343R, M309I+P313S, M309I+Q331G, M309I+A342S, M309I+K343R, P313S+Q331G, P313S+A342S, P313S+K343R, Q331G+A342S, Q331G+K343R, and A342S+K343R; and/or (ii) V187C+Q300Y, V187C+P18S, V187C+A47S, V187C+W140H, V187C+I163V, V187C+L198I, V187C+M241V, V187C+F242C, V187C+M309I, V187C+P313S, V187C+Q331G, V187C+A342S, V187C+K343R, P18S+A47S, P18S+W140H, P18S+I163V, P18S+L198I, P18S+M241V, P18S+F242C, P18S+Q300Y, P18S+M309I, P18S+P313S, P18S+Q331G, P18S+A342S, P18S+K343R, A47S+W140H, A47S+I163V, A47S+L198I, A47S+M241V, A47S+F242C, A47S+Q300Y, A47S+M309I, A47S+P313S, A47S+Q331G, A47S+A342S, A47S+K343R, W140H+I163V, W140H+L198I, W140H+M241V, W140H+F242C, W140H+Q300Y, W140H+M309I, W140H+P313S, W140H+Q331G, W140H+A342S, W140H+K343R, I163V+L198I, I163V+M241V, I163V+F242C, I163V+Q300Y, I163V+M309I, I163V+P313S, I163V+Q331G, I163V+A342S, I163V+K343R, L198I+M241V, L198I+F242C, L198I+Q300Y, L198I+M309I, L198I+P313S, L198I+Q331G, L198I+A342S, L198I+K343R, M241V+F242C, M241V+Q300Y, M241V+M309I, M241V+P313S, M241V+Q331G, M241V+A342S, M241V+K343R, F242C+Q300Y, F242C+M309I, F242C+P313S, F242C+Q331G, F242C+A342S, F242C+K343R, Q300Y+M309I, Q300Y+P313S, Q300Y+Q331G, Q300Y+A342S, Q300Y+K343R, M309I+P313S, M309I+Q331G, M309I+A342S, M309I+K343R, P313S+Q331G, P313S+A342S, P313S+K343R, Q331G+A342S, Q331G+K343R, and A342S+K343R; and/or (iii) V187C+P18S, V187C+A47S, V187C+N, V187C+W140H, V187C+I163V, V187C+G190N, V187C+L198I, V187C+M241V, V187C+F242C, V187C+A273S, V187C+Q300Y, V187C+M309I, V187C+P313V, V187C+Q331G, V187C+A342S, V187C+K343R, P18S+A47S, P18S+W140H, P18S+I163V, P18S+G190N, P18S+L198I, P18S+M241V, P18S+F242C, P18S+A273S, P18S+Q300Y, P18S+M309I, P18S+P313V, P18S+Q331G, P18S+A342S, P18S+K343R, A47S+W140H, A47S+I163V, A47S+G190N, A47S+L198I, A47S+M241V, A47S+F242C, A47S+A273S, A47S+Q300Y, A47S+M309I, A47S+P313V, A47S+Q331G, A47S+A342S, A47S+K343R, W140H+I163V, W140H+G190N, W140H+L198I, W140H+M241V, W140H+F242C, W140H+A273S, W140H+Q300Y, W140H+M309I, W140H+P313V, W140H+Q331G, W140H+A342S, W140H+K343R, I163V+G190N, I163V+L198I, I163V+M241V, I163V+F242C, I163V+A273S, I163V+Q300Y, I163V+M309I, I163V+P313V, I163V+Q331G, I163V+A342S, I163V+K343R, G190N+L198I, G190N+M241V, G190N+F242C, G190N+A273S, G190N+Q300Y, G190N+M309I, G190N+P313V, G190N+Q331G, G190N+A342S, G190N+K343R, L198I+M241V, L198I+F242C, L198I+A273S, L198I+Q300Y, L198I+M309I, L198I+P313V, L198I+Q331G, L198I+A342S, L198I+K343R, M241V+F242C, M241V+A273S, M241V+Q300Y, M241V+M309I, M241V+P313V, M241V+Q331G, M241V+A342S, M241V+K343R, F242C+A273S, F242C+Q300Y, F242C+M309I, F242C+P313V, F242C+Q331G, F242C+A342S, F242C+K343R, A273S+Q300Y, A273S+M309I, A273S+P313V, A273S+Q331G, A273S+A342S, A273S+K343R, Q300Y+M309I, Q300Y+P313V, Q300Y+Q331G, Q300Y+A342S, Q300Y+K343R, M309I+P313V, M309I+Q331G, M309I+A342S, M309I+K343R, P313V+Q331G, P313V+A342S, P313V+K343R, Q331G+A342S, Q331G+K343R, and A342S+K343R; and/or (iv) Q300Y+P18S, Q300Y+A47S, Q300Y+W140H, Q300Y+T147R, Q300Y+I163V, Q300Y+V187C, Q300Y+G190N, Q300Y+L198I, Q300Y+M241V, Q300Y+F242C, Q300Y+A273S, Q300Y+M309I, Q300Y+K343R, Q300Y+P313V, Q300Y+Q331G, Q300Y+A342S, P18S+A47S, P18S+W140H, P18S+T147R, P18S+I163V, P18S+V187C, P18S+G190N, P18S+L198I, P18S+M241V, P18S+F242C, P18S+A273S, P18S+M309I, P18S+K343R, P18S+P313V, P18S+Q331G, P18S+A342S, A47S+W140H, A47S+T147R, A47S+I163V, A47S+V187C, A47S+G190N, A47S+L198I, A47S+M241V, A47S+F242C, A47S+A273S, A47S+M309I, A47S+K343R, A47S+P313V, A47S+Q331G, A47S+A342S, W140H+T147R, W140H+I163V, W140H+V187C, W140H+G190N, W140H+L198I, W140H+M241V, W140H+F242C, W140H+A273S, W140H+M309I, W140H+K343R, W140H+P313V, W140H+Q331G, W140H+A342S, T147R+I163V, T147R+V187C, T147R+G190N, T147R+L198I, T147R+M241V, T147R+F242C, T147R+A273S, T147R+M309I, T147R+K343R, T147R+P313V, T147R+Q331G, T147R+A342S, I163V+V187C, I163V+G190N, I163V+L198I, I163V+M241V, I163V+F242C, I163V+A273S, I163V+M309I, I163V+K343R, I163V+P313V, I163V+Q331G, I163V+A342S, V187C+G190N, V187C+L198I, V187C+M241V, V187C+F242C, V187C+A273S, V187C+M309I, V187C+K343R, V187C+P313V, V187C+Q331G, V187C+A342S, G190N+L198I, G190N+M241V, G190N+F242C, G190N+A273S, G190N+M309I, G190N+K343R, G190N+P313V, G190N+Q331G, G190N+A342S, L198I+M241V, L198I+F242C, L198I+A273S, L198I+M309I, L198I+K343R, L198I+P313V, L198I+Q331G, L198I+A342S, M241V+F242C, M241V+A273S, M241V+M309I, M241V+K343R, M241V+P313V, M241V+Q331G, M241V+A342S, F242C+A273S, F242C+M309I, F242C+K343R, F242C+P313V, F242C+Q331G, F242C+A342S, A273S+M309I, A273S+K343R, A273S+P313V, A273S+Q331G, A273S+A342S, M309I+K343R, M309I+P313V, M309I+Q331G, M309I+A342S, K343R+P313V, K343R+Q331G, K343R+A342S, P313V+Q331G, P313V+A342S, P313V+, Q331G+A342S.

Clause 79: The glucose isomerase of any of the preceding clauses, wherein the amino acid sequence comprises at least two, three, four, five, six, seven, eight, nine, ten, eleven, twelve, thirteen, fourteen, fifteen, sixteen or seventeen substitutions at substitution positions, wherein the amino acid sequence comprises at least two substitution selected from the group consisting of A47S+V187C, A47S+V187W, A47S+V187T, A47S+M241V, A47S+F242C, A47S+F242V, A47S+Q300Y, A47S+Q300S, A47S+Q300C, A47S+Q300F, A47S+M309I, A47S+P313S, A47S+P313V, A47S+A342S, A47S+K343R, A47S+K343N, V187W+M241V, V187W+F242C, V187W+F242V, V187W+Q300Y, V187W+Q300S, V187W+Q300C, V187W+Q300F, V187W+M309I, V187W+P313S, V187W+P313V, V187W+P313G, V187W+A342S, V187W+K343R, V187W+K343N, V187T+M241V, V187T+F242C, V187T+F242V, V187T+Q300Y, V187T+Q300S, V187T+Q300C, V187T+Q300F, V187T+M309I, V187T+P313S, V187T+P313V, V187T+P313G, V187T+A342S, V187T+K343R, V187T+K343N, V187C+M241V, V187C+F242C, V187C+F242V, V187C+Q300Y, V187C+Q300S, V187C+Q300C, V187C+Q300F, V187C+M309I, V187C+P313S, V187C+P313V, V187C+P313G, V187C+A342S, V187C+K343R, V187C+K343N, M241V+F242C, M241V+Q300Y, M241V+Q300F, M241V+Q300C, M241V+Q300S, M241V+M309I, M241V+P313S, M241V+P313V, M241V+P313G, M241V+A342S, M241V+K343R, M241V+K343N, F242C+Q300Y, F242C+Q300S, F242C+Q300C, F242C+Q300F, F242C+M309I, F242C+P313S, F242C+P313V, F242C+P313G, F242C+A342S, F242C+K343R, F242C+K343N, F242V+Q300Y, F242V+Q300S, F242V+Q300C, F242V+Q300F, F242V+M309I, F242V+P313S, F242V+P313V, F242V+P313G, F242V+A342S, F242V+K343R, F242V+K343N, Q300Y+M309I, Q300Y+P313S, Q300Y+P313V, Q300Y+P313G, Q300Y+A342S, Q300Y+K343R, Q300Y+K343N, Q300S+M309I, Q300S+P313S, Q300S+P313V, Q300S+P313G, Q300S+A342S, Q300S+K343R, Q300S+K343N, Q300C+M309I, Q300C+P313S, Q300C+P313V, Q300C+P313G, Q300C+A342S, Q300C+K343R, Q300C+K343N, Q300F+M309I, Q300F+P313S, Q300F+P313V, Q300F+P313G, Q300F+A342S, Q300F+K343R, Q300F+K343N, M309I+P313S, M309I+A342S, M309I+K343R, M309I+K343K, P313S+A342S, P313S+K343R, P313S+K343N, P313V+A342S, P313V+K343R, P313V+K343N, P313G+A342S, P313G+K343R, P313G+K343N, A342S+K343N and A342S+K343R.

Clause 80: The glucose isomerase of any of the preceding clauses, wherein the amino acid sequence comprises at least two, three, four, five, six, seven, eight, nine, ten, eleven, twelve, thirteen, fourteen, fifteen, sixteen or seventeen substitutions at substitution positions, wherein the substitution positions are in any subsequence and are independently of one another selected from the groups consisting of V187 and Q300, and wherein preferably the substitutions are independently of one another selected from V187Q and Q300Y.

Clause 81: The glucose isomerase of any of the preceding clauses, wherein the amino acid sequence comprises at least three, four, five, six, seven, eight, nine, ten, eleven, twelve, thirteen, fourteen, fifteen, sixteen or seventeen substitutions at substitution positions, wherein the amino acid sequence comprises at least three substitution selected from the group consisting of (i) V187C+P313S+K343R, V187C+M241V+F242C, V187C+Q300S+M309I, V187C+A342S+A47S, V187C+P313V+A47S, V187C+F242C+K343R, V187C+P313S+A47S, V187T+F242C+P313S, and V187C+K343R+A47S; preferably selected from the group consisting of V187C+P313S+K343R, V187C+M241V+F242C, V187C+Q300S+M309I, V187C+A342S+A47S, V187C+P313V+A47S, V187C+F242C+K343R, and V187C+P313S+A47S; more preferably selected from the group consisting of V187C+P313S+K343R, V187C+M241V+F242C, V187C+Q300S+M309I, and V187C+A342S+A47S; even more preferably selected from the group consisting of V187C+P313S+K343R, and V187C+M241V+F242C; and/or (ii) V187C+Q300S+M309I, V187C+M241V+F242C, V187T+F242C+P313S, V187C+P313S+K343R, V187C+P313V+A47S, V187C+A342S+A47S, V187C+F242C+K343R, and V187C+P313S+A47S; preferably selected from the group consisting of V187C+Q300S+M309I, V187C+M241V+F242C, V187T+F242C+P313S, V187C+P313S+K343R, V187C+P313V+A47S, and V187C+A342S+A47S; more preferably selected from the group consisting of V187C+Q300S+M309I, V187C+M241V+F242C, V187T+F242C+P313S, and V187C+P313S+K343R; even more preferably selected from the group consisting of V187C+Q300S+M309I, and V187C+M241V+F242C; and/or (iii) V187C+P313S+K343R, V187C+Q300S+M309I, V187C+M241V+F242C, V187C+P313V+A47S, V187C+A342S+A47S, V187C+F242C+K343R, V187T+F242C+P313S, and V187C+P313S+A47S; preferably selected from the group consisting of V187C+P313S+K343R, V187C+Q300S+M309I, V187C+M241V+F242C, V187C+P313V+A47S, V187C+A342S+A47S, and V187C+F242C+K343R; more preferably selected from the group consisting of V187C+P313S+K343R, V187C+Q300S+M309I, V187C+M241V+F242C, and V187C+P313V+A47S; even more preferably selected from the group consisting of V187C+P313S+K343R, and V187C+Q300S+M309I; and/or (iv) V187C+P313S+K343R, V187C+M241V+F242C, V187C+Q300S+M309I, V187C+A342S+A47S, V187C+P313V+A47S, V187C+F242C+K343R, V187T+F242C+P313S, and V187C+P313S+A47S; preferably selected from the group consisting of V187C+P313S+K343R, V187C+M241V+F242C, V187C+Q300S+M309I, V187C+A342S+A47S, V187C+P313V+A47S, and V187C+F242C+K343R; more preferably selected from the group consisting of V187C+P313S+K343R, V187C+M241V+F242C, V187C+Q300S+M309I, and V187C+A342S+A47S; even more preferably selected from the group consisting of V187C+P313S+K343R, and V187C+M241V+F242C; and/or (v) V187C+P313S+K343R, V187C+A342S+A47S, V187C+M241V+F242C, V187C+F242C+K343R, V187C+P313V+A47S, V187C+Q300S+M309I, V187T+F242C+P313S, V187C+K343R+A47S, and V187C+P313S+A47S; preferably selected from the group consisting of V187C+P313S+K343R, V187C+A342S+A47S, V187C+M241V+F242C, V187C+F242C+K343R, V187C+P313V+A47S, V187C+Q300S+M309I, V187T+F242C+P313S, V187C+K343R+A47S, and V187C+P313S+A47S; more preferably selected from the group consisting of V187C+P313S+K343R, V187C+A342S+A47S, V187C+M241V+F242C, and V187C+F242C+K343R; even more preferably select-ed from the group consisting of V187C+P313S+K343R, and V187C+A342S+A47S; most preferably at the three amino acid positions V187C+P313S+K343R.

Clause 82: The glucose isomerase of any of the preceding clauses, wherein the amino acid sequence comprises at least four, five, six, seven, eight, nine, ten, eleven, twelve, thirteen, fourteen, fifteen, sixteen or seventeen substitutions at substitution positions, wherein the amino acid sequence comprises at least four substitution selected from the group consisting of V187C+Q300S+M309I+P313S, V187C+F242C+Q300S+M309I, V187W+F242C+Q300S+A47S, V187C+M241V+F242C+Q300Y, V187W+M241V+F242C+P313V, V187C+F242C+Q300Y+M309I, V187C+F242C+A342S+A47S, V187C+M241V+F242C+A47S, V187C+M309I+P313S+A47S, V187C+F242C+P313S+A342S, V187W+F242C+P313S+A47S, V187C+F242C+M309I+A47S, V187C+M241V+F242C+P313G, V187C+F242C+Q300Y+A47S, V187C+A342S+K343R+A47S, V187C+M309I+P313V+A47S, V187C+P313S+A342S+A47S, V187C+P313V+K343R+A47S, and V187C+M241V+F242C+A342S, preferably selected from the group consisting of V187C+F242C+Q300S+M309I, V187W+M241V+F242C+P313V and V187C+Q300S+M309I+P313S.

Clause 83: The glucose isomerase of any of the preceding clauses, wherein the amino acid sequence comprises at least five, six, seven, eight, nine, ten, eleven, twelve, thirteen, fourteen, fifteen, sixteen or seventeen substitutions at substitution positions, wherein the amino acid sequence comprises at least five substitution selected from the group consisting of V187C+F242C+M309I+K343R+A47S, V187C+F242C+M309I+A342S+A47S, V187C+Q300S+M309I+K343R+A47S, V187C+M241V+F242C+M309I+A47S, V187C+M241V+F242C+Q300S+M309I, V187C+M241V+F242C+A342A+A47S, V187C+M241V+F242C+K343R+A47S, V187C+M241V+F242C+Q300Y+A47S, V187C+F242C+Q300Y+K343N+A47S, V187C+F242C+A342S+K343N+A47S, V187C+M241V+F242C+A342S+A47S, V187C+M241V+F242C+P313S+A47S, V187C+M241V+F242C+Q300S+A47S, V187C+M241V+F242C+M309I+A342S and V187C+F242C+M309I+P313S+A47S preferably selected from the group consisting of V187C+F242C+M309I+K343R+A47S, and V187C+M241V+F242C+M309I+A47S.

Clause 84: The glucose isomerase of any of the preceding clauses, wherein the amino acid sequence comprises at least six, seven, eight, nine, ten, eleven, twelve, thirteen, fourteen, fifteen, sixteen or seventeen substitutions at substitution positions, wherein the amino acid sequence comprises at least six substitutions selected from the group consisting of V187C+M241V+F242C+Q300S+M309I+A47S, V187C+F242C+M309I+P313S+K343R+A47S and V187C+F242C+M309I+A342S+K343R+A47S.

Clause 85: The glucose isomerase of any of the preceding clauses, wherein the amino acid sequence comprises at least seven, eight, nine, ten, eleven, twelve, thirteen, fourteen, fifteen, sixteen or seventeen substitutions at substitution positions, wherein the amino acid sequence comprises at least seven substitutions selected from the group consisting of V187C+M241V+F242C+M309I+P313S+K343R+A47S and V187C+M241V+F242C+M309I+A342S+K343R+A47S.

Clause 86: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions, wherein the amino acid sequence comprises a substitution in at least one further, at least two further, at least three further, at least four further, at least five further, at least six further, at least seven further, at least eight further, at least nine further, at least ten further, at least eleven further, at least twelve further, at least thirteen further, at least fourteen further, at least fifteen further, or at least sixteen further substitution positions selected from the group consisting of substitution positions P18, A47, W140, T147, I163, V187, G190, L198, M241, F242, A273, Q300, M309, P313, Q331, A342, and K343; preferably consisting of A47, V187, M241, F242, Q300, M309, P313, A342 and K343.

Clause 87: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions, wherein the amino acid sequence comprises a substitution in at least one further, at least two further, at least three further, at least four further, at least five further, at least six further, at least seven further, at least eight further, at least nine further, at least ten further, at least eleven further, at least twelve further, at least thirteen further, at least fourteen further, at least fifteen further, or at least sixteen further substitution positions selected from the group consisting of: position 18, the substitution being selected from the group consisting of P18S, P18A, P18R, P18N, P18D, P18C, P18Q, P18E, P18G, P18H, P18I, P18L, P18K, P18M, P18F, P18T, P18W, P18V or P18Y; more preferably P18S, P18T, P18Y; and most preferably P18S; and/or position 47, the substitution being selected from the group consisting of A47S, A47C, A47R, A47N, A47D, A47Q, A47E, A47G, A47H, A47I, A47L, A47K, A47M, A47F, A47P, A47T, A47W, A47V, or A47Y; preferably A47S, A47Q or A47R; and more preferably A273S, A273T or A273Y; and/or position 140, the substitution being selected from the group consisting of W140H, W140C, W140A, W140R, W140N, W140D, W140Q, W140E, W140G, W140I, W140L, W140K, W140M, W140F, W140P, W140S, W140T, W140V or W140Y; preferably W140H, W140R, or W140K; and/or position 147, the substitution being selected from the group consisting of T147R, T147C, T147A, T147N, T147D, T147E, T147G, T147H, T147I, T147L, T147K, T147M, T147F, T147P, T147S, T147W, T147V or T147Y; preferably T147R; and/or position 163, the substitution being selected from the group consisting of I163V, I163C, I163A, I163R, I163N, I163D, I163Q, I163E, I163G, I163H, I163L, I163K, I163M, I163F, I163P, I163S, I163T, I163W, or I163Y; preferably I163V; and/or position 187, the substitution being selected from the group consisting of V187C, V187A, V187R, V187N, V187D, V187Q, V187E, V187G, V187H, V187I, V187L, V187K, V187M, V187F, V187P, V187S, V187T, V187W, or V187Y; preferably V187C or V187T, or V187W; and more preferably V187C; and/or position 190, the substitution being selected from the group consisting of G190N, G190C, G190A, G190R, G190D, G190Q, G190E, G190H, G190I, G190L, G190K, G190M, G190F, G190P, G190S, G190T, G190W, or G190Y; preferably G190Q or G109N; and/or position 198, the substitution being selected from the group consisting of L198I, L198C, L198A, L198R, L198N, L198D, L198Q, L198E, L198G, L198H, L198K, L198M, L198F, L198P, L198S, L198T, L198W, L198V or L198Y; preferably L198I; and/or position 241, the substitution being selected from the group consisting of M241V, M241C, M241A, M241R, M24 IN, M241D, M241Q, M241E, M241G, M241H, M241I, M241L, M241K, M241F, M241P, M241S, M241T, M241W, or M241Y; preferably M241V, M241C, M241R, M241D, M241Q, M241E, M241G, M241H, M241I, M241K, M241F, M241P, M241W, or M241Y; more preferably M241V or M241C; most preferably M241V or M241C; and/or position 242, the substitution being selected from the group consisting of F242C, F242A, F242R, F242N, F242D, F242Q, F242E, F242G, F242H, F242I, F242L, F242K, F242M, F242P, F242S, F242T, F242W, F242V or F242Y; preferably F242C, F242A, F242R, F242N, F242D, F242Q, F242E, F242G, F242I, F242K, F242M, F242P, F242S, F242T, F242W, F242V, or F242Y; more preferably F242C or F242V; most preferably F242C; and/or position 273, the substitution being selected from the group consisting of A273S, A273C, A273R, A273N, A273D, A273Q, A273E, A273G, A273H, A273I, A273L, A273K, A273M, A273F, A273P, A273T, A273W, A273V or A273Y; preferably A273S; and/or position 300, the substitution being selected from the group consisting of Q300Y, Q300C, Q300A, Q300R, Q300N, Q300D, Q300E, Q300G, Q300H, Q300I, Q300L, Q300K, Q300M, Q300F, Q300P, Q300S, Q300T, Q300W or Q300V; preferably Q300Y, Q300C, Q300A, Q300R, Q300N, Q300D, Q300G, Q300H, Q300I, Q300L, Q300K, Q300M, Q300F, Q300P, Q300S, Q300T, Q300W, or Q300V; more preferably Q300Y, Q300S, Q300C or Q300F; most preferably Q300Y; and/or position 309, the substitution being selected from the group consisting of M309I, M309C, M309A, M309R, M309N, M309D, M309Q, M309E, M309G, M309H, M309L, M309K, M309F, M309P, M309S, M309T, M309W, M309V or M309Y; preferably M309I, M309V, M309L, M309P; more preferably M309I or M309P; most preferably M309I; and/or position 313, the substitution being selected from the group consisting of P313S, P313C, P313A, P313R, P313N, P313D, P313Q, P313E, P313G, P313H, P313I, P313L, P313K, P313M, P313F, P313T, P313W, P313V or P313Y; preferably P313S, P313V and P313G; more preferably P313S; and/or position 331, the substitution being selected from the group consisting of Q331G, Q331C, Q331A, Q331R, Q33 IN, Q331D, Q331E, Q331H, Q331I, Q331L, Q331K, Q331M, Q331F, Q331P, Q331S, Q331T, Q331W, Q331V or Q331Y; preferably Q331G; and/or position 342, the substitution being selected from the group consisting of A342S, A342C, A342R, A342N, A342D, A342Q, A342E, A342G, A342H, A342I, A342L, A342K, A342M, A342F, A342P, A342T, A342W, A342V or A342Y; preferably A342S, A342T, A342Y; more preferably A342S; and/or position 343, the substitution being selected from the group consisting of K343R, K343C, K343A, K343N, K343D, K343Q, K343E, K343G, K343H, K343I, K343L, K343M, K343F, K343P, K343S, K343T, K343W, K343V or K343Y; preferably K343R, K343N, K343Q; more preferably K343R or K343N; most preferably K343R.

Clause 88: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions, wherein the amino acid sequence comprises a substitution in at least one further, at least two further, at least three further, at least four further, at least five further, at least six further, at least seven further, at least eight further, at least nine further, at least ten further, at least eleven further, at least twelve further, at least thirteen further, at least fourteen further, at least fifteen further, or at least sixteen further substitution positions selected from the group consisting of: position 18, the substitution being P18S, P18T or P18Y; more preferably P18S; and/or position 47, the substitution being selected from the group consisting of A47S, A47Q or A47R; preferably A47S; and/or position 140, the substitution being W140H; and/or position 147, the substitution being T147R; and/or position 163, the substitution being I163V; and/or position 187, the substitution being selected from the group consisting of V187C, V187T, or V187W; preferably V187C; and/or position 190, the substitution being G190N; and/or position 198, the substitution being L198I; and/or position 241, the substitution being M241V or M241C; preferably M241V; and/or position 242, the substitution being F242C or F242V; preferably F242C; and/or position 273, the substitution being A273S; and/or position 300, the substitution being selected from the group consisting of Q300Y, Q300S, Q300C or Q300F; preferably Q300Y; and/or position 309, the substitution being M309I; and/or position 313, the substitution being selected from the group consisting of P313S, P313V and P313G; preferably P313S; and/or position 331, the substitution being Q331G; and/or position 342, the substitution being A342S; and/or position 343, the substitution being K343R or K343N; preferably K343R.

Clause 89: The glucose isomerase of any of the preceding clauses, wherein the amino acid sequence comprises a substitution in at least one, at least two, at least three, at least four, at least five, at least six, at least seven, at least eight, at least nine, at least ten, at least eleven, at least twelve, at least thirteen, at least fourteen, at least fifteen, at least or sixteen, at least seventeen amino acid positions.

Clause 90: The glucose isomerase of any of the preceding clauses, wherein the amino acid sequence comprises a substitution in one to seventeen, two to seventeen, three to seventeen, four to seventeen, five to seventeen, six to seventeen, seven to seventeen, eight to seventeen, nine to seventeen, ten to seventeen, eleven to seventeen, twelve to seventeen, thirteen to seven-teen, fourteen to seventeen, fifteen to seventeen, sixteen to seventeen, or seventeen amino acid positions.

Clause 91: The glucose isomerase of any of the preceding clauses, wherein the amino acid sequence comprises a substitution in one amino acid position, in two amino acid positions, in three amino acid positions, in four amino acid positions, in five amino acid positions, in six amino acid positions, in seven amino acid positions, in eight amino acid positions, in nine amino acid positions, in ten amino acid positions, in eleven amino acid positions, in twelve amino acid positions, in thirteen amino acid positions, in fourteen amino acid positions, in fifteen amino acid positions, or in sixteen, or in seventeen amino acid positions.

Clause 92: The glucose isomerase of any of the preceding clauses, wherein the amino acid sequence of the glucose isomerase comprises or consists of any one of amino acid sequences with at least 67%, or at least 68%, or at least 70%, or at least 72%, or at least 74%, or at least 76%, or at least 78%, or at least 80%, or at least 82%, or at least 84%, or at least 86%, or at least 88%, or at least 90%, or at least 92%, or at least 94%, or at least 96%, or at least 97%, or at least 98%, or at least 99%, or at least 99.9%, or at least 100% identity to SEQ ID NO: 1.

Clause 93: The glucose isomerase of any of the preceding clauses, wherein the amino acid sequence of the glucose isomerase comprises or consists of any one of amino acid sequences with at least 67%, or at least 68%, or at least 70%, or at least 72%, or at least 74%, or at least 76%, or at least 78%, or at least 80%, or at least 82%, or at least 84%, or at least 86%, or at least 88%, or at least 90%, or at least 92%, or at least 94%, or at least 96%, or at least 98%, or at least 100% identity to SEQ ID NO: 2.

Clause 94: The glucose isomerase of any of the preceding clauses, wherein the amino acid sequence of the glucose isomerase comprises or consists of any one of amino acid sequences with at least 67%, or at least 68%, or at least 70%, or at least 72%, or at least 74%, or at least 76%, or at least 78%, or at least 80%, or at least 82%, or at least 84%, or at least 86%, or at least 88%, or at least 90%, or at least 92%, or at least 94%, or at least 96%, or at least 98%, or at least 100% identity to SEQ ID NO: 7.

Clause 95: The glucose isomerase of any of the preceding clauses, wherein the amino acid sequence of the glucose isomerase comprises or consists of any one of amino acid sequences with at least 80%, preferably at least 85%, more preferably at least 90%, yet more preferably at least 95%, most preferably at least 99.9%, or utmost preferably 100%, to each one of SEQ ID NO: 2, 3, 4, 5, 6, 7, 8, 9, 10, 11 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, or 73.

Clause 96: The glucose isomerase of any of the preceding clauses, wherein the amino acid sequence of the glucose isomerase comprises or consists of any one of amino acid sequences with at least 80%, preferably at least 85%, more preferably at least 90%, yet more preferably at least 95%, most preferably at least 99.9%, or utmost preferably 100%, to each one of SEQ ID NO: 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, or 73.

Clause 97: The glucose isomerase of any of the preceding clauses, wherein the glucose isomerase is capable of catalyzing conversion of an aldose molecule into a ketose molecule; preferably of glucose into fructose, and/or a ketose molecule into an aldose molecule; preferably of fructose into glucose.

Clause 98: The glucose isomerase of any of the preceding clauses, which exhibits an in-creased enzymatic activity in the absence of Ca²⁺ (A₀) in comparison to SEQ ID NO:1 with respect to the conversion of fructose into glucose or vice versa, preferably with respect to the conversion of fructose into glucose.

Clause 99: The glucose isomerase of any of the preceding clauses, which exhibits an enzymatic activity A₀ of at least 0.1%, preferably of at least 0.2%, more preferably at least 10%, still more preferably at least 30%, yet more preferably at least 50%, even more preferably at least 100%, and most preferably at least 150% higher than the enzymatic activity of SEQ ID NO: 1.

Clause 100: The glucose isomerase of any of the preceding clauses, which exhibits an enzymatic activity A₀ of at least 1.001-fold, preferably of at least 1.002-fold, more preferably at least 1.1-fold, still more preferably at least 1.3-fold, yet more preferably at least 1.5-fold, even more preferably at least 2.0-fold, and most preferably at least 2.5-fold higher than the enzymatic activity of SEQ ID NO: 1 with respect to the conversion of glucose into fructose.

Clause 101: The glucose isomerase of any of the preceding clauses, which exhibits an enzymatic activity A₀ of at least 1.001-fold, preferably of at least 1.002-fold, more preferably at least 1.1-fold, still more preferably at least 1.3-fold, yet more preferably at least 1.5-fold, even more preferably at least 2.0-fold, and most preferably at least 2.5-fold higher than the enzymatic activity of SEQ ID NO: 2 with respect to the conversion of glucose into fructose.

Clause 102: The glucose isomerase of any of the preceding clauses, which exhibits an enzymatic activity A₀ of at least 1.001-fold, preferably of at least 1.002-fold, more preferably at least 1.1-fold, still more preferably at least 1.3-fold, yet more preferably at least 1.5-fold, even more preferably at least 2.0-fold, and most preferably at least 2.5-fold higher than the enzymatic activity of SEQ ID NO: 7 with respect to the conversion of glucose into fructose.

Clause 103: The glucose isomerase of any of the preceding clauses, which exhibits (i) an increased enzymatic activity in the presence of Ca²⁺ (A_{Ca2+}) in comparison to SEQ ID NO:1 with respect to the conversion of fructose into glucose or vice versa, preferably with respect to the conversion of fructose into glucose; and/or (ii) an in-creased residual enzymatic activity in the presence of Ca²⁺ (R A_{Ca2+}) in comparison to SEQ ID NO:1 with respect to the conversion of fructose into glucose or vice versa, preferably with respect to the conversion of fructose into glucose.

Clause 104: The glucose isomerase of any of the preceding clauses, wherein the enzymatic activity A_{Ca2+} is deter-mined at a Ca²⁺ concentration of at least 0.5 mM, preferably at least 60 mM, more preferably at least 30 mM, still more preferably at least 20 mM, most preferably at least 10 mM, and utmost preferably at least 5 mM.

Clause 105: The glucose isomerase of any of the preceding clauses, which exhibits an increased enzymatic activity A_{Ca2+} in comparison to SEQ ID NO: 1 within the range of at 5 mM±0.5 mM, or within the range of 4.0 mM to 5 mM, 3 mM to 5 mM, 2 mM to 5 mM, 1 mM to 5 mM, or 0.5 mM to 5 mM; preferably within the range of 5 mM to 10 mM, 4.0 mM to 10 mM, 3 mM to 10 mM, 2 mM to 10 mM, 1 mM to 10 mM, or 0.5 mM to 10 mM; more preferably within the range of 5 mM to 20 mM, 4.0 mM to 20 mM, 3 mM to 20 mM, 2 mM to 20 mM, 1 mM to 20 mM, or 0.5 mM to 20 mM; even more preferably within the range of 5 mM to 30 mM, 4.0 mM to 30 mM, 3 mM to 30 mM, 2 mM to 30 mM, 1 mM to 30 mM, or 0.5 mM to 30 mM; and most preferably within the range of 5 mM to 60 mM, 4.0 mM to 60 mM, 3 mM to 60 mM, 2 mM to 60 mM, 1 mM to 60 mM, or 0.5 mM to 60 mM.

Clause 106: The glucose isomerase of any of the preceding clauses, which exhibits an enzymatic activity A_{Ca2+} of at least 0.1%; preferably of at least 0.2%, more preferably at least 10%, still more preferably at least 30%, yet more preferably at least 50%, even more preferably at least 100%, most preferably at least 150%, and in particular at least 200% higher than the enzymatic activity of SEQ ID NO: 1.

Clause 107: The glucose isomerase of any of the preceding clauses, which exhibits an enzymatic activity A_{Ca2+} of at least 1.001-fold, preferably of at least 1.002-fold, more preferably at least 1.1-fold, still more preferably at least 1.3-fold, yet more preferably at least 1.5-fold, even more preferably at least 2-fold, most preferably at least 2.5-fold, and in particular at least 3-fold higher than the enzymatic activity of SEQ ID NO: 1.

Clause 108: The glucose isomerase of any of the preceding clauses, which exhibits an enzymatic activity A_{Ca2+} of at least 1.001-fold, preferably of at least 1.002-fold, more preferably at least 1.1-fold, still more preferably at least 1.3-fold, yet more preferably at least 1.5-fold, even more preferably at least 2-fold, most preferably at least 2.5-fold, and in particular at least 3-fold higher than the enzymatic activity of SEQ ID NO: 2.

Clause 109: The glucose isomerase of any of the preceding clauses, which exhibits an enzymatic activity A_{Ca2+} of at least 1.001-fold, preferably of at least 1.002-fold, more preferably at least 1.1-fold, still more preferably at least 1.3-fold, yet more preferably at least 1.5-fold, even more preferably at least 2-fold, most preferably at least 2.5-fold, and in particular at least 3-fold higher than the enzymatic activity of SEQ ID NO: 7.

Clause 110: The glucose isomerase of any of the preceding clauses, which exhibits a residual enzymatic activity R A_{Ca2+} in the presence of Ca²⁺ of at least 4.0%, preferably of at least 10%, more preferably of at least 15%, even more preferably of at least 20%, yet more preferably of at least 25% most preferably of at least 30%, and utmost preferably at least 35% in comparison to the enzymatic activity in the absence of Ca²⁺.

Clause 111: The glucose isomerase of any of the preceding clauses, which exhibits an increased enzymatic activity at neutral and acidic pH (A_{pH}) in comparison to SEQ ID NO:1 with respect to the conversion of fructose into glucose or vice versa, preferably with respect to the conversion of fructose into glucose.

Clause 112: The glucose isomerase of any of the preceding clauses, wherein enzymatic activity A_{pH} is determined at a pH value of 7.0±0.5, or 6.5±0.5, or 5.8±0.5, or within the range from 5.0 to 7.0, 5.3 to 7.0, 5.5 to 7.0, 5.8 to 7.0, 6.0 to 7.0, 6.5 to 7.0; preferably within the range from 5.0 to 7.5, 5.3 to 7.5, 5.5 to 7.5, 5.8 to 7.5, 6.0 to 7.5, 6.5 to 7.5; more preferably within the range from 5.0 to 8.0, 5.3 to 8.0, 5.5 to 8.0, 5.8 to 8.0, 6.0 to 8.0, 6.5 to 8.0.

Clause 113: The glucose isomerase of any of the preceding clauses, which exhibits an increased enzymatic activity A_{pH} at a pH value of 7.0±0.5, or 6.5±0.5, or 5.8±0.5, or at any pH value within the range from 5.0 to 7.0, 5.3 to 7.0, 5.5 to 7.0, 5.8 to 7.0, 6.0 to 7.0, 6.5 to 7.0; preferably within the range from 5.0 to 7.5, 5.3 to 7.5, 5.5 to 7.5, 5.8 to 7.5, 6.0 to 7.5, 6.5 to 7.5; more preferably within the range from 5.0 to 8.0, 5.3 to 8.0, 5.5 to 8.0, 5.8 to 8.0, 6.0 to 8.0, 6.5 to 8.0.

Clause 114: The glucose isomerase of any of the preceding clauses, which exhibits an enzymatic activity A_{pH} of at least 0.1%; preferably of at least 0.2%, more preferably at least 10%, still more preferably at least 30%, yet more preferably at least 50%, even more preferably at least 100%, most preferably at least 150%, and in particular at least 200% higher than the enzymatic activity of SEQ ID NO: 1.

Clause 115: The glucose isomerase of any of the preceding clauses, which exhibits an enzymatic activity A_{pH} of at least 1.001-fold, preferably of at least 1.002-fold, more preferably at least 1.1-fold, still more preferably at least 1.3-fold, yet more preferably at least 1.5-fold, even more preferably at least 2-fold, most preferably at least 2.5-fold, and in particular at least 3-fold higher than the enzymatic activity of SEQ ID NO: 1.

Clause 116: The glucose isomerase of any of the preceding clauses, which exhibits an enzymatic activity A_{pH} of at least 1.001-fold, preferably of at least 1.002-fold, more preferably at least 1.1-fold, still more preferably at least 1.3-fold, yet more preferably at least 1.5-fold, even more preferably at least 2-fold, most preferably at least 2.5-fold, and in particular at least 3-fold higher than the enzymatic activity of SEQ ID NO: 2.

Clause 117: The glucose isomerase of any of the preceding clauses, which exhibits an enzymatic activity A_{pH} of at least 1.001-fold, preferably of at least 1.002-fold, more preferably at least 1.1-fold, still more preferably at least 1.3-fold, yet more preferably at least 1.5-fold, even more preferably at least 2-fold, most preferably at least 2.5-fold, and in particular at least 3-fold higher than the enzymatic activity of SEQ ID NO: 7.

Clause 118: The glucose isomerase of any of the preceding clauses, which exhibits an increased enzymatic activity at low temperatures (A_{Temp}) in comparison to SEQ ID NO: 1 with respect to the conversion of fructose into glucose or vice versa, preferably with respect to the conversion of fructose into glucose.

Clause 119: The glucose isomerase of any of the preceding clauses, wherein the enzymatic activity A_{Temp} is deter-mined at a temperature of 35° C.±1.0° C., or 25° C.±1.0° C., or 20° C.±1.0° C., or 15° C.±1.0° C., or within the range from 15° C. to 25° C., preferably in the range from 15° C. to 35° C., more preferably in the range from 10° C. to 35° C., and most preferably in the range from 5° C. to 35° C.

Clause 120: The glucose isomerase of any of the preceding clauses, which exhibits an increased enzymatic activity A_{Temp} at a temperature of 35° C.±1.0° C., or 25° C.±1.0° C., or 20° C.±1.0° C., or 15° C.±1.0° C., or within the range from 15° C. to 25° C., preferably in the range from 15° C. to 35° C., more preferably in the range from 10° C. to 35° C., and most preferably in the range from 5° C. to 35° C.

Clause 121: The glucose isomerase of any of the preceding clauses, which exhibits an enzymatic activity A_{Temp} of at least 0.1%; preferably of at least 0.2%, more preferably at least 10%, still more preferably at least 300%, yet more preferably at least 50%, even more preferably at least 100%, most preferably at least 150%, and utmost preferably at least 200% higher than the enzymatic activity A_{Temp} of SEQ ID NO: 1.

Clause 122: The glucose isomerase of any of the preceding clauses, which exhibits an enzymatic activity A_{Temp} of at least 1.001-fold, preferably of at least 1.002-fold, more preferably at least 1.1-fold, still more preferably at least 1.3-fold, yet more preferably at least 1.5-fold, even more preferably at least 2.0-fold, most preferably at least 2.5-fold, and utmost preferably at least 3.0-fold higher than the enzymatic activity A_{pH} of SEQ ID NO: 1.

Clause 123: The glucose isomerase of any of the preceding clauses, which exhibits an enzymatic activity A_{Temp} of at least 1.001-fold, preferably of at least 1.002-fold, more preferably at least 1.1-fold, still more preferably at least 1.3-fold, yet more preferably at least 1.5-fold, even more preferably at least 2.0-fold, most preferably at least 2.5-fold, and utmost preferably at least 3.0-fold higher than the enzymatic activity A_{pH} of SEQ ID NO: 2.

Clause 124: The glucose isomerase of any of the preceding clauses, which exhibits an enzymatic activity A_{Temp} of at least 1.001-fold, preferably of at least 1.002-fold, more preferably at least 1.1-fold, still more preferably at least 1.3-fold, yet more preferably at least 1.5-fold, even more preferably at least 2.0-fold, most preferably at least 2.5-fold, and utmost preferably at least 3.0-fold higher than the enzymatic activity A_{pH} of SEQ ID NO: 7.

Clause 125: The glucose isomerase of any of the preceding clauses, wherein the enzymatic activity is measured by (i) incubating an aliquot of the glucose isomerase in 50 mM MES buffer ((2-N-morpholino) ethane sulfonic acid) containing 50 mM fructose and allowing the glucose isomerase to enzymatically convert fructose into glucose; (ii) collecting 20 μl samples at different points in time during conversion and stopping the reaction within each sample by adding 20 μL HCl (0.25 M) followed by 60 μL potassium phosphate buffer (50 mM, pH 7.0); (iii) quantifying the glucose in each sample; preferably by using a photometric assay; and (iv) determining enzymatic activity on the basis of the quantified glucose in the samples as collected at different points in time during conversion; preferably in accordance with the Glucose Isomerase Activity Assay as defined herein and the Glucose Detection Assay as defined herein.

Clause 126: The glucose isomerase of any of the preceding clauses, wherein the enzymatic activity is measured in accordance with the Glucose Isomerase Activity Assay as de-fined herein and the Glucose Detection Assay as defined herein.

Clause 127: The glucose isomerase of any of the preceding clauses, wherein for measuring enzymatic activity in the absence of Ca²⁺ (A₀), in step (i) the pH value is pH 5.8 or pH 7.0, preferably pH 5.8; the Ca²⁺ concentration is 0 mM; and the temperature is 25° C. or 35° C., preferably 35° C.

Clause 128: The glucose isomerase of any of the preceding clauses, wherein for measuring enzymatic activity in the presence Ca²⁺ (A_{Ca2+}), in step (i) the pH value is pH 5.8 or pH 7, preferably pH 5.8; the Ca²⁺ concentration is 5 mM or 10 mM, preferably 10 mM; and the temperature is 25° C. or 35° C., preferably 35° C.

Clause 129: The glucose isomerase of any of the preceding clauses, wherein for measuring enzymatic activity at acidic pH (A_{pH}), in step (i) the pH value is pH 5.8 or pH 7, preferably pH 5.8; the Ca²⁺ concentration is 0 mM, 5 mM, 10 mM, 20 mM, 30 mM, or 60 mM, preferably 0 mM, 5 mM, or 10 mM, more preferably 0 mM, or 5 mM; and the temperature is 15° C., 20° C., 25° C., or 35° C., preferably 25° C., or 35° C., and most preferably 35° C.

Clause 130: The glucose isomerase of any of the preceding clauses, wherein the enzymatic activity is measured according to the Specific Assay Conditions (i.e. any one of Conditions 1 to 15 as defined herein).

Clause 131: The glucose isomerase of any of the preceding clauses, wherein in comparison to SEQ ID NO: 1 the glucose isomerase exhibits (i) increased enzymatic activity in the absence of Ca²⁺ (A₀) as well as increased enzymatic activity in the presence Ca²⁺ (A_{Ca2+}); or (ii) increased enzymatic activity in the absence of Ca²⁺ (A₀) as well as increased enzymatic activity at acidic pH (A_{pH}); or (iii) increased enzymatic activity in the presence Ca²⁺ (A_{Ca2+}) as well as increased enzymatic activity at acidic pH (A_{pH}); or (iv) increased enzymatic activity in the absence of Ca²⁺ (A₀) as well as increased enzymatic activity in the presence Ca²⁺ (A_{Ca2+}) as well as increased enzymatic activity at acidic pH (A_{pH}); or (v) increased enzymatic activity in the presence Ca²⁺ (A_{Ca2+}) as well as increased enzymatic activity at acidic pH (A_{pH}) as well as an increased enzymatic activity at low temperature (A_{Temp}).

Clause 132: The glucose isomerase of any of the preceding clauses, which exhibits an improved enzymatic activity in comparison to SEQ ID NO: 1 under one or more Specific Assay Conditions (i.e. any one of Conditions 1 to 15 as defined herein).

Clause 133: The glucose isomerase of any of the preceding clauses, wherein the amino acid sequence comprises at least one, at least two, at least three, at least four, at least five, at least six, at least seven, at least eight, at least nine, at least ten, or at least eleven substitutions at substitution positions selected from the group consisting of V187, Q300, P313, F242, W140, G190, A342, A47, A273, K343, M309, and exhibits an increased enzymatic activity in the absence of Ca²⁺ (A₀) in comparison to SEQ ID NO: 1 with respect to the conversion of fructose into glucose or vice versa, preferably with respect to the conversion of fructose into glucose.

Clause 134: The glucose isomerase of any of the preceding clauses, wherein the amino acid sequence comprises at least one, at least two, at least three, at least four, at least five, at least six, at least seven, at least eight, at least nine, at least ten, or at least eleven substitutions at substitution positions selected from the group consisting of V187, Q300, P313, F242, W140, G190, A342, A47, A273, K343, M309, and exhibits an increased enzymatic activity in the absence of Ca²⁺ (A₀) of at least 1.001-fold, preferably of at least 1.002-fold, more preferably at least 1.1-fold, still more preferably at least 1.3-fold, yet more preferably at least 1.5-fold, even more preferably at least 2.0-fold, and most preferably at least 2.5-fold higher than the enzymatic activity of SEQ ID NO: 1 with respect to the conversion of glucose into fructose.

Clause 135: The glucose isomerase of any of the preceding clauses, wherein the amino acid sequence comprises at least one, at least two, at least three, at least four, at least five, at least six, at least seven, at least eight, at least nine, at least ten, or at least eleven substitution at substitution positions selected from the group consisting of V187, Q300, P313, F242, W140, G190, A342, A47, A273, K343, M309, and exhibits an increased enzymatic activity in the absence of Ca²⁺ (A₀) in comparison to SEQ ID NO: 1 with respect to the conversion of fructose into glucose or vice versa, preferably with respect to the conversion of fructose into glucose, wherein, (i) when the substitution comprises a substitution at position V187, then the substitution at position V187 is selected from the group consisting of the substitutions V187C, V187A, V187R, V187N, V187D, V187Q, V187E, V187G, V187H, V187I, V187L, V187K, V187M, V187F, V187P, V187S, V187T, V187W, or V187Y; preferably V187C or V187T, or V187W; and more preferably V187C; (ii) when the substitution comprises a substitution at position Q300, then the substitution at position Q300 is selected from the group consisting of the substitutions Q300Y, Q300C, Q300A, Q300R, Q300N, Q300D, Q300E, Q300G, Q300H, Q300I, Q300L, Q300K, Q300M, Q300F, Q300P, Q300S, Q300T, Q300W or Q300V; preferably Q300Y, Q300C, Q300A, Q300R, Q300N, Q300D, Q300G, Q300H, Q300I, Q300L, Q300K, Q300M, Q300F, Q300P, Q300S, Q300T, Q300W, or Q300V; more preferably Q300Y, Q300S, Q300C or Q300F; most preferably Q300Y; (iii) when the substitution comprises a substitution at position P313, then the substitution at position P313 is selected from the group consisting of the substitutions P313S, P313C, P313A, P313R, P313N, P313D, P313Q, P313E, P313G, P313H, P313I, P313L, P313K, P313M, P313F, P313T, P313W, P313V or P313Y; preferably P313V, P313S and P313L; more preferably P313V; (iv) when the substitution comprises a substitution at position F242, then the substitution at position F242 is selected from the group consisting of the substitutions F242C, F242A, F242R, F242N, F242D, F242Q, F242E, F242G, F242H, F242I, F242L, F242K, F242M, F242P, F242S, F242T, F242W, F242V or F242Y; preferably F242C, F242A, F242R, F242N, F242D, F242Q, F242E, F242G, F242I, F242K, F242M, F242P, F242S, F242T, F242W, F242V, or F242Y; more preferably F242C or F242V; most preferably F242C; (v) when the substitution comprises a substitution at position W140, then the substitution at position W140 is selected from the group consisting of the substitutions W140H, W140C, W140A, W140R, W140N, W140D, W140Q, W140E, W140G, W140I, W140L, W140K, W140M, W140F, W140P, W140S, W140T, W140V or W140Y; preferably W140H; (vi) when the substitution comprises a substitution at position G190, then the substitution at position G190 is selected from the group consisting of the substitutions G190N, G190C, G190A, G190R, G190D, G190Q, G190E, G190H, G190I, G190L, G190K, G190M, G190F, G190P, G190S, G190T, G190W, or G190Y; preferably G109N; (vii) when the substitution comprises a substitution at position A342, then the substitution at position A342 is selected from the group consisting of the substitutions A342S, A342C, A342R, A342N, A342D, A342Q, A342E, A342G, A342H, A342I, A342L, A342K, A342M, A342F, A342P, A342T, A342W, A342V or A342Y; preferably A342S, A342T, A342Y; more preferably A342S; (viii) when the substitution comprises a substitution at position A47, then the substitution at position A47 is selected from the group consisting of the substitutions A47S, A47C, A47R, A47N, A47D, A47Q, A47E, A47G, A47H, A47I, A47L, A47K, A47M, A47F, A47P, A47T, A47W, A47V, or A47Y; preferably A47S, A47Q or A47R; and more preferably A47S; (ix) when the substitution comprises a substitution at position A273, then the substitution at position A273 is selected from the group consisting of the substitutions A273S, A273C, A273R, A273N, A273D, A273Q, A273E, A273G, A273H, A273I, A273L, A273K, A273M, A273F, A273P, A273T, A273W, A273V or A273Y; preferably A273S, A273T or A273Y, and more preferably A273S; (x) when the substitution comprises a substitution at position K343, then the substitution at position K343 is selected from the group consisting of the substitutions K343R, K343C, K343A, K343N, K343D, K343Q, K343E, K343G, K343H, K343I, K343L, K343M, K343F, K343P, K343S, K343T, K343W, K343V or K343Y; preferably K343R, K343N, K343Q; more preferably K343R or K343N; most preferably K343R; or (xi) when the substitution comprises a substitution at position M309, then the substitution at position M309 is selected from the group consisting of the substitutions M309I, M309C, M309A, M309R, M309N, M309D, M309Q, M309E, M309G, M309H, M309L, M309K, M309F, M309P, M309S, M309T, M309W, M309V or M309Y; preferably M309I, M309V, M309L, M309P; more preferably M309I or M309P; most preferably M309I.

Clause 136: The glucose isomerase of any of the preceding clauses, wherein the amino acid sequence comprises at least one, at least two, at least three, at least four, at least five, at least six, at least seven, at least eight, at least nine, at least ten, or at least eleven positions such that it comprises at least one, at least two, at least three, at least four, at least five, at least six, at least seven, at least eight, at least nine, at least ten, at least eleven, at least twelve, at least thirteen, at least fourteen, at least fifteen, at least sixteen or at least seventeen substitution selected from the group consisting of the substitutions V187C, Q300Y, P313V, Q300S, F242C, P313S, W140H, Q300F, P313L, Q300C, G190N, F242V, A342S, A47S, A273S, K343R, and M309I, and exhibits an increased enzymatic activity in the absence of Ca²⁺ (A₀) in comparison to SEQ ID NO: 1 with respect to the conversion of fructose into glucose or vice versa, preferably with respect to the conversion of fructose into glucose.

Clause 137: The glucose isomerase of any of the preceding clauses, wherein the amino acid sequence comprises at least one, at least two, at least three, at least four, at least five, at least six, at least seven, at least eight, at least nine, at least ten, or at least eleven positions selected from the group consisting of the positions Q300, V187, P313, G190, F242, K343, M309, A342, A47, W140, A273, and exhibits (i) an increased enzymatic activity in the presence of Ca²⁺ (A_{Ca2+}) in comparison to SEQ ID NO: 1 with respect to the conversion of fructose into glucose or vice versa, preferably with respect to the conversion of fructose into glucose; and/or (ii) an increased residual enzymatic activity in the presence of Ca²⁺ (R A_{Ca2+}) in comparison to SEQ ID NO: 1 with respect to the conversion of fructose into glucose or vice versa, preferably with respect to the conversion of fructose into glucose.

Clause 138: The glucose isomerase of any of the preceding clauses, wherein the amino acid sequence is substituted compared to SEQ ID NO: 1 at least one, at least two, at least three, at least four, at least five, at least six, at least seven, at least eight, at least nine, at least ten, or at least eleven positions selected from the group consisting of the positions Q300, V187, P313, G190, F242, K343, M309, A342, A47, W140, A273, and exhibits (i) an increased enzymatic activity in the presence of Ca²⁺ (A_{Ca2+}) and/or (ii) an increased residual enzymatic activity in the presence of Ca²⁺ (R A_{Ca2+}) of at least of at least 1.001-fold, preferably of at least 1.002-fold, more preferably at least 1.1-fold, still more preferably at least 1.3-fold, yet more preferably at least 1.5-fold, even more preferably at least 2-fold, most preferably at least 2.5-fold, and in particular at least 3-fold higher than the enzymatic activity of SEQ ID NO: 1 with respect to the conversion of glucose into fructose.

Clause 139: The glucose isomerase of any of the preceding clauses, wherein the amino acid sequence comprises at least one, at least two, at least three, at least four, at least five, at least six, at least seven, at least eight, at least nine, at least ten, or at least eleven positions selected from the group consisting of the positions Q300, V187, P313, G190, F242, K343, M309, A342, A47, W140, A273, and exhibits (i) an increased enzymatic activity in the presence of Ca²⁺ (A_{Ca2+}) in comparison to SEQ ID NO: 1 with respect to the conversion of fructose into glucose or vice versa, preferably with respect to the conversion of fructose into glucose; and/or (ii) an increased residual enzymatic activity in the presence of Ca²⁺ (R A_{Ca2+}) in comparison to SEQ ID NO: 1 with respect to the conversion of fructose into glucose or vice versa, preferably with respect to the conversion of fructose into glucose, wherein, (i) when the substitution comprises a substitution at position Q300, then the substitution at position Q300 is selected from the group consisting of the substitutions Q300Y, Q300C, Q300A, Q300R, Q300N, Q300D, Q300E, Q300G, Q300H, Q300I, Q300L, Q300K, Q300M, Q300F, Q300P, Q300S, Q300T, Q300W or Q300V; preferably Q300Y, Q300C, Q300A, Q300R, Q300N, Q300D, Q300G, Q300H, Q300I, Q300L, Q300K, Q300M, Q300F, Q300P, Q300S, Q300T, Q300W, or Q300V; more preferably Q300Y, Q300S, Q300C or Q300F; most preferably Q300Y; (ii) when the substitution comprises a substitution at position V187, then the substitution at position V187 is selected from the group consisting of the substitutions V187C, V187A, V187R, V187N, V187D, V187Q, V187E, V187G, V187H, V187I, V187L, V187K, V187M, V187F, V187P, V187S, V187T, V187W, or V187Y; preferably V187C or V187T, or V187W; and more preferably V187C; (iii) when the substitution comprises a substitution at position P313, then the substitution at position P313 is selected from the group consisting of the substitutions P313S, P313C, P313A, P313R, P313N, P313D, P313Q, P313E, P313G, P313H, P313I, P313L, P313K, P313M, P313F, P313T, P313W, P313V or P313Y; preferably P313V, P313S and P313L; more preferably P313V; (ix) when the substitution comprises a substitution at position G190, then the substitution at position G190 is selected from the group consisting of the substitutions G190N, G190C, G190A, G190R, G190D, G190Q, G190E, G190H, G190I, G190L, G190K, G190M, G190F, G190P, G190S, G190T, G190W, or G190Y; preferably G109N; (v) when the substitution comprises a substitution at position F242, then the substitution at position F242 is selected from the group consisting of the substitutions F242C, F242A, F242R, F242N, F242D, F242Q, F242E, F242G, F242H, F242I, F242L, F242K, F242M, F242P, F242S, F242T, F242W, F242V or F242Y; preferably F242C, F242A, F242R, F242N, F242D, F242Q, F242E, F242G, F242I, F242K, F242M, F242P, F242S, F242T, F242W, F242V, or F242Y; more preferably F242C or F242V; most preferably F242C; (vi) when the substitution comprises a substitution at position K343, then the substitution at position K343 is selected from the group consisting of the substitutions K343R, K343C, K343A, K343N, K343D, K343Q, K343E, K343G, K343H, K343I, K343L, K343M, K343F, K343P, K343S, K343T, K343W, K343V or K343Y; preferably K343R, K343N, K343Q; more preferably K343R or K343N; most preferably K343R; (vii) when the substitution comprises a substitution at position M309, then the substitution at position M309 is selected from the group consisting of the substitutions M309I, M309C, M309A, M309R, M309N, M309D, M309Q, M309E, M309G, M309H, M309L, M309K, M309F, M309P, M309S, M309T, M309W, M309V or M309Y; preferably M309I, M309V, M309L, M309P; more preferably M309I or M309P; most preferably M309I; (viii) when the substitution comprises a substitution at position A342, then the substitution at position A342 is selected from the group consisting of the substitutions A342S, A342C, A342R, A342N, A342D, A342Q, A342E, A342G, A342H, A342I, A342L, A342K, A342M, A342F, A342P, A342T, A342W, A342V or A342Y; preferably A342S, A342T, A342Y; more preferably A342S; (ix) when the substitution comprises a substitution at position A47, then the substitution at position A47 is selected from the group consisting of the substitutions A47S, A47C, A47R, A47N, A47D, A47Q, A47E, A47G, A47H, A47I, A47L, A47K, A47M, A47F, A47P, A47T, A47W, A47V, or A47Y; preferably A47S, A47Q or A47R; and more preferably A47S (x) when the substitution comprises a substitution at position W140, then the substitution at position W140 is selected from the group consisting of the substitutions W140H, W140C, W140A, W140R, W140N, W140D, W140Q, W140E, W140G, W140I, W140L, W140K, W140M, W140F, W140P, W140S, W140T, W140V or W140Y; preferably W140H; or (xi) when the substitution comprises a substitution at position A273, then the substitution at position A273 is selected from the group consisting of the substitutions A273S, A273C, A273R, A273N, A273D, A273Q, A273E, A273G, A273H, A273I, A273L, A273K, A273M, A273F, A273P, A273T, A273W, A273V or A273Y; preferably A273S, A273T or A273Y, and more preferably A273S.

Clause 140: The glucose isomerase of any of the preceding clauses, wherein the amino acid sequence comprises at least one, at least two, at least three, at least four, at least five, at least six, at least seven, at least eight, at least nine, at least ten, or at least eleven positions such that it comprises at least one, at least two, at least three, at least four, at least five, at least six, at least seven, at least eight, at least nine, at least ten, at least eleven, at least twelve, at least thirteen, at least fourteen, at least fifteen, at least sixteen or at least seventeen substitution selected from the group consisting of the substitutions Q300Y, V187C, P313V, Q300S, G190N, F242C, P313L, Q300F, K343R, M309I, Q300C, A342S, P313S, F242V, A47S, W140H, A273S, and exhibits an (i) an increased enzymatic activity in the presence of Ca²⁺ (A_{Ca2+}) in comparison to SEQ ID NO:1 with respect to the conversion of fructose into glucose or vice versa, preferably with respect to the conversion of fructose into glucose; and/or (ii) an increased residual enzymatic activity in the presence of Ca²⁺ (R A_{Ca2+}) in comparison to SEQ ID NO:1 with respect to the conversion of fructose into glucose or vice versa, preferably with respect to the conversion of fructose into glucose.

Clause 141: The glucose isomerase of any of the preceding clauses, wherein the amino acid sequence comprises at least one, at least two, at least three, at least four, at least five, at least six, at least seven, at least eight positions selected from the group consisting of the positions V187, F242, M309, W140, P313, Q300, K343, and M241; and exhibits an increased enzymatic activity at neutral and acidic pH (A_{pH}) in comparison to SEQ ID NO: 1 with respect to the conversion of fructose into glucose or vice versa, preferably with respect to the conversion of fructose into glucose.

Clause 142: The glucose isomerase of any of the preceding clauses, wherein the amino acid sequence is substituted compared to SEQ ID NO: 1 at least one, at least two, at least three, at least four, at least five, at least six, at least seven, at least eight, or at least nine positions selected from the group consisting of the positions V187, F242, M309, W140, P313, Q300, Q331, K343, and M241, and exhibits an increased enzymatic activity at neutral and acidic pH (A_{pH}) of at least of at least 1.001-fold, preferably of at least 1.002-fold, more preferably at least 1.1-fold, still more preferably at least 1.3-fold, yet more preferably at least 1.5-fold, even more preferably at least 2-fold, most preferably at least 2.5-fold, and in particular at least 3-fold higher than the enzymatic activity of SEQ ID NO: 1 with respect to the conversion of glucose into fructose.

Clause 143: The glucose isomerase of any of the preceding clauses, wherein the amino acid sequence comprises at least one, at least two, at least three, at least four, at least five, at least six, at least seven, at least eight, or at least nine positions selected from the group consisting of the positions V187, F242, M309, W140, P313, Q300, Q331, K343, and M241, and exhibits and exhibits an increased enzymatic activity at neutral and acidic pH (A_{pH}) in comparison to SEQ ID NO: 1 with respect to the conversion of fructose into glucose or vice versa, preferably with respect to the conversion of fructose into glucose, wherein, (i) when the substitution comprises a substitution at position V187, then the substitution at position V187 is selected from the group consisting of the substitutions V187C, V187A, V187R, V187N, V187D, V187Q, V187E, V187G, V187H, V187I, V187L, V187K, V187M, V187F, V187P, V187S, V187T, V187W, or V187Y; preferably V187C or V187T, or V187W; and more preferably V187C; (ii) when the substitution comprises a substitution at position F242, then the substitution at position F242 is selected from the group consisting of the substitutions F242C, F242A, F242R, F242N, F242D, F242Q, F242E, F242G, F242H, F242I, F242L, F242K, F242M, F242P, F242S, F242T, F242W, F242V or F242Y; preferably F242C, F242A, F242R, F242N, F242D, F242Q, F242E, F242G, F242I, F242K, F242M, F242P, F242S, F242T, F242W, F242V, or F242Y; more preferably F242C or F242V; most preferably F242C; (iii) when the substitution comprises a substitution at position M309, then the substitution at position M309 is selected from the group consisting of the substitutions M309I, M309C, M309A, M309R, M309N, M309D, M309Q, M309E, M309G, M309H, M309L, M309K, M309F, M309P, M309S, M309T, M309W, M309V or M309Y; preferably M309I, M309V, M309L, M309P; more preferably M309I or M309P; most preferably M309I; (iv) when the substitution comprises a substitution at position W140, then the substitution at position W140 is selected from the group consisting of the substitutions W140H, W140C, W140A, W140R, W140N, W140D, W140Q, W140E, W140G, W140I, W140L, W140K, W140M, W140F, W140P, W140S, W140T, W140V or W140Y; preferably W140H; (v) when the substitution comprises a substitution at position P313, then the substitution at position P313 is selected from the group consisting of the substitutions P313S, P313C, P313A, P313R, P313N, P313D, P313Q, P313E, P313G, P313H, P313I, P313L, P313K, P313M, P313F, P313T, P313W, P313V or P313Y; preferably P313V, P313S and P313L; (vi) when the substitution comprises a substitution at position Q300, then the substitution at position Q300 is selected from the group consisting of the substitutions Q300Y, Q300C, Q300A, Q300R, Q300N, Q300D, Q300E, Q300G, Q300H, Q300I, Q300L, Q300K, Q300M, Q300F, Q300P, Q300S, Q300T, Q300W or Q300V; preferably Q300Y, Q300C, Q300A, Q300R, Q300N, Q300D, Q300G, Q300H, Q300I, Q300L, Q300K, Q300M, Q300F, Q300P, Q300S, Q300T, Q300W, or Q300V; more preferably Q300Y, Q300S, Q300C or Q300F; most preferably Q300Y; (vii) when the substitution comprises a substitution at position K343, then the substitution at position K343 is selected from the group consisting of the substitutions K343R, K343C, K343A, K343N, K343D, K343Q, K343E, K343G, K343H, K343I, K343L, K343M, K343F, K343P, K343S, K343T, K343W, K343V or K343Y; preferably K343R, K343N, K343Q; more preferably K343R or K343N; most preferably K343R; or (viii) when the substitution comprises a substitution at position M241, then the substitution at position M241 is selected from the group consisting of the substitutions M241V, M241C, M241A, M241R, M241N, M241D, M241Q, M241E, M241G, M241H, M241I, M241L, M241K, M241F, M241P, M241S, M241T, M241W, or M241Y; preferably M241V, M241C, M241R, M241D, M241Q, M241E, M241G, M241H, M241I, M241K, M241F, M241P, M241W, or M241Y; more preferably M241V or M241C; most preferably M241V.

Clause 144: The glucose isomerase of any of the preceding clauses, wherein the amino acid sequence comprises at least one, at least two, at least three, at least four, at least five, at least six, at least seven, at least eight, or at least nine substitutions selected from the group consisting of the substitutions V187C, F242C, M309I, F242V, W140H, P313S, P313V, P313L, Q300Y, Q331G, K343R, M241V, and exhibits an increased enzymatic activity at neutral and acidic pH (A_{pH}) in comparison to SEQ ID NO: 1 with respect to the conversion of fructose into glucose or vice versa, preferably with respect to the conversion of fructose into glucose.

Clause 145: The glucose isomerase of any of the preceding clauses, wherein the amino acid sequence comprises at least two, at least three, at least four, at least five, at least six, at least seven, at least eight, or at least nine positions selected from the group consisting of the positions V187, A47, M241, F242, Q300, M309, P313, A342 and K343, and exhibits an increased enzymatic activity in the absence of Ca²⁺ (A₀) in comparison to SEQ ID NO:7 with respect to the conversion of fructose into glucose or vice versa, preferably with respect to the conversion of fructose into glucose.

Clause 146: The glucose isomerase of any of the preceding clauses, wherein the amino acid sequence comprises at least two, at least three, at least four, at least five, at least six, at least seven, at least eight, or at least nine positions selected from the group consisting of the positions V187, A47, M241, F242, Q300, M309, P313, A342 and K343, and exhibits an increased enzymatic activity in the absence of Ca²⁺ (A₀) of at least 1.001-fold, preferably of at least 1.002-fold, more preferably at least 1.1-fold, still more preferably at least 1.3-fold, yet more preferably at least 1.5-fold, even more preferably at least 2.0-fold, and most preferably at least 2.5-fold higher than the enzymatic activity of SEQ ID NO: 7 with respect to the conversion of glucose into fructose.

Clause 147: The glucose isomerase of any of the preceding clauses, wherein the amino acid sequence comprises at least two, at least three, at least four, at least five, at least six, at least seven, at least eight, or at least nine positions selected from the group consisting of the positions V187, A47, M241, F242, Q300, M309, P313, A342 and K343, and exhibits an increased enzymatic activity in the absence of Ca²⁺ (A₀) in comparison to SEQ ID NO:7 with respect to the conversion of fructose into glucose or vice versa, preferably with respect to the conversion of fructose into glucose, wherein, (i) when the substitution comprises a substitution at position V187, then the substitution at position V187 is selected from the group consisting of the substitutions V187C, V187A, V187R, V187N, V187D, V187Q, V187E, V187G, V187H, V187I, V187L, V187K, V187M, V187F, V187P, V187S, V187T, V187W, or V187Y; preferably V187C or V187T, or V187W; (ii) when the substitution comprises a substitution at position A47, then the substitution at position A47 is selected from the group consisting of the substitutions A47S, A47C, A47R, A47N, A47D, A47Q, A47E, A47G, A47H, A47I, A47L, A47K, A47M, A47F, A47P, A47T, A47W, A47V, or A47Y; preferably A47S, A47Q or A47R; and more preferably A47S; (iii) when the substitution comprises a substitution at position M241, then the substitution at position M241 is selected from the group consisting of the substitutions position 241, the substitution being selected from the group consisting of M241V, M241C, M241A, M241R, M241N, M241D, M241Q, M241E, M241G, M241H, M241I, M241L, M241K, M241F, M241P, M241S, M241T, M241W, or M241Y; preferably M241V, M241C, M241R, M241D, M241Q, M241E, M241G, M241H, M241I, M241K, M241F, M241P, M241W, or M241Y; more preferably M241V or M241C; most preferably M241V; (iv) when the substitution comprises a substitution at position F242, then the substitution at position F242 is selected from the group consisting of the substitutions F242C, F242A, F242R, F242N, F242D, F242Q, F242E, F242G, F242H, F242I, F242L, F242K, F242M, F242P, F242S, F242T, F242W, F242V or F242Y; preferably F242C, F242A, F242R, F242N, F242D, F242Q, F242E, F242G, F242I, F242K, F242M, F242P, F242S, F242T, F242W, F242V, or F242Y; more preferably F242C or F242V; most preferably F242C; (v) when the substitution comprises a substitution at position Q300, then the substitution at position Q300 is selected from the group consisting of the substitutions Q300Y, Q300C, Q300A, Q300R, Q300N, Q300D, Q300E, Q300G, Q300H, Q300I, Q300L, Q300K, Q300M, Q300F, Q300P, Q300S, Q300T, Q300W or Q300V; preferably Q300Y, Q300C, Q300A, Q300R, Q300N, Q300D, Q300G, Q300H, Q300I, Q300L, Q300K, Q300M, Q300F, Q300P, Q300S, Q300T, Q300W, or Q300V; more preferably Q300Y, Q300S, Q300C or Q300F; most preferably Q300Y or Q300S; (vi) when the substitution comprises a substitution at position P313, then the substitution at position P313 is selected from the group consisting of the substitutions P313S, P313C, P313A, P313R, P313N, P313D, P313Q, P313E, P313G, P313H, P313I, P313L, P313K, P313M, P313F, P313T, P313W, P313V or P313Y; preferably P313V, P313S and P313L; more preferably P313V or P313S; (vii) when the substitution comprises a substitution at position A342, then the substitution at position A342 is selected from the group consisting of the substitutions A342S, A342C, A342R, A342N, A342D, A342Q, A342E, A342G, A342H, A342I, A342L, A342K, A342M, A342F, A342P, A342T, A342W, A342V or A342Y; preferably A342S, A342T, A342Y; more preferably A342S; (viii) when the substitution comprises a substitution at position M309, then the substitution at position M309 is selected from the group consisting of the substitutions M309I, M309C, M309A, M309R, M309N, M309D, M309Q, M309E, M309G, M309H, M309L, M309K, M309F, M309P, M309S, M309T, M309W, M309V or M309Y; preferably M309I, M309V, M309L, M309P; more preferably M309I or M309P; most preferably M309I; or (ix) when the substitution comprises a substitution at position K343, then the substitution at position K343 is selected from the group consisting of the substitutions K343R, K343C, K343A, K343N, K343D, K343Q, K343E, K343G, K343H, K343I, K343L, K343M, K343F, K343P, K343S, K343T, K343W, K343V or K343Y; preferably K343R, K343N, K343Q; more preferably K343R or K343N; most preferably K343R.

Clause 148: The glucose isomerase of any of the preceding clauses, wherein the amino acid sequence comprises at least one, at least two, at least three, at least four, at least five, at least six, at least seven, at least eight, or at least nine substitution selected from the group consisting of the substitutions V187C, V187W, V187T, A47S, M241V, F242C, Q300Y, Q300S, M309I, P313V, P313S, A342S, K343R, and K343N, and exhibits an increased enzymatic activity in the absence of Ca²⁺ (A₀) in comparison to SEQ ID NO:7 with respect to the conversion of fructose into glucose or vice versa, preferably with respect to the conversion of fructose into glucose.

Clause 149: The glucose isomerase of any of the preceding clauses, wherein the amino acid sequence comprises at least two, at least three, at least four, at least five, at least six, at least seven, at least eight, or at least nine positions selected from the group consisting of the positions V187, A47, M241, F242, Q300, M309, P313, A342 and K343, and exhibits (i) an increased enzymatic activity in the presence of Ca²⁺ (A_{Ca2+}) in comparison to SEQ ID NO: 7 with respect to the conversion of fructose into glucose or vice versa, preferably with respect to the conversion of fructose into glucose; and/or (ii) an increased residual enzymatic activity in the presence of Ca²⁺ (R A_{Ca2+}) in comparison to SEQ ID NO:7 with respect to the conversion of fructose into glucose or vice versa, preferably with respect to the conversion of fructose into glucose.

Clause 150: The glucose isomerase of any of the preceding clauses, wherein the amino acid sequence comprises at least two, at least three, at least four, at least five, at least six, at least seven, at least eight, or at least nine positions selected from the group consisting of the positions V187, A47, M241, F242, Q300, M309, P313, A342 and K343, and exhibits (i) an increased enzymatic activity in the presence of Ca²⁺ (A_{Ca2+}) and/or (ii) an increased residual enzymatic activity in the presence of Ca²⁺ (R A_{Ca2+}) of at least 1.001-fold, preferably of at least 1.002-fold, more preferably at least 1.1-fold, still more preferably at least 1.3-fold, yet more preferably at least 1.5-fold, even more preferably at least 2.0-fold, and most preferably at least 2.5-fold higher than the enzymatic activity of SEQ ID NO: 7 with respect to the conversion of glucose into fructose or vice versa, preferably with respect to the conversion of fructose into glucose.

Clause 151: The glucose isomerase of any of the preceding clauses, wherein the amino acid sequence comprises at least two, at least three, at least four, at least five, at least six, at least seven, at least eight, or at least nine positions selected from the group consisting of the positions V187, A47, M241, F242, Q300, M309, P313, A342 and K343, and exhibits (i) an increased enzymatic activity in the presence of Ca²⁺ (A_{Ca2+}) and/or (ii) an increased residual enzymatic activity in the presence of Ca²⁺ (R A_{Ca2+}) in comparison to SEQ ID NO:7 with respect to the conversion of fructose into glucose or vice versa, preferably with respect to the conversion of fructose into glucose, wherein, (i) when the substitution comprises a substitution at position V187, then the substitution at position V187 is selected from the group consisting of the substitutions V187C, V187A, V187R, V187N, V187D, V187Q, V187E, V187G, V187H, V187I, V187L, V187K, V187M, V187F, V187P, V187S, V187T, V187W, or V187Y; preferably V187C or V187T, or V187W; (ii) when the substitution comprises a substitution at position A47, then the substitution at position A47 is selected from the group consisting of the substitutions A47S, A47C, A47R, A47N, A47D, A47Q, A47E, A47G, A47H, A47I, A47L, A47K, A47M, A47F, A47P, A47T, A47W, A47V, or A47Y; preferably A47S, A47Q or A47R; and more preferably A47S; (iii) when the substitution comprises a substitution at position M241, then the substitution at position M241 is selected from the group consisting of the substitutions position 241, the substitution being selected from the group consisting of M241V, M241C, M241A, M241R, M241N, M241D, M241Q, M241E, M241G, M241H, M241I, M241L, M241K, M241F, M241P, M241S, M241T, M241W, or M241Y; preferably M241V, M241C, M241R, M241D, M241Q, M241E, M241G, M241H, M241I, M241K, M241F, M241P, M241W, or M241Y; more preferably M241V or M241C; most preferably M241V; (iv) when the substitution comprises a substitution at position F242, then the substitution at position F242 is selected from the group consisting of the substitutions F242C, F242A, F242R, F242N, F242D, F242Q, F242E, F242G, F242H, F242I, F242L, F242K, F242M, F242P, F242S, F242T, F242W, F242V or F242Y; preferably F242C, F242A, F242R, F242N, F242D, F242Q, F242E, F242G, F242I, F242K, F242M, F242P, F242S, F242T, F242W, F242V, or F242Y; more preferably F242C or F242V; most preferably F242C; (v) when the substitution comprises a substitution at position Q300, then the substitution at position Q300 is selected from the group consisting of the substitutions Q300Y, Q300C, Q300A, Q300R, Q300N, Q300D, Q300E, Q300G, Q300H, Q300I, Q300L, Q300K, Q300M, Q300F, Q300P, Q300S, Q300T, Q300W or Q300V; preferably Q300Y, Q300C, Q300A, Q300R, Q300N, Q300D, Q300G, Q300H, Q300I, Q300L, Q300K, Q300M, Q300F, Q300P, Q300S, Q300T, Q300W, or Q300V; more preferably Q300Y, Q300S, Q300C or Q300F; most preferably Q300Y or Q300S; (vi) when the substitution comprises a substitution at position P313, then the substitution at position P313 is selected from the group consisting of the substitutions P313S, P313C, P313A, P313R, P313N, P313D, P313Q, P313E, P313G, P313H, P313I, P313L, P313K, P313M, P313F, P313T, P313W, P313V or P313Y; preferably P313V, P313S and P313L; more preferably P313V or P313S; (vii) when the substitution comprises a substitution at position A342, then the substitution at position A342 is selected from the group consisting of the substitutions A342S, A342C, A342R, A342N, A342D, A342Q, A342E, A342G, A342H, A342I, A342L, A342K, A342M, A342F, A342P, A342T, A342W, A342V or A342Y; preferably A342S, A342T, A342Y; more preferably A342S; (viii) when the substitution comprises a substitution at position M309, then the substitution at position M309 is selected from the group consisting of the substitutions M309I, M309C, M309A, M309R, M309N, M309D, M309Q, M309E, M309G, M309H, M309L, M309K, M309F, M309P, M309S, M309T, M309W, M309V or M309Y; preferably M309I, M309V, M309L, M309P; more preferably M309I or M309P; most preferably M309I; or (ix) when the substitution comprises a substitution at position K343, then the substitution at position K343 is selected from the group consisting of the substitutions K343R, K343C, K343A, K343N, K343D, K343Q, K343E, K343G, K343H, K343I, K343L, K343M, K343F, K343P, K343S, K343T, K343W, K343V or K343Y; preferably K343R, K343N, K343Q; more preferably K343R or K343N; most preferably K343R.

Clause 152: The glucose isomerase of any of the preceding clauses, wherein the amino acid sequence comprises at least one, at least two, at least three, at least four, at least five, at least six, at least seven, at least eight, or at least nine substitution selected from the group consisting of the substitutions V187C, V187W, V187T, A47S, M241V, F242C, Q300Y, Q300S, M309I, P313V, P313S, A342S, K343R, and K343N, and exhibits (i) an increased enzymatic activity in the presence of Ca²⁺ (A_{Ca2+}) and/or (ii) an increased residual enzymatic activity in the presence of Ca²⁺ (R A_{Ca2+}) in comparison to SEQ ID NO:7 with respect to the conversion of fructose into glucose or vice versa, preferably with respect to the conversion of fructose into glucose.

Clause 153: The glucose isomerase of any of the preceding clauses, wherein the amino acid sequence comprises at least two, at least three, at least four, at least five, at least six, at least seven, at least eight, or at least nine positions selected from the group consisting of the positions V187, A47, M241, F242, Q300, M309, P313, A342 and K343, and exhibits an increased enzymatic activity at low temperatures (A_{Temp}) in comparison to SEQ ID NO: 7 with respect to the conversion of fructose into glucose or vice versa, preferably with respect to the conversion of fructose into glucose.

Clause 154: The glucose isomerase of any of the preceding clauses, wherein the amino acid sequence comprises at least two, at least three, at least four, at least five, at least six, at least seven, at least eight, or at least nine positions selected from the group consisting of the positions V187, A47, M241, F242, Q300, M309, P313, A342 and K343, and exhibits an increased enzymatic activity at low temperatures (A_{Temp}) of at least 1.001-fold, preferably of at least 1.002-fold, more preferably at least 1.1-fold, still more preferably at least 1.3-fold, yet more preferably at least 1.5-fold, even more preferably at least 2.0-fold, and most preferably at least 2.5-fold higher than the enzymatic activity of SEQ ID NO: 7 with respect to the conversion of glucose into fructose or vice versa, preferably with respect to the conversion of fructose into glucose.

Clause 155: The glucose isomerase of any of the preceding clauses, wherein the amino acid sequence comprises at least two, at least three, at least four, at least five, at least six, at least seven, at least eight, or at least nine positions selected from the group consisting of the positions V187, A47, M241, F242, Q300, M309, P313, A342 and K343, and exhibits increased enzymatic activity at low temperatures (A_{Temp}) in comparison to SEQ ID NO:7 with respect to the conversion of fructose into glucose or vice versa, preferably with respect to the conversion of fructose into glucose, wherein, (i) when the substitution comprises a substitution at position V187, then the substitution at position V187 is selected from the group consisting of the substitutions V187C, V187A, V187R, V187N, V187D, V187Q, V187E, V187G, V187H, V187I, V187L, V187K, V187M, V187F, V187P, V187S, V187T, V187W, or V187Y; preferably V187C or V187T, or V187W; (ii) when the substitution comprises a substitution at position A47, then the substitution at position A47 is selected from the group consisting of the substitutions A47S, A47C, A47R, A47N, A47D, A47Q, A47E, A47G, A47H, A47I, A47L, A47K, A47M, A47F, A47P, A47T, A47W, A47V, or A47Y; preferably A47S, A47Q or A47R; and more preferably A47S; (iii) when the substitution comprises a substitution at position M241, then the substitution at position M241 is selected from the group consisting of the substitutions position 241, the substitution being selected from the group consisting of M241V, M241C, M241A, M241R, M241N, M241D, M241Q, M241E, M241G, M241H, M241I, M241L, M241K, M241F, M241P, M241S, M241T, M241W, or M241Y; preferably M241V, M241C, M241R, M241D, M241Q, M241E, M241G, M241H, M241I, M241K, M241F, M241P, M241W, or M241Y; more preferably M241V or M241C; most preferably M241V; (iv) when the substitution comprises a substitution at position F242, then the substitution at position F242 is selected from the group consisting of the substitutions F242C, F242A, F242R, F242N, F242D, F242Q, F242E, F242G, F242H, F242I, F242L, F242K, F242M, F242P, F242S, F242T, F242W, F242V or F242Y; preferably F242C, F242A, F242R, F242N, F242D, F242Q, F242E, F242G, F242I, F242K, F242M, F242P, F242S, F242T, F242W, F242V, or F242Y; more preferably F242C or F242V; most preferably F242C; (v) when the substitution comprises a substitution at position Q300, then the substitution at position Q300 is selected from the group consisting of the substitutions Q300Y, Q300C, Q300A, Q300R, Q300N, Q300D, Q300E, Q300G, Q300H, Q300I, Q300L, Q300K, Q300M, Q300F, Q300P, Q300S, Q300T, Q300W or Q300V; preferably Q300Y, Q300C, Q300A, Q300R, Q300N, Q300D, Q300G, Q300H, Q300I, Q300L, Q300K, Q300M, Q300F, Q300P, Q300S, Q300T, Q300W, or Q300V; more preferably Q300Y, Q300S, Q300C or Q300F; most preferably Q300Y or Q300S; (vi) when the substitution comprises a substitution at position P313, then the substitution at position P313 is selected from the group consisting of the substitutions P313S, P313C, P313A, P313R, P313N, P313D, P313Q, P313E, P313G, P313H, P313I, P313L, P313K, P313M, P313F, P313T, P313W, P313V or P313Y; preferably P313V, P313S and P313L; more preferably P313V or P313S; (vii) when the substitution comprises a substitution at position A342, then the substitution at position A342 is selected from the group consisting of the substitutions A342S, A342C, A342R, A342N, A342D, A342Q, A342E, A342G, A342H, A342I, A342L, A342K, A342M, A342F, A342P, A342T, A342W, A342V or A342Y; preferably A342S, A342T, A342Y; more preferably A342S; (viii) when the substitution comprises a substitution at position M309, then the substitution at position M309 is selected from the group consisting of the substitutions M309I, M309C, M309A, M309R, M309N, M309D, M309Q, M309E, M309G, M309H, M309L, M309K, M309F, M309P, M309S, M309T, M309W, M309V or M309Y; preferably M309I, M309V, M309L, M309P; more preferably M309I or M309P; most preferably M309I; or (ix) when the substitution comprises a substitution at position K343, then the substitution at position K343 is selected from the group consisting of the substitutions K343R, K343C, K343A, K343N, K343D, K343Q, K343E, K343G, K343H, K343I, K343L, K343M, K343F, K343P, K343S, K343T, K343W, K343V or K343Y; preferably K343R, K343N, K343Q; more preferably K343R or K343N; most preferably K343R.

Clause 156: The glucose isomerase of any of the preceding clauses, wherein the amino acid sequence comprises at least one, at least two, at least three, at least four, at least five, at least six, at least seven, at least eight, or at least nine substitution selected from the group consisting of the substitutions V187C, V187W, V187T, A47S, M241V, F242C, Q300Y, Q300S, M309I, P313V, P313S, A342S, K343R, and K343N, and exhibits increased enzymatic activity at low temperatures (A_{Temp}) in comparison to SEQ ID NO:7 with respect to the conversion of fructose into glucose or vice versa, preferably with respect to the conversion of fructose into glucose.

Clause 157: A process for the conversion of an aldose molecule into a ketose molecule said method comprising the step of contacting said aldose molecule with a glucose isomerase as defined in any of the preceding clauses.

Clause 158: The process of clause 157, wherein the aldose molecule is glucose.

Clause 159: The process of clause 157 or 158, wherein the process takes place in a liquid reaction medium, said liquid reaction medium being preferably characterized by: a pH of at least 4.5; more preferably of at least 5.8; and/or a pH of at most 8.0; more preferably of at most 7.0; and/or a pH of in the range of 5.0 to 8.0, or 5.3 to 8.0, or 5.5 to 8.0, or 5.8 to 8.0, or 6.0 to 8.0, or 6.4 to 8.0, or 6.6 to 8.0, or 6.9 to 8.0, preferably within the range of 5.0 to 7.5, or 5.3 to 7.5, or 5.5 to 7.5, or 5.8 to 7.5, or 6.0 to 7.5, or 6.4 to 7.5, or 6.6 to 7.5, or 6.9 to 7.5, more preferably in the range of 5.0 to 7.0, or 5.3 to 7.0, or 5.5 to 7.0, or 5.8 to 7.0, or 6.0 to 7.0, or 6.4 to 7.0, or 6.6 to 7.0, or 6.9 to 7.0; and/or a calcium ion concentration of at most 60.0 mM; preferably at most 30.0 mM; preferably at most 20.0 mM; most preferably of at most 10.0 mM; and/or a calcium ion concentration of 0 mM or in the range of 0.5 mM to 60 mM, 1 mM to 60 mM, 2 mM to 60 mM, 3 mM to 60 mM, 4.0 mM to 60 mM, 5 mM to 60 mM, 10 mM to 60 mM, 20 mM to 60 mM, or 30 mM to 60 mM, or 0.5 mM to 30 mM, 1 mM to 30 mM, 2 mM to 30 mM, 3 mM to 30 mM, 4.0 mM to 30 mM, 5 mM to 30 mM, 10 mM to 30 mM, 20 mM to 30 mM, 0.5 mM to 20 mM, 1 mM to 20 mM, 2 mM to 20 mM, 3 mM to 20 mM, 4.0 mM to 20 mM, 5 mM to 20 mM, 10 mM to 20 mM, or 0.5 mM to 10 mM, 1 mM to 10 mM, 2 mM to 10 mM, 3 mM to 10 mM, 4.0 mM to 10 mM, 5 mM to 10 mM; a temperature of at least at least 2.0° C.; preferably at least 4.0° C.; more preferably of at least 10° C.; and/or a temperature of at most at most 100.0° C., or at most 50.0° C.; preferably at most 45.0° C.; and/or a temperature within the range of 2.0° C. to 99.5° C., or 3.0° C. to 95.0° C., or 4.0° C. to 90.0° C., or 5.0° C. to 85.0° C., or 6.0° C. to 80.0° C., or 6.0° C. to 75.0° C., or 8.0° C. to 70.0° C., or 9.0° C. to 65.0° C., or 10.0° C. to 60.0° C., or 10.0° C. to 55.0° C., or 10.0° C. to 50.0° C., or 10.0° C. to 45.0° C., preferably within the range of 2.0° C. to 99.5° C., or 3.0° C. to 95.0° C., or 4.0° C. to 90.0° C., or 5.0° C. to 85.0° C., or 6.0° C. to 80.0° C., or 6.0° C. to 75.0° C., or 8.0° C. to 70.0° C., or 9.0° C. to 65.0° C., or 10.0° C. to 60.0° C., or 10.0° C. to 55.0° C., or 10.0° C. to 50.0° C., or 10.0° C. to 45.0° C.; more preferably within the range of 2.0° C. to 99.5° C., or 3.0° C. to 95.0° C., or 4.0° C. to 90.0° C., or 5.0° C. to 85.0° C., or 6.0° C. to 80.0° C., or 6.0° C. to 75.0° C., or 8.0° C. to 70.0° C., or 9.0° C. to 65.0° C., or 10.0° C. to 60.0° C., or 10.0° C. to 55.0° C., or 10.0° C. to 50.0° C., or 10.0° C. to 45.0° C.; and/or an aldose concentration at least 0.01% (w/w), preferably at least 0.1% (w/w) more preferably of at least 0.2% (w/w); and/or an aldose concentration at most 50% (w/w), preferably at most 10% (w/w) more preferably of at most 5% (w/w); and/or an aldose concentration in the range of 0.6±0.5% (w/w), or 0.8±0.5% (w/w), or 1.0±0.5% (w/w), or 2.0±0.5% (w/w), or 2.5±0.5% (w/w), 3.0±0.5% (w/w), or 3.5±0.5% (w/w), 4.0±0.5% (w/w), or 4.5±0.5% (w/w), or 5.0±0.5% (w/w), or 5.5±0.5% (w/w), or 6.0±0.5% (w/w), or 6.5±0.5% (w/w), 7.0±0.5% (w/w), or 7.5±0.5% (w/w), 8.0±0.5% (w/w), or 8.5±0.5% (w/w), 9.0±0.5% (w/w), or 9.5±0.5% (w/w), or 10.0±0.5% (w/w), 12.0±0.5% (w/w), or 14.0±0.5% (w/w), 16.0±0.5% (w/w), 18.0±0.5% (w/w), or 20.0±0.5% (w/w), or 25.0±0.5% (w/w), or 30.0±0.5% (w/w), or 35.0±0.5% (w/w), or 40.0±0.5% (w/w), or 45.0±0.5% (w/w), or 49.5±0.5 mM; preferably within the range of 0.6±0.2% (w/w), or 0.8±0.2% (w/w), or 1.0±0.2% (w/w), or 2.0±0.2% (w/w), or 2.5±0.2% (w/w), 3.0±0.2% (w/w), or 3.5±0.2% (w/w), 4.0±0.2% (w/w), or 4.5±0.2% (w/w), or 5.0±0.2% (w/w), or 5.5±0.2% (w/w), or 6.0±0.2% (w/w), or 6.5±0.2% (w/w), 7.0±0.2% (w/w), or 7.5±0.2% (w/w), 8.0±0.2% (w/w), or 8.5±0.2% (w/w), 9.0±0.2% (w/w), or 9.5±0.2% (w/w), or 10.0±0.2% (w/w), 12.0±0.2% (w/w), or 14.0±0.2% (w/w), 16.0±0.2% (w/w), 18.0±0.2% (w/w), or 20.0±0.2% (w/w), or 25.0±0.2% (w/w), or 30.0±0.2% (w/w), or 35.0±0.2% (w/w), or 40.0±0.2% (w/w), or 45.0±0.2% (w/w), or 49.5±0.2 mM; more preferably within the range of 0.6±0.1% (w/w), or 0.8±0.1% (w/w), or 1.0±0.1% (w/w), or 2.0±0.1% (w/w), or 2.5±0.1% (w/w), 3.0±0.1% (w/w), or 3.5±0.1% (w/w), 4.0±0.1% (w/w), or 4.5±0.1% (w/w), or 5.0±0.1% (w/w), or 5.5±0.1% (w/w), or 6.0±0.1% (w/w), or 6.5±0.1% (w/w), 7.0±0.1% (w/w), or 7.5±0.1% (w/w), 8.0±0.1% (w/w), or 8.5±0.1% (w/w), 9.0±0.1% (w/w), or 9.5±0.1% (w/w), or 10.0±0.1% (w/w), 12.0±0.1% (w/w), or 14.0±0.1% (w/w), 16.0±0.1% (w/w), 18.0±0.1% (w/w), or 20.0±0.1% (w/w), or 25.0±0.1% (w/w), or 30.0±0.1% (w/w), or 35.0±0.1% (w/w), or 40.0±0.1% (w/w), or 45.0±0.1% (w/w), or 49.5±0.1 mM.

Clause 160: Use of a glucose isomerase to produce in a liquid reaction medium (i) a ketose molecule by conversion of an aldose molecule; and/or (ii) an aldose molecule by conversion of a ketose molecule; wherein the glucose isomerase is a glucose isomerase as defined in any of clauses 1 to 156; and wherein the liquid reaction medium is defined as in clause 159.

Clause 161: The use of clause 160, wherein (i) the ketose molecule fructose is converted to the aldose molecule glucose; and/or (ii) the aldose molecule glucose is converted to the ketose molecule fructose; preferably wherein the aldose molecule glucose is converted to the ketose molecule fructose.

Clause 162: The use of clause 160 or 161, wherein the liquid reaction medium is characterized by: (i) a pH of in the range of 5.0 to 8.0, or 5.3 to 8.0, or 5.5 to 8.0, or 5.8 to 8.0, or 6.0 to 8.0, or 6.4 to 8.0, or 6.6 to 8.0, or 6.9 to 8.0, preferably within the range of 5.0 to 7.5, or 5.3 to 7.5, or 5.5 to 7.5, or 5.8 to 7.5, or 6.0 to 7.5, or 6.4 to 7.5, or 6.6 to 7.5, or 6.9 to 7.5, more preferably in the range of 5.0 to 7.0, or 5.3 to 7.0, or 5.5 to 7.0, or 5.8 to 7.0, or 6.0 to 7.0, or 6.4 to 7.0, or 6.6 to 7.0, or 6.9 to 7.0; and/or (ii) a calcium ion concentration of 0 mM or in the range between 0.5 mM to 60 mM, 1 mM to 60 mM, 2 mM to 60 mM, 3 mM to 60 mM, 4.0 mM to 60 mM, 5 mM to 60 mM, 10 mM to 60 mM, 20 mM to 60 mM, or 30 mM to 60 mM, or 0.5 mM to 30 mM, 1 mM to 30 mM, 2 mM to 30 mM, 3 mM to 30 mM, 4.0 mM to 30 mM, 5 mM to 30 mM, 10 mM to 30 mM, 20 mM to 30 mM, 0.5 mM to 20 mM, 1 mM to 20 mM, 2 mM to 20 mM, 3 mM to 20 mM, 4.0 mM to 20 mM, 5 mM to 20 mM, 10 mM to 20 mM, or 0.5 mM to 10 mM, 1 mM to 10 mM, 2 mM to 10 mM, 3 mM to 10 mM, 4.0 mM to 10 mM, 5 mM to 10 mM.

Further preferred embodiments of the invention are summarized as clauses 1 to 15 hereinafter:

Clause 1: A glucose isomerase comprising an amino acid sequence which has at least 65% identity to SEQ ID NO: 1 and which compared to SEQ ID NO: 1 comprises at least one, two, three, four, five, six, seven, eight, nine, ten, eleven, twelve, thirteen, fourteen, fifteen, sixteen or seventeen substitutions at substitution positions, which are located in-subsequence R3 ranging from position N185 to W199 of SEQ ID NO: 1, and preferably individually and independently selected from the groups consisting of substitution positions V187, G190, and L198; and/or—subsequence R1 ranging from position L266 to R346 of SEQ ID NO:1, and preferably individually and independently selected from the groups consisting of substitution positions P313, Q300, A273, M309, Q331, A342, and K343; and/or—subsequence R2 ranging from position L231 to Q243 of SEQ ID NO: 1, and preferably individually and independently selected from the groups consisting of substitution positions M241 and F242; and/or—subsequence R4 ranging from position L139 to Y164 of SEQ ID NO:1, and preferably individually and independently selected from the groups consisting of substitution positions W140, T147, and I163; and/or—subsequence R5 ranging from position G17 to R67 of SEQ ID NO:1, and preferably individually and independently selected from the groups consisting of substitution positions A47, and P18.

Clause 2: The glucose isomerase according to clause 1, wherein the substitution positions are in any subsequence, and wherein the substitutions are individually and independently selected from the groups consisting of—R1-B, R1-D and R3-A; or —R1-B, R1-D and R5-B; or —R1-B, R1-D, R3-A and R5-B; or —R1-B, R1-D, R3-A, R2 and R5-B.

Clause 3: The glucose isomerase of clause 1 or 2, wherein the substitution positions are individually and independently selected from the group consisting of V187, P313, Q300, P18, A47, W140, T147, I163, G190, L198, M241, F242, A273, M309, Q331, A342, and K343; and preferably selected from the group consisting of V187, P313, Q300, A47, M241, F242, M309, A342, and K343, and more preferably selected from the group consisting of V187, P313, and Q300.

Clause 4: The glucose isomerase of any of the preceding clauses, wherein the amino acid sequence comprises at least one, two, three, four, five, six, seven, eight, nine, ten, eleven, twelve, thirteen, fourteen, fifteen, sixteen or seventeen substitutions at substitution positions individually and independently selected from the group consisting of—position 18, the substitution being selected from the group consisting of P18S, P18A, P18R, P18N, P18D, P18C, P18Q, P18E, P18G, P18H, P18I, P18L, P18K, P18M, P18F, P18T, P18W, P18V or P18Y; more preferably P18S, P18T, P18Y; and most preferably P18S; and/or—position 47, the substitution being selected from the group consisting of A47S, A47C, A47R, A47N, A47D, A47Q, A47E, A47G, A47H, A47I, A47L, A47K, A47M, A47F, A47P, A47T, A47W, A47V, or A47Y; preferably A47S, A47Q or A47R; and more preferably A47S; and/or—position 140, the substitution being selected from the group consisting of W140H, W140C, W140A, W140R, W140N, W140D, W140Q, W140E, W140G, W140I, W140L, W140K, W140M, W140F, W140P, W140S, W140T, W140V or W140Y; preferably W140H, W140R, W140K; more preferably W140H; and/or—position 147, the substitution being selected from the group consisting of T147R, T147C, T147A, T147N, T147D, T147E, T147G, T147H, T147I, T147L, T147K, T147M, T147F, T147P, T147S, T147W, T147V or T147Y; preferably T147R; and/or—position 163, the substitution being selected from the group consisting of I163V, I163C, I163A, I163R, I163N, I163D, I163Q, I163E, I163G, I163H, I163L, I163K, I163M, I163F, I163P, I163S, I163T, I163W, or I163Y; preferably I163V; and/or—position 187, the substitution being selected from the group consisting of V187C, V187A, V187R, V187N, V187D, V187Q, V187E, V187G, V187H, V187I, V187L, V187K, V187M, V187F, V187P, V187S, V187T, V187W, or V187Y; preferably V187C or V187T, or V187W; and more preferably V187C; and/or—position 190, the substitution being selected from the group consisting of G190N, G190C, G190A, G190R, G190D, G190Q, G190E, G190H, G190I, G190L, G190K, G190M, G190F, G190P, G190S, G190T, G190W, or G190Y; preferably G190Q or G109N; and/or—position 198, the substitution being selected from the group consisting of L198I, L198C, L198A, L198R, L198N, L198D, L198Q, L198E, L198G, L198H, L198K, L198M, L198F, L198P, L198S, L198T, L198W, L198V or L198Y; preferably L198I; and/or—position 241, the substitution being selected from the group consisting of M241V, M241C, M241A, M241R, M24 IN, M241D, M241Q, M241E, M241G, M241H, M241I, M241L, M241K, M241F, M241P, M241S, M241T, M241W, or M241Y; preferably M241V or M241C; more preferably M241V; and/or—position 242, the substitution being selected from the group consisting of F242C, F242A, F242R, F242N, F242D, F242Q, F242E, F242G, F242H, F242I, F242L, F242K, F242M, F242P, F242S, F242T, F242W, F242V or F242Y preferably F242C or F242V; more preferably F242C; and/or—position 273, the substitution being selected from the group consisting of A273S, A273C, A273R, A273N, A273D, A273Q, A273E, A273G, A273H, A273I, A273L, A273K, A273M, A273F, A273P, A273T, A273W, A273V or A273Y; preferably A273S, A273T or A273Y; and/or—position 300, the substitution being selected from the group consisting of Q300Y, Q300C, Q300A, Q300R, Q300N, Q300D, Q300E, Q300G, Q300H, Q300I, Q300L, Q300K, Q300M, Q300F, Q300P, Q300S, Q300T, Q300W or Q300V; preferably Q300Y, Q300S, Q300C or Q300F; more preferably Q300Y; and/or—position 309, the substitution being selected from the group consisting of M309I, M309C, M309A, M309R, M309N, M309D, M309Q, M309E, M309G, M309H, M309L, M309K, M309F, M309P, M309S, M309T, M309W, M309V or M309Y; preferably M309I, M309V, M309L, M309P; more preferably M309I; and/or—position 313, the substitution being selected from the group consisting of P313S, P313C, P313A, P313R, P313N, P313D, P313Q, P313E, P313G, P313H, P313I, P313L, P313K, P313M, P313F, P313T, P313W, P313V or P313Y; preferably P313S, P313V and P313G; more preferably P313S; and/or—position 331, the substitution being selected from the group consisting of Q331G, Q331C, Q331A, Q331R, Q33 IN, Q331D, Q331E, Q331H, Q331I, Q331L, Q331K, Q331M, Q331F, Q331P, Q331S, Q331T, Q331W, Q331V or Q331Y; preferably Q331G; and/or—position 342, the substitution being selected from the group consisting of A342S, A342C, A342R, A342N, A342D, A342Q, A342E, A342G, A342H, A342I, A342L, A342K, A342M, A342F, A342P, A342T, A342W, A342V or A342Y; preferably A342S, A342T, A342Y; more preferably A342S; and/or—position 343, the substitution being selected from the group consisting of K343R, K343C, K343A, K343N, K343D, K343Q, K343E, K343G, K343H, K343I, K343L, K343M, K343F, K343P, K343S, K343T, K343W, K343V or K343Y; preferably K343R, K343N, K343Q; more preferably K343R or K343N; most preferably K343R.

Clause 5: The glucose isomerase of any of the preceding clauses, wherein the substitution positions are individually and independently selected from the groups consisting of V187C, V187T, V187W, P313S, P313V, P313G, Q300Y, Q300S, Q300C, Q300F, P18S, A47S, W140H, T147R, I163V, G190N, L198I, M241V, F242C, F242V, A273S, M309I, Q331G, A342S, K343N and K343R; and preferably selected from the group consisting of V187C, V187T, V187W, P313S, P313V, P313G, Q300Y, Q300S, Q300C, Q300F, A47S, M241V, F242C, F242V, M309I, A342S, K343N and K343R; and more preferably selected from the group consisting of V187C, V187T, V187W, P313S, P313V, P313G, Q300Y, Q300S, Q300C, and Q300F.

Clause 6: The glucose isomerase according to any of the preceding clauses, wherein the amino acid sequence has at least 67%, or at least 68%, or at least 70%, or at least 72%, or at least 74%, or at least 76%, or at least 78%, or at least 80%, or at least 82%, or at least 84%, or at least 86%, or at least 88%, or at least 90%, or at least 92%, or at least 94%, or at least 96%, or at least 97%, or at least 98%, or at least 99%, or at least 99.9%, or at least 100% identity to SEQ ID NO: 1.

Clause 7: The glucose isomerase according to any of the preceding clauses, wherein the amino acid sequence has at least 67%, or at least 68%, or at least 70%, or at least 72%, or at least 74%, or at least 76%, or at least 78%, or at least 80%, or at least 82%, or at least 84%, or at least 86%, or at least 88%, or at least 90%, or at least 92%, or at least 94%, or at least 96%, or at least 98%, or at least 100% identity to SEQ ID NO: 2.

Clause 8: The glucose isomerase according to any of the preceding clauses, which is capable of catalyzing conversion of—an aldose molecule into a ketose molecule; preferably of glucose into fructose, and/or—a ketose molecule into an aldose molecule; preferably of fructose into glucose.

Clause 9: The glucose isomerase according to any of the preceding clauses, which exhibits (A) an increased enzymatic activity in the absence of Ca²⁺ (A₀) in comparison to SEQ ID NO:1 with respect to the conversion of fructose into glucose or vice versa, preferably with respect to the conversion of fructose into glucose; and/or (B) an increased enzymatic activity in the presence of Ca²⁺ (A_{Ca2+}) in comparison to SEQ ID NO: 1 with respect to the conversion of fructose into glucose or vice versa, preferably with respect to the conversion of fructose into glucose; and/or an increased residual enzymatic activity in the presence of Ca²⁺ (RA_{Ca2+}) in comparison to SEQ ID NO: 1 with respect to the conversion of fructose into glucose or vice versa, preferably with respect to the conversion of fructose into glucose; and/or (C) an increased enzymatic activity at neutral and acidic pH (A_{pH}) in comparison to SEQ ID NO: 1 with respect to the conversion of fructose into glucose or vice versa, preferably with respect to the conversion of fructose into glucose; and/or (D) an increased enzymatic activity at low temperatures (A_{Temp}) in comparison to SEQ ID NO: 1 with respect to the conversion of fructose into glucose or vice versa, preferably with respect to the conversion of fructose into glucose.

Clause 10: A process for the conversion of an aldose molecule into a ketose molecule said method comprising the step of contacting said aldose molecule with a glucose isomerase according to any of clauses 1 to 9.

Clause 11: The process according to clause 10, wherein the aldose molecule is glucose.

Clause 12: The process according to clause 10 or 11, wherein contacting is performed in a liquid reaction medium that is characterized by (i) a pH of in the range of 5.0 to 8.0, or 5.3 to 8.0, or 5.5 to 8.0, or 5.8 to 8.0, or 6.0 to 8.0, or 6.4 to 8.0, or 6.6 to 8.0, or 6.9 to 8.0; and/or (ii) a calcium ion concentration of 0 mM or in the range between 0.5 mM to 60 mM, 1 mM to 60 mM, 2 mM to 60 mM, 3 mM to 60 mM, 4.0 mM to 60 mM, 5 mM to 60 mM, 10 mM to 60 mM, 20 mM to 60 mM, or 30 mM to 60 mM.

Clause 13: Use of a glucose isomerase to produce in a liquid reaction medium—a ketose molecule by conversion of an aldose molecule; and/or—an aldose molecule by conversion of a ketose molecule; wherein the glucose isomerase is a glucose isomerase according to any of clauses 1 to 9 and wherein the liquid reaction medium is defined as in clause 12.

Clause 14: The use according to clause 13, wherein the aldose molecule is glucose that is converted to the ketose molecule fructose. The use according to clause 13 or 14, wherein the liquid reaction medium is characterized by: —a pH of in the range of 5.0 to 8.0, or 5.3 to 8.0, or 5.5 to 8.0, or 5.8 to 8.0, or 6.0 to 8.0, or 6.4 to 8.0, or 6.6 to 8.0, or 6.9 to 8.0, preferably within the range of 5.0 to 7.5, or 5.3 to 7.5, or 5.5 to 7.5, or 5.8 to 7.5, or 6.0 to 7.5, or 6.4 to 7.5, or 6.6 to 7.5, or 6.9 to 7.5, more preferably in the range of 5.0 to 7.0, or 5.3 to 7.0, or 5.5 to 7.0, or 5.8 to 7.0, or 6.0 to 7.0, or 6.4 to 7.0, or 6.6 to 7.0, or 6.9 to 7.0; and/or—a calcium ion concentration of 0 mM or in the range between 0.5 mM to 60 mM, 1 mM to 60 mM, 2 mM to 60 mM, 3 mM to 60 mM, 4.0 mM to 60 mM, 5 mM to 60 mM, 10 mM to 60 mM, 20 mM to 60 mM, or 30 mM to 60 mM, or 0.5 mM to 30 mM, 1 mM to 30 mM, 2 mM to 30 mM, 3 mM to 30 mM, 4.0 mM to 30 mM, 5 mM to 30 mM, 10 mM to 30 mM, 20 mM to 30 mM, 0.5 mM to 20 mM, 1 mM to 20 mM, 2 mM to 20 mM, 3 mM to 20 mM, 4.0 mM to 20 mM, 5 mM to 20 mM, 10 mM to 20 mM, or 0.5 mM to 10 mM, 1 mM to 10 mM, 2 mM to 10 mM, 3 mM to 10 mM, 4.0 mM to 10 mM, 5 mM to 10 mM.

The following examples further illustrate the invention but are not to be construed as limiting its scope:

### EXAMPLE 1—GENERAL METHODS

### Molecular Biology Methods:

The glucose isomerase wild type was cloned into the expression vector pLE1A17 or pLE1A27 (both derivatives of pRSF-1b, Novagen). The resulting plasmids were used for transformation of E. coli BL21 (DE3) cells.

Mutants of the glucose isomerase SEQ ID NO: 1 were created by standard site directed mutagenesis technologies as known to the skilled person and as described in the state of the art.

Expression of recombinant glucose isomerases: Recombinant glucose isomerases were routinely expressed by inoculating Medium I (4.6 g/L yeast extract, 9.3 g/L peptone, 25 mM Na2HPO4*12H2O, 25 mM KH2PO4, 50 mM NH4Cl2, Na2SO4, 5 g/L glycerol, 0.5 g/L glucose*1H2O, 2 mM MgSO4, 50 μg/mL kanamycin) with a fresh overnight culture. Cultures were grown at 37° C. up to an optical density at 600 nm of 0.6-0.8. Cultures were induced with 0.1 mM IPTG final concentration. Expression was at 30° C. overnight.

Preparation of glucose isomerase preparations: Preparation of cell free extract was done using procedures well known as described elsewhere. Cells were harvested by centrifugation and suspended in a buffer containing 50 mM potassium phosphate buffer pH 7, 2 mM MgCl2, 0.5 mg/mL lysozyme and 20 U/mL nuclease. Cell disruption was achieved by sonication or repeated freeze/thaw cycles. Cell free extract containing soluble enzyme was separated from the debris by centrifugation.

The glucose isomerase manufactured accordingly carries two Mg²⁺ catalytic cations in each of the monomers of the homo-tetrameric enzyme

### Enzyme Activity Measurements—Glucose Isomerase Activity Assay:

The activity of a respective glucose isomerase was measured from crude extracts from lysed expression cells containing the recombinant specific glucose isomerase by using fructose as substrate and measuring the amount of glucose produced under any specific reaction condition (Conditions as outlined below) over a defined time period. Specifically, the biotransformation reaction of a respective glucose isomerase was performed by incubating 20 μL of the respective crude extract with 80 μL of fructose-containing substrate buffer with a certain buffer, pH value and Ca²⁺ concentration, thereby resulting in the final buffer conditions outlined for any of the Specific Assay Conditions outlined below, and incubating the mixtures according to the required temperature for such Specific Assay Condition. 20 μL samples of the biotransformation reaction were taken at different points in time (usually 2, 4, 20 h after reaction start). The reaction was stopped by addition of 20 μL HCl (0.25 M) followed by addition of 60 μL potassium phosphate buffer (50 mM, pH 7). The glucose concentration of the stopped biotransformation reaction and the amount of glucose formed from fructose was then quantified. The turnover is expressed as the ratio of glucose formed per reaction time, whereby for calculation of conversion only such samples may be used for analysis where the conversion of fructose to glucose in that specific samples is within a range of 5-25% conversion, to ensure reliable detection of the turnover.

Enzyme Activity measurements—Specific Assay Conditions:

The Glucose Isomerase Activity Assays are accomplished in accordance with either Specific Assay Conditions:
Example 2a: Condition 1: 50 mM fructose, 50 mM MES buffer ((2-N-morpholino)ethansulfonic acid), pH 7.0) at 35° C.;
Example 2b: Condition 2: 50 mM fructose, 50 mM MES buffer ((2-N-morpholino)ethansulfonic acid), pH 7.0), 5 mM CaCl₂) at 35° C.;
Example 3a: Condition 3: 50 mM fructose, 50 mM MES buffer ((2-N-morpholino)ethansulfonic acid), pH 5.8) at 35° C.;
Example 3b: Condition 4: 50 mM fructose, 50 mM MES buffer ((2-N-morpholino)ethansulfonic acid), pH 5.8), 5 mM CaCl₂ at 35° C.;
Example 4a: Condition 5: 50 mM fructose, 50 mM MES buffer ((2-N-morpholino)ethansulfonic acid), pH 5.8) at 25° C.;
Example 4b: Condition 6: 50 mM fructose, 50 mM MES buffer ((2-N-morpholino)ethansulfonic acid), pH 5.8), 5 mM CaCl₂ at 25° C.;
Example 4c: Condition 7: 50 mM fructose, 50 mM MES buffer ((2-N-morpholino)ethansulfonic acid), pH 5.8), 10 mM CaCl₂) at 25° C.;
Example 5a: Condition 8: 200 mM fructose, 50 mM MES buffer ((2-N-morpholino)ethansulfonic acid), pH 6.5), 0 mM CaCl₂ at 40° C.;
Example 5b: Condition 9: 200 mM fructose, 50 mM MES buffer ((2-N-morpholino)ethansulfonic acid), pH 6.5), 5 mM CaCl₂ at 40° C.;
Example 5c: Condition 10: 200 mM fructose, 50 mM MES buffer ((2-N-morpholino)ethansulfonic acid), pH 6.5), 10 mM CaCl₂ at 40° C.;
Example 5d: Condition 11: 200 mM fructose, 50 mM MES buffer ((2-N-morpholino)ethansulfonic acid), pH 6.5), 20 mM CaCl₂) at 40° C.;
Example 5e: Condition 12: 200 mM fructose, 50 mM MES buffer ((2-N-morpholino)ethansulfonic acid), pH 6.5), 30 mM CaCl₂ at 40° C.;
Example 5f: Condition 13: 200 mM fructose, 50 mM MES buffer ((2-N-morpholino)ethansulfonic acid), pH 6.5), 60 mM CaCl₂ at 40° C.;
Example 6a: Condition 5: 50 mM fructose, 50 mM MES buffer ((2-N-morpholino)ethansulfonic acid), pH 5.8), 5 mM CaCl₂ at 25° C.;
Example 6b: Condition 14: 50 mM fructose, 50 mM MES buffer ((2-N-morpholino)ethansulfonic acid), pH 5.8), 5 mM CaCl₂) at 20° C.;
Example 6c: Condition 15: 50 mM fructose, 50 mM MES buffer ((2-N-morpholino)ethansulfonic acid), pH 5.8), 5 mM CaCl₂) at 15° C.;

### Glucose Detection Assays:

For quantification of glucose formation, a commercial assay kit (K-GLUHK-220A) from Megazymes has been used, which measures the glucose concentration photometrically at 340 nm. The assay reagent is prepared according to the assay kit manual. In a transparent flat bottom microtiter plate, 100 μL of the assay reagent is mixed with 15 μL of an appropriately diluted sample (with a glucose concentration of less than 5 mM) from a stopped biotransformation reaction. The mixture is incubated at 30° C. for 10 min and the absorption is measured at 340 nm using a microtiter plate photometer. The glucose concentration in [mM] is calculated according to the assay kit manual by comparison to a calibration standard.

### EXAMPLE 2—SINGLE VARIANTS WITH INCREASED RELATIVE ACTIVITY IN PRESENCE OF CALCIUM (A_{Ca2+}) AT NEUTRAL PH

SEQ ID NO: 1 and single variants thereof have been measured according to the Glucose Isomerase Standard Assay at 50 mM fructose, at neutral pH (pH 7) and at 35° C. either
Example 2a: at 0 mM Ca²⁺ and neutral pH (Specific Assay Condition 1); or
Example 2b: at 5 mM Ca²⁺ and neutral pH (Specific Assay Condition 2).

Samples were collected after 3.5 h (Specific Assay Condition 3) or 19.5 h (Specific Assay Condition 4), stopped, and the glucose formed has been quantified by the Glucose Detection Assay, and the enzymatic activity has been calculated from the glucose formed and the reaction time. The relative enzymatic activity of each variant has been calculated by dividing the enzyme activity of each variant by the enzyme activity of SEQ ID NO:1

TABLE 3

| | Relative activity | Relative activity |
|---|---|---|
| SEQ | Ca²⁺_{(A0)} | Ca²⁺_{(A5mM)} |
| ID NO: | pH 7.0 | pH 7.0 |
| 1 | 1.0 | 1.0 |
| 2 | 1.1 | 1.1 |
| 3 | 1.4 | 1.3 |
| 4 | 1.9 | 1.2 |
| 5 | — | 1.1 |
| 6 | 1.1 | 1.1 |
| 7 | 2.3 | 2.1 |
| 8 | 1.6 | 1.9 |
| 9 | 1.1 | 1.1 |
| 11 | 2.0 | 1.7 |
| 12 | 1.5 | 1.3 |
| 13 | 1.4 | 1.1 |
| 14 | 2.3 | 2.2 |
| 15 | 2.1 | 2.0 |
| 16 | 1.8 | 1.5 |
| 17 | 1.9 | 1.6 |
| 18 | 1.1 | 1.6 |
| 19 | 2.0 | 1.4 |
| 20 | 2.2 | 2.0 |
| 21 | 1.9 | 1.7 |
| 22 | 1.1 | 1.2 |
| 23 | 1.5 | 1.5 |
| 24 | 1.4 | 1.6 |

### EXAMPLE 3—SINGLE VARIANTS WITH INCREASED RELATIVE ACTIVITY IN PRESENCE OF CALCIUM (A_{Ca2+}) AT ACIDIC PH

SEQ ID NO: 1 and single variants thereof have been measured according to the Glucose Isomerase Standard Assay at 50 mM fructose, acidic pH (pH 5.8) and at 35° C. either
Example 3a: at 0 mM Ca²⁺ (Specific Assay Conditions 3); or
Example 3b: at 5 mM Ca²⁺ (Specific Assay Conditions 4).

Samples were collected after 3.5 h, stopped, and the glucose formed has been quantified by the Glucose Detection Assay, and the enzymatic activity has been calculated from the glucose formed and the reaction time. The relative enzymatic activity of each variant has been calculated by dividing the enzyme activity of each variant by the enzyme activity of SEQ ID NO:1.

TABLE 4

| | Relative activity | Relative activity |
|---|---|---|
| SEQ | Ca²⁺_{(0mM)} | Ca²⁺_{(5mM)} |
| ID NO: | pH 5.8.0 | pH 5.8 |
| 1 | 1.0 | 1.0 |
| 2 | 1.1 | — |
| 3 | 1.1 | 1.2 |
| 4 | 1.8 | 1.1 |
| 5 | — | 1.1 |
| 6 | 1.1 | 1.1 |
| 7 | 2.8 | 2.1 |
| 8 | — | 1.1 |
| 9 | 1.1 | — |
| 10 | 1.2 | 1.1 |
| 11 | 1.7 | 1.5 |
| 12 | 1.3 | 1.2 |
| 14 | 1.2 | 1.4 |
| 15 | — | 1.3 |
| 18 | 1.4 | 1.9 |
| 19 | 1.3 | 1.4 |
| 20 | 1.3 | 1.3 |
| 21 | 1.3 | 1.2 |
| 22 | 1.2 | 1.1 |
| 23 | — | 1.3 |
| 24 | 1.1 | 1.3 |

### EXAMPLE 4—MULTIPLE VARIANTS WITH INCREASED RELATIVE ACTIVITY IN PRESENCE OF CALCIUM (A_{Ca2+}) (AT ACIDIC PH)

SEQ ID NO:7 and multiple variants thereof are measured according to the Glucose Isomerase Standard Assay at 50 mM fructose, at acidic pH (pH 5.8) and at 25° C. either
Example 4a: at 0 mM Ca²⁺ (Specific Assay Conditions 5); or
Example 4b: at 5 mM Ca²⁺ (Specific Assay Conditions 6); or
Example 4c: at 10 mM Ca²⁺ (Specific Assay Conditions 7).

Samples were collected after 21 h, stopped, and the glucose formed has been quantified by the Glucose Detection Assay, and the enzymatic activity has been calculated from the glucose formed and the reaction time. The relative enzymatic activity of each variant has been calculated by dividing the enzyme activity of each variant by the enzyme activity of SEQ ID NO:7.

TABLE 5

| | Relative activity | Relative activity | Relative activity |
|---|---|---|---|
| | Ca²⁺_{(0mM)} | Ca²⁺_{(5mM)} | Ca²⁺_{(10mM)} |
| | pH 5.8 | pH 5.8 | pH 5.8 |
| | (compared | (compared | (compared |
| SEQ | to SEQ ID | to SEQ ID | to SEQ ID |
| ID NO: | NO: 7) | NO: 7) | NO: 7) |
| 7 | 1.0 | 1.0 | 1.0 |
| 25 | 1.6 | 2.9 | 2.6 |
| 26 | 1.4 | 2.5 | 2.4 |
| 27 | 1.2 | 2.4 | 1.9 |
| 28 | 1.7 | 2.5 | 2.1 |
| 29 | 1.2 | 2.2 | 1.7 |
| 30 | 1.3 | 2.4 | 1.9 |
| 31 | 1.7 | 2.4 | 2.3 |
| 32 | 1.5 | 1.8 | 1.7 |
| 33 | 0.1 | 1.0 | — |
| 34 | 1.1 | 1.9 | 1.6 |
| 35 | 1.1 | 1.8 | 1.7 |
| 36 | 1.7 | 3.3 | 3.0 |
| 37 | 1.3 | 2.5 | 2.2 |
| 38 | 1.4 | 2.3 | 1.7 |
| 39 | 1.8 | 2.9 | 2.6 |
| 40 | 0.7 | 1.6 | 1.6 |
| 41 | 0.8 | 1.7 | 1.2 |
| 42 | 1.1 | 2.2 | 1.5 |
| 43 | 1.1 | 1.8 | 1.5 |
| 44 | 0.3 | 1.4 | 1.1 |
| 45 | 0.4 | 1.1 | — |
| 46 | 0.2 | 1.2 | — |
| 47 | 1.6 | 1.7 | 1.2 |
| 48 | 0.3 | 1.0 | 1.0 |
| 49 | 0.2 | 1.4 | 1.1 |
| 50 | 0.4 | 1.4 | 1.2 |
| 51 | 0.1 | 1.3 | — |
| 52 | 1.2 | 1.6 | 1.5 |
| 53 | 1.8 | 3.0 | 2.5 |
| 54 | 1.3 | 2.8 | 2.5 |
| 55 | 0.8 | 2.0 | 1.8 |
| 56 | 2.1 | 2.6 | 2.1 |
| 57 | 0.6 | 1.2 | 1.3 |
| 58 | 0.6 | 1.7 | 1.1 |
| 59 | 1.0 | 1.7 | 1.4 |
| 60 | 1.0 | 2.0 | 1.7 |
| 61 | 1.4 | 1.9 | 1.5 |
| 62 | 1.5 | 2.4 | 1.9 |
| 63 | 0.5 | 1.2 | 1.0 |
| 64 | 0.6 | 1.2 | 1.0 |
| 65 | 0.9 | 1.4 | 1.0 |
| 66 | 1.3 | 1.6 | 1.2 |
| 67 | 0.9 | 1.3 | — |
| 68 | 2.1 | 2.8 | 2.3 |
| 69 | 1.3 | 1.7 | 1.4 |
| 70 | 0.9 | 1.9 | 1.6 |
| 71 | 1.2 | 1.5 | 1.2 |
| 72 | 1.2 | 2.5 | 2.0 |

### EXAMPLE 5—MULTIPLE VARIANTS WITH INCREASED RESIDUAL ACTIVITY IN PRESENCE OF CALCIUM AT ACIDIC PH (RA_{Ca2+})

SEQ ID NO:7 and multiple variants thereof have been measured according to the Glucose Isomerase Standard Assay at 200 mM Fructose, acidic pH (pH 6.5) and at 40° C. either
Example 5a: at 0 mM Ca²⁺ (Specific Assay Conditions 8); or
Example 5b: at 5 mM Ca²⁺ (Specific Assay Conditions 9); or
Example 5c: at 10 mM Ca²⁺ (Specific Assay Conditions 10); or
Example 5d: at 20 mM Ca²⁺ (Specific Assay Conditions 11); or
Example 5e: at 30 mM Ca²⁺ (Specific Assay Conditions 12); or.
Example 5f: at 60 mM Ca²⁺ (Specific Assay Conditions 13).

Samples were collected after 4 h, stopped, and the glucose formed has been quantified by the Glucose Detection Assay, and the enzymatic activity has been calculated from the glucose formed and the reaction time.

For calculating the residual activity of each individual variant of SEQ ID NO: at a certain Ca²⁺-concentration, the detected enzymatic activity at such Ca²⁺ concentration is normalized by division through the enzymatic activity of such variant by the enzymatic activity of such variant at a CA²⁺-activity at 0 mM (residual activity compared to [Ca²⁺]=0 mM).

All multiple mutants show a higher residual activity upon increase of Ca²⁺ when compared to the residual activities of SEQ ID NO: 1 or the single mutant SEQ ID NO:7 at the same calcium concentration

TABLE 6

| SEQ ID NO: | 0 mM Ca²⁺ | 5 mM Ca²⁺ | 10 mM Ca²⁺ | 20 mM Ca²⁺ | 30 mM Ca²⁺ | 60 mM Ca²⁺ |
|---|---|---|---|---|---|---|
| 53 | 100.0% | 32.7% | 17.8% | 9.1% | 6.2% | n.d. |
| 56 | 100.0% | 38.1% | 22.0% | 13.1% | 9.5% | 5.3% |
| 42 | 100.0% | 35.5% | 18.8% | 10.0% | 6.6% | 3.9% |
| 43 | 100.0% | 44.9% | 26.0% | 14.3% | 9.8% | 4.3% |
| 45 | 100.0% | 42.0% | 26.0% | 15.4% | 11.2% | 6.0% |
| 7 | 100.0% | 22.3% | 12.2% | 7.9% | 5.5% | 4.2% |
| 1 | 100.0% | 17.0% | 10.3% | 5.3% | n.d. | n.d |

n.d. = not detectable

### EXAMPLE 6—MULTIPLE VARIANTS WITH INCREASED RELATIVE COLD ACTIVITY AT DECREASED TEMPERATURES (ATEMP)

SEQ ID NO:7 and multiple variants thereof are measured according to the Glucose Isomerase Standard Assay at 50 mM fructose, at acidic pH (pH 5.8) and at 5 mM Ca²⁺ either
Example 6a: at 25° C. (Specific Assay Conditions 5); or
Example 6b: at 20° C. (Specific Assay Conditions 14); or
Example 6c: at 15° C. (Specific Assay Conditions 15).

Samples were collected after 21 h, stopped, and the glucose formed has been quantified by the Glucose Detection Assay, and the enzymatic activity has been calculated from the glucose formed and the reaction time. The relative enzymatic activity of each variant has been calculated by dividing the enzyme activity of each variant by the enzyme activity of SEQ ID NO:7.

TABLE 7

| | Relative activity | Relative activity | Relative activity |
|---|---|---|---|
| | 25° C.+_{(5mM)} | 20° C.+_{(5mM)} | 15° C.+_{(5mM)} |
| | pH 6.5 | pH 6.5 | pH 6.5 |
| | (compared to | (compared to | (compared to |
| SEQ | SEQ ID | SEQ ID | SEQ ID |
| ID NO: | NO: 7) | NO: 7) | NO: 7) |
| 7 | 1.0 | 1.0 | 1.0 |
| 25 | 2.9 | 3.7 | 3.8 |
| 26 | 2.5 | 3.3 | 4.1 |
| 27 | 2.4 | 2.7 | 3.1 |
| 28 | 2.5 | 3.2 | 3.1 |
| 29 | 2.2 | 2.6 | 3.0 |
| 30 | 2.4 | 2.7 | 3.0 |
| 31 | 2.4 | 2.8 | 2.2 |
| 32 | 1.8 | 2.3 | 2.0 |
| 33 | 1.0 | 1.0 | 1.5 |
| 34 | 1.9 | 2.1 | 1.5 |
| 35 | 1.8 | 2.7 | 3.3 |
| 36 | 3.3 | 3.9 | 3.1 |
| 37 | 2.5 | 3.0 | 2.9 |
| 38 | 2.3 | 3.1 | 2.8 |
| 39 | 2.9 | 3.3 | 2.7 |
| 40 | 1.6 | 2.4 | 2.4 |
| 41 | 1.7 | 1.8 | 2.4 |
| 42 | 2.2 | 2.6 | 2.3 |
| 43 | 1.8 | 2.2 | 2.3 |
| 44 | 1.4 | 1.7 | 2.0 |
| 45 | 1.1 | 1.1 | 1.9 |
| 46 | 1.2 | 1.2 | 1.9 |
| 47 | 1.7 | 1.9 | 1.8 |
| 48 | 1.0 | 1.3 | 1.7 |
| 49 | 1.4 | 1.9 | 1.7 |
| 50 | 1.4 | 1.8 | 1.7 |
| 51 | 1.3 | 1.6 | 1.3 |
| 52 | 1.6 | 2.0 | 1.3 |
| 53 | 3.0 | 3.4 | 3.9 |
| 54 | 2.8 | 3.1 | 3.6 |
| 55 | 2.0 | 3.0 | 3.2 |
| 56 | 2.6 | 3.5 | 3.0 |
| 57 | 1.2 | 1.2 | 2.4 |
| 58 | 1.7 | 1.7 | 2.3 |
| 59 | 1.7 | 2.3 | 2.2 |
| 60 | 2.0 | 2.3 | 2.2 |
| 61 | 1.9 | 2.0 | 2.1 |
| 62 | 2.4 | 2.8 | 2.1 |
| 63 | 1.2 | 1.2 | 2.0 |
| 64 | 1.2 | 1.2 | 1.8 |
| 65 | 1.4 | 1.4 | 1.5 |
| 66 | 1.6 | 1.6 | 1.2 |
| 67 | 1.3 | 1.3 | 1.1 |
| 68 | 2.8 | 3.4 | 3.3 |
| 69 | 1.7 | 1.9 | 2.5 |
| 70 | 1.9 | 2.2 | 2.4 |
| 71 | 1.5 | 1.9 | 2.0 |
| 72 | 2.5 | 2.8 | 2.9 |
| 73 | 1.0 | 1.0 | 1.2 |

1. A glucose isomerase comprising an amino acid sequence having at least 80% identity to SEQ ID NO: 1 and comprising at least one substitution selected from the group consisting of V187C, V187T, F242C, P313S, and A47S,
wherein the glucose isomerase has at least one of characteristics (A), (B), (C), (D) and (E), or any combination thereof,
wherein
characteristic (A) is an increased enzymatic activity in the absence of Ca²⁺ (A₀) in comparison to SEQ ID NO:1 with respect to the conversion of fructose into glucose or vice versa;
characteristic (B) is an increased enzymatic activity in the presence of Ca²⁺ (A_{Ca2+}) in comparison to SEQ ID NO: 1 with respect to the conversion of fructose into glucose or vice versa;
characteristic (C) is an increased residual enzymatic activity in the presence of Ca²⁺ (RA_{Ca2+}) in comparison to SEQ ID NO:1 with respect to the conversion of fructose into glucose or vice versa;
characteristic (D) is an increased enzymatic activity at neutral and acidic pH (A_{pH}) in comparison to SEQ ID NO: 1 with respect to the conversion of fructose into glucose or vice versa; and
characteristic (E) is an increased enzymatic activity at low temperatures (A_{Temp}) in comparison to SEQ ID NO:1 with respect to the conversion of fructose into glucose or vice versa.

2. The glucose isomerase of claim 1, wherein the at least one substitution is selected from the group of consisting of V187C and F242C.

3. The glucose isomerase according to claim 1, wherein the at least one substitution is V187C.

4. The glucose isomerase according to claim 1, wherein the amino acid sequence further comprises at least one, at least two, at least three, at least four, at least five, at least six, at least seven, or at least eight substitutions at a position selected from the group consisting of V187, Q300, K343, A342, M241, F242, P313, A47, and M309.

5. The glucose isomerase according to claim 4, wherein the amino acid sequence comprises at least one, at least two, at least three, at least four, at least five, at least six, at least seven, at least eight, or at least nine substitutions selected from the group consisting of V187C, V187T, Q300Y, Q300S, P313S, P313V, K343R, A342S, A47S, M241V, F242C, and M309I.

6. The glucose isomerase according to claim 1, wherein the amino acid sequence has at least 80%, or at least 82%, or at least 84%, or at least 86%, or at least 88%, or at least 90%, or at least 92%, or at least 94%, or at least 96%, or at least 97%, or at least 98%, or at least 99%, or at least 99.9% identity to SEQ ID NO: 1.

7. The glucose isomerase according to claim 1, wherein the amino acid sequence has at least 80%, or at least 82%, or at least 84%, or at least 86%, or at least 88%, or at least 90%, or at least 92%, or at least 94%, or at least 96%, or at least 98%, or at least 100% identity to an amino acid sequence selected from the group consisting of SEQ ID NO: 7, SEQ ID NO: 3, SEQ ID NO: 11, and SEQ ID NO: 19.

8. The glucose isomerase according to claim 1, wherein the amino acid sequence has at least 80%, or at least 82%, or at least 84%, or at least 86%, or at least 88%, or at least 90%, or at least 92%, or at least 94%, or at least 96%, or at least 98%, or at least 100% identity to an amino acid sequence selected from the groups consisting of
SEQ ID NO: 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, and 73; or
SEQ ID NO: 28, 29, 32, 36, 37, 38, 39, 40, 41, 42, 44, 45, 46, 47, 53, 54, 56, 57, 58, 59, 60, 61, 62, 63, 64, 65, 66, 67, 68, 69, 70, 71, 72, and 73; or
SEQ ID NO: 26, 32, 34, 35, 43, 44, 51, 62, 64, 66, 70, and 72; or
SEQ ID NO: 27, 30, 33, 34, 37, 41, 42, 43, 44, 45, 47, 48, 49, 50, 51, 53, 54, 55, 56, 58, 59, 60, 61, 63, 64, 65, 66, 68, 69, 70, 71, 72, and 73.

9. The glucose isomerase according to claim 1, which is adapted to catalyze conversion of
an aldose molecule into a ketose molecule, and/or
a ketose molecule into an aldose molecule.

10. The glucose isomerase according to claim 1, which exhibits
(A) an increased enzymatic activity in the absence of Ca²⁺ (A₀) in comparison to SEQ ID NO:7 with respect to the conversion of fructose into glucose or vice versa; and/or
(B) an increased enzymatic activity in the presence of Ca²⁺ (A_{Ca2+}) in comparison to SEQ ID NO:7 with respect to the conversion of fructose into glucose or vice versa; and/or
(C) an increased residual enzymatic activity in the presence of Ca²⁺ (RA_{Ca2+}) in comparison to SEQ ID NO:7 with respect to the conversion of fructose into glucose or vice versa; and/or
(D) an increased enzymatic activity at neutral and acidic pH (A_{pH}) in comparison to SEQ ID NO:7 with respect to the conversion of fructose into glucose or vice versa; and/or
(E) an increased enzymatic activity at low temperatures (A_{Temp}) in comparison to SEQ ID NO:7 with respect to the conversion of fructose into glucose or vice versa.

11. A process for conversion of an aldose molecule into a ketose molecule said method comprising:
providing the glucose isomerase of claim 1, and
contacting said aldose molecule with the glucose isomerase, wherein the aldose molecule is converted into the ketose molecule.

12. The process according to claim 11, wherein the aldose molecule is glucose.

13. The process according to claim 11, wherein the contacting is performed in a liquid reaction medium that is characterized by
(i) a pH of in the range of 5.0 to 8.0; and/or
(ii) a calcium ion concentration of 0 mM or in the range between 0.5 mM to 60 mM.

14. A method comprising
providing:
the glucose isomerase of claim 1,
a liquid reaction medium having a pH of in the range of 5.0 to 8.0 and/or a calcium ion concentration of 0 mM or in the range between 0.5 mM to 60 mM, and
(i) an aldose molecule, wherein the glucose isomerase coverts the aldose molecule in the liquid reaction medium into a ketose molecule and/or
(ii) a ketose molecule, wherein the glucose isomerase coverts the ketose molecule in the liquid reaction medium into an aldose molecule.

15. The method according to claim 14, wherein the aldose molecule is glucose that is converted to the ketose molecule fructose.

16. The method according to claim 13, wherein the liquid reaction medium is characterized by:
a pH of in the range of 5.3 to 8.0, or 5.5 to 8.0, or 5.8 to 8.0, or 6.0 to 8.0, or 6.4 to 8.0, or 6.6 to 8.0, or 6.9 to 8.0, preferably within the range of 5.0 to 7.5, or 5.3 to 7.5, or 5.5 to 7.5, or 5.8 to 7.5, or 6.0 to 7.5, or 6.4 to 7.5, or 6.6 to 7.5, or 6.9 to 7.5; preferably in the range of 5.0 to 7.0, or 5.3 to 7.0, or 5.5 to 7.0, or 5.8 to 7.0, or 6.0 to 7.0, or 6.4 to 7.0, or 6.6 to 7.0, or 6.9 to 7.0; and/or
a calcium ion concentration in the range between 1 mM to 60 mM, or 2 mM to 60 mM, or 3 mM to 60 mM, or 4.0 mM to 60 mM, or 5 mM to 60 mM, or 10 mM to 60 mM, or 20 mM to 60 mM, or 30 mM to 60 mM, or 0.5 mM to 30 mM, or 1 mM to 30 mM, or 2 mM to 30 mM, or 3 mM to 30 mM, or 4.0 mM to 30 mM, or 5 mM to 30 mM, or 10 mM to 30 mM, or 20 mM to 30 mM, or 0.5 mM to 20 mM, or 1 mM to 20 mM, or 2 mM to 20 mM, or 3 mM to 20 mM, or 4.0 mM to 20 mM, or 5 mM to 20 mM, or 10 mM to 20 mM, or 0.5 mM to 10 mM, or 1 mM to 10 mM, or 2 mM to 10 mM, or 3 mM to 10 mM, or 4.0 mM to 10 mM, or 5 mM to 10 mM.

17. The glucose isomerase according to claim 1, wherein the characteristic (A), (B), (C), (D) and/or (E) is the increased enzymatic activity with respect to the conversion of fructose into glucose.

18. The glucose isomerase according to claim 9, wherein the ketose molecule is fructose and the aldose molecule is glucose.

19. The glucose isomerase according to claim 10, wherein the increased enzymatic activity in (A), (B), (C), (D) and/or (E) is with respect to the conversion of fructose into glucose.

20. The process according to claim 13, wherein the pH is of in the range of 5.3 to 8.0, or 5.5 to 8.0, or 5.8 to 8.0, or 6.0 to 8.0, or 6.4 to 8.0, or 6.6 to 8.0, or 6.9 to 8.0; and/or the calcium ion concentration is in the range of between 1 mM to 60 mM, or 2 mM to 60 mM, or 3 mM to 60 mM, or 4.0 mM to 60 mM, or 5 mM to 60 mM, or 10 mM to 60 mM, or 20 mM to 60 mM, or 30 mM to 60 mM.


SEQUENCE LISTING (73 sequences)

SEQ ID NO: 1 - protein, 449 aa, Bifidobacterium sp.
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYVFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 2 - protein, 449 aa, synthetic construct
MGLWDIEKIPYVGREKGSQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYVFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 3 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYVFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 4 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLHNTSSLFTNPRFVSGASTSPFADIYAYAGGQLKHSLEIAKRLGAENYVFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 5 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFRNPRFVSGASTSPFADIYAYAGGQLKHSLEIAKRLGAENYVFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 6 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADVYAYAGGQLKHSLEIAKRLGAENYVFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 7 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 8 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYVFWNGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 9 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYVFWGGREGYENIWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 10 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYVFWGGREGYENLWNTQMKREQEHMAKFFHMCHDYAKEIGLDAQFLIEPKAKEPTVFQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 11 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYVFWGGREGYENLWNTQMKREQEHMAKFFHMCHDYAKEIGLDAQFLIEPKAKEPTMCQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 12 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYVFWGGREGYENLWNTQMKREQEHMAKFFHMCHDYAKEIGLDAQFLIEPKAKEPTMVQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 13 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYVFWGGREGYENLWNTQMKREQEHMAKFFHMCHDYAKEIGLDAQFLIEPKAKEPTMFQYDFDAATAINFLRTYDLMDVFKLNLEGNHSNLAGHTYQHEIRTAREAGVLGSLDANQGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 14 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYVFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANYGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 15 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYVFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANSGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 16 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYVFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANCGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 17 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYVFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANFGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 18 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYVFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDIDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 19 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYVFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDMDEFSTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 20 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYVFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDMDEFVTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 21 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYVFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDMDEFLTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 22 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYVFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDMDEFPTDLYETSTVMWEVLAEGGIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 23 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYVFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDSKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 24 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYVFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDARPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 25 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANYGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 26 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDMDEFSTDLYETSTVMWEVLAEGQIGPHGGLNFDARPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 27 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDSKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 28 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTVCQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 29 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMCQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDARPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 30 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDMDEFVTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 31 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANSGDKLIGWDIDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 32 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYTFWGGREGYENLWNTQMKREQEHMAKFFHMCHDYAKEIGLDAQFLIEPKAKEPTMCQYDFDAATAINFLRTYDLMDVFKLNLEGNHANLAGHTYQHEIRTAREAGVLGSLDANQGDKLIGWDMDEFSTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 33 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDARPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 34 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDMDEFSTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 35 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANSGDKLIGWDIDEFSTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 36 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMCQYDFDAATAINFLRTYDLMDVFKLNLEGNHANLAGHTYQHEIRTAREAGVLGSLDANSGDKLIGWDIDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 37 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYWFWGGREGYENLWNTQMKREQEHMAKFFHMCHDYAKEIGLDAQFLIEPKAKEPTMCQYDFDAATAINFLRTYDLMDVFKLNLEGNHANLAGHTYQHEIRTAREAGVLGSLDANSGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 38 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTVCQYDFDAATAINFLRTYDLMDVFKLNLEGNHANLAGHTYQHEIRTAREAGVLGSLDANYGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 39 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYWFWGGREGYENLWNTQMKREQEHMAKFFHMCHDYAKEIGLDAQFLIEPKAKEPTVCQYDFDAATAINFLRTYDLMDVFKLNLEGNHANLAGHTYQHEIRTAREAGVLGSLDANQGDKLIGWDMDEFVTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 40 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMCQYDFDAATAINFLRTYDLMDVFKLNLEGNHANLAGHTYQHEIRTAREAGVLGSLDANYGDKLIGWDIDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 41 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMCQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDSKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 42 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTVCQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 43 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDIDEFSTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 44 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYWFWGGREGYENLWNTQMKREQEHMAKFFHMCHDYAKEIGLDAQFLIEPKAKEPTMCQYDFDAATAINFLRTYDLMDVFKLNLEGNHANLAGHTYQHEIRTAREAGVLGSLDANQGDKLIGWDMDEFSTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 45 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMCQYDFDAATAINFLRTYDLMDVFKLNLEGNHANLAGHTYQHEIRTAREAGVLGSLDANQGDKLIGWDIDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 46 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTVCQYDFDAATAINFLRTYDLMDVFKLNLEGNHANLAGHTYQHEIRTAREAGVLGSLDANQGDKLIGWDMDEFGTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 47 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMCQYDFDAATAINFLRTYDLMDVFKLNLEGNHANLAGHTYQHEIRTAREAGVLGSLDANYGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 48 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDMDEFVTDLYETSTVMWEVLAEGQIGPHGGLNFDARPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 49 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDSRPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 50 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDIDEFVTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 51 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANQGDKLIGWDMDEFSTDLYETSTVMWEVLAEGQIGPHGGLNFDSKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 52 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANSGDKLIGWDMDEFPTDLYETSTVMWEVLAEGQIGPHGGLNFDSKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 53 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMCQYDFDAATAINFLRTYDLMDVFKLNLEGNHANLAGHTYQHEIRTAREAGVLGSLDANQGDKLIGWDIDEFPTDLYETSTVMWEVLAEGQIGPHGGLNFDARPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 54 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMCQYDFDAATAINFLRTYDLMDVFKLNLEGNHANLAGHTYQHEIRTAREAGVLGSLDANQGDKLIGWDIDEFPTDLYETSTVMWEVLAEGQIGPHGGLNFDSKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 55 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMFQYDFDAATAINFLRTYDLMDVFKLNLEGNHANLAGHTYQHEIRTAREAGVLGSLDANSGDKLIGWDIDEFPTDLYETSTVMWEVLAEGQIGPHGGLNFDARPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 56 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTVCQYDFDAATAINFLRTYDLMDVFKLNLEGNHANLAGHTYQHEIRTAREAGVLGSLDANQGDKLIGWDIDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 57 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTVCQYDFDAATAINFLRTYDLMDVFKLNLEGNHANLAGHTYQHEIRTAREAGVLGSLDANSGDKLIGWDIDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 58 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTVCQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDARPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 59 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTVCQYDFDAATAINFLRTYDLMDVFKLNLEGNHANLAGHTYQHEIRTAREAGVLGSLDANYGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 60 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMCQYDFDAATAINFLRTYDLMDVFKLNLEGNHANLAGHTYQHEIRTAREAGVLGSLDANYGDKLIGWDMDEFPTDLYETSTVMWEVLAEGQIGPHGGLNFDANPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 61 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMCQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDSNPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 62 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMCQYDFDAATAINFLRTYDLMDVFKLNLEGNHANLAGHTYQHEIRTAREAGVLGSLDANQGDKLIGWDMDEFSTDLYETSTVMWEVLAEGQIGPHGGLNFDSKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 63 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTVCQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDSKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 64 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTVCQYDFDAATAINFLRTYDLMDVFKLNLEGNHANLAGHTYQHEIRTAREAGVLGSLDANQGDKLIGWDMDEFSTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 65 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTVCQYDFDAATAINFLRTYDLMDVFKLNLEGNHANLAGHTYQHEIRTAREAGVLGSLDANSGDKLIGWDMDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 66 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMCQYDFDAATAINFLRTYDLMDVFKLNLEGNHANLAGHTYQHEIRTAREAGVLGSLDANQGDKLIGWDIDEFSTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 67 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTVCQYDFDAATAINFLRTYDLMDVFKLNLEGNHANLAGHTYQHEIRTAREAGVLGSLDANQGDKLIGWDMDEFPTDLYETSTVMWEVLAEGQIGPHGGLNFDSKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 68 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTVCQYDFDAATAINFLRTYDLMDVFKLNLEGNHANLAGHTYQHEIRTAREAGVLGSLDANSGDKLIGWDIDEFPTDLYETSTVMWEVLAEGQIGPHGGLNFDAKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 69 - protein, 449 aa, synthetic construct
MGLWDIEKIPYVGREKGPQEGLAFHYYDADKVVAGKKMKDWLRFGVAWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTVCQYDFDAATAINFLRTYDLMDVFKLNLEGNHANLAGHTYQHEIRTAREAGVLGSLDANQGDKLIGWDIDEFPTDLYETSTVMWEVLAEGQIGPHGGLNFDSKPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 70 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMCQYDFDAATAINFLRTYDLMDVFKLNLEGNHANLAGHTYQHEIRTAREAGVLGSLDANQGDKLIGWDIDEFSTDLYETSTVMWEVLAEGQIGPHGGLNFDARPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 71 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTMCQYDFDAATAINFLRTYDLMDVFKLNLEGNHANLAGHTYQHEIRTAREAGVLGSLDANQGDKLIGWDIDEFPTDLYETSTVMWEVLAEGQIGPHGGLNFDSRPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 72 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTVCQYDFDAATAINFLRTYDLMDVFKLNLEGNHANLAGHTYQHEIRTAREAGVLGSLDANQGDKLIGWDIDEFSTDLYETSTVMWEVLAEGQIGPHGGLNFDARPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA

SEQ ID NO: 73 - protein, 449 aa, synthetic construct
MGLWDIEKIPYVGREKGPQEGLAFHYYDADKVVAGKKMKDWLRFGVSWWHTFDQELVDPFGTGTAQRPWYGKYSNAEDEALAKVDYAFEFFQKLGVEYFCFHDRDIAPEGDTLRETDKNLDKVVDKIEENMKSTGIKLLWNTSSLFTNPRFVSGASTSPFADIYAYAGGQLKHSLEIAKRLGAENYCFWGGREGYENLWNTQMKREQEHMAKFFHMCHDYAKEIGLDAQFLIEPKAKEPTVCQYDFDAATAINFLRTYDLMDVFKLNLEGNHANLAGHTYQHEIRTAREAGVLGSLDANQGDKLIGWDIDEFPTDLYETSTVMWEVLAEGQIGPHGGLNFDSRPRRTSFAAEDLFRSHIAGMDSFAAGLLVAAKMHEDKVIENLQAERYSSFDSGIGATVENGTASLASLEEYALDIPQSKLIEATKSDHLESVKATINNYMIDALAEA